(19) United States
(12) Patent Application Publication
(10) Pub. No.: US 2022/0009906 A1
(43) Pub. Date: Jan. 13, 2022

(54) ISOINDOLIN-1-ONE DERIVATIVES USEFUL AS GRK2 INHIBITORS

(71) Applicant: Janssen Pharmaceutica NV, Beerse (BE)

(72) Inventors: Guozhang Xu, Chesterbrook, PA (US); Eugene B. Grant, Flemington, NJ (US); Gee-Hong Kuo, Scotch Plains, NJ (US); Micheal D. Gaul, Apex, NC (US)

(21) Appl. No.: 17/356,575

(22) Filed: Jun. 24, 2021

Related U.S. Application Data
(60) Provisional application No. 63/043,239, filed on Jun. 24, 2020.

(51) Int. Cl. C07D 403/04; C07D 409/14; C07D 403/14; C07D 405/14; C07D 401/14; C07D 471/10; C07D 491/107; C07D 498/10

(52) U.S. Cl. CPC C07D 403/04; C07D 409/14; C07D 403/14; C07D 498/10; C07D 401/14; C07D 471/10; C07D 491/107; C07D 405/14

(57) ABSTRACT

The present invention is directed to isoindolin-1-one derivatives, pharmaceutical compositions containing them and their use in the treatment of disorders and conditions modulated by GRK2, including, but not limited to, cardiac failure, cardiac hypertrophy, hypertension, Type II diabetes Mellitus, NASH, NAFLD, end stage chronic kidney disease, kidney failure, etc.

### CROSS REFERENCE TO RELATED APPLICATIONS

This application claims the benefit of U.S. Provisional Application No. 63/043,239, filed on Jun. 24, 2020, which is incorporated by reference herein in its entirety.

### FIELD OF THE INVENTION

The present invention is directed to isoindolin-1-one derivatives, pharmaceutical compositions containing them and their use in the treatment of disorders and conditions modulated by GRK2, including, but not limited to, cardiac failure, cardiac hypertrophy, hypertension, Type II diabetes Mellitus, NASH, NAFLD, end stage chronic kidney disease, kidney failure, etc.

### BACKGROUND OF THE INVENTION

G-protein-coupled receptor kinase 2 (GRK2) is a G-protein-coupled receptor kinase that is ubiquitously expressed in many tissues and regulates various intracellular mechanisms. The up- or down-regulation of GRK2 correlates with several pathological disorders. GRK2 plays an important role in the maintenance of heart structure and function; thus, this kinase is involved in many cardiovascular diseases. GRK2 up-regulation can worsen cardiac ischemia; furthermore, increased kinase levels occur during the early stages of heart failure and in hypertensive subjects. GRK2 up-regulation can lead to changes in the insulin signaling cascade, which can translate to insulin resistance. Increased GRK2 levels also correlate with the degree of cognitive impairment that is typically observed in Alzheimer's disease. (GUCCIONE, M., et al., “G-Protein-Coupled Receptor Kinase 2 (GRK2) Inhibitors: Current Trends and Future Perspectives”, J. Med. Chem, 2016, pp 9277-9294, Vol 59 (20)).

GRK2 is a prototypic GRK. This cytosolic protein is ubiquitously expressed in many tissues, but it is particularly important for embryonic development and heart function. GRK2 plays a key role in several signal transduction pathways. This protein can trigger receptor desensitization and internalization through R-arrestin binding to activated GPCRs. GRK2 can also phosphorylate different effectors involved in signal transduction. Moreover, the expression and/or function of GRK2 is altered in several pathological conditions, including cardiovascular and inflammatory pathologies.

Heart failure (HF) is the most common disease for hospitalization in the elderly, with approximately 10% of men and 8% of women over the age of 60 affected. The prevalence of HF is growing with the rise of an aging population in developed countries. There remains an intense need for novel beneficial HF therapies, with more than 3 million people in the United States diagnosed per year, and HF related mortality and rehospitalization rates remaining high despite the modest improvement in survival rates seen from advances in device therapy and pharmacological therapy (angiotensin II receptor blockers, angiotensin converting enzyme inhibitors, and β-blockers). A plethora of research into HF has revealed it to be a complex disease associated with various pathogenetic mechanisms, including ventricular remodeling, excessive neurohormonal stimulation, abnormal Ca²⁺ handling, and proliferation of the extracellular matrix. Although an overstimulation of the sympathetic nervous system (SNS) initially compensates for cardiac dysfunction, the subsequent release of catecholamine ultimately promotes disease progression via long-term exposure. Activation of the SNS is mediated by adrenergic receptors (AR), and chronic β-AR activation induces β-AR desensitization and downregulation, subsequently leading to the reduction of β-AR signaling. G-protein receptor kinase (GRK) 2 phosphorylates agonist-occupied β-AR, promotes the binding of β-AR arrestin to the Gβγ subunit of the G-protein, facilitates the G-protein uncoupling from β-AR, and results in β-AR desensitization and downregulation. In the hearts of HF patients, GRK2 expression levels and activity were elevated, accompanied by lowered β-AR density and signaling. Moreover, GRK2 inhibition by overexpression of the βARKct, the peptide inhibitor of GRK2, or cardiac specific GRK2 gene ablation, improved cardiac function and survival with the increases in β-AR density and β-AR responses in several HF models. These results suggest that GRK2 has a strong relationship with HF, and inhibition of GRK2 is a promising mechanism for the treatment of HF (OKAWA, T., et al., J. Med. Chem., 2017, pp 6942-6990, Vol. 60).

G protein-coupled receptor kinase 2 (GRK2) is emerging as a pivotal signalling hub able to integrate different transduction cascades. This ability appears to underlie its central role in different physiological and pathological conditions. Key mediators of cardiovascular function (such as catecholamines or angiotensin II) and components of the systemic milieu altered in insulin resistance conditions converge in increasing GRK2 levels in diverse cardiovascular cell types. In turn, GRK2 would simultaneously modulate several cardiovascular regulatory pathways, including GPCR and insulin signalling cascades, NO bioavailability and mitochondrial function. This fact can help explain the contribution of increased GRK2 levels to maladaptive cardiovascular function and remodeling. It also unveils GRK2 as a link between cardiovascular pathologies and co-morbidities such as obesity or type 2 diabetes. On the other hand, enhanced GRK2 expression, as observed in adipose tissues, liver or skeletal muscle during insulin resistance-related pathologies, could modify the orchestration of GPCR and insulin signaling in these crucial metabolic organs, and contribute to key features of the obese and insulin-resistant phenotype (MAYOR, Jr., F., et al., Cellular Signaling, 2018, pp 25-32, Vol. 41)

There remains a need for GRK2 inhibitor compounds that have pharmacokinetic and pharmacodynamic properties suitable for use as human pharmaceuticals for the treatment of for example, cardiac failure, cardiac hypertrophy, hypertension, Type II diabetes Mellitus, NASH, NAFLD, end stage chronic kidney disease, kidney failure, etc.

### SUMMARY OF THE INVENTION

The present invention is directed to compounds of formula (I)

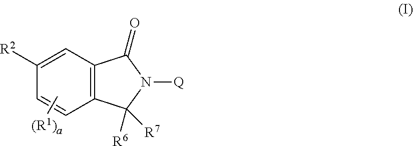

wherein

a is an integer from 0 to 3;

R¹ is selected from the group consisting of halogen, hydroxy, C₁₋₄alkyl, fluorinated C₁₋₂alkyl, C₁₋₄alkoxy, fluorinated C₁₋₂alkoxy, cyano, phenyl, pyridinyl, pyrimidinyl, pyrrolidinyl, piperidinyl, piperazinyl, and tetrahydropyranyl;

wherein the phenyl, pyridinyl, pyrimidinyl, pyrrolidinyl, piperidinyl, piperazinyl, and tetrahydropyranyl is optionally substituted with one or more substituents independently selected from the group consisting of halogen, hydroxy, C₁₋₄alkyl, fluorinated C₁₋₄alkyl, hydroxy substituted C₁₋₄alkyl, C₁₋₄alkoxy, fluorinated C₁₋₄alkoxy and NR^{J}R^{K}; wherein R^{J} and R^{K} are each independently selected from the group consisting of hydrogen, methyl and ethyl;

provided that when R¹ is selected from the group consisting of phenyl, pyridinyl, pyrimidinyl, pyrrolidinyl, piperidinyl, piperazinyl, and tetrahydropyranyl, wherein the phenyl, pyridinyl, pyrimidinyl, pyrrolidinyl, piperidinyl, piperazinyl, and tetrahydropyranyl are optionally substituted, then a is 1 and the R¹ group is bound at the 6-position of the isoindolin-2-one;

R² is selected from the group consisting of 5 to 10 membered heteroaryl and 5 to 10 membered heterocycloalkyl;

wherein the 5 to 10 membered heteroaryl or 5 to 10 membered heterocycloalkyl is optionally substituted with one or more substituents independently selected from the group consisting of oxo, —NR^{A}R^{B} and —(O)—NR^{A}R^{B}; wherein R^{A} and R^{B} are each independently selected from the group consisting of hydrogen and C₁₋₂alkyl;

Q is

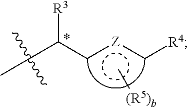

wherein R³ is selected from the group consisting of hydrogen, —C₁₋₄alkyl, —C₁₋₄alkoxy, —(C₁₋₂alkyl)-OH, —(C₁₋₂alkyl)-CO₂H, —(C₁₋₂alkyl)-O—(C₁₋₂alkyl), —(C₁₋₂alkyl)-O—(C₁₋₂alkyl)-OH, —(C₁₋₂alkyl)-O—(C₁₋₂alkyl)-CO₂H, —(C₁₋₂alkyl)-O-phenyl, —(C₁₋₂alkyl)-O—(C₁₋₂alkyl)-phenyl, —(C₁₋₂alkyl)-NR^{P}R^{Q}, —(C₁₋₂alkyl)-O—(C₁₋₂alkyl)-C(O)—NR^{P}R^{Q}, —CO₂H, —C(O)O—(C₁₋₂alkyl), —C(O)—NR^{P}R^{Q}, —C(O)-phenyl, C₃-6cycloalkyl, 1,2,3,5-tetrazol-4-yl and —(C₁₋₂alkyl)-1,2,3,5-tetrazol-4-yl;

wherein R^{P} and R^{Q} are each independently selected from the group consisting of hydrogen, methyl and ethyl;

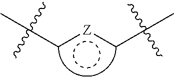

is selected from the group consisting of

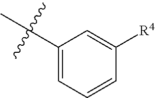

(wherein Z is CH),

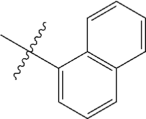 [SMILES: *c1cccc2ccccc12]

(wherein Z is CH and R⁴ is H),

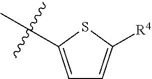

(wherein Z is S),

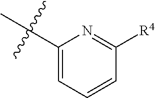

(wherein Z is N),

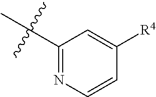

(wherein Z is CH),

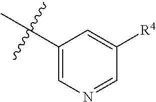

(wherein Z is CH) and

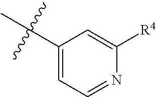

(wherein Z is CH);

R⁴ is selected from the group consisting of hydrogen, halogen, hydroxy, C₁₋₄alkyl, fluorinated C₁₋₂alkyl, —(C₁₋₂alkyl)-NR^{S}R^{T}, —(C₁₋₂alkyl)-CO₂H, —(C₁₋₂alkyl)-C(O)O—(C₁₋₄alkyl), C₁₋₄alkoxy, fluorinated C₁₋₂alkoxy, —O—(C₁₋₂alkyl)-CN, —O—(C₁₋₂alkyl)-CO₂H, —O—(C₁₋₂alkyl)-C(O)—O—(C₁₋₂alkyl), —O-phenyl, —O—(C₁₋₂alkyl)-phenyl, —O—(C₁₋₂alkyl)-(1,2,3,5-tetrazol-4-yl), —O-(oxetan-3-yl), —O-(tetrahydro-furan-3-yl), —O—C(O)—(C₁₋₂alkyl), —O—C(O)—C₃₋₆cycloalkyl, —O—C(O)—NR^{S}R^{T}, —O—C(O)—(C₁₋₂alkyl)-O—C(O)—(C₁₋₂alkyl), —CO₂H, —C(O)—O—(C₁₋₄alkyl), —C(O)—NR^{S}R^{T}, —C(O)—NH-(phenyl), —C(O)—NH-(benzyl), —C(O)—NH-(pyridinyl), —C(O)—NH—(C₁₋₂alkyl)-(pyridinyl), —C(O)—NH-((1R,2S,4S)-bicyclo[2.2.1]heptan-2-yl), —C(O)—NH-((1R,2R,4R)-bicyclo[2.2.1]heptan-2-yl), —NH—SO₂—(C₁₋₂alkyl), —(C₁₋₂alkyl)-SO₂—NR^{S}R^{T}, and pyrazol-1-yl;

wherein the phenyl, benzyl or pyridinyl, whether alone or as part of a substituent group is optionally substituted with one to two substituents independently selected from the group consisting of halogen, C₁₋₄alkyl and C₁₋₄alkoxy; and wherein R^{S} and R^{T} are each independently selected from the group consisting of hydrogen and C₁₋₄alkyl;

b is an integer from 0 to 4;

each R⁵ is independently selected from the group consisting of halogen, C₁₋₄alkyl and C₁₋₄alkoxy;

R⁶ and R⁷ are the same and are selected from the group consisting of hydrogen, C₁₋₂alkyl and hydroxy substituted C₁₋₂alkyl;

alternatively, R⁶ is hydrogen and R⁷ is selected from the group consisting of —(C₁₋₂alkyl)-CN, —(C₁₋₂alkyl)-OH, —(C₁₋₂alkyl)-CO₂H, —(C₁₋₂alkyl)-(1,2,3,5-tetrazol-4-yl) and —(C₁₋₂alkyl)-(isoindolin-2-yl-1,3-dione);

alternatively, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3,3-diyl, tetrahydro-pyran-4′,4′-diyl, 2,3-dihydro-inden-1′1′-diyl, hexahydro-2H-cyclopenta[d]oxazol-4′,4′-diyl-2-one and hexahydro-2H-cyclopenta[d]oxazol-6′,6′-diyl-2-one;

wherein the cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3′,3′-diyl, tetrahydro-pyran-4′,4′-diyl or 2,3-dihydro-inden-1′1′-diyl is optionally substituted with one to two substituents independently selected from the group consisting of hydroxy, C₁₋₄alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen, methyl and ethyl;

and stereoisomers, tautomers, isotopologues, and pharmaceutically acceptable salts thereof.

More particularly, the present invention is directed to compounds of formula (I-P)

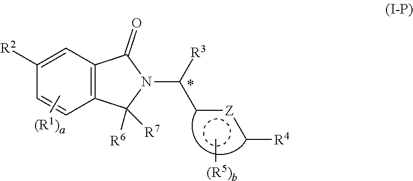

and stereoisomers, tautomers, isotopologues, and pharmaceutically acceptable salts thereof.

The present invention is further directed to processes for the preparation of the compounds of formula (I). The present invention is further directed to a compound of formula (I) prepared according to any of the process(es) described herein.

Illustrative of the invention are pharmaceutical compositions comprising a pharmaceutically acceptable carrier and a compound of formula (I) as described herein. An illustration of the invention is a pharmaceutical composition made by mixing a compound of formula (I) as described herein and a pharmaceutically acceptable carrier. Illustrating the invention is a process for making a pharmaceutical composition comprising mixing a compound of formula (I) as described herein and a pharmaceutically acceptable carrier.

Exemplifying the invention are methods of treating a disease, disorder, or condition mediated by GRK2 activity as described herein, comprising administering to a subject in need thereof a therapeutically effective amount of any of the compounds or pharmaceutical compositions described above.

Exemplifying the invention are methods of treating a disease, disorder, or condition mediated by GRK2 activity such as obesity, excess weight, impaired glucose tolerance (IGT), impaired fasting glucose (IFT), gestational diabetes, Type diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD) and renal disorders (including, but not limited to end stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure, a measured GFR equal or greater than 125 mL/min/1.73 m² (for example, a measured GFR equal or greater than 140 mL/min/1.73 m²)), comprising administering to a subject in need thereof a therapeutically effective amount of any of the compounds or pharmaceutical compositions described above.

In an embodiment, the present invention is directed to a compound of formula (I) for use as a medicament. In another embodiment, the present invention is directed to a compound of formula (I) for use in the treatment of a disorder mediated GRK2 activity such as obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), end stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m². In another embodiment, the present invention is directed to a composition comprising a compound of formula (I) for the treatment of a disorder mediated by GRK2 activity such as obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), end stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m2.

Another example of the invention is the use of any of the compounds described herein in the preparation of a medicament for treating: (a) obesity, (b) excess weight, (c) impaired glucose tolerance (IGT), (d) impaired fasting glucose (IFT), (e) gestational diabetes, (f) Type II diabetes mellitus, (g) Syndrome X (also known as Metabolic Syndrome), (h) nephropathy, (i) neuropathy, (j) retinopathy, in a subject in need thereof.

Another example of the invention is the use of any of the compounds described herein in the preparation of a medicament for treating: (a) cardiac failure, (b) cardiac hypertrophy, (c) cardiac fibrosis, (d) hypertension, (e) angina, (f) atherosclerosis, (g) heart disease, (h) heart attack, (i) ischemia, (j) stroke, (k) nerve damage or poor blood flow in the feet and (I) sepsis-associated encephalopathy (SAE), in a subject in need thereof.

Another example of the invention is the use of any of the compounds described herein in the preparation of a medicament for treating: (a) non-alcoholic steatohepatitis (NASH) and (b) non-alcoholic fatty liver disease (NAFLD), in a subject in need thereof.

Another example of the invention is the use of any of the compounds described herein in the preparation of a medicament for treating a disorder as described herein. Another example of the invention is the use of any of the compounds described herein in the preparation of a medicament for treating: (a) end stage chronic kidney disease, (b) chronic kidney disease, (c) acute renal failure, (d) nephrotic syndrome, (e) renal hyperfiltrative injury, (f) hyperfiltrative diabetic nephropathy, (g) renal hyperfiltration, (h) glomerular hyperfiltration, (i) renal allograft hyperfiltration, (j) compensatory hyperfiltration, (k) hyperfiltrative chronic kidney disease, (I) hyperfiltrative acute renal failure and (m) a measured GFR equal or greater than 125 mL/min/1.73 m², in a subject in need thereof.

In another example, the present invention is directed to a compound as described herein, for use in a method for treating a disorder as described herein. In another example, the present invention is directed to a compound as described herein, for use in a methods for treating a disorder selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), end stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m², in a subject in need thereof.

### DETAILED DESCRIPTION OF THE INVENTION

The present invention is directed to compounds of formula (I)

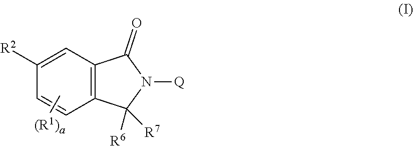

wherein a, R¹, R², Q, R⁶ and R⁷ are as herein defined, and stereoisomers, tautomers, isotopologues, and pharmaceutically acceptable salts thereof.

The compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD) and renal disorders (including, but not limited to end stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure, a measured GFR equal or greater than 125 mL/min/1.73 m² (for example, a measured GFR equal or greater than 140 mL/min/1.73 m²)).

In an embodiment, the compounds of the present invention are useful in the treatment of diseases, disorders and complications associated with GRK2 activity selected from the group consisting of (a) obesity, (b) excess weight, (c) impaired glucose tolerance (IGT), (d) impaired fasting glucose (IFT), (e) gestational diabetes, (f) Type II diabetes mellitus, (g) Syndrome X (also known as Metabolic Syndrome), (h) nephropathy, (i) neuropathy, (j) retinopathy, (k) cardiac failure, (I) cardiac hypertrophy, (m) cardiac fibrosis, (n) hypertension, (o) angina, (p) atherosclerosis, (q) heart disease, (r) heart attack, (s) ischemia, (t) stroke, (u) nerve damage or poor blood flow in the feet, (v) sepsis-associated encephalopathy (SAE), (w) non-alcoholic steatohepatitis (NASH), (x) non-alcoholic fatty liver disease (NAFLD) (y) end stage chronic kidney disease, (z) chronic kidney disease, (aa) acute renal failure, (ab) nephrotic syndrome, (ac) renal hyperfiltrative injury, (ad) hyperfiltrative diabetic nephropathy, (ae) renal hyperfiltration, (af) glomerular hyperfiltration, (ag) renal allograft hyperfiltration, (ah) compensatory hyperfiltration, (ai) hyperfiltrative chronic kidney disease, (aj) hyperfiltrative acute renal failure and (ak) a measured GFR equal or greater than 125 mL/min/1.73 m2.

In an embodiment, the compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, cardiac failure, cardiac hypertrophy, hypertension, angina, atherosclerosis, non-alcoholic steatohepatitis (NASH), non-alcoholic fatty liver disease (NAFLD), end stage chronic kidney disease, chronic kidney disease, acute renal failure, and a measured GFR equal or greater than 125 mL/min/1.73 m2.

In another embodiment, the compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, non-alcoholic steatohepatitis (NASH), non-alcoholic fatty liver disease (NAFLD), end stage chronic kidney disease, chronic kidney disease, acute renal failure, and a measured GFR equal or greater than 125 mL/min/1.73 m2.

In an embodiment, the compounds of the present invention are useful in the treatment of diseases, disorders and complications associated with GRK2 activity selected from the group consisting of obesity, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy and diabetic retinopathy.

In another embodiment, the compounds of the present invention are useful in the treatment of diseases, disorders and complications associated with GRK2 activity selected from the group consisting of cardiac failure, cardiac hypertrophy, hypertension and atherosclerosis.

In another embodiment, the compounds of the present invention are useful in the treatment of diseases, disorders and complications associated with GRK2 activity selected from the group consisting of non-alcoholic steatohepatitis (NASH) and non-alcoholic fatty liver disease (NAFLD).

In another embodiment, the compounds of the present invention are useful in the treatment of renal diseases, disorders and complications associated with GRK2 activity selected from the group consisting of end stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m² (for example, a measured GFR equal or greater than 140 mL/min/1.73 m²)).

In certain embodiments of the present invention, a is an integer from 0 to 2. In certain embodiments of the present invention, a is an integer from 0 to 1. In certain embodiments of the present invention, a is 1. In certain embodiments of the present invention a is 0.

In certain embodiments of the present invention, R¹ is selected from the group consisting of halogen, C₁₋₄alkyl, fluorinated C₁₋₂alkyl, C₁₋₄alkoxy, fluorinated C₁₋₂alkoxy, phenyl, pyridinyl, pyrimidinyl, pyrrolidinyl, piperidinyl, piperazinyl, and tetrahydro-pyranyl; wherein the phenyl, pyridinyl, pyrimidinyl, pyrrolidinyl, piperidinyl, piperazinyl, and tetrahydro-pyranyl is optionally substituted with one or more substituents independently selected from the group halogen, hydroxy, C₁₋₄alkyl, fluorinated C₁₋₄alkyl, hydroxy substituted C₁₋₄alkyl, C₁₋₄alkoxy, fluorinated C₁₋₄alkoxy and NR^{J}R^{K}; wherein R^{J} and R^{K} are each independently selected from the group consisting of hydrogen, methyl and ethyl; provided that when R¹ is selected from the group consisting of phenyl, pyridinyl, pyrimidinyl, pyrrolidinyl, piperidinyl, piperazinyl, and tetrahydropyranyl, wherein the phenyl, pyridinyl, pyrimidinyl, pyrrolidinyl, piperidinyl, piperazinyl, or tetrahydropyranyl are optionally substituted, then a is 1 and the R¹ group is bound at the 6-position of the isoindolin-2-one.

In certain embodiments of the present invention, R¹ is selected from the group consisting of halogen, phenyl, pyridinyl, pyrimidinyl, pyrrolidinyl, piperidinyl, piperazinyl, and tetrahydro-pyranyl; wherein the phenyl, pyridinyl, pyrimidinyl, pyrrolidinyl, piperidinyl, piperazinyl, and tetrahydro-pyranyl is optionally substituted with one to four substituents independently selected from the group halogen, hydroxy, C₁₋₄alkyl, fluorinated C₁₋₂alkyl, hydroxy substituted C₁₋₄alkyl, C₁₋₂alkoxy, and amino; provided that when R¹ is selected from the group consisting of phenyl, pyridinyl, pyrimidinyl, pyrrolidinyl, piperidinyl, piperazinyl, and tetrahydro-pyranyl, wherein the phenyl, pyridinyl, pyrimidinyl, pyrrolidinyl, piperidinyl, piperazinyl, and tetrahydro-pyranyl is optionally substituted, then a is 1 and the R¹ group is bound at the 6-position of the isoindolin-2-one.

In certain embodiments of the present invention, R¹ is selected from the group consisting of 5-fluoro, 7-fluoro, 6-(3-hydroxy-phenyl), 6-(4-hydroxy-phenyl), 6-(3-methoxy-phenyl), 6-(pyridin-2-yl), 6-(pyridin-3-yl), 6-(5-fluoro-pyridin-3-yl), 6-(5-t-butyl-pyridin-3-yl), 6-(5-methoxy-pyridin-3-yl), 6-(6-methoxy-pyridin-3-yl), 6-(6-(trifluoromethyl)-pyridin-3-yl), 6-(2-amino-pyridin-3-yl), 6-(6-(1-hydroxy-isopropyl)-pyridin-3-yl), 6-(pyridin-4-yl), 6-(2-t-butyl-pyridin-4-yl), 6-(4,6-dimethyl-pyridin-4-yl), 6-(pyrimidin-4-yl), 6-(pyrimidin-5-yl), 6-(pyrrolidin-3-yl), 6-(piperidin-3-yl), 6-(piperidin-4-yl), 6-(2,2,6,6-tetramethyl-piperidin-4-yl), 6-(piperazin-1-yl), 6-(4-methyl-piperazin-1-yl) and 6-(tetrahydro-pyran-4-yl).

In certain embodiments of the present invention, R¹ is selected from the group consisting of 5-fluoro, 7-fluoro, 6-(3-hydroxy-phenyl), 6-(4-hydroxy-phenyl), 6-(pyridin-3-yl), 6-(5-fluoro-pyridin-3-yl), 6-(5-methoxy-pyridin-3-yl), 6-(6-methoxy-pyridin-3-yl), 6-(2-amino-pyridin-3-yl), 6-(6-(1-hydroxy-isopropyl)-pyridin-3-yl), 6-(pyridin-4-yl), 6-(4,6-dimethyl-pyridin-4-yl), 6-(pyrimidin-5-yl), 6-(pyrrolidin-3-yl), 6-(piperazin-1-yl) and 6-(4-methyl-piperazin-1-yl). In certain embodiments of the present invention, R¹ is selected from the group consisting of 5-fluoro, 7-fluoro, 6-(3-hydroxy-phenyl), 6-(4-hydroxy-phenyl), 6-(pyridin-3-yl), 6-(5-fluoro-pyridin-3-yl), 6-(5-methoxy-pyridin-3-yl), 6-(6-methoxy-pyridin-3-yl), 6-(2-amino-pyridin-3-yl), 6-(6-(1-hydroxy-isopropyl)-pyridin-3-yl), 6-(pyridin-4-yl), 6-(4,6-dimethyl-pyridin-4-yl), 6-(pyrimidin-5-yl), 6-(pyrrolidin-3-yl), 6-(piperazin-1-yl) and 6-(4-methyl-piperazin-1-yl). In certain embodiments of the present invention, R¹ is selected from the group consisting of 5-fluoro, 7-fluoro, 6-(3-hydroxy-phenyl), 6-(pyridin-3-yl), 6-(5-fluoro-pyridin-3-yl), 6-(pyridin-4-yl), 6-(4,6-dimethyl-pyridin-4-yl), 6-(pyrimidin-5-yl), 6-(pyrrolidin-3-yl) and 6-(piperazin-1-yl). In certain embodiments of the present invention, R¹ is selected from the group consisting of 5-fluoro, 7-fluoro, 6-(3-hydroxy-phenyl), 6-(pyridin-3-yl), 6-(4,6-dimethyl-pyridin-4-yl) and 6-(pyrimidin-5-yl).

In certain embodiments of the present invention, R² is selected from the group consisting of 5 to 10 membered heteroaryl and 5 to 10 membered heterocycloalkyl; wherein the 5 to 10 membered heteroaryl or 5 to 10 membered heterocycloalkyl is optionally substituted with one to two substituents independently selected from the group consisting of oxo, —NR^{A}R^{B} and —C(O)—NR^{A}R^{B}; wherein R^{A} and R^{B} are each independently selected from the group consisting of hydrogen and C₁₋₂alkyl. In certain embodiments of the present invention, R² is selected from the group consisting of 5 to 6 membered heteroaryl, 9 to 10 membered heteroaryl, 5 to 6 membered heterocycloalkyl and 9 to 10 membered heterocycloalkyl (preferably, 5 to 6 membered heteroaryl and 9 to 10 membered heteroaryl); wherein the heteroaryl or heterocycloalkyl is optionally substituted with a substituent selected from the group consisting of oxo, —NR^{A}R^{B} and —C(O)—NR^{A}R^{B}; wherein R^{A} and R^{B} are each independently selected from the group consisting of hydrogen and C₁₋₂alkyl.

In certain embodiments of the present invention, R² is 5 to 10 membered heteroaryl; wherein the 5 to 10 membered heteroaryl is optionally substituted with one to two substituents independently selected from the group consisting of oxo, —NR^{A}R^{B} and —C(O)—NR^{A}R^{B}; wherein R^{A} and R^{B} are each independently selected from the group consisting of hydrogen and C₁₋₂alkyl. In certain embodiments of the present invention, R² is selected from the group consisting of 5 to 6 membered heteroaryl and 9 to 10 membered heteroaryl; wherein the 5 to 6 membered heteroaryl or 9 to 10 membered heteroaryl is optionally substituted with a substituent selected from the group consisting of oxo, —NR^{A}R^{B} and —C(O)—NR^{A}R^{B}; wherein R^{A} and R^{B} are each independently selected from the group consisting of hydrogen and C₁₋₂alkyl.

In certain embodiments of the present invention, R² is selected from the group consisting of pyrazol-4-yl, pyridin-3-yl, 6-(methyl-amino-carbonyl)-pyridin-3-yl, pyridin-4-yl, 6-(methyl-amino-carbonyl)-pyridin-4-yl, pyrimidin-2-yl, 2-amino-pyrimidin-4-yl, 2-(methyl-amino-carbonyl)-pyrimidin-4-yl, 1,2,3-triazol-4-yl, 2,4-dihydro-3H-1,2,4-triazol-4-yl-3-one, 1H-pyrrolo[2,3-b]pyridin-3-yl and 1H-pyrrolo[2,3-b]pyridin-4-yl.

In certain embodiments of the present invention, R² is selected from the group consisting of pyrazol-4-yl, pyridin-3-yl, pyridin-4-yl, 2-amino-pyrimidin-4-yl and 1,2,3-triazol-4-yl. In certain embodiments of the present invention, R² is pyrazol-4-yl.

In certain embodiments, R² is selected from the group consisting of optionally substituted pyrazol-4-yl, pyridin-3-yl, pyridin-3-yl, pyridin-4-yl, pyrimidin-2-yl, pyrimidin-4-yl, 1,2,3-triazol-4-yl, 2,4-dihydro-3H-1,2,4-triazol-4-yl-3-one, 1H-pyrrolo[2,3-b]pyridin-3-yl and 1H-pyrrolo[2,3-b]pyridin-4-yl. In certain embodiments, R² is selected from the group consisting of optionally substituted pyrazol-4-yl, pyridin-3-yl, pyridin-3-yl, pyridin-4-yl, pyrimidin-2-yl, 1,2,3-triazol-4-yl, 2,4-dihydro-3H-1,2,4-triazol-4-yl-3-one, 1H-pyrrolo[2,3-b]pyridin-3-yl and 1H-pyrrolo[2,3-b]pyridin-4-yl. In certain embodiments, R² is selected from the group consisting of optionally substituted pyrazol-4-yl, pyridin-3-yl, pyridin-3-yl, pyridin-4-yl, 1,2,3-triazol-4-yl, 2,4-dihydro-3H-1,2,4-triazol-4-yl-3-one, 1H-pyrrolo[2,3-b]pyridin-3-yl and 1H-pyrrolo[2,3-b]pyridin-4-yl.

In certain embodiments of the present invention, R² is selected from the group consisting of pyrazol-4-yl, pyridin-3-yl, 6-(methyl-amino-carbonyl)-pyridin-3-yl, pyridin-4-yl, 6-(methyl-amino-carbonyl)-pyridin-4-yl, pyrimidin-2-yl, 1,2,3-triazol-4-yl, 2,4-dihydro-3H-1,2,4-triazol-4-yl-3-one, 1H-pyrrolo[2,3-b]pyridin-3-yl and 1H-pyrrolo[2,3-b]pyridin-4-yl. In certain embodiments of the present invention, R² is selected from the group consisting of pyrazol-4-yl, pyridin-3-yl, 6-(methyl-amino-carbonyl)-pyridin-3-yl, pyridin-4-yl, 6-(methyl-amino-carbonyl)-pyridin-4-yl, 1,2,3-triazol-4-yl, 2,4-dihydro-3H-1,2,4-triazol-4-yl-3-one, 1H-pyrrolo[2,3-b]pyridin-3-yl and 1H-pyrrolo[2,3-b]pyridin-4-yl. In certain embodiments of the present invention, R² is selected from the group consisting of pyrazol-4-yl, pyridin-3-yl, pyridin-4-yl and 1,2,3-triazol-4-yl. In certain embodiments of the present invention, R² is pyrazol-4-yl.

In certain embodiments of the present invention, R^{A} and R^{B} are each independently selected from the group consisting of hydrogen and C₁₋₂alkyl. In certain embodiments of the present invention, R^{A} and R^{B} are each independently selected from the group consisting of hydrogen and methyl. In certain embodiments of the present invention, R^{A} and R^{B} are each independently selected from the group consisting of hydrogen and ethyl. In certain embodiments of the present invention, R^{A} and R^{B} are each independently selected from the group consisting of methyl and ethyl.

In certain embodiments of the present invention, R³ is selected from the group consisting of hydrogen, —C₁₋₄alkyl, —C₁₋₄alkoxy, —(C₁₋₂alkyl)-OH, —(C₁₋₂alkyl)-CO₂H, —(C₁₋₂alkyl)-O—(C₁₋₂alkyl), —(C₁₋₂alkyl)-O—(C₁₋₂alkyl)-OH, —(C₁₋₂alkyl)-O—(C₁₋₂alkyl)-CO₂H, —(C₁₋₂alkyl)-O-phenyl, —(C₁₋₂alkyl)-O—(C₁₋₂alkyl)-phenyl, —(C₁₋₂alkyl)-NR^{P}R^{Q}, —(C₁₋₂alkyl)-O—(C₁₋₂alkyl)-C(O)—NR^{P}R^{Q}, —CO₂H, —C(O)O—(C₁₋₂alkyl), —C(O)—NR^{P}R^{Q}, —C(O)-phenyl, C₃₋₆cycloalkyl, 1,2,3,5-tetrazol-4-yl and —(C₁₋₂alkyl)-1,2,3,5-tetrazol-4-yl; wherein R^{P} and R^{Q} are each independently selected from the group consisting of hydrogen, methyl and ethyl. In certain embodiments of the present invention, R³ is selected from the group consisting of hydrogen, —C₁₋₂alkyl, —C₁₋₄alkoxy, —(C₁₋₂alkyl)-OH, —(C₁₋₂alkyl)-CO₂H, —(C₁₋₂alkyl)-O—(C₁₋₂alkyl), —(C₁₋₂alkyl)-O—(C₁₋₂alkyl)-OH, —(C₁₋₂alkyl)-O—(C₁₋₂alkyl)-CO₂H, —(C₁₋₂alkyl)-O—(C₁₋₂alkyl)-phenyl, —(C₁₋₂alkyl)-NR^{P}R^{Q}, —(C₁₋₂alkyl)-O—(C₁₋₂alkyl)-C(O)—NR^{P}R^{Q}, —CO₂H, —C(O)O—(C₁₋₂alkyl), —C(O)—NR^{P}R^{Q}, —C(O)-phenyl, C₃₋₆cycloalkyl, 1,2,3,5-tetrazol-4-yl and —(C₁₋₂alkyl)-1,2,3,5-tetrazol-4-yl; wherein R^{P} and R^{Q} are each independently selected from the group consisting of hydrogen and methyl.

In certain embodiments of the present invention, R³ is selected from the group consisting of hydrogen, methyl, R-methyl, S-methyl, hydroxy-methyl-, R-(hydroxy-methyl-), S-(hydroxy-methyl-), S*-(hydroxy-methyl-), methoxy-methyl-, 2-hydroxy-ethoxy-methyl-, carboxy-methyl-, carboxy-methoxy-methyl-, amino-carbonyl-methoxy-methyl-, dimethylamino-methyl-, 2-carboxy-ethyl-, carboxy, R-carboxy, S-carboxy, methoxy-carbonyl-, S-(methoxy-carbonyl-), R-(methoxy-carbonyl-), R-cyclopropyl, benzyloxy-methyl-, 1,2,3,4-tetrazol-5-yl, 1,2,3,4-tetrazol-5-yl-methyl-, dimethylamino-carbonyl- and phenyl-carbonyl-.

In certain embodiments of the present invention, R³ is selected from the group consisting of hydrogen, R-methyl, hydroxy-methyl-, S-(hydroxy-methyl-), S*-(hydroxy-methyl-), methoxy-methyl-, 2-hydroxy-ethoxy-methyl-, carboxy-methoxy-methyl-, amino-carbonyl-methoxy-methyl-, dimethylamino-methyl-, R-cyclopropyl, benzyloxy-methyl- and dimethylamino-carbonyl-. In certain embodiments of the present invention, R³ is selected from the group consisting of hydrogen, R-methyl, hydroxy-methyl-, S-(hydroxy-methyl-), S*-(hydroxy-methyl-), methoxy-methyl-, 2-hydroxy-ethoxy-methyl-, carboxy-methoxy-methyl-, amino-carbonyl-methoxy-methyl-, dimethylamino-methyl-, R-cyclopropyl and benzyloxy-methyl-. In certain embodiments of the present invention, R³ is selected from the group consisting of hydrogen, R-methyl, hydroxy-methyl-, S-(hydroxy-methyl-), S*-(hydroxy-methyl-), methoxy-methyl-, 2-hydroxy-ethoxy-methyl- and amino-carbonyl-methoxy-methyl-. In certain embodiments of the present invention, R³ is selected from the group consisting of hydrogen, R-methyl, hydroxy-methyl- and S*-(hydroxy-methyl-).

In certain embodiments of the present invention, R^{P} and R^{Q} are each independently selected from the group consisting of hydrogen, methyl and ethyl. In certain embodiments of the present invention, R^{P} and R^{Q} are each independently selected from the group consisting of hydrogen and methyl. In certain embodiments of the present invention, R^{P} and R^{Q} are each independently selected from the group consisting of hydrogen and ethyl. In certain embodiments of the present invention, R^{P} and R^{Q} are each independently selected from the group consisting of methyl and ethyl

In certain embodiments of the present invention,

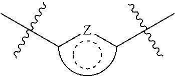

is

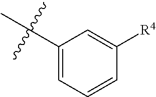

(wherein Z is CH). In certain embodiments of the present invention,

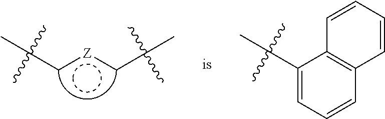

(wherein Z is CH and R⁴ is H). In certain embodiments of the present invention, is

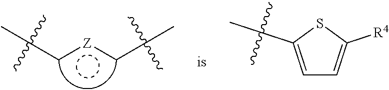

(wherein Z is S). In certain embodiments of the present invention

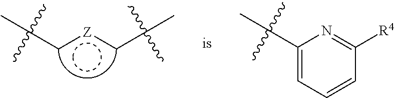

(wherein Z is N). In certain embodiments of the present invention,

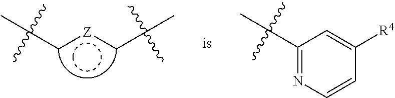

(wherein Z is CH). In certain embodiments of the present invention,

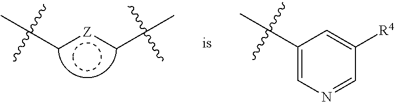

(wherein Z is CH). In certain embodiments of the present invention

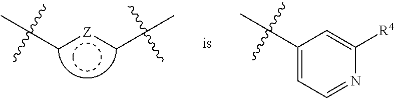

(wherein Z is CH).

In certain embodiments of the present invention,

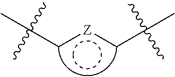

is selected from the group consisting of

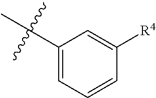

(wherein Z is CH),

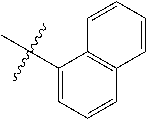 [SMILES: *c1cccc2ccccc12]

(wherein Z is CH and R⁴ is H),

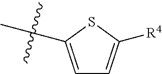

(wherein Z is S),

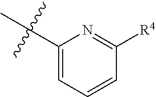

(wherein Z is N),

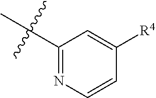

(wherein Z is CH),

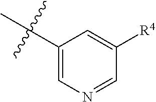

(wherein Z is CH) and

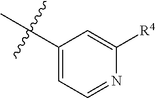

(wherein Z is CH).

In certain embodiments of the present invention,

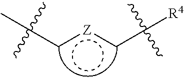

is selected from the group consisting of

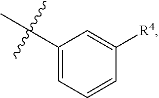

wherein Z is CH;

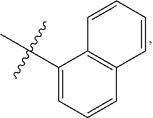

wherein Z is CH, R⁴ is H;

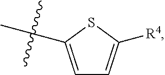

wherein Z is S;

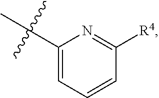

wherein Z is N; and

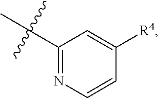

wherein Z is CH.

In certain embodiments of the present invention,

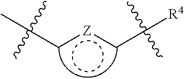

is selected from the group consisting of

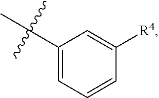

wherein Z is CH;

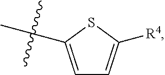

wherein Z is S; and

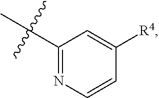

wherein Z is CH. In certain embodiments of the present invention,

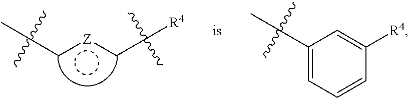

wherein Z is CH.

In certain embodiments of the present invention, R⁴ is selected from the group consisting of hydrogen, halogen, hydroxy, C₁₋₄alkyl, fluorinated C₁₋₂alkyl, —(C₁₋₂alkyl)-NR^{S}R^{T}, —(C₁₋₂alkyl)-CO₂H, —(C₁₋₂alkyl)-C(O)O—(C₁₋₄alkyl), C₁₋₄alkoxy, fluorinated C₁₋₂alkoxy, —O—(C₁₋₂alkyl)-CN, —O—(C₁₋₂alkyl)-CO₂H, —O—(C₁₋₂alkyl)-C(O)—O—(C₁₋₂alkyl), —O-phenyl, —O—(C₁₋₂alkyl)-phenyl, —O—(C₁₋₂alkyl)-(1,2,3,5-tetrazol-4-yl), —O-(oxetan-3-yl), —O-(tetrahydro-furan-3-yl), —O—C(O)—(C₁₋₂alkyl), —O—C(O)—C₃₋₆cycloalkyl, —O—C(O)—NR^{S}R^{T}, O—C(O)—(C₁₋₂alkyl)-O—C(O)—(C₁₋₂alkyl), —CO₂H, —C(O)—O—(C₁₋₄alkyl), —C(O)—NR^{S}R^{T}, —C(O)—NH-(phenyl), —C(O)—NH-(benzyl), —C(O)—NH-(pyridinyl), —C(O)—NH—(C₁₋₂alkyl)-(pyridinyl), —C(O)—NH-((1R,2S,4S)-bicyclo[2.2.1]heptan-2-yl), —C(O)—NH-((1R,2R,4R)-bicyclo[2.2.1]heptan-2-yl), —NH—SO₂—(C₁₋₂alkyl), —(C₁₋₂alkyl)-SO₂—NR^{S}R^{T}, and pyrazol-1-yl; wherein the phenyl, benzyl or pyridinyl, whether alone or as part of a substituent group is optionally substituted with one to two substituents independently selected from the group consisting of halogen, C₁₋₄alkyl and C₁₋₄alkoxy; and wherein R^{S} and R^{T} are each independently selected from the group consisting of hydrogen and C₁₋₄alkyl.

In certain embodiments of the present invention, R⁴ is selected from the group consisting of hydrogen, halogen, hydroxy, —(C₁₋₂alkyl)-NR^{S}R^{T}, C₁₋₄alkoxy, —O—(C₁₋₂alkyl)-CN, —O—(C₁₋₂alkyl)-CO₂H, —O—(C₁₋₂alkyl)-C(O)—O—(C₁₋₂alkyl), —O— phenyl, —O—(C₁₋₂alkyl)-phenyl, —O—(C₁₋₂alkyl)-(1,2,3,5-tetrazol-4-yl), —O-(oxetan-3-yl), —O-(tetrahydro-furan-3-yl), —O—C(O)—(C₁₋₂alkyl), —O—C(O)—C₃₋₆cycloalkyl, —O—C(O)—NR^{S}R^{T}, —O—C(O)—(C₁₋₂alkyl)-O—C(O)—(C₁₋₂alkyl), —CO₂H, —C(O)—O—(C₁₋₄alkyl), —C(O)—NR^{S}R^{T}, —C(O)—NH-(phenyl), —C(O)—NH-(benzyl), —C(O)—NH—(C₁₋₂alkyl)-(pyridinyl), —C(O)—NH-((1R,2S,4S)-bicyclo[2.2.1]heptan-2-yl), —C(O)—NH-((1R,2R,4R)-bicyclo[2.2.1]heptan-2-yl), —NH—SO₂—(C₁₋₂alkyl) and pyrazol-1-yl; wherein the phenyl, benzyl or pyridinyl, whether alone or as part of a substituent group is optionally substituted with one to two substituents independently selected from the group consisting of halogen and C₁₋₂alkyl; and wherein R^{S} and R^{T} are each independently selected from the group consisting of hydrogen and C₁₋₄alkyl.

In certain embodiments of the present invention, R⁴ is selected from the group consisting of hydrogen, fluoro, hydroxy, methyl, methoxy, ethoxy, isopropyloxy, phenyloxy, benzyloxy, oxetan-3-yl-oxy, tetrahydrofuran-3-yl-oxy-, carboxy, methoxy-carbonyl-, t-butoxy-carbonyl-, carboxy-methoxy-, methoxy-carbonyl-methoxy-, cyano-methoxy-, 1,2,3,5-tetrazol-4-yl-methoxy-, isopropyl-amino-carbonyl-, (3,4-difluoro-phenyl)-amino-carbonyl-, (2,6-dimethyl-benzyl)-amino-carbonyl-, methyl-carbonyl-oxy, methyl-carbonyl-oxy-methyl-carbonyl-oxy, amino-carbonyl-oxy-, cyclopropyl-carbonyl-oxy-, amino-methyl-, (4-methyl-pyridin-3-yl)-methyl-amino-carbonyl-, (1R,2S,4S)-bicyclo[2.2.1]hept-2-yl-amino-carbonyl-, (1R,2R,4R)-bicyclo[2.2.1]hept-2-yl-amino-carbonyl-, methyl-sulfonyl-amino- and pyrazol-1-yl.

In certain embodiments of the present invention, R⁴ is selected from the group consisting of hydrogen, fluoro, hydroxy, methoxy, ethoxy, isopropyloxy, phenyloxy, benzyloxy, t-butoxy-carbonyl-, carboxy-methoxy-, methoxy-carbonyl-methoxy-, cyano-methoxy-, 1,2,3,5-tetrazol-4-yl-methoxy-, isopropyl-amino-carbonyl-, (3,4-difluoro-phenyl)-amino-carbonyl-, (2,6-dimethyl-benzyl)-amino-carbonyl-, methyl-carbonyl-oxy, methyl-carbonyl-oxy-methyl-carbonyl-oxy, cyclopropyl-carbonyl-oxy-, (4-methyl-pyridin-3-yl)-methyl-amino-carbonyl-, (1R,2S,4S)-bicyclo[2.2.1]hept-2-yl-amino-carbonyl- and (1R,2R,4R)-bicyclo[2.2.1]hept-2-yl-amino-carbonyl-.

In certain embodiments of the present invention, R⁴ is selected from the group consisting of fluoro, hydroxy, methoxy, ethoxy, isopropyloxy, phenyloxy, carboxy-methoxy-, methoxy-carbonyl-methoxy-, cyano-methoxy-, 1,2,3,5-tetrazol-4-yl-methoxy-, isopropyl-amino-carbonyl-, (3,4-difluoro-phenyl)-amino-carbonyl-, (2,6-dimethyl-benzyl)-amino-carbonyl-, methyl-carbonyl-oxy, methyl-carbonyl-oxy-methyl-carbonyl-oxy, cyclopropyl-carbonyl-oxy-, (4-methyl-pyridin-3-yl)-methyl-amino-carbonyl-, (1R,2S,4S)-bicyclo[2.2.1]hept-2-yl-amino-carbonyl- and (1R,2R,4R)-bicyclo[2.2.1]hept-2-yl-amino-carbonyl-.

In certain embodiments of the present invention, R⁴ is selected from the group consisting of hydroxy, methoxy, ethoxy, isopropyloxy, methoxy-carbonyl-methoxy-, cyano-methoxy-, isopropyl-amino-carbonyl-, (3,4-difluoro-phenyl)-amino-carbonyl-, (2,6-dimethyl-benzyl)-amino-carbonyl-, methyl-carbonyl-oxy-methyl-carbonyl-oxy, (4-methyl-pyridin-3-yl)-methyl-amino-carbonyl-, (1R,2S,4S)-bicyclo[2.2.1]hept-2-yl-amino-carbonyl- and (1R,2R,4R)-bicyclo[2.2.1]hept-2-yl-amino-carbonyl-.

In certain embodiments of the present invention, R⁴ is selected from the group consisting of hydroxy, methoxy, methoxy-carbonyl-methoxy-, cyano-methoxy-, isopropyl-amino-carbonyl-, (2,6-dimethyl-benzyl)-amino-carbonyl-, methyl-carbonyl-oxy-methyl-carbonyl-oxy, (4-methyl-pyridin-3-yl)-methyl-amino-carbonyl-, (1R,2S,4S)-bicyclo[2.2.1]hept-2-yl-amino-carbonyl- and (1R,2R,4R)-bicyclo[2.2.1]hept-2-yl-amino-carbonyl-.

In certain embodiments of the present invention, R^{S} and R^{T} are each independently selected from the group consisting of hydrogen and C₁₋₄alkyl. In certain embodiments of the present invention, R^{S} and R^{T} are each independently selected from the group consisting of hydrogen, methyl, ethyl, n-propyl isopropyl and t-butyl. In certain embodiments of the present invention, R^{S} and R^{T} are each independently selected from the group consisting of hydrogen, methyl, ethyl isopropyl and t-butyl. In certain embodiments of the present invention, R^{S} and R^{T} are each independently selected from the group consisting of hydrogen, methyl and ethyl.

In certain embodiments of the present invention, b is an integer from 0 to 2. In certain embodiments of the present invention, b is an integer from 0 to 1. In certain embodiments of the present invention, b is 1. In certain embodiments of the present invention, b is 0.

In certain embodiments of the present invention, each R⁵ is independently selected from the group consisting of halogen, C₁₋₄alkyl and C₁₋₄alkoxy. In certain embodiments of the present invention, each R⁵ is independently selected from C₁₋₂alkoxy. In certain embodiments of the present invention, R⁵ is 6-methoxy.

In certain embodiments of the present invention, R⁶ and R⁷ are the same and are selected from the group consisting of hydrogen, C₁₋₂alkyl and hydroxy substituted C₁₋₂alkyl; alternatively, R⁶ is hydrogen and R⁷ is selected from the group consisting of —(C₁₋₂alkyl)-CN, —(C₁₋₂alkyl)-OH, —(C₁₋₂alkyl)-CO₂H, —(C₁₋₂alkyl)-(1,2,3,5-tetrazol-4-yl) and —(C₁₋₂alkyl)-(isoindolin-2-yl-1,3-dione); alternatively, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3,3-diyl, tetrahydro-pyran-4′,4′-diyl, 2,3-dihydro-inden-1′1′-diyl, hexahydro-2H-cyclopenta[d]oxazol-4′,4′-diyl-2-one and hexahydro-2H-cyclopenta[d]oxazol-6′,6′-diyl-2-one; wherein the cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3′,3′-diyl, tetrahydro-pyran-4′,4′-diyl or 2,3-dihydro-inden-1′1′-diyl is optionally substituted with one to two substituents independently selected from the group consisting of hydroxy, C₁₋₄alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen, methyl and ethyl.

In certain embodiments of the present invention, R⁶ and R⁷ are the same and are selected from the group consisting of hydrogen, C₁₋₂alkyl and hydroxy substituted C₁₋₂alkyl; alternatively, R⁶ is hydrogen and R⁷ is selected from the group consisting of —(C₁₋₂alkyl)-CN, —(C₁₋₂alkyl)-OH, —(C₁₋₂alkyl)-CO₂H, —(C₁₋₂alkyl)-(1,2,3,5-tetrazol-4-yl) and —(C₁₋₂alkyl)-(isoindolin-2-yl-1,3-dione); alternatively, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, piperidin-4′,4′-diyl, tetrahydro-furan-3,3-diyl, tetrahydro-pyran-4′,4′-diyl, 2,3-dihydro-inden-1′1′-diyl and hexahydro-2H-cyclopenta[d]oxazol-4′,4′-diyl-2-one; wherein the cyclopent-1′1′-diyl is optionally substituted with one to two substituents independently selected from the group consisting of hydroxy, C₁₋₂alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen and methyl.

In certain embodiments of the present invention, R⁶ and R⁷ are the same and are selected from the group consisting of hydrogen, methyl and 2-hydroxy-eth-1-yl; alternatively, R⁶ is hydrogen and R⁷ is selected from the group consisting of 2-hydroxy-eth-1-yl, cyano-methyl-, carboxy-methyl-, S*-carboxy-methyl-, R*-carboxy-methyl-, (1,2,3,5-tetrazol-4-yl)-methyl- and (isoindolin-2-yl-1,3-dione)-ethyl-; alternatively, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 2-carboxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 2-(carboxy-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4,4,-diyl, 2,3-dihydro-1H-inden-1′1′-diyl-4-ol and hexahydro-2H-cyclopenta[d]oxazol-4′,4′-diyl-2-one.

In certain embodiments of the present invention, R⁶ and R⁷ are the same and are selected from the group consisting of hydrogen, methyl and 2-hydroxy-eth-1-yl; alternatively, R⁶ is hydrogen and R⁷ is selected from the group consisting of 2-hydroxy-eth-1-yl, cyano-methyl-, carboxy-methyl-, S*-carboxy-methyl- and (1,2,3,5-tetrazol-4-yl)-methyl-; alternatively, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4,4,-diyl and hexahydro-2H-cyclopenta[d]oxazol-4′,4′-diyl-2-one.

In certain embodiments of the present invention, R⁶ and R⁷ are the same and are selected from the group consisting of hydrogen, methyl and 2-hydroxy-eth-1-yl; alternatively, R⁶ is hydrogen and R⁷ is selected from the group consisting of 2-hydroxy-eth-1-yl, cyano-methyl-, carboxy-methyl-, S*-carboxy-methyl- and (1,2,3,5-tetrazol-4-yl)-methyl-; alternatively, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4′,4′-diyl and hexahydro-2H-cyclopenta[d]oxazol-4′,4′-diyl-2-one.

In certain embodiments of the present invention, R⁶ and R⁷ are the same and are selected from the group consisting of hydrogen and methyl; alternatively, R⁶ is hydrogen and R⁷ is selected from the group consisting of 2-hydroxy-eth-1-yl and cyano-methyl-; alternatively, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, 1′1′-diylpiperidin-4′,4′-diyl, tetrahydro-furan-3,3-diyl and tetrahydro-pyran-4′,4′-diyl.

In certain embodiments of the present invention, R⁶ and R⁷ are the same and are selected from the group consisting of hydrogen and methyl; alternatively, R⁶ is hydrogen and R⁷ is selected from the group consisting of 2-hydroxy-eth-1-yl and cyano-methyl-; alternatively, R⁶ and R⁷ are taken together with the carbon atom to which they are bound to form a ring structure selected from the group consisting of cyclopent-1′1′-diyl, 3-carboxy-cyclopent-1′1′-diyl, 3-(methyl-amino)-cyclopent-1′1′-diyl, 3-(amino-carbonyl)-cyclopent-1′1′-diyl and 1′1′-diylpiperidin-4′,4′-diyl.

In certain embodiments of the present invention, R⁶ and R⁷ are the same and are selected from the group consisting of hydrogen, C₁₋₂alkyl and hydroxy substituted C₁₋₂alkyl. In certain embodiments of the present invention, R⁶ and R⁷ are the same and are selected from the group consisting of hydrogen, methyl and 2-hydroxy-eth-1-yl. In certain embodiments of the present invention, R⁶ and R⁷ are the same and are selected from the group consisting of hydrogen and methyl.

In certain embodiments of the present invention, R⁶ is hydrogen and R⁷ is selected from the group consisting of —(C₁₋₂alkyl)-CN, —(C₁₋₂alkyl)-OH, —(C₁₋₂alkyl)-CO₂H, —(C₁₋₂alkyl)-(1,2,3,5-tetrazol-4-yl) and —(C₁₋₂alkyl)-(isoindolin-2-yl-1,3-dione). In certain embodiments of the present invention, R⁶ is hydrogen and R⁷ is selected from the group consisting of 2-hydroxy-eth-1-yl, cyano-methyl-, carboxy-methyl-, S*-carboxy-methyl-, R*-carboxy-methyl-, (1,2,3,5-tetrazol-4-yl)-methyl- and (isoindolin-2-yl-1,3-dione)-ethyl-. In certain embodiments of the present invention, R⁶ is hydrogen and R⁷ is selected from the group consisting of 2-hydroxy-eth-1-yl, cyano-methyl-, carboxy-methyl-, S*-carboxy-methyl- and (1,2,3,5-tetrazol-4-yl)-methyl-. In certain embodiments of the present invention, R⁶ is hydrogen and R⁷ is selected from the group consisting of 2-hydroxy-eth-1-yl and cyano-methyl-.

In certain embodiments of the present invention, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3,3-diyl, tetrahydro-pyran-4′,4′-diyl, 2,3-dihydro-inden-1′1′-diyl, hexahydro-2H-cyclopenta[d]oxazol-4′,4′-diyl-2-one and hexahydro-2H-cyclopenta[d]oxazol-6′,6′-diyl-2-one; wherein the cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3′,3′-diyl, tetrahydro-pyran-4′,4′-diyl or 2,3-dihydro-inden-1′1′-diyl is optionally substituted with one to two substituents independently selected from the group consisting of hydroxy, C₁₋₄alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen, methyl and ethyl.

In certain embodiments of the present invention, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3,3-diyl, tetrahydro-pyran-4′,4′-diyl, 2,3-dihydro-inden-1′1′-diyl, hexahydro-2H-cyclopenta[d]oxazol-4′,4′-diyl-2-one and hexahydro-2H-cyclopenta[d]oxazol-6′,6′-diyl-2-one; wherein the cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3′,3′-diyl, tetrahydro-pyran-4′,4′-diyl or 2,3-dihydro-inden-1′1′-diyl is optionally substituted with one to two substituents independently selected from the group consisting of hydroxy, C₁₋₄alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen, methyl and ethyl.

In certain embodiments of the present invention, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, piperidin-4′,4′-diyl, tetrahydro-furan-3,3-diyl, tetrahydro-pyran-4′,4′-diyl, 2,3-dihydro-inden-1′1′-diyl and hexahydro-2H-cyclopenta[d]oxazol-4′,4′-diyl-2-one; wherein the cyclopent-1′1′-diyl is optionally substituted with one to two substituents independently selected from the group consisting of hydroxy, C₁₋₂alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen and methyl.

In certain embodiments of the present invention, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 2-carboxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 2-(carboxy-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4,4,-diyl, 2,3-dihydro-1H-inden-1′1′-diyl-4-ol and hexahydro-2H-cyclopenta[d]oxazol-4′,4′-diyl-2-one.

In certain embodiments of the present invention, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4,4,-diyl and hexahydro-2H-cyclopenta[d]oxazol-4′,4′-diyl-2-one;

In certain embodiments of the present invention, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4′,4′-diyl and hexahydro-2H-cyclopenta[d]oxazol-4′,4′-diyl-2-one;

In certain embodiments of the present invention, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, 1′1′-diylpiperidin-4′,4′-diyl, tetrahydro-furan-3,3-diyl and tetrahydro-pyran-4′,4′-diyl;

In certain embodiments of the present invention, R⁶ and R⁷ are taken together with the carbon atom to which they are bound to form a ring structure selected from the group consisting of cyclopent-1′1′-diyl, 3-carboxy-cyclopent-1′1′-diyl, 3-(methyl-amino)-cyclopent-1′1′-diyl, 3-(amino-carbonyl)-cyclopent-1′1′-diyl and 1′1′-diylpiperidin-4′,4′-diyl;

In certain embodiments of the present invention, R^{X} and R^{Y} are each independently selected from the group consisting of hydrogen, methyl and ethyl. In certain embodiments of the present invention, R^{X} and R^{Y} are each independently selected from the group consisting of hydrogen methyl. wherein R^{X} and R^{Y} are each independently selected from the group consisting of hydrogen and ethyl.

In another embodiment, the present invention is directed to a compound of formula (I) selected from the group consisting of
2-[(3-methoxyphenyl)methyl]-6-(1H-pyrazol-4-yl)isoindolin-1-one;
2-[(1R)-1-(3-methoxyphenyl)ethyl]-6-(1H-pyrazol-4-yl)isoindolin-1-one;
N-isopropyl-3-[[1-oxo-6-(1H-pyrazol-4-yl)isoindolin-2-yl]methyl]benzamide;
2-[(1R)-1-(3-ethoxyphenyl)ethyl]-6-(1H-pyrazol-4-yl)isoindolin-1-one;
7-fluoro-2-[(1R)-1-(3-methoxyphenyl)ethyl]-6-(1H-pyrazol-4-yl)isoindolin-1-one;
5-fluoro-2-[(3-methoxyphenyl)methyl]-6-(1H-pyrazol-4-yl)isoindolin-1-one;
2′-[(3-methoxyphenyl)methyl]-6′-(1H-pyrazol-4-yl)spiro[cyclopentane-1,3′-isoindoline]-1′-one;
2-[(1S*)-2-hydroxy-1-(3-methoxyphenyl)ethyl]-6-(1H-pyrazol-4-yl)isoindolin-1-one;
2-[(1R)-1-(3-methoxyphenyl)ethyl]-6-(1H-pyrazol-4-yl)-4-pyrimidin-5-yl-isoindolin-1-one;
2-[(1R)-1-(3-methoxyphenyl)ethyl]-4-piperazin-1-yl-6-(1H-pyrazol-4-yl)isoindolin-1-one;

and stereoisomers and pharmaceutically acceptable salts thereof.

In certain embodiments of the present invention, R² is other than optionally substituted pyrimidin-4-yl. In certain embodiments of the present invention, R² is other than optionally substituted pyrimidinyl. In certain embodiments of the present invention, R² is other than optionally substituted imidazol-1-yl. In certain embodiments of the present invention, R² is other than optionally substituted imidazolyl.

In certain embodiments of the present invention, R² is other than optionally substituted 1,2,4-oxadiazol-3-yl, 1,3,4-oxadizol-2-yl or 1,2,5-thiadiazol-3-yl. In certain embodiments of the present invention, R² is other than optionally substituted oxadiazolyl, oxadizolyl or thiadiazolyl. In certain embodiments of the present invention, R² is other than optionally substituted piperidin-1-yl. In certain embodiments of the present invention, R² is other than optionally substituted piperidinyl.

In certain embodiments of the present invention, R² is selected from the group consisting of 5 to 10 membered heteroaryl and 5 to 10 membered heterocycloalkyl; wherein the 5 to 10 membered heteroaryl or 5 to 10 membered heterocycloalkyl is optionally substituted as herein defined; wherein the optionally substituted 5 to 10 membered heteroaryl is other than pyrimidin-4-yl, 1,2,4-oxadiazol-3-yl, 1,3,4-oxadizol-2-yl or 1,2,5-thiadiazol-3-yl; and wherein the optionally substituted 5 to 10 membered heterocycloalkyl is other than piperidin-1-yl.

In certain embodiments of the present invention, R² is selected from the group consisting of 5 to 10 membered heteroaryl and 5 to 10 membered heterocycloalkyl; wherein the 5 to 10 membered heteroaryl or 5 to 10 membered heterocycloalkyl is optionally substituted as herein defined; wherein the optionally substituted 5 to 10 membered heteroaryl is other than pyrimidinyl, oxadiazolyl, oxadizolyl or thiadiazolyl; and wherein the optionally substituted 5 to 10 membered heterocycloalkyl is other than piperidinyl.

In certain embodiments of the present invention, R² is other than optionally substituted imidazol-1-yl and R⁴ is other than —O—(C₁₋₂alkyl-1,2,3,5-tetrazol-4-yl). In certain embodiments of the present invention, R⁴ is other than —O—(C₁₋₂alkyl-1,2,3,5-tetrazol-4-yl).

Additional embodiments of the present invention, include those wherein the substituents selected for one or more of the variables defined herein (i.e. a, b, R¹, R², R³, R⁴, R⁵, R⁶, R⁷, Q, Z,

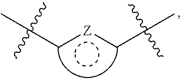

etc.) are independently selected to be any individual substituent or any subset of substituents selected from the complete list as defined herein. Additional embodiments of the present invention, include those wherein the substituents selected for one or more of the variables defined herein (i.e. a, b, R¹, R², R³, R⁴, R⁵, R⁶, R⁷, Q, Z,

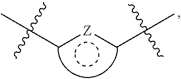

etc.) are independently selected to correspond to any of the embodiments as defined herein.

In another embodiment of the present invention is any single compound or subset of compounds selected from the representative compounds listed in Tables 1-3, below.

Representative compounds of the present invention are as listed in Tables 1-3, below. Unless otherwise noted, wherein a stereogenic center is present in the listed compound, the compound was prepared as a mixture of stereo-configurations. Where a stereogenic center is present and an S* or R* designation is noted, the S* and R* designations indicate that the compound was prepared in an enantiomeric excess of one of the stereoisomers, although the exact stereo-configuration of the designated center was not determined. Where a stereogenic center is present and an S or R designation is noted, the S and R designations indicate that the compound was prepared in an enantiomeric excess of one of the corresponding stereoisomers, and further that the exact stereo-configuration of the designated center was determined to be S or R, as noted.

TABLE 1
Representative Compounds of Formula (I)

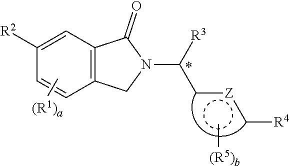

| ID NO. | (R¹)ₐ | R² | R³ | R⁴ | (R⁵)_{b} | 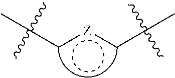 |
|---|---|---|---|---|---|---|
| 1 | a = 0 | pyrazol-4-yl | H | methoxy | b = 0 | phenyl |
| 2 | a = 0 | pyrazol-4-yl | H | ethoxy | b = 0 | phenyl |
| 3 | a = 0 | 1H-pyrrolo[2,3-b] | H | methoxy | b = 0 | phenyl |
| | | pyridin-3-yl | | | | |
| 4 | a = 0 | 2-amino- | H | methoxy | b = 0 | phenyl |
| | | pyrimidin-4-yl | | | | |
| 9 | a = 0 | pyrazol-4-yl | H | methyl- | b = 0 | phenyl |
| | | | | sulfonyl- | | |
| | | | | amino- | | |
| 10 | a = 0 | 1H-pyrrolo[2,3- | H | methyl- | b = 0 | phenyl |
| | | b]pyridin-4-yl | | sulfonyl- | | |
| 11 | a = 0 | 2-amino- | H | ethoxy | b = 0 | phenyl |
| | | pyrimidin- | | | | |
| | | 4-yl | | | | |
| 13 | a = 0 | pyrazol-4-yl | R-methyl | methoxy | b = 0 | phenyl |
| 16 | a = 0 | pyrazol-4-yl | H | fluoro | b = 0 | phenyl |
| 18 | a = 0 | pyrazol-4-yl | H | carboxy | b = 0 | phenyl |
| 19 | a = 0 | pyrazol-4-yl | H | isopropyl- | b = 0 | phenyl |
| | | | | amino- | | |
| | | | | carbonyl- | | |
| 20 | a = 0 | 1,2,3- | H | methoxy | b = 0 | phenyl |
| | | triazol-4-yl | | | | |
| 22 | a = 0 | pyrazol-4-yl | H | amino- | b = 0 | phenyl |
| | | | | methyl- | | |
| 23 | a = 0 | 6-(methyl- | H | methoxy | b = 0 | phenyl |
| | | amino- | | | | |
| | | pyridin-3-yl | | | | |
| 24 | a = 0 | pyrazol-4-yl | R-methyl | H | b = 0 | 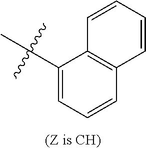 [SMILES: *c1cccc2ccccc12] |
| 25 | a = 0 | pyrazol-4-yl | R-methyl | methyl | b = 0 | 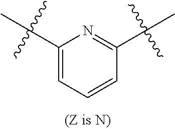 [SMILES: *c1cccc(*)n1] |
| 26 | a = 0 | pyrazol-4-yl | R-methyl | carboxy | b = 0 | phenyl |
| 27 | a = 0 | pyrazol-4-yl | H | methyl | b = 0 | 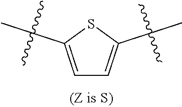 [SMILES: *c1ccc(*)s1] |
| 28 | a = 0 | pyrazol-4-yl | H | H | b = 0 | 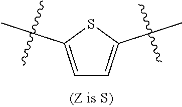 [SMILES: *c1ccc(*)s1] |
| 29 | a = 0 | pyrazol-4-yl | R-methyl | ethoxy | b = 0 | phenyl |
| 30 | 7-fluoro | pyrazol-4-yl | R-methyl | methoxy | b = 0 | phenyl |
| 31 | a = 0 | pyrazol-4-yl | R-methyl | isopropyl- | b = 0 | phenyl |
| | | | | amino- | | |
| | | | | carbonyl- | | |
| 32 | a = 0 | pyrazol-4-yl | R-methyl | (3,4- | b = 0 | phenyl |
| | | | | difluoro- | | |
| | | | | phenyl)- | | |
| | | | | amino- | | |
| | | | | carbonyl- | | |
| 34 | a = 0 | pyrazol-4-yl | H | (3,4- | b = 0 | phenyl |
| | | | | difluoro- | | |
| | | | | phenyl)- | | |
| | | | | amino- | | |
| | | | | carbonyl- | | |
| 35 | 5-fluoro | pyrazol-4-yl | H | methoxy | b = 0 | phenyl |
| 36 | 5-fluoro | 2-amino- | H | methoxy | b = 0 | phenyl |
| | | pyrimidin-4-yl | | | | |
| 37 | a = 0 | 2-(methyl- | H | methoxy | b = 0 | phenyl |
| | | amino- | | | | |
| | | carbonyl)- | | | | |
| | | pyridin-4-yl | | | | |
| 38 | a = 0 | pyrazol-4-yl | hydroxy- | methoxy | b = 0 | phenyl |
| | | | methyl- | | | |
| 40 | a = 0 | 2,4- | R-methyl | methoxy | b = 0 | phenyl |
| | | dihydro- | | | | |
| | | 3H-1,2,4- | | | | |
| | | triazol-4- | | | | |
| | | yl-3-one | | | | |
| 43 | a = 0 | pyrazol-4-yl | H | fluoro | b = 0 | 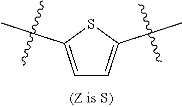 [SMILES: *c1ccc(*)s1] |
| 44 | a = 0 | 2,4-dihydro- | H | methoxy | b = 0 | phenyl |
| | | 3H-1,2,4- | | | | |
| | | triazol-4- | | | | |
| | | yl-3-one | | | | |
| 47 | a = 0 | 2-(methyl- | H | methoxy | b = 0 | phenyl |
| | | amino- | | | | |
| | | carbonyl)- | | | | |
| | | pyrimidin-4-yl | | | | |
| 48 | 7-fluoro | pyrazol-4- | R-methyl | ethoxy | b = 0 | phenyl |
| | | yl | | | | |
| 50 | a = 0 | 1H- | H | methoxy | b = 0 | phenyl |
| | | pyrrolo[2,3- | | | | |
| | | b]pyridin-4-yl | | | | |
| 51 | a = 0 | 2-amino- | R-methyl | ethoxy | b = 0 | phenyl |
| | | pyridin-4-yl | | | | |
| 52 | a = 0 | pyrazol-4-yl | hydroxy- | methoxy | 6- | phenyl |
| | | | methyl- | | methoxy | |
| 53 | a = 0 | pyrazol-4-yl | hydroxy- | ethoxy | b = 0 | phenyl |
| | | | methyl- | | | |
| 54 | a = 0 | 1H- | H | methoxy- | b = 0 | phenyl |
| | | pyrrolo[2,3- | | carbonyl- | | |
| | | b]pyridin-4-yl | | methoxy- | | |
| 55 | a = 0 | pyrazol-4-yl | H | carboxy- | b = 0 | phenyl |
| | | | | methoxy- | | |
| 56 | a = 0 | pyrazol-4-yl | | methoxy- | b = 0 | phenyl |
| | | | | carbonyl- | | |
| | | | | methoxy- | | |
| 57 | a = 0 | 1H- | H | carboxy- | b = 0 | phenyl |
| | | pyrrolo[2,3- | | methoxy- | | |
| | | b]pyridin-4-yl | | | | |
| 59 | a = 0 | pyrazol-4-yl | methoxy- | methoxy | b = 0 | phenyl |
| | | | methyl- | | | |
| 60 | a = 0 | pyrazol-4-yl | carboxy- | methoxy | b = 0 | phenyl |
| | | | methoxy- | | | |
| | | | methyl- | | | |
| 62 | a = 0 | pyrazol-4-yl | amino- | methoxy | b = 0 | phenyl |
| | | | carbonyl- | | | |
| | | | methoxy- | | | |
| | | | methyl- | | | |
| 63 | a = 0 | pyrazol-4-yl | 2- | methoxy | b = 0 | phenyl |
| | | | hydroxy- | | | |
| | | | ethoxy- | | | |
| | | | methyl- | | | |
| 64 | a = 0 | 2-amino- | hydroxy- | methoxy | b = 0 | phenyl |
| | | pyrimidin-4-yl | methyl- | | | |
| 66 | a = 0 | pyridin-4-yl | H | methoxy | b = 0 | phenyl |
| 67 | a = 0 | pyrazol-4-yl | R-carboxy | methoxy | b = 0 | phenyl |
| 68 | a = 0 | pyrazol-4-yl | H | amino- | b = 0 | phenyl |
| | | | | carbonyl- | | |
| | | | | oxy- | | |
| 69 | a = 0 | pyrazol-4-yl | H | isopropyl- | b = 0 | phenyl |
| | | | | oxy- | | |
| 70 | a = 0 | pyrazol-4-yl | methoxy- | methoxy | b = 0 | phenyl |
| | | | carbonyl- | | | |
| 71 | a = 0 | pyrazol-4-yl | carboxy- | methoxy | b = 0 | phenyl |
| 72 | a = 0 | pyrazol-4-yl | S- | methoxy | b = 0 | phenyl |
| | | | (methoxy- | | | |
| | | | carbonyl-) | | | |
| 73 | a = 0 | pyrazol-4-yl | R- | ethoxy | b = 0 | phenyl |
| | | | (methoxy- | | | |
| | | | carbonyl-) | | | |
| 74 | a = 0 | pyrazol-4-yl | R-carboxy | ethoxy | b = 0 | phenyl |
| 76 | a = 0 | pyridin-4-yl | R- | ethoxy | b = 0 | phenyl |
| | | | (methoxy- | | | |
| | | | carbonyl-) | | | |
| 77 | a = 0 | pyridin-4-yl | R-carboxy | ethoxy | b = 0 | phenyl |
| 80 | a = 0 | pyrazol-4-yl | dimethyl- | methoxy | b = 0 | phenyl |
| | | | amino- | | | |
| | | | methyl- | | | |
| 83 | a = 0 | pyridin-3-yl | hydroxy- | methoxy | b = 0 | phenyl |
| | | | methyl- | | | |
| 84 | a = 0 | pyrazol-4-yl | 1,2,3,4- | methoxy | b = 0 | phenyl |
| | | | tetrazol-5- | | | |
| | | | yl-methyl- | | | |
| 85 | a = 0 | pyrazol-4-yl | H | benzyloxy | b = 0 | phenyl |
| 86 | a = 0 | pyrazol-4-yl | benzyloxy- | methoxy | b = 0 | phenyl |
| | | | methyl- | | | |
| 87 | a = 0 | pyrazol-4-yl | H | cyano- | b = 0 | phenyl |
| | | | | methoxy- | | |
| 88 | a = 0 | pyrazol-4-yl | H | 1,2,3,5- | b = 0 | phenyl |
| | | | | tetrazol-4- | | |
| | | | | yl- | | |
| | | | | methoxy | | |
| 90 | a = 0 | pyrazol-4-yl | H | tetrahydro- | b = 0 | phenyl |
| | | | | furan-3- | | |
| | | | | yl-oxy | | |
| 91 | a = 0 | pyrazol-4-yl | H | oxetan-3- | b = 0 | phenyl |
| | | | | yl-oxy | | |
| 92 | a = 0 | pyrazol-4-yl | phenyl- | methoxy | b = 0 | phenyl |
| | | | carbonyl- | | | |
| 94 | a = 0 | pyrazol-4-yl | 1,2,3,5- | methoxy | b = 0 | phenyl |
| | | | tetrazol-4- | | | |
| | | | yl | | | |
| 96 | a = 0 | pyrazol-4-yl | R-cyclo- | methoxy | b = 0 | phenyl |
| | | | propyl | | | |
| 99 | a = 0 | pyrazol-4-yl | S-methyl | methoxy | b = 0 | phenyl |
| 100 | a = 0 | pyrazol-4-yl | carboxy- | methoxy | b = 0 | phenyl |
| | | | methyl- | | | |
| 103 | a = 0 | pyrazol-4-yl | H | phenyloxy | b = 0 | phenyl |
| 104 | a = 0 | pyrazol-4-yl | methyl | phenyloxy | b = 0 | phenyl |
| 105 | a = 0 | pyrazol-4-yl | H | methoxy | b = 0 | 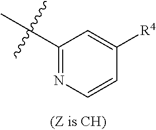 |
| 106 | a = 0 | pyrimidin-yl | H | phenyloxy | b = | phenyl |
| 112 | a = 0 | pyrazol-4-yl | R- | ethoxy | b = 0 | phenyl |
| | | | (hydroxy- | | | |
| | | | methyl) | | | |
| 113 | a = 0 | pyrazol-4-yl | S*- | methoxy | b = 0 | phenyl |
| | | | (hydroxy- | | | |
| | | | methyl) | | | |
| 114 | a = 0 | pyrazol-4-yl | S- | ethoxy | b = 0 | phenyl |
| | | | (hydroxy- | | | |
| | | | methyl-) | | | |
| 117 | a = 0 | pyrazol-4-yl | 2- | methoxy | b = 0 | phenyl |
| | | | carboxy- | | | |
| | | | eth-1-yl | | | |
| 119 | a = 0 | pyrazol-4-yl | H | pyrazol-1- | b = 0 | phenyl |
| | | | | yl | | |
| 120 | 6-(5-fluoro- | pyrazol-4-yl | R-methyl | methoxy | b = 0 | phenyl |
| | pyridin-3- | | | | | |
| | yl) | | | | | |
| 121 | 6-(pyridin- | pyrazol-4-yl | R-methyl | methoxy | b = 0 | phenyl |
| | 4-yl) | | | | | |
| 124 | 6-(pyridin- | pyrazol-4-yl | R-methyl | methoxy | b = 0 | phenyl |
| | 3-yl) | | | | | |
| 125 | 6-(2- | pyrazol-4-yl | R-methyl | methoxy | b = 0 | phenyl |
| | amino- | | | | | |
| | pyridin-3- | | | | | |
| | yl) | | | | | |
| 126 | 6-(5- | pyrazol-4-yl | R-methyl | methoxy | b = 0 | phenyl |
| | methoxy- | | | | | |
| | pyridin-3- | | | | | |
| | yl) | | | | | |
| 127 | 6-(6- | pyrazol-4-yl | R-methyl | methoxy | b = 0 | phenyl |
| | methoxy- | | | | | |
| | pyridin-3- | | | | | |
| | yl) | | | | | |
| 128 | a = 0 | pyrazol-4-yl | H | ((1R,2S,4S)- | b = 0 | phenyl |
| | | | | bicyclo[2.2.1] | | |
| | | | | heptan-2-yl)- | | |
| | | | | amino- | | |
| | | | | carbonyl- | | |
| 129 | 6-(4- | pyrazol-4-yl | R-methyl | methoxy | b = 0 | phenyl |
| | hydroxy- | | | | | |
| | phenyl) | | | | | |
| 130 | 6- | pyrazol-4-yl | R-methyl | methoxy | b = 0 | phenyl |
| | (pyrimidin- | | | | | |
| | 5-yl) | | | | | |
| 131 | a = 0 | pyrazol-4-yl | H | (2,6- | b = 0 | phenyl |
| | | | | dimethyl- | | |
| | | | | benzyl)- | | |
| | | | | amino- | | |
| | | | | carbonyl | | |
| 132 | 6-(4,6- | pyrazol-4-yl | R-methyl | methoxy | b = 0 | phenyl |
| | dimethyl- | | | | | |
| | pyridin-4- | | | | | |
| | yl) | | | | | |
| 133 | 6-(pyridin- | pyrazol-4-yl | hydroxy- | methoxy | b = 0 | phenyl |
| | 4-yl) | | methyl- | | | |
| 135 | a = 0 | pyrazol-4-yl | H | hydroxy | b = 0 | phenyl |
| 136 | a = 0 | pyrazol-4-yl | H | methyl- | b = 0 | phenyl |
| | | | | carbonyl- | | |
| | | | | oxy- | | |
| 137 | a = 0 | pyrazol-4-yl | H | cyclopropyl- | b = 0 | phenyl |
| | | | | yl- | | |
| | | | | carbonyl- | | |
| | | | | oxy- | | |
| 138 | a = 0 | pyrazol-4-yl | H | methyl- | b = 0 | phenyl |
| | | | | carbonyl- | | |
| | | | | oxy- | | |
| | | | | methyl- | | |
| | | | | carbonyl- | | |
| | | | | oxy- | | |
| 139 | 6-(3- | pyrazol-4-yl | R-methyl | methoxy | b = 0 | phenyl |
| | hydroxy- | | | | | |
| | phenyl) | | | | | |
| 140 | 6-(3- | pyrazol-4-yl | R-methyl | methoxy | b = 0 | phenyl |
| | methoxy- | | | | | |
| | phenyl) | | | | | |
| 141 | 6-(6-(1- | pyrazol-4-yl | R-methyl | methoxy | b = 0 | phenyl |
| | hydroxy- | | | | | |
| | isopropyl)- | | | | | |
| | pyridin-3- | | | | | |
| | yl) | | | | | |
| 142 | 6-(pyridin- | pyrazol-4-yl | hydroxy- | methoxy | b = 0 | phenyl |
| | 3-yl) | | methyl- | | | |
| 143 | 6-(3- | pyrazol-4-yl | hydroxy- | methoxy | b = 0 | phenyl |
| | hydroxy- | | methyl- | | | |
| | phenyl) | | | | | |
| 144 | 6- | pyrazol-4-yl | R-methyl | methoxy | b = 0 | phenyl |
| | (pyrrolidin- | | | | | |
| | 3-yl) | | | | | |
| 145 | 6-(6- | pyrazol-4-yl | R-methyl | methoxy | b = 0 | phenyl |
| | (trifluoro- | | | | | |
| | methyl)- | | | | | |
| | pyridin-3- | | | | | |
| | yl) | | | | | |
| 146 | 6- | pyrazol-4-yl | R-methyl | methoxy | b = 0 | phenyl |
| | (piperidin- | | | | | |
| | 3-yl) | | | | | |
| 147 | 6- | pyrazol-4-yl | R-methyl | methoxy | b = 0 | phenyl |
| | (piperidin- | | | | | |
| | 4-yl) | | | | | |
| 148 | 6- | pyrazol-4-yl | R-methyl | methoxy | b = 0 | phenyl |
| | (tetrahydro- | | | | | |
| | pyran-4-yl) | | | | | |
| 149 | 6- | pyrazol-4-yl | R-methyl | methoxy | b = 0 | phenyl |
| | (pyrrolidin- | | | | | |
| | 3-yl) | | | | | |
| 152 | 6-(5-fluoro- | pyrazol-4-yl | hydroxy- | methoxy | b = 0 | phenyl |
| | pyridin-3- | | methyl- | | | |
| | yl) | | | | | |
| 153 | 6-(4- | pyrazol-4-yl | hydroxy- | methoxy | b = 0 | phenyl |
| | hydroxy- | | methyl- | | | |
| | phenyl) | | | | | |
| 154 | 6-(2-t- | pyrazol-4-yl | R-methyl | methoxy | b = 0 | phenyl |
| | butyl- | | | | | |
| | pyridin-4- | | | | | |
| | yl) | | | | | |
| 155 | 6- | pyrazol-4-yl | R-methyl | methoxy | b = 0 | phenyl |
| | (piperazin- | | | | | |
| | 1-yl) | | | | | |
| 156 | 6-(4- | pyrazol-4-yl | R-methyl | methoxy | b = 0 | phenyl |
| | methyl- | | | | | |
| | piperazin- | | | | | |
| | 1-yl) | | | | | |
| 158 | 6- | pyrazol-4-yl | hydroxy- | methoxy | b = 0 | phenyl |
| | (pyrimidin- | | methyl- | | | |
| | 5-yl) | | | | | |
| 159 | 6-(5-t- | pyrazol-4-yl | R-methyl | methoxy | b = 0 | phenyl |
| | butyl- | | | | | |
| | pyrdin-3-yl) | | | | | |
| 160 | 6-(2,2,6,6- | pyrazol-4-yl | R-methyl | methoxy | b = 0 | phenyl |
| | tetramethyl- | | | | | |
| | piperidin- | | | | | |
| | 4-yl) | | | | | |
| 161 | 6-(pyridin- | pyrazol-4-yl | hydroxy- | methoxy | b = 0 | phenyl |
| | 2-yl) | | methyl- | | | |
| 162 | 6- | pyrazol-4-yl | hydroxy- | methoxy | b = 0 | phenyl |
| | (pyrimidin- | | methyl- | | | |
| | 4-yl) | | | | | |
| 163 | a = 0 | pyrazol-4-yl | hydroxy- | phenyloxy | b = 0 | phenyl |
| | | | methyl- | | | |
| 164 | a = 0 | pyrazol-4-yl | H | t-butoxy- | b = 0 | phenyl |
| | | | | carbonyl- | | |
| 165 | a = 0 | pyrazol-4-yl | R-methyl | methoxy- | b = 0 | phenyl |
| | | | | carbonyl- | | |
| 166 | 5-fluoro | pyrazol-4-yl | H | isopropyl- | b = 0 | phenyl |
| | | | | amino- | | |
| | | | | carbonyl- | | |
| 167 | a = 0 | 1H- | R-methyl | ethoxy | b = 0 | phenyl |
| | | pyrrolo[2,3- | | | | |
| | | b]pyridin-4-yl | | | | |
| 168 | a = 0 | pyrazol-4-yl | dimethyl- | methoxy | b = 0 | phenyl |
| | | | amino- | | | |
| | | | carbonyl- | | | |
| 169 | a = 0 | pyrazol-4-yl | S-carboxy | methoxy | b = 0 | phenyl |
| 171 | a = 0 | pyrimidin- | R-methyl | ethoxy | b = 0 | phenyl |
| | | 2-yl | | | | |
| 172 | a = 0 | pyrazol-4-yl | R- | methoxy | b = 0 | phenyl |
| | | | hydroxy- | | | |
| | | | methyl- | | | |
| 174 | a = 0 | pyrazol-4-yl | H | (1R,2R,4R)- | b = 0 | phenyl |
| | | | | bicyclo | | |
| | | | | [2.2.1]hept- | | |
| | | | | 2-yl- | | |
| | | | | amino- | | |
| | | | | carbonyl- | | |

TABLE 2
Representative Compounds of Formula (I)

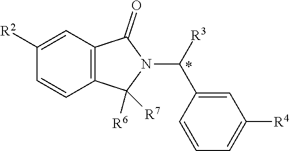

| ID | | | | | |
|---|---|---|---|---|---|
| NO. | R² | R³ | R⁴ | R⁶ | R⁷ |
| 7 | pyrazol-4-yl | H | methoxy | methyl | methyl |
| 12 | pyrazol-4-yl | H | ethoxy | H | (isoindolin-2-yl- |
| | | | | | 1,3-dione)-ethyl- |
| 15 | pyrazol-4-yl | H | methoxy | H | cyano-methyl- |
| 17 | 2-amino- | H | methoxy | methyl | methyl |
| | pyrimidin-4- | | | | |
| | yl | | | | |
| 21 | 1,2,3-triazol- | H | methoxy | methyl | methyl |
| | 4-yl | | | | |
| 33 | 2-amino- | H | methoxy | H | cyano-methyl- |
| | pyrimidin-4- | | | | |
| | yl | | | | |
| 39 | 1,2,3-triazol- | H | ethoxy | methyl | methyl |
| | 4-yl | | | | |
| 41 | pyrazol-4-yl | H | methoxy | 2-hydroxy- | 2-hydroxy- |
| | | | | eth-1-yl | eth-1-yl |
| 45 | 2,4-dihydro- | H | ethoxy | methyl | methyl |
| | 3H-1,2,4- | | | | |
| | triazol-4-yl-3- | | | | |
| | one | | | | |
| 49 | pyrazol-4-yl | R- | methoxy | H | 2-hydroxy- |
| | | methyl | | | eth-1-yl |
| 61 | pyrazol-4-yl | H | methoxy | H | carboxy-methyl- |
| 65 | pyrazol-4-yl | H | ethoxy | H | cyano-methyl- |
| 93 | pyrazol-4-yl | H | methoxy | H | 1,2,3,5-tetrazol-4- |
| | | | | | yl-methyl- |
| 97 | pyrazol-4-yl | R- | methoxy | H | S*-carboxy- |
| | | methyl | | | methyl- |
| 98 | pyrazol-4-yl | R- | methoxy | H | R*-carboxy- |
| | | methyl | | | methyl- |

TABLE 3
Representative Compounds of Formula (I)

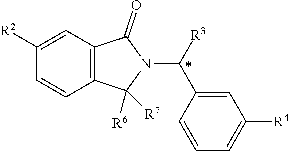

| ID | | | | R⁶ and R⁷ taken |
|---|---|---|---|---|
| NO. | R² | R³ | R⁴ | together with C atom |
| 6 | pyrazol-4-yl | H | methoxy | cyclpent-3-en-1′1′-diyl |
| 8 | pyrazol-4-yl | H | methoxy | 3,4-dihydroxy- |
| | | | | cyclopent-1′1′-diyl |
| 14 | pyrazol-4-yl | H | methoxy | piperidin-4′,4′-diyl |
| 42 | pyrazol-4-yl | H | methoxy | cyclopent-1′1′-diyl |
| 46 | pyrazol-4-yl | H | methoxy | 3-hydroxy-cyclopent- |
| | | | | 1′1′-diyl |
| 75 | pyrazol-4-yl | H | methoxy | tetrahydro-furan-3,3- |
| | | | | diyl |
| 78 | pyrazol-4-yl | hydroxy- | methoxy | cyclopent-3-en-1′1′-diyl |
| | | methyl- | | |
| 79 | pyrazol-4-yl | hydroxy- | methoxy | cyclopent-1′1′-diyl |
| | | methyl- | | |
| 81 | pyrazol-4-yl | H | methoxy | cyclohex-1′1′-diyl |
| 82 | pyrazol-4-yl | H | methoxy | tetrahydro-pyran-4′,4′- |
| | | | | diyl |
| 89 | pyrazol-4-yl | H | methoxy | 2,3-dihydro-1H-inden- |
| | | | | 1′1′-diyl-4-ol |
| 102 | pyrazol-4-yl | H | methoxy | 2,3-dihydroxy- |
| | | | | cyclopent-1′1′-diyl |
| 107 | pyrazol-4-yl | H | methoxy | 3-carboxy-cyclopent- |
| | | | | 1′1′-diyl |
| 108 | pyrazol-4-yl | H | methoxy | 3-(amino-carbonyl)- |
| | | | | cyclopent-1′1′-diyl |
| 109 | pyrazol-4-yl | H | methoxy | 3-(methoxy-carbonyl- |
| | | | | amino)-cyclopent-1′1′- |
| | | | | diyl |
| 110 | pyrazol-4-yl | H | methoxy | 2-carboxy-cyclopent- |
| | | | | 1′1′-diyl |
| 111 | pyrazol-4-yl | H | methoxy | 1-(amino-carbonyl)- |
| | | | | cyclopent-1′1′-diyl |
| 115 | pyrazol-4-yl | H | methoxy | 2-(carboxy-amino)- |
| | | | | cyclopent-1′1′-diyl |
| 118 | pyrazol-4-yl | H | methoxy | 3-(methyl-amino)- |
| | | | | cyclopent-1′1′-diyl |
| 122 | pyrazol-4-yl | R-methyl | methoxy | cyclopent-1′1′-diyl |
| 123 | pyrazol-4-yl | H | methoxy | 2-amino-cyclopent-1′1′- |
| | | | | diyl |
| 134 | pyrazol-4-yl | H | (4-methyl- | cyclopent-1′1′-diyl |
| | | | pyridin-3-yl)- | |
| | | | methyl-amino- | |
| | | | carbonyl- | |
| 150 | pyrazol-4-yl | H | methoxy | hexahydro-2H- |
| | | | | cyclopenta[d]oxazol- |
| | | | | 6′,6′-diyl-2-one |
| 151 | pyrazol-4-yl | H | methoxy | hexahydro-2H- |
| | | | | cyclopenta[d]oxazol- |
| | | | | 4′,4′-diyl-2-one |
| 170 | pyrazol-4-yl | H | methoxy | 3-methoxy-cyclopent- |
| | | | | 1′1′-diyl |
| 173 | pyrazol-4-yl | R-methyl | methoxy | 3-hydroxy-cyclopent- |
| | | | | 1′1′-diyl |

In certain embodiments, the present invention is directed to one or more compounds of formula (I) selected from the group consisting of
2-[(3-methoxyphenyl)methyl]-6-(1H-pyrazol-4-yl)isoindolin-1-one;
2-[(1R)-1-(3-methoxyphenyl)methyl]-6-(1H-pyrazol-4-yl)isoindolin-1-one;
N-isopropyl-3-[[1-oxo-6-(1H-pyrazol-4-yl)isoindolin-2-yl]methyl]benzamide;
2-[(1R)-1-(3-ethoxyphenyl)ethyl]-6-(1H-pyrazol-4-yl)isoindolin-1-one;
7-fluoro-2-[(1R)-1-(3-methoxyphenyl)ethyl]-6-(1H-pyrazol-4-yl)isoindolin-1-one;
5-fluoro-2-[(3-methoxyphenyl)methyl]-6-(1H-pyrazol-4-yl)isoindolin-1-one;
2′-[(3-methoxyphenyl)methyl]-6′-(1H-pyrazol-4-yl)spiro[cyclopentane-1,3′-isoindoline]-1′-one;
2-[(1S*)-2-hydroxy-1-(3-methoxyphenyl)ethyl]-6-(1H-pyrazol-4-yl)isoindolin-1-one;
2-[(1R)-1-(3-methoxyphenyl)ethyl]-6-(1H-pyrazol-4-yl)-4-pyrimidin-5-yl-isoindolin-1-one;
2-[(1R)-1-(3-methoxyphenyl)ethyl]-4-piperazin-1-yl-6-(1H-pyrazol-4-yl)isoindolin-1-one;

and pharmaceutically acceptable salts thereof.

### Definitions

As used herein, unless otherwise noted, “halogen” shall mean chloro, bromo, fluoro and iodo, preferably bromo, fluoro or chloro.

As used herein, unless otherwise noted, the term “oxo” shall mean s functional group of the structure ═O (i.e. a substituent oxygen atom connected to another atom by a double bond).

As used herein, unless otherwise noted, the term “C_{X-Y}alkyl” wherein X and Y are integers, whether used alone or as part of a substituent group, include straight and branched chains containing between X and Y carbon atoms. For example, C₁₋₄alkyl radicals include straight and branched chains of between 1 and 4 carbon atoms, including methyl, ethyl, propyl, isopropyl, butyl, isobutyl, sec-butyl and t-butyl.

One skilled in the art will recognize that the terms “—(C_{X-Y}alkyl)- and —C_{X-Y}alkyl-” wherein X and Y are integers, shall denote any C_{X-Y}alkyl carbon chain as herein defined, wherein said C_{X-Y}alkyl chain is divalent and is further bound through two points of attachment, preferably through two terminal carbon atoms.

As used herein, unless otherwise noted, the term “fluorinated C_{X-Y}alkyl” shall mean any C_{X-Y}alkyl group as defined above substituted with at least one fluorine atom, preferably one to three fluorine atoms. In an example, “fluorinated C₁₋₄alkyl” include, but are not limited, to —CH₂F, —CF₂H, —CF₃, —CH₂—CF₃, —CF₂—CF₂—CF₂—CF₃, and the like.

As used herein, unless otherwise noted, the term “hydroxy substituted C_{X-Y}alkyl” shall mean C_{X-Y}alkyl group as defined above substituted with at least one hydroxy group. Preferably, the C_{X-Y}alkyl group is substituted with at least one, preferably one to three, more preferably one to two, more preferably, one hydroxy group. Preferably, the C_{X-Y}alkyl group is substituted with a hydroxy group at a terminal carbon. For example, hydroxy substituted C₁₋₄alkyl include, but are not limited to, —CH₂(OH), —CH₂—CH₂(OH), —CH(OH)—CH₃, —CH(OH)—CH₂(OH), —CH₂—CH(OH)—CH₂, —CH₂—CH₂—CH₂—CH₂(OH), —C(CH₃)₂(CH₂OH), and the like.

As used herein, unless otherwise noted, “C_{X-Y}alkoxy” wherein X and Y are integers, shall mean an oxygen ether radical of the above described straight or branched chain C_{X-Y}alkyl groups containing between X and Y carbon atoms. For example, C₁₋₄alkoxy shall include methoxy, ethoxy, n-propoxy, isopropoxy, n-butyloxy, iso-butyloxy, sec-butyloxy and tert-butyloxy.

As used herein, unless otherwise noted, the term “fluorinated C_{X-Y}alkoxy” shall mean any C_{X-Y}alkoxy group as defined above substituted with at least one fluorine atom, preferably one to three fluorine atoms. For example, “fluorinated C₁₋₄alkoxy” include, but are not limited, —OCH₂F, —OCF₂H, —OCF₃, —OCH₂—CF₃, —OCF₂—CF₂—CF₂—CF₃, and the like.

As used herein, unless otherwise noted, the term “C_{X-Y}cycloalkyl”, wherein X and Y are integers, shall mean any stable X- to Y-membered monocyclic, bicyclic, polycyclic, bridged or spiro-cyclic saturated ring system, preferably a monocyclic, bicyclic, bridged or spiro-cyclic saturated ring system. For example, the term “C₃₋₈cycloalkyl” includes, but is not limited to cyclopropyl, cyclobutyl, cyclopentyl, cyclohexyl, cycloheptyl, bicyclo[2.2.1]hept-2-yl, cyclooctyl, bicyclo[2.2.2]octan-2-yl, and the like.

As used herein, unless otherwise noted, “5 to 10 membered heteroaryl” denotes any five to ten membered, monocyclic, bicyclic, fused, bridged or spiro-cyclic aromatic ring structure containing at least one heteroatom selected from the group consisting of O, N and S, optionally containing one to four additional heteroatoms independently selected from the group consisting of O, N and S. The 5 to 10 membered heteroaryl group may be attached at any heteroatom or carbon atom of the ring such that the result is a stable structure.

Preferably, the 5 to 10 membered heteroaryl is any five or six membered monocyclic aromatic ring structure containing at least one heteroatom selected from the group consisting of O, N and S, optionally containing one to three additional heteroatoms independently selected from the group consisting of O, N and S; or any nine or ten membered bicyclic, fused, bridged or spiro-cyclic aromatic ring structure containing at least one heteroatom selected from the group consisting of O, N and S, optionally containing one to four additional heteroatoms independently selected from the group consisting of O, N and S.

Examples of suitable 5 to 10 membered heteroaryl groups include, but are not limited to, pyrrolyl, furyl, thienyl, oxazolyl, imidazolyl, pyrazolyl, isoxazolyl, isothiazolyl, triazolyl, oxadiazolyl, thiadiazolyl, pyridinyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, 1H-pyrrolo[2,3-b]pyridine, and the like.

As used herein, the term “5 to 10 membered heterocycloalkyl” denotes any five to ten membered saturated or partially unsaturated monocyclic ring structure or any saturated, partially unsaturated or partially aromatic bicyclic, fused, bridged or spiro-cyclic ring system containing at least one heteroatom selected from the group consisting of O, N and S, optionally containing one to four additional heteroatoms independently selected from the group consisting of O, N and S. The 5 to 10 membered heterocycloalkyl group may be attached at any heteroatom or carbon atom of the ring such that the result is a stable structure

Preferably, the 5 to 10 membered heterocycloalkyl is any five to eight membered monocyclic, saturated or partially unsaturated ring structure containing at least one heteroatom selected from the group consisting of O, N and S, optionally containing one to three additional heteroatoms independently selected from the group consisting of O, N and S; or a nine to ten membered saturated, partially unsaturated or partially aromatic bicyclic, fused, bridged or spiro-cyclic ring system containing at least one heteroatom selected from the group consisting of O, N and S, optionally containing one to four additional heteroatoms independently selected from the group consisting of O, N and S.

Examples of suitable 5 to 10 membered heterocycloalkyl groups include, but are not limited to, pyrrolinyl, pyrrolidinyl, dioxalanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 3,4-methylenedioxyphenyl, 2,3-dihydrobenzofuryland the like.

When a particular group is “substituted” (e.g. C_{X-Y}alkyl, C_{X-Y}alkoxy, C_{X-Y}cycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom).

For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (D₂O or ²H₂O), “super-heavy water” or tritiated water (T₂O or ³H₂O), where the hydrogen atoms are replaced with tritium (³H) isotopes, two heavy-oxygen water isotopologues (H₂¹⁸O and H₂¹⁷O) and isotopologues where the hydrogen and oxygen atoms may each independently be replaced by isotopes, for example the doubly labeled water isotopologue D₂¹⁸O.

It is intended that within the scope of the present invention, any one or more element(s), in particular when mentioned in relation to a compound of formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

In certain embodiments, isotopologues include “isotopomers” which shall mean isomers with isotopic atoms, having the same number of each isotope of each element but differing in their position. Isotopomers include both constitutional isomers and stereoisomers solely based on isotopic location. For example, CH₃CHDCH₃ and CH₃CH₂CH₂D are a pair of constitutional isotopomers of n-propane; whereas (R)—CH₃CHDOH and (S)—CH₃CHDOH or (Z)—CH₃CH═CHD and (E)-CH₃CH═CHD are examples of isotopic stereoisomers of ethanol and n-propene, respectively.

It is further intended that the present invention includes the compounds described herein, including all isomers thereof (including, but not limited to stereoisomers, enantiomers, diastereomers, tautomers, isotopologues, isotopomers, and the like).

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

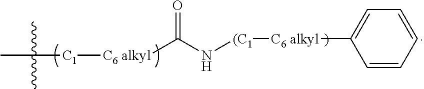

Abbreviations used in the specification, particularly the Schemes and Examples, are as listed in the Table A, below:

TABLE A
Abbreviations

| | |
|---|---|
| ACN = | Acetonitrile |
| AcOH = | Acetic Acid |
| ADP = | Adenosine Diphosphate |
| Alexa633 tracer = | Alexa Fluor ® 633 Hydrazide Tracer |
| | (ThermoFisher) |
| BSA = | Bovine Serum Albumin |
| ACN or MeCN = | Acetonitrile |
| ATP = | Adenosine Triphosphate |
| BF₃•Et₂O = | Boron trifluoride etherate |
| BINAP = | (2,2′-Bis(diphenylphosphino)-1,1′-binaphthyl) |
| Brij ™-35 = | Polyethylene glycol hexadecyl ether |
| t-BuOH = | tert-Butanol |
| DCM = | Dichloromethane |
| DIPEA or DIEA = | Diisopropylethylamine |
| DMF = | N,N-Dimethylformamide |
| DMSO = | Dimethylsulfoxide |
| dppf = | 1,1′-Bis(diphenylphosphino)ferrocene |
| DTT = | Dithiothietol |
| EtOAc = | Ethyl Acetate |
| EDTA = | Ethylenediaminetetracetic acid |
| eGFR = | Estimated Glomular Filtration Rate |
| EtOH = | Ethanol |
| Et₂O = | Diethyl ether |
| Et₃N or TEA = | Triethylamine |
| F12 medium = | Gibco ® F12 Nutrient Medium (ThermoFisher) |
| FBS = | Fetal Bovine Serum |
| G418 = | Geneticin ® (G418) Sulfate |
| GFR = | Glomular Filtration Rate |
| GLP-1 = | Glucagon-like peptide 1 |
| GRK2 = | G protein-coupled Receptor Kinase 2 |
| HATU = | (1-[Bis(dimethylamino)methylene-1H-1,2,3- |
| | triazolo[4,5-b]pyridinium 3-oxid |
| | hexafluorophosphate |
| HBSS = | GIBCO ® Hank's Balanced Salt Solution |
| HEPES = | 4-(2-Hydroxyethyl)-1-Piperizine Ethane Sulfonic |
| | Acid |
| HPLC = | High Pressure Liquid Chromatography |
| HTRF = | Homogeneous Time Resolved Fluorescence |
| IFG = | Impaired fasting glucose |
| IGT = | Impaired glucose tolerance |
| KOAc = | Potassium Acetate |
| KOt-Bu = | Potassium tert-Butoxide |
| LCMS or LC/MS = | Liquid chromatography-mass spectrometry |
| LDA = | Lithium diisopropylamide |
| LiHMDS = | Lithium bis(trimethylsilyl)amide |
| MeOH = | Methanol |
| mesyl or Ms = | Methylsulfonyl (i.e. —SO₂—CH₃) |
| mesylate or OMs = | Methanesulfonate (i.e. —O—SO₂—CH₃) |
| MOM = | Methoxy methyl |
| Ms or mesyl = | —SO₂—CH₃ |
| MsCl = | Mesyl Chloride (i.e. CH₃—SO₂—Cl) |
| NaBH(OAc)₃ = | Sodium triacetoxyborohydride |
| NAFLD = | Non-alcoholic fatty liver disease |
| NaOt-Bu = | Sodium tert-Butoxide |
| NASH = | Non-alcoholic steatohepatitis |
| NH(PMB)₃ = | tris(4-methoxybenzyl)-λ⁴-azane |
| NMO = | 4-Methylmorpholine N-oxide |
| NMP = | N-Methyl-2-pyrrolidone |
| NMR = | Nuclear Magnetic Resonance |
| OMs or mesylate = | Methanesulfonate (i.e. —O—SO₂—CH₃) |
| OTf or triflate = | Trifluoromethanesulfonyl (i.e. |
| | —O—SO═—CF₃) |
| OTs or tosylate = | p-Toluenesulfonate (i.e. —O—SO₂-(p- |
| | methylphenyl)) |
| Pd/C = | Palladium on Carbon (catalyst) |
| Pd(dba)₂ = | Bis(dibenzylideneacetone)dipalladium(0) |
| Pd(dppf)Cl₂ = | [1,1′-Bis(diphenylphosphino)ferrocene] Palladium |
| | (II) Dichloride |
| Pd(dppf)Cl•CHCl₃ = | [1,1′-Bis(diphenylphosphino) |
| | ferrocene]chloropalladium complex with |
| | chloroform |
| Pd(OAc)₂ = | Palladium II acetate |
| Pd(OH)₂ = | Palladium hydroxide |
| Pd(OH)₂/C = | Palladium hydroxide on carbon (Pearlman's |
| | Catalyst) |
| Pd(PPh₃)₄ = | Tetrakistriphenylphosphine palladium (0) |
| PMB = | 4-Methoxybenzyl ether |
| PPh₃ = | Triphenylphosphine |
| RuPhos = | 2-Dicyclohexylphosphino-2′,6′- |
| | diisopropoxybiphenyl |
| SAE = | Sepsis Associated Encephalopathy |
| SNS = | Sympathetic Nervous System |
| TBAB = | Tetra-n-butylammonium bromide |
| TBAF = | Tetra-n-butylammonium fluoride |
| TBAI = | Tetrabutylammonium iodide |
| TBSOTf = | Trifluoromethanesulfonic acid tert- |
| | butyldimethylsilylester |
| TEA or Et₃N = | Triethylamine |
| Tf or trifyl = | Trifluoromethylsulfonyl (i.e. —SO₂—CF₃) |
| TFA = | Trifluoroacetic Acid |
| THF = | Tetrahydrofuran |
| THP = | Tetrahydropyran |
| TLC = | Thin Layer Chromatography |
| TMS = | Trimethylsilyl |
| TMSN₃ = | Tris(trimethylsilyl)amine |
| Tosylate or OTs = | p-Toluenesulfonate (i.e. —O—SO₂-(p- |
| | methylphenyl)) |
| Ts or tosyl = | —SO₂-(p-methylphenyl) |
| Tween-20 ® = | Nonionic detergent (Sigma Aldrich) |

As used herein, unless otherwise noted, the term “isolated form” shall mean that the compound is present in a form which is separate from any solid mixture with another compound(s), solvent system or biological environment. In an embodiment of the present invention, the compound of formula (I) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s).

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, slow the progression of the disease or disorder, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

One skilled in the art will recognize that wherein a reaction step of the present invention may be carried out in a variety of solvents or solvent systems, said reaction step may also be carried out in a mixture of the suitable solvents or solvent systems.

One skilled in the art will further recognize that the reaction or process step(s) as herein described are allowed to proceed for a sufficient period of time until the reaction is complete, as determined by any method known to one skilled in the art, for example, chromatography (e.g. HPLC). In this context a “completed reaction or process step” shall mean that the reaction mixture contains a significantly diminished amount of the starting material(s)/reagent(s) and a significantly reduced amount of the desired product(s), as compared to the amounts of each present at the beginning of the reaction.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α-obs]/[α-max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### General Synthesis Schemes

Compounds of formula (I) of the present invention may be synthesized according to the general synthesis schemes and synthesis examples which follow hereinafter. The preparation of the starting materials used in the synthesis schemes and synthesis examples which follow hereinafter is well within the skill of persons versed in the art.

Compounds of formula (I) (for example, compounds of formula (I) wherein R⁶ and R⁷ are each hydrogen) may be prepared as described in Scheme 1, below.

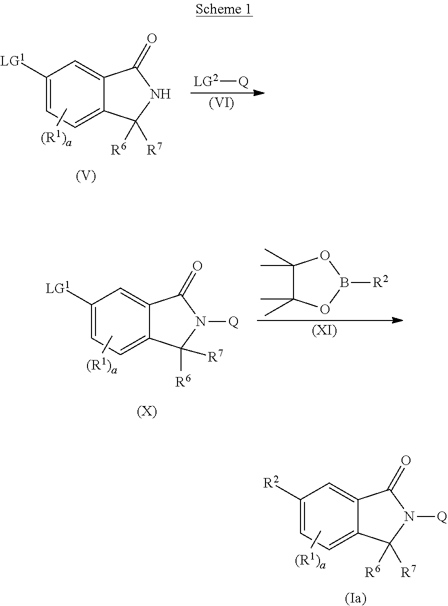

Accordingly, a suitably substituted compound of formula (V) wherein LG¹ is a suitably selected leaving group such as Br, Cl, OTf, and the like, a known compound or compound prepared by known methods; is reacted with a suitably substituted compound of formula (VI), wherein LG² is a suitably selected leaving group such as Br, OMs, OTs, and the like, a known compound or compound prepared by known methods; in the presence of a suitably selected acid such as NaH, KOt-Bu, LiN(Si(CH₃)₃)₂, and the like; in a suitably selected solvent such as THF, DMF, and the like; to yield the corresponding compound of formula (X).

The compound of formula (X) is reacted with a suitably substituted compound of formula (XI), a known compound or compound prepared by known methods; in the presence of a suitably selected catalyst such as Pd(PPH₃)₄, Pd(dppf)Cl₂, and the like; in a suitably selected solvent such as 1,4-dioxane, toluene, DMF, and the like; to yield the corresponding compound of formula (Ia).

Compounds of formula (I) wherein R⁶ and R⁷ are each hydrogen may alternatively be prepared as described in Scheme 2, below.

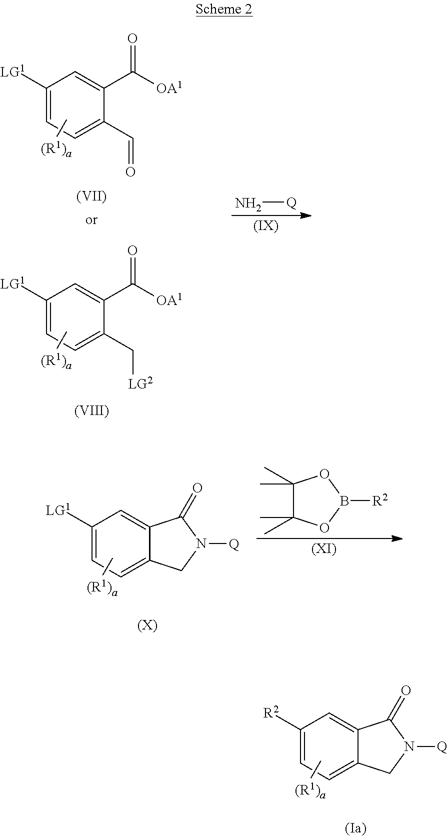

Accordingly, a suitably substituted compound of formula (VII), wherein LG¹ is a suitably selected leaving group such as Br, I, OTf, and the like, and wherein A¹ is a C₁₋₄alkyl, a known compound or compound prepared by known methods; is reacted with a suitably substituted compound of formula (IX), a known compound or compound prepared by known methods; in the presence of a suitably selected reducing agent such as NaBH(OAc)₃, NaBH₃CN, NaBH₄, and the like; in a suitably selected solvent such as DCM, methanol, and the like; to yield the corresponding compound of formula (X).

Alternatively, a suitably substituted compound of formula (VIII), wherein LG¹ is a suitably selected leaving group such as Br, I, OTf, and the like, wherein LG² is a second suitably selected leaving group such as Br, Cl, OTs, and the like, and wherein A¹ is a C₁₋₄alkyl, a known compound or compound prepared by known methods; is reacted with a suitably substituted compound of formula (IX), a known compound or compound prepared by known methods; in the presence of a suitably selected base such as TEA, DIPEA, pyridine, and the like; in a suitably selected solvent such as DCM, EtOAc, and the like; to yield the corresponding compound of formula (X).

The compound of formula (X) is reacted with a suitably substituted compound of formula (XI), a known compound or compound prepared by known methods; in the presence of a suitably selected coupling agent such as Pd(PPH₃)₄, Pd(dppf)Cl₂, Pd₂dba₃, and the like; in a suitably selected solvent such as DMF, 1,4-dioxane, THF, and the like; to yield the corresponding compound of formula (Ia).

Compounds of formula (X), wherein the R¹ substituent is bound at the 6-position of the isoinsolin-2-one may alternatively be prepared as described in Scheme 3, below.

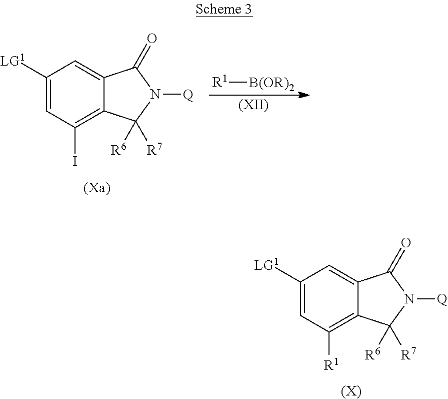

Accordingly, a suitably substituted compound of formula (Xa), a compound of formula (X) wherein R¹ is I, prepared for example as described in Scheme 1 or 2 above, is reacted with a suitably substituted compound of formula (XII), wherein both R groups are the same and are hydrogen or are taken together with the atoms to which they are bound to form

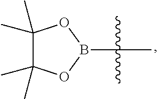 [SMILES: *B1OC(C)(C)C(C)(C)O1]

a known compound or compound prepared by known methods; in the presence of a suitably selected coupling agent such as Pd(PPH₃)₄, Pd(dppf)Cl₂, Pd(OAc)₂, and the like; in a suitably selected solvent such as 1,4-dioxane, THF, DMF, and the like; to yield the corresponding compound of formula (X).

Compounds of formula (I) wherein R² is 2,4-dihydro-3H-1,2,4-triazol-4-yl-3-one may alternatively be prepared as described in Scheme 4, below.

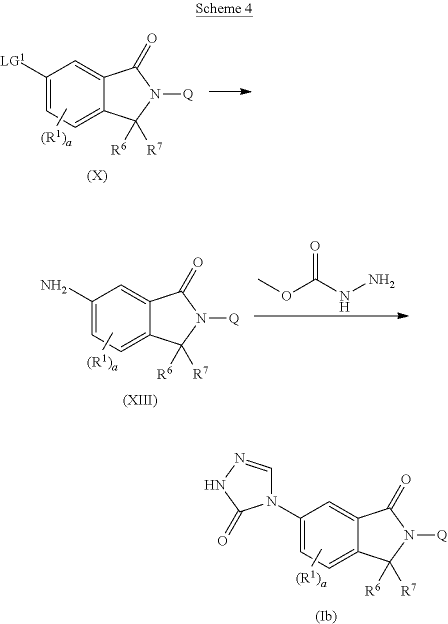

Accordingly, a suitably substituted compound of formula (X), prepared for example as described herein, is reacted with a suitably selected amino agent such as NH(PMB)₂, NH₃, NH₄OH, and the like; in the presence of a suitably selected coupling agent such as Pd(dba)₂, CuI, Cu₂O, and the like; in the presence of a suitably selected ligand such as BINAP, L-ascorbic acid, S-proline, and the like; in a suitably selected solvent such as DMSO, NMP, DMF, and the like; optionally followed by reaction with TFA or DDQ (when the amino reagent is for example NH(PMB)₂ this step removes the PMB protecting groups); to yield the corresponding compound of formula (XIII).

The compound of formula (XIII) is reacted with methyl hydrazinecarboxylate, a known compound; in the presence of a suitably selected agent such as trimethyl orthoformate, triethylorthoformate, and the like; in a suitably selected solvent such as MeOH, EtOH, and the like; to yield the corresponding compound of formula (Ib).

Compounds of formula (I) wherein one of R⁶ and R⁷ is other than hydrogen may be prepared as described in Scheme 5, below.

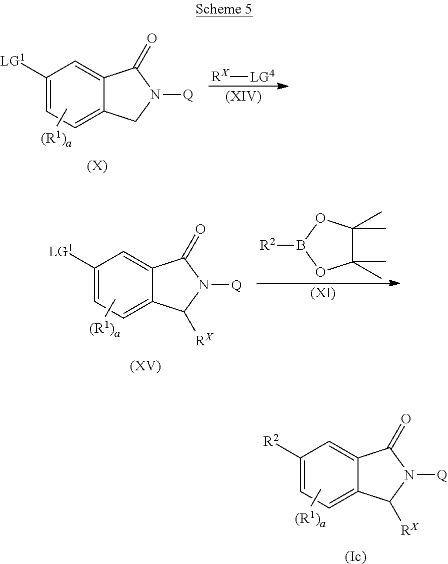

Accordingly, a suitably substituted compound of formula (X), wherein LG¹ is a suitably selected leaving group such as Br, Cl, I, and the like, prepared for example as described herein, is reacted with a suitably substituted compound of formula (XIV), wherein LG⁴ is a suitably selected leaving group such as Br, OMs, OTs, and the like, and wherein R^{X} is an R⁶ or R⁷ substituent as herein described; in the presence of a suitably selected acid such as NaH, LiN(Si(CH₃)₃)₂, NaOt-Bu, and the like; in a suitably selected solvent such as THF, Et₂O, DMF, and the like; to yield the corresponding compound of formula (XV).

The compound of formula (XV) is reacted with a suitably substituted compound of formula (XI), a known compound or compound prepared by known methods; in the presence of a suitably selected coupling agent such as Pd(PPH₃)₄, Pd(dppf)Cl₂, Pd₂(dba)₃, and the like; in a suitably selected solvent such as 1,4-dioxane, DMF, THF, and the like; to yield the corresponding compound of formula (Ic).

One skilled in the art will recognize that wherein R⁶ and R⁷ are the same and are selected from the group consisting of C₁₋₂alkyl and hydroxy substituted C₁₋₂alkyl, then the desired compound may be prepared as described in Scheme 5 above, reacting the compound of formula (X) with a suitably substituted compound of formula (XIV), wherein the compound of formula (XIV) is present in an amount greater than or equal to about 2 molar equivalents.

Compounds of formula (I) wherein R⁶ and R⁷ are taken together with the carbon atom to which they are attached to form cyclopent-1′1′-diyl or cyclopent-3-en-1′1′-diyl may be prepared as described in Scheme 6, below.

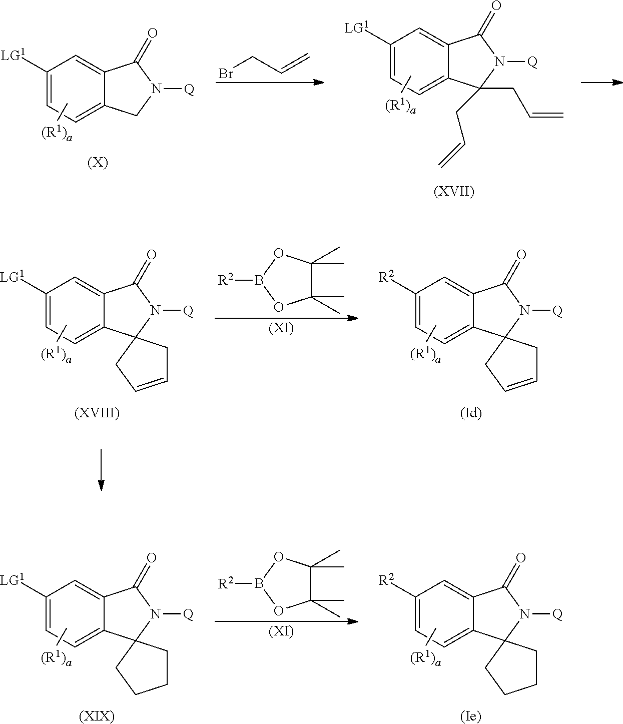

Accordingly, a suitably substituted compound of formula (X), Accordingly, a suitably substituted compound of formula (X), wherein LG¹ is a suitably selected leaving group such as Br, I, and the like, prepared for example as described herein, is reacted with 3-bromoprop-1-ene, a known compound or compound prepared by known methods, wherein the 3-bromoprop-1-ene is preferably present in an amount of greater than or equal to about 2 molar equivalents (relative to the moles of the compound of formula (X)); in the presence of a suitably selected acid such as NaH, LiHMS, LDA, and the like; in a suitably selected solvent such as THF, DMF, 1,4-dioxane, and the like; to yield the corresponding compound of formula (XVII).

The compound of formula (XVII) is reacted with, for example, Grubb's II catalyst; in a suitably selected solvent such as CH₂Cl₂, and the like; to yield the corresponding compound of formula (XVIII).

The compound of formula (XVIII) is reacted with a suitably substituted compound of formula (IX), a known compound or compound prepared by known methods, in the presence of a suitably selected coupling agent such as Pd(PPH₃)₄, Pd(dppf)Cl₂, and the like; in a suitably selected solvent such as dioxane, DMF, and the like; to yield the corresponding compound of formula (Id), wherein R⁶ and R⁷ are taken together with the carbon atom to which they are bound to form cyclopent-3-en-1′1′-diyl.

Alternatively, the compound of formula (XVIII) is reacted with H_{2(g)} in the presence of a suitably selected catalyst such as Pd/C, and the like; in a suitably selected solvent such as MeOH, EtOH, and the like; to yield the corresponding compound of formula (XIX).

The compound of formula (XIX) is reacted with a suitably substituted compound of formula (IX), a known compound or compound prepared by known methods, in the presence of a suitably selected coupling agent such as Pd(PPH₃)₄, Pd(dppf)Cl₂, and the like; in a suitably selected solvent such as 1,4-dioxane, DMF, and the like; to yield the corresponding compound of formula (Ie), wherein R⁶ and R⁷ are taken together with the carbon atom to which they are bound to form cyclopent-1′1′-diyl.

Compounds of formula (I) wherein R⁶ and R⁷ are taken together with the carbon atom to which they are attached to form an optionally 3-substituted cyclopent-1′1′-diyl may be prepared as described in Scheme 7, below.

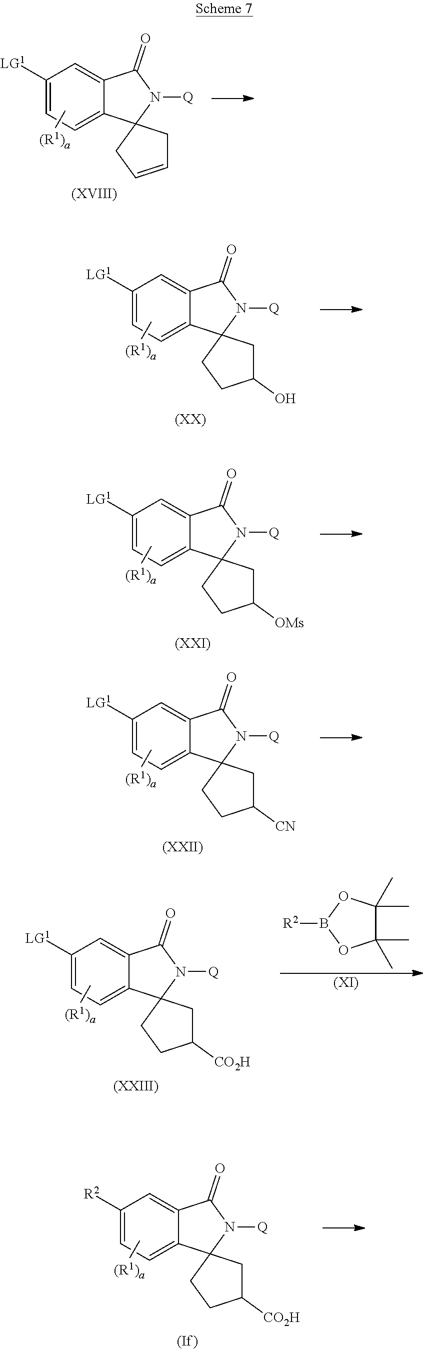

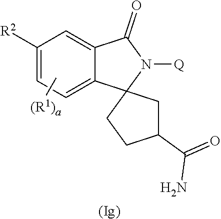

Accordingly, a suitably substituted compound of formula (XVIII), prepared for example as described in Scheme 6 above, is reacted with, for example, borane in the presence of peroxide; in a suitably selected solvent such as THF, 1,4-dioxane, and the like; to yield the corresponding compound of formula (XX).

The compound of formula (XX) is reacted with mesylchloride, a known compound; in the presence of a suitably selected organic amine base such as TEA, DIPEA, pyridine, and the like; in a suitably selected solvent such as CH₂Cl₂, THF, DMF, and the like; to yield the corresponding compound of formula (XXI).

The compound of formula (XXI) is reacted with a suitably selected source of CN such as KCN, NaCN, and the like; in a suitably selected solvent such as DMF, and the like; to yield the corresponding compound of formula (XXII).

The compound of formula (XXII) is reacted with a suitably selected source of acid such as HCl, and the like; in a suitably selected solvent such as water, and the like; to yield the corresponding compound of formula (XXIII).

The compound of formula (XXIII) is reacted with a suitably substituted compound of formula (IX), a known compound or compound prepared by known methods, in the presence of a suitably selected coupling agent such as Pd(PPH₃)₄, Pd(dppf)Cl₂, and the like; in a suitably selected solvent such as THF, 1,4-dioxane, and the like; to yield the corresponding compound of formula (If), wherein R⁶ and R⁷ are taken together with the carbon atom to which they are bound to form 3-hydroxy-cyclopent-1′1′-diyl.

The compound of formula (If) is further, optionally reacted with a suitably selected source of amine such as NH₄Cl, and the like; in the presence of a suitably selected coupling agent such as HATU, and the like; in a suitably selected solvent such as DMF, DCM, and the like; to yield the corresponding compound of formula (Ig), wherein R⁶ and R⁷ are taken together with the carbon atom to which they are bound to form 3-aminocarbonyl-cyclopent-1′1′-diyl.

One skilled in the art will recognize that any of the compounds of formula (XX), formula (XXI), formula (XXII) or formula (XXIII) may be reacted with a suitably substituted compound of formula (IX), a known compound or compound prepared by known methods, in the presence of a suitably selected coupling agent such as Pd(PPH₃)₄, Pd(dppf)Cl₂, and the like; in a suitably selected solvent such as THF, 1,4-dioxane, DMF, and the like; to yield the corresponding compound of formula (I), wherein R⁶ and R⁷ are taken together with the carbon atom to which they are bound to form the corresponding 3-substituted-cyclopent-1′1′-diyl.

Compounds of formula (I) wherein R⁶ and R⁷ are taken together with the carbon atom to which they are bound to form the corresponding 3-substituted, preferably 3-NR^{X}R^{Y}-substituted cyclopent-1′1′-diyl may be prepared as described in Scheme 8, below.

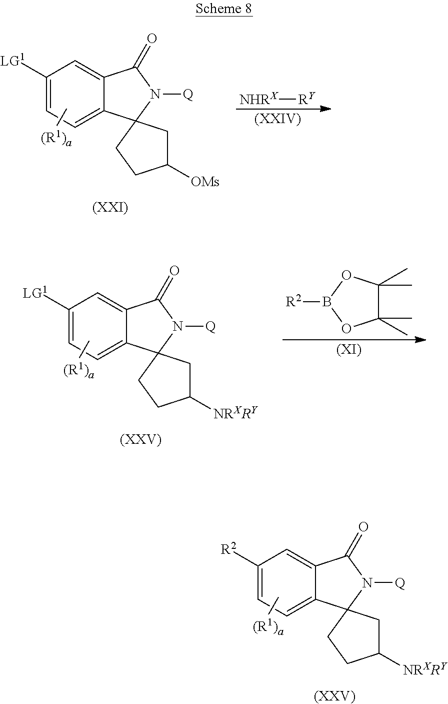

Accordingly, a suitably substituted compound of formula (XXI), prepared for example, as described in Scheme 7 above, is reacted with a suitably substituted amine of formula (XXIV), a known compound or compound prepared by known methods; in the presence of a suitably selected amine such as dimethylamine, diethylamine, and the like; in a suitably selected solvent such as DMF, and the like; to yield the corresponding compound of formula (XXV).

The compound of formula (XXV) is reacted with a suitably substituted compound of formula (IX), a known compound or compound prepared by known methods, in the presence of a suitably selected coupling agent such as Pd(PPH₃)₄, Pd(dppf)Cl₂, and the like; in a suitably selected solvent such as 1,4-dioxane, DMF, and the like; to yield the corresponding compound of formula (I), wherein R⁶ and R⁷ are taken together with the carbon atom to which they are bound to form the corresponding 3-NR^{X}R^{Y}-cyclopent-1′1′-diyl.

Compounds of formula (I) wherein R⁶ and R⁷ are taken together with the carbon atom to which they are bound to form hexahydro-2H-cyclopenta[d]oxazol-4′,4′-diyl-2-one may be prepared as described in Scheme 9, below.

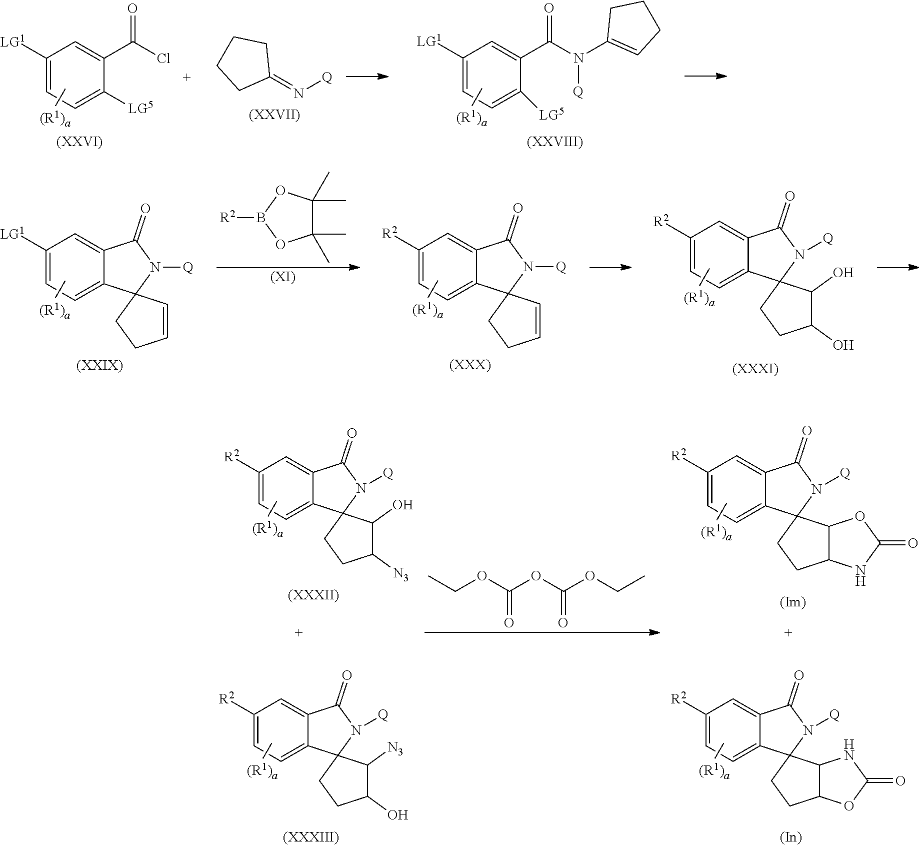

Accordingly, a suitably substituted compound of formula (XXVI), wherein LG¹ is a suitably selected leaving group such as Br, and the like, and wherein LG⁵ is a second suitably selected leaving group such as I, and the like, wherein LG¹ and LG⁵ are selected such that 5-bromo-2-iodobenzoyl chloride, a known compound or compound prepared by known methods, is reacted with a suitably selected compound of formula (XXVII), a known compound or compound prepared by known methods; in the presence of a suitably selected organic base such as TEA, DIPEA, pyridine, and the like; in a suitably selected solvent such as DCM, 1,4-dioxane, and the like; to yield the corresponding compound of formula (XXVIII).

The compound of formula (XXVIII) is reacted with a suitably selected ring closure reagent or reagent system such as Pd(OAc)₂ in the presence of PPH₃, TBAB and K₂CO₃; to yield the corresponding compound of formula (XXIX).

The compound of formula (XXIX) is reacted with a suitably substituted compound of formula (IX), a known compound or compound prepared by known methods; in the presence of a suitably selected coupling agent such as Pd(PPH₃)₄, Pd(dppf)Cl₂, and the like; in a suitably selected solvent such as dioxane, and the like; to yield the corresponding compound of formula (XXX).

The compound of formula (XXX) is reacted with oxone, a known compound; in the presence of a suitably selected alkene in a suitably selected solvent such as acetone, and the like; to yield the corresponding compound of formula (XXXI).

The compound of formula (XXXI) is reacted with a suitably selected source of azide such as NaN₃, TMSN₃, and the like; in the presence of a suitably selected solvent such as DMF, and the like; to yield a mixture of the corresponding compound of formula (XXXII) and the corresponding compound of formula (XXXIII).

The mixture of the compound of formula (XXXII) and the compound of formula (XXXIII) is reacted diethyl dicarbonate, a known compound; in the presence of H₂ gas; in the presence of a suitably selected catalyst such as Pd(OH)₂, Pd/C, and the like; in a suitably selected solvent such as EtOH, and the like;

and then reacted with a suitably selected alkyloxide such as NaOCH₃, and the like; in a suitably selected solvent such as MeOH, and the like; to yield a mixture of the corresponding compound of formula (Im) (where R⁶ and R⁷ are taken together with to form hexahydro-2H-cyclopenta[d]oxazol-6,6-diyl-2-one) and the corresponding compound of formula (In) (where R⁶ and R⁷ are taken together with to form hexahydro-2H-cyclopenta[d]oxazol-4′,4′-diyl-2-one).

Compounds of formula (I) wherein R⁶ and R⁷ are taken together with the carbon atom to which they are bound to form 2-substituted cyclopent-1′1′-diyl may be prepared as described in Scheme 9, below (see also GRIGG, R., et al., Tetrahedron, 1990, pp 4003-4018, Vol. 46).

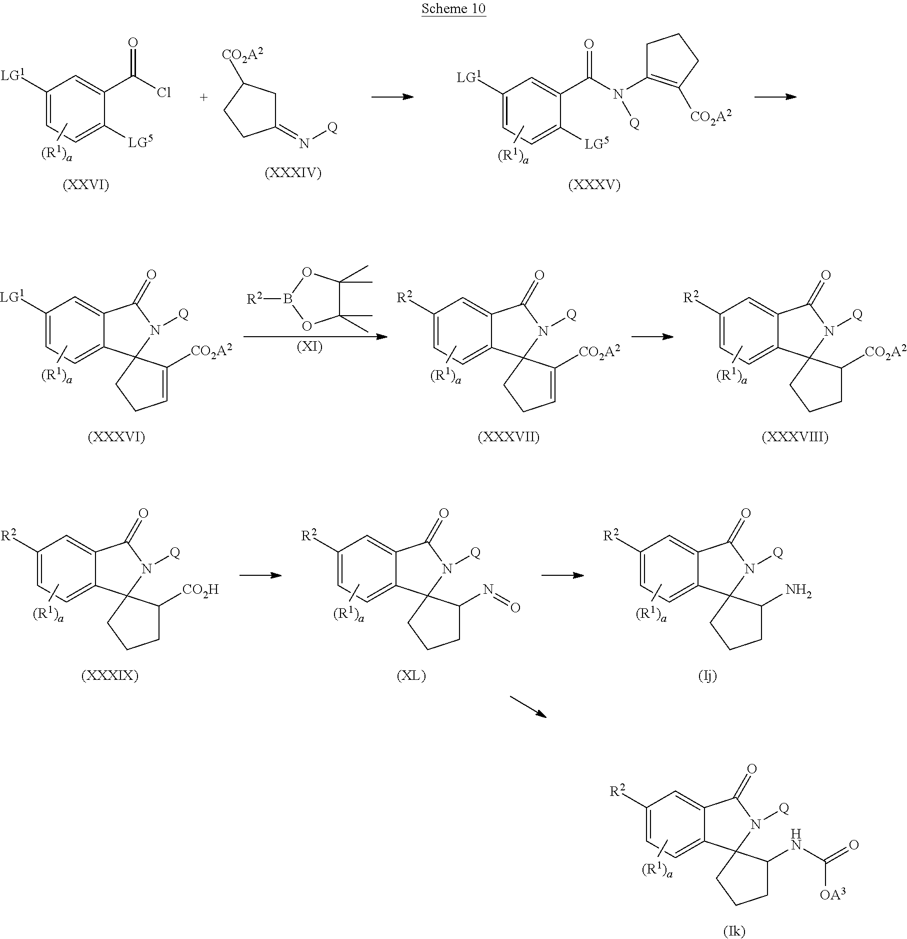

Accordingly, a suitably substituted compound of formula (XXVI), wherein LG¹ is a suitably selected leaving group such as Br, I, and the like, and wherein LG⁵ is a second suitably selected leaving group such as I, and the like, wherein LG¹ and LG⁵ are selected such that 5-bromo-2-iodobenzoyl chloride, a known compound or compound prepared by known methods, is reacted with a suitably selected compound of formula (XXXIV), wherein A² is C₁₋₄alkyl, a known compound or compound prepared by known methods; in the presence of a suitably selected organic base such as TEA, DIPEA, pyridine, and the like; in a suitably selected solvent such as DCM, 1,4-dioxane, and the like; to yield the corresponding compound of formula (XXXV).

The compound of formula (XXXV) is reacted with a suitably selected ring closure reagent or reagent system such as Pd(OAc)₂ in the presence of PPH₃, TBAB and K₂CO₃, to yield the corresponding compound of formula (XXXVI).

The compound of formula (XXXVI) is reacted with a suitably substituted compound of formula (IX), a known compound or compound prepared by known methods; in the presence of a suitably selected coupling agent such as Pd(PPH₃)₄, Pd(dppf)Cl₂, and the like; in a suitably selected solvent such as dioxane, and the like; to yield the corresponding compound of formula (XXXVII).

The compound of formula (XXXVII) is reacted with a suitably selected reducing agent such as hydrogen gas; in the presence of a suitably selected catalyst such as Pd/C, and the like; in a suitably selected solvent such as MeOH and the like; to yield the corresponding compound of formula (XXXVIII).

The compound of formula (XXXVIII) is reacted with a suitably selected base such as LiOH, and the like; in a suitably selected solvent such as THF/MeOH/water, and the like; to yield the corresponding compound of formula (XXXIX).

The compound of formula (XXXIX) is reacted with N₃PO(OPhenyl)₂, a known compound; in the presence of a suitably selected carboxylic acid in a suitably selected solvent such as toluene, and the like; to yield the corresponding compound of formula (XL).

The compound of formula (XL) is reacted according to known methods, to convert the —N═O substituent to the corresponding amine compound of formula (Ij) (by reacting with water) or the corresponding alkoxy-amide compound of formula (Ik) (by reacting with a suitably selected alcohol such as MeOH, t-BuOH, EtOH, and the like).

Compounds of formula (I) wherein R⁶ and R⁷ are taken together with the carbon atom to which they are bound to form a ring structure (for example, optionally substituted cyclohex-1′1′-diyl, cyclohex-2-en-1′1′-diyl, cyclohex-3-en-1′1′-diyl, piperidin-4′,4′-diyl, tetrahydro-furan-3,3-diyl, tetrahydro-pyran-4′,4′-diyl, or 2,3-dihydro-inden-1′1′-diyl) may be similarly prepared as described in Scheme 10 above, by reacting the compound of formula (XXVI) with a suitably substituted compound of formula (XLI)

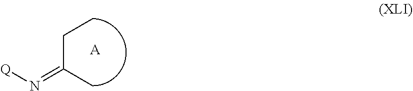

wherein the A ring represents R⁶ and R⁷ taken together with the carbon atom to which they are bound, a known compound or compound prepared by known methods (substituting the compound of formula (XLI) for the compound of formula (XXXIV)) to effect coupling of the compound of formula (XXVI) with the compound of formula (XLI); in the presence of a suitably selected organic base such as TEA, DIPEA, pyridine, and the like; in a suitably selected solvent such as DCM, 1,4-dioxane, and the like) to yield the intermediate compound of formula (XLII)

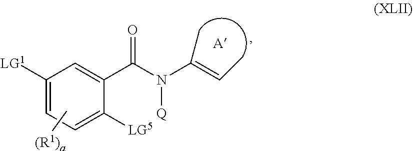

wherein A′ represents the corresponding unsaturated A ring structure. The intermediate compound of formula (XLII) is then reacted as described in Scheme 9 to effect ring closure of the isoindolin-2-one (reacting with a ring closure reagent or reagent system such as Pd(OAc)₂ in the presence of PPH₃, TBAB and K₂CO₃), followed by substitution with the desired R² group (by reacting with a suitably substituted compound of formula (XI) in the presence of a suitably selected coupling agent such as Pd(PPH₃)₄, Pd(dppf)Cl₂, and the like; in a suitably selected solvent such as dioxane, and the like), to yield the corresponding compound of formula (XLIII)

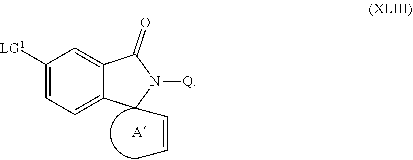

The compound of formula (XLIII) is then reacted with for example, hydrogen gas; in the presence of a catalyst such as Pd/C; followed by the reaction with an acid such as TFA, to yield the corresponding compound of formula (Ip)

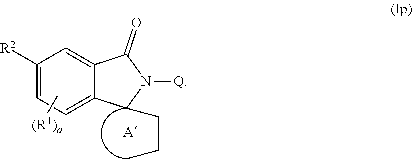

which may be further de-protected or functionalizes, as needed or desired, as would be recognized by those skilled in the art.

Compounds of formula (IX)

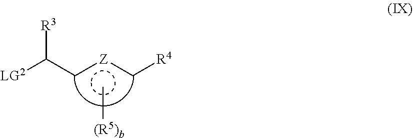

are known compounds or compounds which may be prepared according to known methods. For example, compounds of formula (IX) may be prepared as described in SPRENGELER, P. A., et al., PCT Publication WO 2017/075394 A1, published 4 May 2017, and/or AHMED, S., et al., PCT Publication WO 2007/029076 A1, published 15 Mar. 2007.

### Pharmaceutical Compositions

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus, for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.05 mg/day to about 300 mg/day, or any amount or range therein, preferably from about 0.1 mg/day to about 100 mg/day, or any amount or range therein, preferably from about 1 mg/day to about 50 mg/day, or any amount or range therein. The dosages, however, may be varied depending upon the requirement of the patients, the severity of the condition being treated, and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form yielding the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders mediated by GRK2 activity, described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein, preferably from about 0.05 mg to about 300 mg of the compound, or any amount or range therein, more preferably from about 0.1 mg to about 100 mg of the compound, or any amount or range therein, more preferably from about 0.1 mg to about 50 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methylcellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders mediated by GRK2 activity, is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug may be ordinarily supplied at a dosage level of from about 0.005 mg/kg to about 10 mg/kg of body weight per day, or any amount or range therein. Preferably, the range is from about 0.01 to about 5.0 mg/kg of body weight per day, or any amount or range therein, more preferably, from about 0.1 to about 1.0 mg/kg of body weight per day, or any amount or range therein, more preferably, from about 0.1 to about 0.5 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### 2-(3-methoxybenzyl)-6-(1H-pyrazol-4-yl)isoindolin-1-one

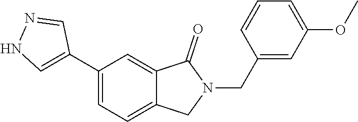 [SMILES: COc1cccc(CN2Cc3ccc(-c4cn[nH]c4)cc3C2=O)c1]

### Step 1: 6-Bromo-2-(3-methoxybenzyl)isoindolin-1-one

A mixture of methyl 5-bromo-2-formylbenzoate (872 mg, 3.59 mmol), (3-methoxyphenyl)methanamine (738 mg, 5.38 mmol) and NaBH(OAc)₃ (1065 mg, 5.02 mmol) in CH₂Cl₂ (20 mL) was stirred at 25° C. for 4 h. The reaction was quenched with 3N NaOH solution and the mixture was extracted with CH₂Cl₂ (3×25 mL). The CH₂Cl₂ extract was dried (Na₂SO₄), filtered and concentrated in vacuo to yield a light brown oil. Purification of the resulting residue by silica gel column chromatography (12 g, 0-20% EtOAc/heptane) yielded 6-bromo-2-(3-methoxybenzyl)isoindolin-1-one as a white solid. LCMS: Calculated: for C16H14BrNO2: 332, Measured: 332 [M]+.

### Alternate Step 1: 6-bromo-2-(3-methoxybenzyl)isoindolin-1-one

To a solution of 6-bromoisoindolin-1-one (10 g, 0.047 mol) in DMF (100 mL), THE (100 mL) was added NaH (60%, 2.264 g, 0.057 mol) at 0° C., and the resulting mixture stirred for 30 min at 25° C. TBAI (3.484 g, 0.009 mol) and 1-(bromomethyl)-3-methoxybenzene (11.378 g, 0.057 mol) were then added at 0° C. The resulting mixture was stirred at 25° C. overnight. The reaction was quenched with H₂O (800 mL). The resulting mixture was extracted with EtOAc (3×200 mL). The organic layers were combined, dried (Na₂SO₄), filtered and concentrated in vacuo. The residue was purified by silica gel chromatography (0-100% EtOAc/heptane) to yield 6-bromo-2-(3-methoxybenzyl)isoindolin-1-one as a yellow solid. LCMS Calculated: for C16H14BrNO2: 332, Measured: 334 [M+2H]+.

### Step 2: 6-Bromo-2-(3-hydroxybenzyl)isoindolin-1-one

A solution of 6-bromo-2-(3-methoxybenzyl)isoindolin-1-one (1 g, 0.003 mol) in DCM (30 mL) was treated with boron tribromide (1.51 g, 6.0 mmol) at room temperature under nitrogen atmosphere. After 2 h, the resulting mixture was quenched with H₂O, diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded 6-bromo-2-(3-hydroxybenzyl)isoindolin-1-one as a yellow solid.

### Step 3: 2-(3-Hydroxybenzyl)-6-(1-(tetrahydro-2H-pyran-2-yl)-1H-pyrazol-4-yl)isoindolin-1-one

Under a nitrogen atmosphere, a mixture of 6-bromo-2-(3-hydroxybenzyl)isoindolin-1-one (0.6 g, 1.89 mmol), 1-(tetrahydro-2H-pyran-2-yl)-4-(4,4,5,5-tetramethyl-1,3,2-dioxaborolan-2-yl)-1H-pyrazole (1.05 g, 3.77 mmol) and Pd(PPh₃)₄ (0.109 g, 0.094 mmol) in DMF (15 mL) was added a solution of K₂CO₃ (0.781 g, 5.66 mmol) in water (3 mL). The resulting solution was warmed to 100° C. After 12 h, the reaction mixture was allowed to cool to room temperature, diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded 2-(3-hydroxybenzyl)-6-(1-(tetrahydro-2H-pyran-2-yl)-1H-pyrazol-4-yl)isoindolin-1-one as a white solid. LCMS: Calculated: for C23H23N3O3: 389.45, Measured: 390.1 [M+H]+.

### Step 4: Methyl 3-((6-bromo-1-oxoisoindolin-2-yl)methyl)benzoate

Methyl 3-((6-bromo-1-oxoisoindolin-2-yl)methyl)benzoate was prepared by analogous procedure to that described above, substituting methyl 3-(bromomethyl)benzoate in place of 1-(bromomethyl)-3-methoxybenzene. LCMS: Calculated: for C17H14BrNO3: 360, Measured: 361 [M+H]+.

### Step 5: 2-(3-Methoxybenzyl)-6-(1H-pyrazol-4-yl)isoindolin-1-one

Under a nitrogen atmosphere, to a mixture of -bromo-2-(3-methoxybenzyl)isoindolin-1-one (242 mg, 0.73 mmol), tert-butyl 4-(4,4,5,5-tetramethyl-1,3,2-dioxaborolan-2-yl)-1H-pyrazole-1-carboxylate (642.8 mg, 2.185 mmol) and Pd(PPh₃)₄ (42 mg, 0.036 mmol) in DMF (15 mL) was added a solution of K₂CO₃ (302 mg, 2.185 mmol) in water (2 mL). The resulting solution was warmed to 110° C. After 12 h, the reaction mixture was allowed to cool to room temperature, diluted with EtOAc (50 mL), washed with brine (3×15 mL), dried (Na₂SO₄), and concentrated in vacuo. Purification of the resulting residue by silica gel chromatography (24 g column, 0-100% EtOAc/Heptane) yielded a second residue which was re-crystallized from methanol to yield 2-(3-methoxybenzyl)-6-(1H-pyrazol-4-yl)isoindolin-1-one as a white solid.

¹H NMR (300 MHz, METHANOL-d4) 8.35 (bs, 1H), 8.05 (bs, 1H), 7.96 (s, 1H), 7.85 (d, J=1.8 Hz, 1H), 7.68-7.62 (m, 1H), 7.56-7.52 (m, 1H), 7.48 (d, J=7.9 Hz, 1H), 7.28 (t, J=7.8 Hz, 1H), 6.94-6.82 (m, 2H), 4.76 (s, 2H), 4.35 (s, 2H), 3.75 (s, 3H); LCMS: Calculated: for C19H17N3O2: 319, Measured: 320 [M+H]+.

### Example 2

### (R)-2-(1-(3-methoxyphenyl)ethyl)-6-(1H-pyrazol-4-yl)isoindolin-1-one

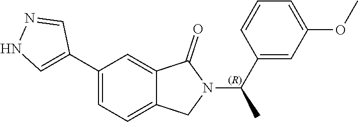

### Step 1: (R)-6-bromo-2-(1-(3-methoxyphenyl)ethyl)isoindolin-1-one

Methyl 5-bromo-2-(bromomethyl)benzoate (0.927 g, 3.01 mmol) was dissolved in acetonitrile (10 mL), then N,N-diisopropylethylamine (0.648 g, 5.02 mmol) and (R)-1-(3-methoxyphenyl)ethylamine (0.379 g, 2.51 mmol) were added at 25° C. The resulting mixture was warmed to 80° C. After 16 h, the reaction mixture was diluted with 1N HCl (15 mL) and the organic layer was separated. The aqueous layer was extracted with EtOAc (3×25 mL). The combined organic layer was washed with brine (1×50 mL), dried (Na₂SO₄), filtered and concentrated in vacuo. Purification of the resulting residue by silica gel column chromatography (80 g, 0-100% EtOAc/heptane) yielded (R)-6-bromo-2-(1-(3-methoxyphenyl)ethyl)isoindolin-1-one as a light yellow solid. LCMS: Calculated: for C17H16BrNO2: 346, Measured: 347 [M+H]+.

### Step 2: (R)-2-(1-(3-methoxyphenyl)ethyl)-6-(1H-pyrazol-4-yl)isoindolin-1-one

(R)-2-(1-(3-methoxyphenyl)ethyl)-6-(1H-pyrazol-4-yl)isoindolin-1-one was prepared according to the procedure described in Example 1, Step 5, substituting (R)-6-bromo-2-(1-(3-methoxyphenyl)ethyl)isoindolin-1-one in place of 6-bromo-2-(3-methoxybenzyl)isoindolin-1-one.

¹H NMR (400 MHz, DMSO-d6) δ 13.01 (s, 1H), 8.34 (s, 1H), 8.03 (s, 1H), 7.93 (d, J=1.8 Hz, 1H), 7.84 (m, 1H), 7.52 (d, J=7.9 Hz, 1H), 7.28 (t, J=7.8 Hz, 1H), 6.94-6.82 (m, 3H), 5.52 (m, 1H), 4.51 (d, J=17.7 Hz, 1H), 4.10 (d, J=17.6 Hz, 1H), 3.35 (m, 1H), 1.63 (d, J=7.2 Hz, 3H); LCMS: Calculated: for C20H19N3O2: 333.384, Measured: MH+: 334.1 [M+H]+.

The following compounds were similarly prepared according to the procedures described herein, selecting and substitution suitable reagents and starting materials as would be readily recognized by those skilled in the art.

### Example 3

### 2-(3-ethoxybenzyl)-6-(1H-pyrazol-4-yl)isoindolin-1-one

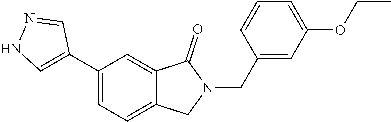 [SMILES: CCOc1cccc(CN2Cc3ccc(-c4cn[nH]c4)cc3C2=O)c1]

¹H NMR (400 MHz, CHLOROFORM-d) Shift 7.91-8.08 (m, 3H), 7.60-7.79 (m, 1H), 7.40 (d, J=7.58 Hz, 1H), 6.77-6.95 (m, 2H), 4.79 (s, 2H), 4.29 (s, 2H), 4.00 (q, J=7.07 Hz, 2H), 1.39 (t, J=6.82 Hz, 3H); LCMS: Calculated: for C20H19N3O2: 333, Measured: 334 [M+H]+.

### Example 4

### 2-(3-Isopropoxybenzyl)-6-(1H-pyrazol-4-yl)isoindolin-1-one

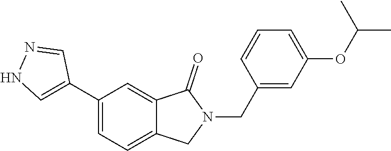 [SMILES: CC(C)Oc1cccc(CN2Cc3ccc(-c4cn[nH]c4)cc3C2=O)c1]

¹H NMR (400 MHz, METHANOL-d4) δ 8.12 (br s, 2H), 7.98 (d, J=1.01 Hz, 1H), 7.83 (dd, J=1.52, 8.08 Hz, 1H), 7.52 (d, J=8.08 Hz, 1H), 7.24 (t, J=7.74 Hz, 1H), 6.81-6.87 (m, 3H), 4.77 (s, 2H), 4.57 (td, J=6.06, 12.13 Hz, 1H), 4.39 (s, 2H), 2.01-2.04 (m, 1H), 1.27 (d, J=6.06 Hz, 6H); LCMS: Calculated: for C21H21N3O2: 347, Measured: 348 [M+H]+.

### Example 5

### 6-(1H-Pyrazol-4-yl)-2-(thiophen-2-ylmethyl)isoindolin-1-one

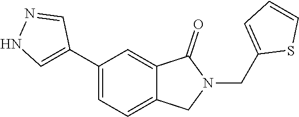 [SMILES: O=C1c2cc(-c3cn[nH]c3)ccc2CN1Cc1cccs1]

¹H NMR (400 MHz, METHANOL-d4) δ 8.10 (br s, 1H), 7.96 (s, 1H), 7.84 (d, J=8.08 Hz, 1H), 7.53 (d, J=7.58 Hz, 1H), 7.35 (d, J=5.05 Hz, 1H), 7.11 (d, J=3.03 Hz, 1H), 6.99 (dd, J=3.28, 5.31 Hz, 1H), 5.00 (s, 1H), 4.45 (s, 1H); LCMS: Calculated: for C16H13N3OS: 295, Measured: 296 [M+H]+.

### Example 6

### 2-(3-Fluorobenzyl)-6-(1H-pyrazol-4-yl)isoindolin-1-one

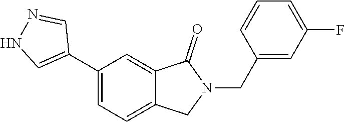 [SMILES: O=C1c2cc(-c3cn[nH]c3)ccc2CN1Cc1cccc(F)c1]

¹H NMR (400 MHz, METHANOL-d4) δ 8.04-8.23 (m, 1H), 7.99 (s, 1H), 7.85 (dd, J=1.52, 8.08 Hz, 1H), 7.53 (d, J=8.08 Hz, 1H), 7.31-7.40 (m, 1H), 6.95-7.16 (m, 2H), 4.83 (s, 1H), 4.42 (s, 1H); LCMS: Calculated: for C18H14FN3O: 307, Measured: 308 [M+H]+.

### Example 7

### 2-(3-(Aminomethyl)benzyl)-6-(1H-pyrazol-4-yl)isoindolin-1-one

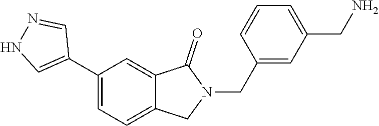 [SMILES: NCc1cccc(CN2Cc3ccc(-c4cn[nH]c4)cc3C2=O)c1]

¹H NMR (400 MHz, METHANOL-d4) δ 8.10 (br s, 1H), 7.98 (s, 1H), 7.84 (d, J=7.58 Hz, 1H), 7.34-7.56 (m, 3H), 4.86 (s, 1H), 4.42 (s, 1H), 4.10 (s, 1H), 2.61-2.77 (m, 2H); LCMS: Calculated: for C19H18N4O: 318, Measured: 319 [M+H]+.

### Example 8

### 6-(1H-Pyrazol-4-yl)-2-(thiophen-2-ylmethyl)isoindolin-1-one

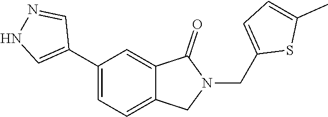 [SMILES: Cc1ccc(CN2Cc3ccc(-c4cn[nH]c4)cc3C2=O)s1]

1H NMR (400 MHz, METHANOL-d4) δ 8.10 (br s, 1H), 7.95 (d, J=1.01 Hz, 1H), 7.83 (dd, J=1.52, 7.58 Hz, 1H), 7.53 (d, J=8.08 Hz, 1H), 6.87 (d, J=3.03 Hz, 1H), 6.49-6.75 (m, 1H), 4.43 (s, 1H), 2.41 (s, 3H); LCMS: Calculated: for C17H15N3OS: 309, Measured: 310 [M+H]+.

### Example 9

### 2-((5-Fluorothiophen-2-yl)methyl)-6-(1H-pyrazol-4-yl)isoindolin-1-one

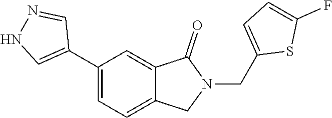 [SMILES: O=C1c2cc(-c3cn[nH]c3)ccc2CN1Cc1ccc(F)s1]

1H NMR (400 MHz, METHANOL-d4) δ 8.10 (s, 1H), 7.96 (d, J=1.01 Hz, 1H), 7.85 (dd, J=1.52, 8.08 Hz, 1H), 7.55 (d, J=8.08 Hz, 1H), 6.78 (t, J=3.79 Hz, 1H), 6.42 (dd, J=1.77, 3.79 Hz, 1H), 4.85 (d, J=2.53 Hz, 1H), 4.46 (s, 1H); LCMS: Calculated: for C16H12FN3OS: 313, Measured: 314 [M+H]+.

### Example 10

### 2-(3-(Prop-2-yn-1-yloxy)benzyl)-6-(1H-pyrazol-4-yl)isoindolin-1-one

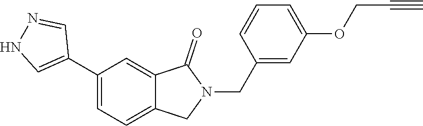 [SMILES: C#CCOc1cccc(CN2Cc3ccc(-c4cn[nH]c4)cc3C2=O)c1]

### Step 1: 2-(3-(Prop-2-ynyloxy)benzyl)-6-(1-(tetrahydro-2H-pyran-2-yl)-1H-pyrazol-4-yl)isoindolin-1-one

To a solution of 2-(3-hydroxybenzyl)-6-(1-(tetrahydro-2H-pyran-2-yl)-1H-pyrazol-4-yl)isoindolin-1-one (150 mg, 0.385 mmol) in DMF (8 mL) was added K₂CO₃ (0.159 g, 1.155 mmol), 3-bromoprop-1-yne (0.069 g, 0.578 mmol) and the resulting mixture was stirred at room temperature. After 12 h, the reaction mixture was diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded 2-(3-(prop-2-ynyloxy)benzyl)-6-(1-(tetrahydro-2H-pyran-2-yl)-1H-pyrazol-4-yl)isoindolin-1-one as a yellow oil. LCMS: Calculated: for C26H25N3O3: 427, Measured: 428 [M+H]+.

### Step 2: 2-(3-((1-Oxo-6-(1-(tetrahydro-2H-pyran-2-yl)-1H-pyrazol-4-yl)isoindolin-2-yl)methyl)phenoxy) acetonitrile

To a solution of 2-(3-hydroxybenzyl)-6-(1-(tetrahydro-2H-pyran-2-yl)-1H-pyrazol-4-yl)isoindolin-1-one (450 mg, 1.16 mmol) in DMF (10 mL) were added K₂CO₃ (0.478 g, 3.47 mmol), 2-bromoacetonitrile (0.208 g, 1.73 mmol), and the resulting mixture was stirred at room temperature. After 12 h, the reaction mixture was diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded 2-(3-((1-oxo-6-(1-(tetrahydro-2H-pyran-2-yl)-1H-pyrazol-4-yl)isoindolin-2-yl)methyl)phenoxy) acetonitrile as a yellow oil. LCMS: Calculated: for C25H24N4O3: 428, Measured: 429 [M+H]+.

### Step 3: 2-(3-((2H-Tetrazol-5-yl)methoxy)benzyl)-6-(1-(tetrahydro-2H-pyran-2-yl)-1H-pyrazol-4-yl)isoindolin-1-one

A solution of 2-(3-((1-oxo-6-(1-(tetrahydro-2H-pyran-2-yl)-1H-pyrazol-4-yl)isoindolin-2-yl)methyl)phenoxy) acetonitrile (0.150 g, 0.350 mmol), TMSN₃ (0.121 g, 1.050 mmol) in toluene (10 mL) was treated with dibutylstannanone (9 mg, 0.035 mmol) and then warmed to 110° C. After 12 h, the resulting mixture was concentrated in vacuo. Purification of the resulting residue by silica gel chromatography (40 g column, 0-20% MeOH/DCM yielded 2-(3-((2H-tetrazol-5-yl)methoxy)benzyl)-6-(1-(tetrahydro-2H-pyran-2-yl)-1H-pyrazol-4-yl)isoindolin-1-one as a yellow solid. LCMS: Calculated: for C25H25N7O3: 471, Measured: 494 [M+Na]+.

### Step 4: 2-(3-(Prop-2-yn-1-yloxy)benzyl)-6-(1H-pyrazol-4-yl)isoindolin-1-one

To a solution of 2-(3-(prop-2-ynyloxy)benzyl)-6-(1-(tetrahydro-2H-pyran-2-yl)-1H-pyrazol-4-yl)isoindolin-1-one (90 mg, 0.211 mmol) in DCM (2 mL) was added TFA (0.7 mL). The resulting mixture was stirred for 2 h at 25° C. The resulting mixture was concentrated in vacuo. Purification of the resulting residue by C18 reverse column chromatography (80 g, 0%-100% ACN/H₂O (containing 0.05% TFA)) yielded 4: 2-(3-(prop-2-yn-1-yloxy)benzyl)-6-(1H-pyrazol-4-yl)isoindolin-1-one as a white solid.

¹H NMR (300 MHz, DMSO-d6) δ 12.97 (s, 1H), 8.32 (s, 1H), 7.98-8.06 (m, 1H), 7.93 (d, J=1.7 Hz, 1H), 7.83 (dd, J=7.9, 1.7 Hz, 1H), 7.51 (d, J=8.0 Hz, 1H), 7.27 (t, J=7.9 Hz, 1H), 6.85-6.91 (m, 3H), 4.70-4.76 (m, 4H), 4.34 (s, 2H); LCMS: Calculated: for C21H17N3O2: 343, Measured: 344 [M+H]+.

The following compounds were similarly prepared according to the procedures described herein, selecting and substitution suitable reagents and starting materials as would be readily recognized by those skilled in the art.

### Example 11

### 2-(3-((2H-Tetrazol-5-yl)methoxy)benzyl)-6-(1H-pyrazol-4-yl)isoindolin-1-one

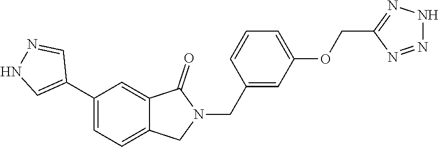 [SMILES: O=C1c2cc(-c3cn[nH]c3)ccc2CN1Cc1cccc(OCc2nn[nH]n2)c1]

2-(3-((2H-Tetrazol-5-yl)methoxy)benzyl)-6-(1H-pyrazol-4-yl)isoindolin-1-one was prepared according to the procedure described in Example 10, Step 4, substituting 2-(3-((2H-tetrazol-5-yl)methoxy)benzyl)-6-(1-(tetrahydro-2H-pyran-2-yl)-1H-pyrazol-4-yl)isoindolin-1-one for 2-(3-(prop-2-ynyloxy)benzyl)-6-(1-(tetrahydro-2H-pyran-2-yl)-1H-pyrazol-4-yl)isoindolin-1-one.

¹H NMR (400 MHz, DMSO-d6) δ 12.97 (s, 1H), 8.05-8.37 (m, 2H), 7.96 (s, 1H), 7.86 (d, J=7.9 Hz, 1H), 7.54 (d, J=7.9 Hz, 1H), 7.33 (t, J=7.8 Hz, 1H), 6.91-7.01 (m, 3H), 5.49 (s, 2H), 4.73 (s, 2H), 4.37 (s, 2H); LCMS: Calculated: for C20H17N7O2 387, Measured: 388 [M+H]+.

### Example 12

### 2-amino-2′-(3-methoxybenzyl)-5′-(1H-pyrazol-4-yl)spiro[cyclopentane-1,1′-isoindolin]-3′-one

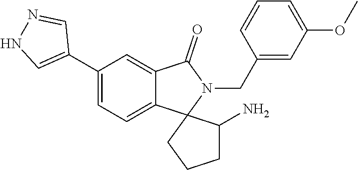 [SMILES: COc1cccc(CN2C(=O)c3cc(-c4cn[nH]c4)ccc3C23CCCC3N)c1]

2-Amino-2′-(3-methoxybenzyl)-5′-(1H-pyrazol-4-yl)spiro[cyclopentane-1,1′-isoindolin]-3′-one was prepared according to the procedure described in Example 104, substituting lithium hydroxide in THE for sodium methoxide in methanol.

¹H NMR (400 MHz, DMSO-d6) δ 8.24 (d, J=8.7 Hz, 2H), 8.02 (d, J=1.7 Hz, 1H), 7.90-8.00 (m, 1H), 7.58-7.76 (m, 3H), 7.02-7.32 (m, 2H), 6.73-6.90 (m, 3H), 4.27-5.34 (m, 2H), 3.99 (s, 1H), 3.69 (d, J=4.7 Hz, 3H), 1.56-2.37 (m, 6H); LCMS: Calculated: for C23H24N4O2: 388, Measured: 389 [M+H]+.

### Example 13

### 4-(2-(4-methoxybenzyl)-3-oxoisoindolin-5-yl)-N-methylpyrimidine-2-carboxamide

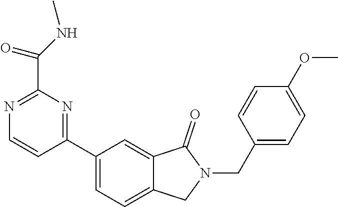 [SMILES: CNC(=O)c1nccc(-c2ccc3c(c2)C(=O)N(Cc2ccc(OC)cc2)C3)n1]

### Step 1: 6-Bromo-2-(4-methoxybenzyl)isoindolin-1-one

6-Bromo-2-(4-methoxybenzyl)isoindolin-1-one was prepared as described in Example 1, Alternate Step 1, substituting 1-(bromomethyl)-4-methoxybenzene in place of 6-bromo-2-(3-methoxybenzyl)isoindolin-1-one. LCMS: Calculated: for C16H14BrNO2: 332, Measured: 334 [M+2H]+

### Step 2: 2-(4-Methoxybenzyl)-6-(4,4,5,5-tetramethyl-1,3,2-dioxaborolan-2-yl)isoindolin-1-one

2-(4-Methoxybenzyl)-6-(4,4,5,5-tetramethyl-1,3,2-dioxaborolan-2-yl)isoindolin-1-one was prepared according to the procedure described in Example 70, Step 1, substituting 6-bromo-2-(4-methoxybenzyl)isoindolin-1-one was used in place of 6-bromo-2-(3-methoxybenzyl)isoindolin-1-one.

### Step 3: 6-(2-Chloropyrimidin-4-yl)-2-(4-methoxybenzyl)isoindolin-1-one

A solution of 2-(4-methoxybenzyl)-6-(4,4,5,5-tetramethyl-1,3,2-dioxaborolan-2-yl)isoindolin-1-one (500 mg, 1.318 mmol), 2,4-dichloropyrimidine (196.4 mg, 1.318 mmol), Pd(PPh₃)₄ (152.4 mg, 0.132 mmol) and potassium carbonate (555 mg, 3.96 mmol, 3.00 eq) in 1,4-dioxane/H₂O (8/0.8 mL) was warmed to 80° C. under nitrogen atmosphere of nitrogen. After 16 h, the reaction mixture was diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded 6-(2-chloropyrimidin-4-yl)-2-(4-methoxybenzyl)isoindolin-1-one as a light yellow solid. LCMS: Calculated: for C20H16ClN3O2: 365, Measured: 366 [M+H]+.

### Step 4: Methyl 4-(2-(4-methoxybenzyl)-3-oxoisoindolin-5-yl) pyrimidine-2-carboxylate

A solution of 6-(2-chloropyrimidin-4-yl)-2-(4-methoxybenzyl)isoindolin-1-one (250 mg, 0.683 mmol), Pd(dppf)Cl.CHCl₃ (49.1 mg, 0.068 mmol) and TEA (207.5 mg, 2.050 mmol) in DMF/MeOH (4/4 mL) was stirred at 80° C. under 5 atm of CO. After 16 h, the reaction mixture was diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded methyl 4-(2-(4-methoxybenzyl)-3-oxoisoindolin-5-yl) pyrimidine-2-carboxylate as a light yellow solid. LCMS: Calculated: for C22H19N3O4: 389, Measured: 390 [M+H]+.

### Step 5: 4-(2-(4-Methoxybenzyl)-3-oxoisoindolin-5-yl)-N-methylpyrimidine-2-carboxamide

A solution of methyl 4-(2-(4-methoxybenzyl)-3-oxoisoindolin-5-yl) pyrimidine-2-carboxylate (80 mg, 0.205 mmol) in MeNH₂/THF (5 mL) was stirred at 80° C. under a nitrogen atmosphere. After 3 h, the reaction mixture was poured into water, extracted with EtOAc (2×25 mL), washed with brine, dried (Na₂SO₄), and concentrated in vacuo. Purification of the resulting residue by Prep-TLC with 5% MeOH/DCM yielded 4-(2-(4-methoxybenzyl)-3-oxoisoindolin-5-yl)-N-methylpyrimidine-2-carboxamide as an off-white solid.

¹H NMR (300 MHz, DMSO-d6) δ 8.98 (t, J=4.4 Hz, 2H), 8.69-8.53 (m, 2H), 8.33 (d, J=5.4 Hz, 1H), 7.74 (d, J=8.1 Hz, 1H), 7.25 (t, J=8.2 Hz, 1H), 6.83 (d, J=6.4 Hz, 3H), 4.71 (s, 2H), 4.45 (s, 2H), 3.70 (s, 3H), 2.84 (d, J=4.7 Hz, 3H); LCMS: Calculated: for C22H20N4O3: 388, Measured: 389 [M+H]+.

### Example 14

### 2-(2-hydroxy-1-(3-methoxyphenyl)ethyl)-6-(1H-pyrazol-4-yl)isoindolin-1-one

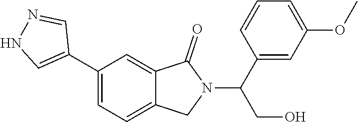 [SMILES: COc1cccc(C(CO)N2Cc3ccc(-c4cn[nH]c4)cc3C2=O)c1]

### Step 1: 6-Bromo-2-(2-hydroxy-1-(3-methoxyphenyl)ethyl)isoindolin-1-one

(2-amino-2-(3-methoxyphenyl)ethanol (1.5 g, 8.971 mmol) was dissolved in acetonitrile (15 ml), then N,N-diisopropylethylamine (4.448 mL, 26.913 mmol) and methyl 5-bromo-2-(bromomethyl)benzoate (3.039 g, 9.868 mmol) were added and stirred at 85° C. After 12 h, the reaction mixture was treated with water, and concentrated in vacuo. Purification of the resulting residue by silica gel chromatography (40 g column, 0-100% EtOAc/Heptane) yielded 6-bromo-2-(2-hydroxy-1-(3-methoxyphenyl)ethyl)isoindolin-1-one as a yellow oil. 1H NMR (300 MHz, DMSO-d6) δ 7.78-7.83 (m, 2H), 7.57 (d, J=8.1 Hz, 1H), 7.24-7.30 (m, 1H), 6.84-6.90 (m, 3H), 5.32 (dd, J=8.6, 5.6 Hz, 1H), 5.11 (t, J=5.5 Hz, 1H), 4.58-4.68 (m, 1H), 4.32 (d, J=18.1 Hz, 1H), 3.90-4.10 (m, 2H), 3.74 (s, 3H); LCMS: Calculated: for C17H16BrNO3: 362, Measured: 364 [M+2H]+.

### Step 2: 2-(2-Hydroxy-1-(3-methoxyphenyl)ethyl)-6-(1-(tetrahydro-2H-pyran-2-yl)-1H-pyrazol-4-yl)isoindolin-1-one

2-(2-Hydroxy-1-(3-methoxyphenyl)ethyl)-6-(1-(tetrahydro-2H-pyran-2-yl)-1H-pyrazol-4-yl)isoindolin-1-one was prepared according to the procedure described in Example 60, Step 1, substituting 6-bromo-2-(2-hydroxy-1-(3-methoxyphenyl)ethyl)isoindolin-1-one for (R)-4-bromo-6-iodo-2-(1-(3-methoxyphenyl)ethyl)isoindolin-1-one. LCMS: Calculated: for C25H27N3O4: 433, Measured: 434 [M+H]+.

### Step 3: 2-(3-Methoxybenzyl)-6-(1H-pyrazol-4-yl)isoindolin-1-one)

2-(3-Methoxybenzyl)-6-(1H-pyrazol-4-yl)isoindolin-1-one was prepared according to the procedure described in Example 60, Step 3, substituting 2-(2-Hydroxy-1-(3-methoxyphenyl)ethyl)-6-(1-(tetrahydro-2H-pyran-2-yl)-1H-pyrazol-4-yl)isoindolin-1-one for 4-Bromo-2-((R)-1-(3-methoxyphenyl)ethyl)-6-(1-(tetrahydro-2H-pyran-2-yl)-1H-pyrazol-4-yl)isoindolin-1-one.

¹H NMR (300 MHz, DMSO-d6) δ 12.94 (s, 1H), 8.29 (s, 1H), 8.00 (s, 1H), 7.88 (d, J=1.6 Hz, 1H), 7.81 (dd, J=8.0, 1.7 Hz, 1H), 7.51 (d, J=7.9 Hz, 1H), 7.23 (td, J=7.3, 2.0 Hz, 1H), 6.78-6.89 (m, 3H), 5.31 (dd, J=8.3, 5.8 Hz, 1H), 5.06 (t, J=5.5 Hz, 1H), 4.57 (d, J=17.7 Hz, 1H), 4.26 (d, J=17.6 Hz, 1H), 3.93 (dq, J=11.5, 6.5, 5.7 Hz, 2H), 3.69 (s, 3H); LCMS: Calculated: for C20H19N3O3: 349, Measured: 350 [M+H]+.

### Example 15

### 2-(2-(dimethylamino)-1-(3-methoxyphenyl)ethyl)-6-(1H-pyrazol-4-yl)isoindolin-1-one

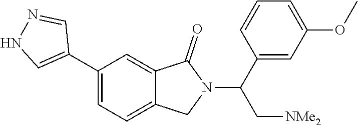 [SMILES: COc1cccc(C(CN(C)C)N2Cc3ccc(-c4cn[nH]c4)cc3C2=O)c1]

### Step 1: 2-(3-Methoxyphenyl)-2-(1-oxo-6-(1-(tetrahydro-2H-pyran-2-yl)-1H-pyrazol-4-yl)isoindolin-2-yl)ethyl methanesulfonate

A mixture of 2-(2-hydroxy-1-(3-methoxyphenyl)ethyl)-6-(1-(tetrahydro-2H-pyran-2-yl)-1H-pyrazol-4-yl)isoindolin-1-one (280 mg, 0.646 mmol) in DCM (5 mL), triethylamine (164 mg, 1.615 mmol) was treated with methanesulfonyl chloride (81 mg, 0.710 mmol) at room temperature. After 2 h, the reaction mixture was poured into water, extracted with EtOAc (2×25 mL), washed with brine, dried (Na₂SO₄), and concentrated in vacuo. Purification of the resulting residue by silica gel chromatography (40 g column, 0-100% EtOAc/Heptane) yielded 2-(3-methoxyphenyl)-2-(1-oxo-6-(1-(tetrahydro-2H-pyran-2-yl)-1H-pyrazol-4-yl)isoindolin-2-yl)ethyl methanesulfonate as a white solid. LCMS: Calculated: for C26H29N3O6S: 511, Measured: 512 [M+H]+.

### Step 2: 2-(Azido(3-methoxyphenyl)methyl)-6-(1-(tetrahydro-2H-pyran-2-yl)-1H-pyrazol-4-yl)isoindolin-1-one

A mixture of sodium azide (171.6 mg, 2.64 mmol) was added to 2-(3-methoxyphenyl)-2-(1-oxo-6-(1-(tetrahydro-2H-pyran-2-yl)-1H-pyrazol-4-yl)isoindolin-2-yl)ethyl methanesulfonate (450 mg, 0.88 mmol 1.00 eq) in DCM, and the resulting mixture was stirred at room temperature for 30 min, then stirred at 50° C. for 12 h. The reaction was quenched with saturated sodium thiosulfate solution, extracted with EtOAc (2×25 mL), and concentrated in vacuo to yield 2-(azido(3-methoxyphenyl)methyl)-6-(1-(tetrahydro-2H-pyran-2-yl)-1H-pyrazol-4-yl)isoindolin-1-one as a light yellow oil. LCMS: Calculated: for C25H26N6O3: 458, Measured: 459 [M+H]+.

### Step 3: 2-(Amino(3-methoxyphenyl)methyl)-6-(1-(tetrahydro-2H-pyran-2-yl)-1H-pyrazol-4-yl)isoindolin-1-one

A solution of 2-(azido(3-methoxyphenyl)methyl)-6-(1-(tetrahydro-2H-pyran-2-yl)-1H-pyrazol-4-yl)isoindolin-1-one (300 mg, 0.654 mmol) and triphenylphosphine (514.8 mg, 1.96 mmol) in THF/H₂O (3/3 mL) was stirred at room temperature. After 16 h, the reaction mixture was poured into water, extracted with EtOAc (2×25 mL), washed with brine, dried (Na₂SO₄), and concentrated in vacuo. Purification of the resulting residue by silica gel chromatography (40 g column, 0-10% MeOH/DCM) yielded 2-(amino(3-methoxyphenyl)methyl)-6-(1-(tetrahydro-2H-pyran-2-yl)-1H-pyrazol-4-yl)isoindolin-1-one as a light yellow oil. LCMS: Calculated: for C25H28N4O3: 432, Measured: 433 [M+H]+.

### Step 4: 2-(2-(Dimethylamino)-1-(3-methoxyphenyl)ethyl)-6-(1-(tetrahydro-2H-pyran-2-yl)-1H-pyrazol-4-yl)isoindolin-1-one

Under a nitrogen atmosphere, a solution of 2-(amino(3-methoxyphenyl)methyl)-6-(1-(tetrahydro-2H-pyran-2-yl)-1H-pyrazol-4-yl)isoindolin-1-one (200 mg, 0.478 mmol), formaldehyde (151.0 mg, 1.912 mmol) and acetic acid (0.1 mL) in MeOH (5 mL) was stirred at 20° C. After 30 min, sodium cyanoborohydride (90.1 mg, 1.434 mmol) was added and the reaction mixture was stirred at room temperature. After 3 h, the reaction mixture was extracted with EtOAc (2×25 mL), washed with brine, dried (Na₂SO₄), and concentrated in vacuo. Purification of the resulting residue by silica gel chromatography (40 g column, 0-100% EtOAc/Heptane) yielded 2-(2-(dimethylamino)-1-(3-methoxyphenyl)ethyl)-6-(1-(tetrahydro-2H-pyran-2-yl)-1H-pyrazol-4-yl)isoindolin-1-one as a light yellow oil. LCMS: Calculated: for C27H32N4O3: 460, Measured: 461 [M+H]+.

### Step 5: 2-(2-(Dimethylamino)-1-(3-methoxyphenyl)ethyl)-6-(1H-pyrazol-4-yl)isoindolin-1-one

2-(2-(Dimethylamino)-1-(3-methoxyphenyl)ethyl)-6-(1H-pyrazol-4-yl)isoindolin-1-one was prepared according to the procedure described in Example 14, Step 3, substituting 2-(2-(dimethylamino)-1-(3-methoxyphenyl)ethyl)-6-(1-(tetrahydro-2H-pyran-2-yl)-1H-pyrazol-4-yl)isoindolin-1-one for 2-(2-hydroxy-1-(3-methoxyphenyl)ethyl)-6-(1-(tetrahydro-2H-pyran-2-yl)-1H-pyrazol-4-yl)isoindolin-1-one.

¹H NMR (300 MHz, DMSO-d6) δ 12.97 (s, 1H), 8.31 (s, 1H), 8.01 (s, 1H), 7.92-7.77 (m, 2H), 7.51 (d, J=7.9 Hz, 1H), 7.26 (t, J=7.8 Hz, 1H), 6.97-6.80 (m, 3H), 5.49 (m, 1H), 4.54 (d, J=17.6 Hz, 1H), 4.23 (d, J=17.6 Hz, 1H), 3.72 (s, 3H), 3.21-3.06 (m, 1H), 2.58 (m, 1H), 2.21 (s, 6H); LCMS: Calculated: for C22H24N4O2: 376, Measured: 377 [M+H]+.

### Example 16

### 2-(2-Hydroxy-1-(3-ethoxyphenyl)ethyl)-6-(1H-pyrazol-4-yl)isoindolin-1-one

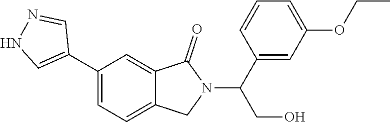 [SMILES: CCOc1cccc(C(CO)N2Cc3ccc(-c4cn[nH]c4)cc3C2=O)c1]

### Step 1: 2-(2-hydroxy-1-(3-ethoxyphenyl)ethyl)-6-(1H-pyrazol-4-yl)isoindolin-1-one

2-(2-Hydroxy-1-(3-ethoxyphenyl)ethyl)-6-(1H-pyrazol-4-yl)isoindolin-1-one was prepared according to the procedure described in Example 14, Step 3, substituting (2-amino-2-(3-ethoxyphenyl)ethanol for (2-amino-2-(3-methoxyphenyl)ethanol.

¹H NMR (400 MHz, DMSO-d6) δ 12.98 (s, 1H), 8.33 (s, 1H), 8.02 (s, 1H), 7.91 (s, 1H), 7.84 (dd, J=7.9, 1.7 Hz, 1H), 7.54 (d, J=7.9 Hz, 1H), 7.18-7.30 (m, 1H), 6.76-6.91 (m, 3H), 5.34 (dd, J=8.5, 5.7 Hz, 1H), 5.08 (t, J=5.5 Hz, 1H), 4.60 (d, J=17.7 Hz, 1H), 4.29 (d, J=17.6 Hz, 1H), 3.89-4.08 (m, 4H), 1.30 (t, J=6.9 Hz, 3H); LCMS: Calculated: for C21H21N3O3: 363, Measured: 364 [M+H]+.

### Example 17

### 2-(2-hydroxy-1-(3-phenoxyphenyl)ethyl)-6-(1H-pyrazol-4-yl)isoindolin-1-one

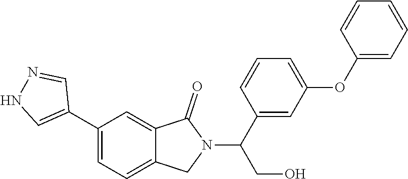 [SMILES: O=C1c2cc(-c3cn[nH]c3)ccc2CN1C(CO)c1cccc(Oc2ccccc2)c1]

### Step 1: Methyl 2-(6-bromo-1-oxoisoindolin-2-yl)-2-(3-phenoxyphenyl)acetate

Methyl 2-(6-bromo-1-oxoisoindolin-2-yl)-2-(3-phenoxyphenyl)acetate was prepared according to the procedure described in Example 1, Step 1 Alternate, substituting methyl 2-bromo-2-(3-phenoxyphenyl)acetate for 1-(bromomethyl)-3-methoxybenzene. LCMS: Calculated: for C23H18BrNO4: 452, Measured: 452 [M]+.

### Step 2: 6-Bromo-2-(2-hydroxy-1-(3-phenoxyphenyl)ethyl)isoindolin-1-one

Lithium tetrahydroborate (14.5 g, 0.663 mmol) was added to a solution of methyl 2-(6-bromo-1-oxoisoindolin-2-yl)-2-(3-phenoxyphenyl)acetate (100 mg, 0.221 mmol) in THE (2 mL) at 0° C. After 2 h, the reaction mixture was treated with saturated NH₄Cl solution, extracted with EtOAc (2×25 mL), washed with brine dried (Na₂SO₄), and concentrated in vacuo. Purification of the resulting residue by silica gel chromatography (24 g column, 0-100% EtOAc/Heptane) yielded 6-bromo-2-(2-hydroxy-1-(3-phenoxyphenyl)ethyl)isoindolin-1-one as a light yellow oil. LCMS: Calculated: for C22H18BrNO3: 424, Measured: 423 [M−H]+.

### Step 3: 2-(2-Hydroxy-1-(3-phenoxyphenyl)ethyl)-6-(1H-pyrazol-4-yl)isoindolin-1-one

2-(2-Hydroxy-1-(3-phenoxyphenyl)ethyl)-6-(1H-pyrazol-4-yl)isoindolin-1-one was prepared according to the procedure described in Example 1, Step 5, substituting 6-bromo-2-(2-hydroxy-1-(3-phenoxyphenyl)ethyl)isoindolin-1-one for 6-bromo-2-(3-methoxybenzyl)isoindolin-1-one.

¹H NMR (300 MHz, DMSO-d6) δ 12.96 (s, 1H), 8.30 (s, 1H), 7.99 (s, 1H), 7.91-7.76 (m, 2H), 7.51 (d, J=7.9 Hz, 1H), 7.40-7.25 (m, 3H), 7.14-6.91 (m, 5H), 6.83 (m, 1H), 5.32 (t, J=7.0 Hz, 1H), 5.09 (t, J=5.5 Hz, 1H), 4.57 (d, J=17.7 Hz, 1H), 4.27 (d, J=17.7 Hz, 1H), 4.05-3.86 (m, 2H); LCMS: Calculated: for C25H21N3O3: 411, Measured: 412 [M+H]+.

### Example 18

### 2-(2-methoxy-1-(3-methoxyphenyl)ethyl)-6-(1H-pyrazol-4-yl)isoindolin-1-one

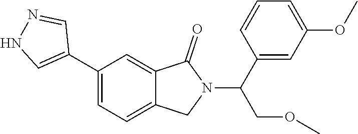 [SMILES: COCC(c1cccc(OC)c1)N1Cc2ccc(-c3cn[nH]c3)cc2C1=O]

### Step 1: 6-Bromo-2-(2-methoxy-1-(3-methoxyphenyl)ethyl)isoindolin-1-one

A mixture of 6-bromo-2-(2-hydroxy-1-(3-methoxyphenyl)ethyl)isoindolin-1-one (180 mg, 0.497 mmol) and Ag₂O (346 mg, 1.493 mmol) in acetonitrile (20 mL) was treated with iodomethane (353 mg, 2.487 mmol) at room temperature. After 12 h, the reaction mixture was concentrated in vacuo. Purification of the resulting residue by silica gel chromatography (12 g column, 0-100% EtOAc/Heptane) yielded 6-bromo-2-(2-methoxy-1-(3-methoxyphenyl)ethyl)isoindolin-1-one as a light yellow oil. LCMS: Calculated: for C18H18BrNO3: 376, Measured: 377 [M+H]+.

### Step 2: 2-(2-Methoxy-1-(3-methoxyphenyl)ethyl)-6-(1H-pyrazol-4-yl)isoindolin-1-one

2-(2-Methoxy-1-(3-methoxyphenyl)ethyl)-6-(1H-pyrazol-4-yl)isoindolin-1-one was prepared according to the procedure described in Example 1, Step 5, substituting 6-bromo-2-(2-methoxy-1-(3-methoxyphenyl)ethyl)isoindolin-1-one for 6-bromo-2-(3-methoxybenzyl)isoindolin-1-one.

¹H NMR (400 MHz, DMSO-d6) δ 12.99 (brs, 1H), 8.34 (brs, 1H), 8.03 (brs, 1H), 7.92 (s, 1H), 7.84-7.86 (m, 1H), 7.54 (d, J=7.8 Hz, 1H), 7.28 (t, J=8.2 Hz, 1H), 6.87-6.91 (m, 3H), 5.53 (dd, J=8.9, 5.3 Hz, 1H), 4.53 (d, J=17.5 Hz, 1H), 4.31 (d, J=17.5 Hz, 1H), 4.03 (t, J=9.7 Hz, 1H), 3.87 (dd, J=10.6, 5.2 Hz, 1H), 3.74 (s, 3H), 3.33 (s, 3H); LCMS: Calculated: for C21H21N3O3: 363, Measured: 364 [M+H]+.

### Example 19

### 2-(1-(3-methoxyphenyl)-2-phenoxyethyl)-6-(1H-pyrazol-4-yl)isoindolin-1-one

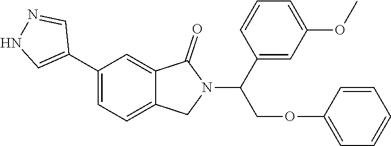 [SMILES: COc1cccc(C(COc2ccccc2)N2Cc3ccc(-c4cn[nH]c4)cc3C2=O)c1]

2-(1-(3-Methoxyphenyl)-2-phenoxyethyl)-6-(1H-pyrazol-4-yl)isoindolin-1-one was prepared according to the procedure described in Example 14, Step 3, substituting (2-amino-2-(3-phenoxyphenyl)ethanol for (2-amino-2-(3-methoxyphenyl)ethanol.

¹H NMR (400 MHz, DMSO-d6) δ 12.99 (s, 1H), 8.33 (s, 1H), 8.04 (d, J=17.5 Hz, 1H), 7.93 (s, 1H), 7.90-7.80 (m, 1H), 7.53 (d, J=7.9 Hz, 1H), 7.28 m, 3H), 7.02-6.85 (m, 6H), 5.71 (t, J=6.9 Hz, 1H), 4.60 (m, 3H), 4.33 (d, J=17.6 Hz, 1H), 3.73 (s, 3H), 2.48 (s, 2H); LCMS: Calculated: for C26H23N3O3: 425, Measured: 426 [M+H]+.

### Example 20

### 2-(2-(3-methoxyphenyl)-2-(1-oxo-6-(1H-pyrazol-4-yl)isoindolin-2-yl)ethoxy)acetic acid

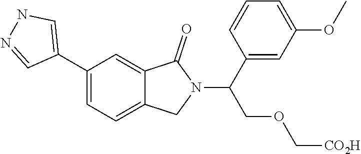 [SMILES: COc1cccc(C(COCC(=O)O)N2Cc3ccc(-c4cn[nH]c4)cc3C2=O)c1]

### Step 1: Ethyl 2-(2-(6-bromo-1-oxoisoindolin-2-yl)-2-(3-methoxyphenyl)ethoxy)acetate

A mixture of 6-bromo-2-(2-hydroxy-1-(3-methoxyphenyl)ethyl)isoindolin-1-one (300 mg, 0.828 mmol) in DCM (15 mL) was treated with ethyl diazoacetate (283 mg, 2.48 mmol) followed by dropwise addition of BF₃.Et₂O (352 mg, 2.48 mmol) dropwise at 0° C. After the BF₃.Et₂O was added, the reaction mixture was allowed to warm to room temperature. After 12 h, the reaction mixture was treated with satd. aq. NaHCO₃ (30 mL), extracted with EtOAc (3×15 mL), and the combined organic layer was dried (Na₂SO₄), filtered and concentrated in vacuo. Purification of the resulting residue by a silica gel chromatography (40 g, 0-100% EtOAc/heptane) yielded ethyl 2-(2-(6-bromo-1-oxoisoindolin-2-yl)-2-(3-methoxyphenyl)ethoxy)acetate as a yellow oil.

### Step 2: 2-(2-(3-Methoxyphenyl)-2-(1-oxo-6-(1H-pyrazol-4-yl)isoindolin-2-yl)ethoxy)acetic acid

Under nitrogen atmosphere, a mixture of ethyl 2-(2-(6-bromo-1-oxoisoindolin-2-yl)-2-(3-methoxyphenyl)ethoxy)acetate (200 mg, 0.446 mmol), tert-butyl 4-(4,4,5,5-tetramethyl-1,3,2-dioxaborolan-2-yl)-1H-pyrazole-1-carboxylate (262 mg, 0.891 mmol), K₂CO₃ (185 mg, 1.339 mmol) and Pd(PPh₃)₄ (26 mg, 0.022 mmol) in DMF (15 mL) and H₂O (1.5 mL) was stirred at 100° C. After 12 h, the reaction mixture was allowed to cool to room temperature, diluted with H₂O, pH was adjusted to 6 with aq. HCl (1N). The resulting solution was extracted with EtOAc (3×15 mL), the organic layers were combined, washed with brine (3×15 mL), dried (Na₂SO₄), filtered and concentrated in vacuo. Purification of the resulting residue by recrystallization with EtOAc/Hexane (1/1) yielded 2-(2-(3-methoxyphenyl)-2-(1-oxo-6-(1H-pyrazol-4-yl)isoindolin-2-yl)ethoxy)acetic acid as a white solid.

¹H NMR (400 MHz, DMSO-d6) δ 12.79 (brs, 2H), 8.17 (s, 2H), 7.91 (d, J=1.6 Hz, 1H), 7.83 (dd, J=7.9, 1.7 Hz, 1H), 7.53 (d, J=7.9 Hz, 1H), 7.26 (t, J=7.9 Hz, 1H), 6.84-6.92 (m, 3H), 5.53 (dd, J=8.7, 5.0 Hz, 1H), 4.68 (d, J=17.7 Hz, 1H), 4.28 (d, J=17.7 Hz, 1H), 4.14-4.22 (m, 1H), 4.11 (s, 2H), 4.00 (dd, J=10.2, 5.1 Hz, 1H), 3.72 (s, 3H); LCMS Calculated: for C22H21N3O5: 407, Measured: 408 [M+H]+.

### Example 21

### 2-(2-(3-Methoxyphenyl)-2-(1-oxo-6-(1H-pyrazol-4-yl)isoindolin-2-yl)ethoxy)acetamide

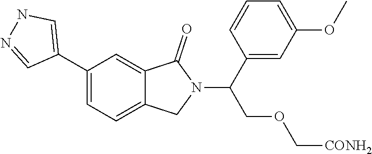 [SMILES: COc1cccc(C(COCC(N)=O)N2Cc3ccc(-c4cn[nH]c4)cc3C2=O)c1]

A mixture 2-(2-(3-methoxyphenyl)-2-(1-oxo-6-(1H-pyrazol-4-yl)isoindolin-2-yl)ethoxy)acetic acid (60 mg, 0.147 mmol), NH₄Cl (47 mg, 0.879 mmol) and HATU (279 mg, 0.734 mmol) in DMF (10 mL) was treated with DIPEA (95 mg, 0.735 mmol) and the reaction mixture was stirred at room temperature. After 12 h, the resulting solution was diluted with EtOAc (30 mL), washed with brine (3×15 mL), dried (Na₂SO₄), filtered and concentrated in vacuo. Purification of the resulting residue by recrystallization with EtOAc/Hexane (1/1) yielded 2-(2-(3-methoxyphenyl)-2-(1-oxo-6-(1H-pyrazol-4-yl)isoindolin-2-yl)ethoxy)acetamide as a white solid.

¹H NMR (400 MHz, DMSO-d6) δ 12.97 (brs, 1H), 8.31 (brs, 1H), 8.03 (brs, 1H), 7.90 (d, J=1.6 Hz, 1H), 7.84 (dd, J=7.9, 1.7 Hz, 1H), 7.53 (d, J=7.9 Hz, 1H), 7.26 (t, J=8.1 Hz, 1H), 7.10-7.23 (m, 2H), 6.85-6.90 (m, 3H), 5.56 (dd, J=9.1, 5.0 Hz, 1H), 4.65 (d, J=17.7 Hz, 1H), 4.29 (d, J=17.7 Hz, 1H), 4.17 (t, J=9.7 Hz, 1H), 3.98 (dd, J=10.4, 5.0 Hz, 1H), 3.90 (s, 2H), 3.72 (s, 3H); LCMS: Calculated: for C22H22N4O4: 406, Measured: 407 [M+H]+

### Example 22

### 2-(2-(2-Hydroxyethoxy)-1-(3-methoxyphenyl)ethyl)-6-(1H-pyrazol-4-yl)isoindolin-1-one

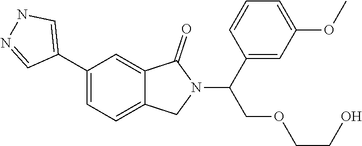 [SMILES: COc1cccc(C(COCCO)N2Cc3ccc(-c4cn[nH]c4)cc3C2=O)c1]

A mixture of 2-(2-(3-methoxyphenyl)-2-(1-oxo-6-(1H-pyrazol-4-yl)isoindolin-2-yl)ethoxy)acetic acid (60 mg, 0.147 mmol) in THE (10 mL) was treated with dropwise addition of BH₃ (1M in THF, 0.441 mL, 0.441 mmol) at 0° C. After 2 h, the reaction mixture was poured into ice/water (20 mL) and extracted with EtOAc (3×25 mL) washed with brine (3×15 mL), dried (Na₂SO₄), filtered and concentrated in vacuo. Purification of the resulting residue by preparative TLC (0-10% MeOH/DCM) yielded 2-(2-(2-Hydroxyethoxy)-1-(3-methoxyphenyl)ethyl)-6-(1H-pyrazol-4-yl)isoindolin-1-one as a white solid.

¹H NMR (400 MHz, DMSO-d6) δ 12.98 (brs, 1H), 8.33 (brs, 1H), 8.02 (brs, 1H), 7.91 (d, J=1.6 Hz, 1H), 7.85 (dd, J=7.9, 1.7 Hz, 1H), 7.54 (d, J=7.9 Hz, 1H), 7.27 (t, J=8.2 Hz, 1H), 6.86-6.91 (m, 3H), 5.50 (dd, J=8.8, 5.2 Hz, 1H), 4.57-4.62 (m, 2H), 4.35 (d, J=17.7 Hz, 1H), 4.09 (dd, J=10.6, 8.8 Hz, 1H), 3.96 (dd, J=10.6, 5.2 Hz, 1H), 3.74 (s, 3H), 3.49-3.57 (m, 4H); LCMS: Calculated: for C22H23N3O4: 393, Measured: 394 [M+H]+

### Example 23

### 2-((3-methoxyphenyl)(2H-tetrazol-5-yl)methyl)-6-(1H-pyrazol-4-yl)isoindolin-1-one

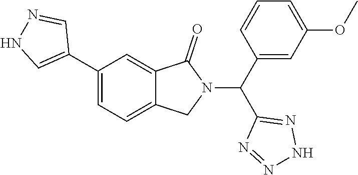 [SMILES: COc1cccc(C(c2nn[nH]n2)N2Cc3ccc(-c4cn[nH]c4)cc3C2=O)c1]

### Step 1: 2-(6-Bromo-1-oxo-2,3-dihydro-1H-isoindol-2-yl)-2-(3-methoxyphenyl)acetonitrile

A solution of 2-amino-2-(3-methoxyphenyl)acetonitrile (500 mg, 3.083 mmol), methyl 5-bromo-2-(bromomethyl)benzoate (1.139 g, 3.699 mmol) in acetonitrile (5 mL) and N,N-diisopropylethylamine (1.195 g, 9.248 mmol) was stirred at room temperature. After 1 h, the resulting solution was warmed to 85° C. After 12 h, the reaction mixture was allowed to cool to room temperature, poured into water, extracted with EtOAc (2×25 mL), washed with brine, dried (Na₂SO₄), and concentrated in vacuo. Purification of the resulting residue by silica gel chromatography (40 g column, 0-100% EtOAc/Heptane) yielded 2-(6-bromo-1-oxo-2,3-dihydro-1H-isoindol-2-yl)-2-(3-methoxyphenyl)acetonitrile as a yellow oil. LCMS: Calculated: for C17H13BrN2O2: 357, Measured: 357 [M]+.

### Step 2: 2-(3-Methoxyphenyl)-2-{6-[1-(oxan-2-yl)-1H-pyrazol-4-yl]-1-oxo-2,3-dihydro-1H-isoindol-2-yl}acetonitrile

A solution of 2-(6-bromo-1-oxo-2,3-dihydro-1H-isoindol-2-yl)-2-(3-methoxyphenyl)acetonitrile (350 mg, 0.980 mmol), 1-(oxan-2-yl)-4-(tetramethyl-1,3,2-dioxaborolan-2-yl)-1H-pyrazole (327.0 mg, 1.18 mmol), potassium carbonate (270.4 mg, 1.96 mmol) and Pd(PPh₃)₄ (113.2 mg, 0.09 mmol) in DMF/H₂O (6 mL, 5/1) was warmed to 100° C. After 12 h, the reaction mixture was poured into water, extracted with EtOAc (2×25 mL), washed with brine, dried (Na₂SO₄), and concentrated in vacuo. Purification of the resulting residue by silica gel chromatography (40 g column, 0-100% EtOAc/Heptane) yielded 2-(3-methoxyphenyl)-2-{6-[1-(oxan-2-yl)-1H-pyrazol-4-yl]-1-oxo-2,3-dihydro-1H-isoindol-2-yl}acetonitrile as a yellow oil. LCMS: Calculated: for C25H24N4O3: 428, Measured: 429 [M+H]+.

### Step 3: 2-((3-Methoxyphenyl)(2H-tetrazol-5-yl)methyl)-6-(1-(tetrahydro-2H-pyran-2-yl)-1H-pyrazol-4-yl)isoindolin-1-one

A solution of 2-(3-Methoxyphenyl)-2-{6-[1-(oxan-2-yl)-1H-pyrazol-4-yl]-1-oxo-2,3-dihydro-1H-isoindol-2-yl}acetonitrile (300 mg, 0.70 mmol), azidotrimethylsilane (161.3 mg, 1.40 mmol), dibutyltin oxide (17.429 mg, 0.070 mmol) in toluene (5 mL) was warmed to 110° C. After 12 h, the reaction mixture was poured into water, extracted with EtOAc (2×25 mL), washed with brine, dried (Na₂SO₄), and concentrated in vacuo. Purification of the resulting residue by silica gel chromatography (40 g column, 0-20% MeOH/DCM) yielded 2-((3-methoxyphenyl)(2H-tetrazol-5-yl)methyl)-6-(1-(tetrahydro-2H-pyran-2-yl)-1H-pyrazol-4-yl)isoindolin-1-one as a yellow oil. LCMS: Calculated: for C25H25N7O3: 471, Measured: 472 [M+H]+.

### Step 4: 2-((3-Methoxyphenyl)(2H-tetrazol-5-yl)methyl)-6-(1H-pyrazol-4-yl)isoindolin-1-one

2-((3-methoxyphenyl)(2H-tetrazol-5-yl)methyl)-6-(1H-pyrazol-4-yl)isoindolin-1-one was prepared according to the procedure described in Example 14, Step 3, substituting 2-((3-methoxyphenyl)(2H-tetrazol-5-yl)methyl)-6-(1-(tetrahydro-2H-pyran-2-yl)-1H-pyrazol-4-yl)isoindolin-1-one for 2-(2-hydroxy-1-(3-methoxyphenyl)ethyl)-6-(1-(tetrahydro-2H-pyran-2-yl)-1H-pyrazol-4-yl)isoindolin-1-one.

¹H NMR (300 MHz, DMSO-d6) δ 12.98 (s, 1H), 8.28-8.36 (m, 1H), 8.02 (s, 1H), 7.94 (d, J=1.6 Hz, 1H), 7.85 (dd, J=7.8, 1.7 Hz, 1H), 7.54 (d, J=8.0 Hz, 1H), 7.30 (t, J=8.2 Hz, 1H), 6.87-6.99 (m, 2H), 6.74-6.82 (m, 2H), 4.72 (d, J=17.6 Hz, 1H), 4.17 (d, J=17.7 Hz, 1H), 3.70 (s, 3H); LCMS: Calculated: for C20H17N7O2: 387, Measured: 388 [M+H]+.

### Example 24

### 2-(1-(3-methoxyphenyl)-2-oxo-2-phenylethyl)-6-(1H-pyrazol-4-yl)isoindolin-1-one

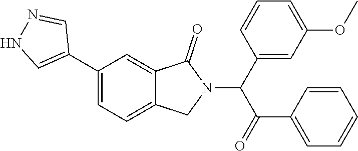 [SMILES: COc1cccc(C(C(=O)c2ccccc2)N2Cc3ccc(-c4cn[nH]c4)cc3C2=O)c1]

### Step 1: 6-Bromo-2-(1-(3-methoxyphenyl)-2-oxo-2-phenylethyl)isoindolin-1-one

6-Bromo-2-(1-(3-methoxyphenyl)-2-oxo-2-phenylethyl)isoindolin-1-one was prepared according to the procedure described in Example 1, Step 1 Alternate substituting methyl 2-bromo-2-(3-methoxyphenyl)-1-phenylethanone for 1-(bromomethyl)-3-methoxybenzene. LCMS: Calculated: for C23H18BrNO3: 436, Measured: 458 [M+Na]+

### Step 2: 2-(1-(3-Methoxyphenyl)-2-oxo-2-phenylethyl)-6-(1H-pyrazol-4-yl)isoindolin-1-one

2-(1-(3-Methoxyphenyl)-2-oxo-2-phenylethyl)-6-(1H-pyrazol-4-yl)isoindolin-1-one was prepared according to the procedure described in Example 1, Step 5, substituting 6-bromo-2-(1-(3-methoxyphenyl)-2-oxo-2-phenylethyl)isoindolin-1-one for 6-bromo-2-(3-methoxybenzyl)isoindolin-1-one.

¹H NMR (400 MHz, DMSO-d6) δ 12.98 (s, 1H), 8.34 (s, 1H), 7.71-8.18 (m, 4H), 7.20-7.70 (m, 6H), 6.99-7.07 (m, 2H), 6.90-6.98 (m, 2H), 4.61 (d, J=17.4 Hz, 1H), 3.91 (d, J=17.5 Hz, 1H), 3.73 (s, 3H); LCMS: Calculated: for C26H21N3O3: 423, Measured: 424 [M+H]+.

### Example 25

### 2-(1-(2,5-dimethoxyphenyl)-2-hydroxyethyl)-6-(1H-pyrazol-4-yl)isoindolin-1-one

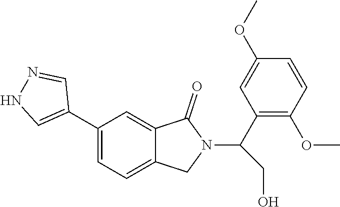 [SMILES: COc1ccc(OC)c(C(CO)N2Cc3ccc(-c4cn[nH]c4)cc3C2=O)c1]

2-(1-(2,5-Dimethoxyphenyl)-2-hydroxyethyl)-6-(1H-pyrazol-4-yl)isoindolin-1-one was similarly prepared according to the procedures described herein, selecting and substituting suitable reagents and starting materials, as would be readily recognized by those skilled in the art.

¹H NMR (400 MHz, MeOH) δ 8.01-8.25 (m, 1H), 7.94 (s, 1H), 7.82 (dd, J=1.26, 7.83 Hz, 1H), 7.28-7.61 (m, 1H), 7.02-7.23 (m, 1H), 6.78-6.95 (m, 1H), 5.68 (dd, J=5.05, 8.08 Hz, 1H), 4.54-4.73 (m, 1H), 4.35 (d, J=17.68 Hz, 1H), 4.15-4.23 (m, 1H), 4.04-4.15 (m, 1H), 3.82-3.86 (m, 1H), 3.76-3.79 (m, 1H), 3.72-3.75 (m, 1H), 3.30-3.40 (m, 6H); LCMS: Calculated: for C21H21N3O4: 379, Measured: 380 [M+H]+.

### Example 26

### 3-(3-methoxyphenyl)-3-(1-oxo-6-(1H-pyrazol-4-yl)isoindolin-2-yl)propanoic acid

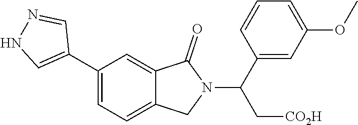 [SMILES: COc1cccc(C(CC(=O)O)N2Cc3ccc(-c4cn[nH]c4)cc3C2=O)c1]

### And Example 27

### 3-(3-methoxyphenyl)-3-(1-oxo-5-(1H-pyrazol-4-yl)isoindolin-2-yl)propanoic acid

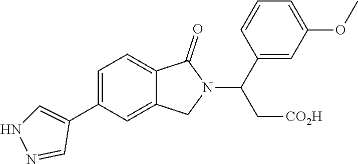 [SMILES: COc1cccc(C(CC(=O)O)N2Cc3cc(-c4cn[nH]c4)ccc3C2=O)c1]

### Step 1: (4-(1-(Tetrahydro-2H-pyran-2-yl)-1H-pyrazol-4-yl)-1,2-phenylene)dimethanol

A solution of 4-bromo-1,2-phenylene)dimethanol (1 g, 4.61 mmol), 1-(oxan-2-yl)-4-(tetramethyl-1,3,2-dioxaborolan-2-yl)-1H-pyrazole (1.54 g, 5.53 mmol), potassium carbonate (5.09 g, 36.9 mmol), Pd(PPh₃)₄ (0.532 g, 0.46 mmol) in DMF/H₂O (10 mL, 5/1) was warmed to 100° C. After 12 h, the reaction mixture was poured into water, extracted with EtOAc (2×25 mL), washed with brine, dried (Na₂SO₄), and concentrated in vacuo. Purification of the resulting residue by silica gel chromatography (40 g column, 0-20% MeOH/DCM yielded (4-(1-(tetrahydro-2H-pyran-2-yl)-1H-pyrazol-4-yl)-1,2-phenylene)dimethanol as a orange oil. LCMS: Calculated: for C16H20N2O3: 288, Measured: 311 [M+Na]+.

### Step 2: 4-(1-(Tetrahydro-2H-pyran-2-yl)-1H-pyrazol-4-yl)phthalaldehyde

Oxalyl chloride (2.5 mL, 5 mmol), was added to a solution of dimethyl sulfoxide (0.404 mL, 5.69 mmol) in DCM (45 ml) at −78° C. After 30 min, (4-(1-(tetrahydro-2H-pyran-2-yl)-1H-pyrazol-4-yl)-1,2-phenylene)dimethanol (1 g, 3.47 mmol) in DCM (11.24 ml) was added slowly at −78° C. After addition was complete, the resulting mixture was allowed to stir at −78° C. After 1.5 h, the reaction mixture was treated with water, allowed to warm to room temperature, diluted with DCM (25 mL), washed with brine, dried (Na₂SO₄), and concentrated in vacuo. Purification of the resulting residue by silica gel chromatography (40 g column, 0-10% MeOH/DCM) yielded 4-(1-(tetrahydro-2H-pyran-2-yl)-1H-pyrazol-4-yl)phthalaldehyde as an orange oil. LCMS: Calculated: for C16H16N2O3: 284, Measured: 285 [M+H]+.

### Step 3: Methyl 3-(3-methoxyphenyl)-3-(1-oxo-6-(1-(tetrahydro-2H-pyran-2-yl)-1H-pyrazol-4-yl)isoindolin-2-yl)propanoate and Methyl 3-(3-methoxyphenyl)-3-(1-oxo-5-(1-(tetrahydro-2H-pyran-2-yl)-1H-pyrazol-4-yl)isoindolin-2-yl)propanoate

A solution of methyl 3-amino-3-(3-methoxyphenyl)propanoate (0.324 g, 1.32 mmol) in DCM (10 mL) was treated with AcOH (1.188 g, 0.020 mol), and 4-(1-(tetrahydro-2H-pyran-2-yl)-1H-pyrazol-4-yl)phthalaldehyde (0.3 g, 1.06 mmol). The resulting mixture was stirred at 40° C. After 2 h, the reaction mixture was allowed to cool to room temperature, poured into water, extracted with EtOAc (2×25 mL), washed with brine, dried (Na₂SO₄), and concentrated in vacuo. Purification of the resulting residue by silica gel chromatography (40 g column, 0-100% EtOAc/Heptane) to yielded as a mixture of methyl 3-(3-methoxyphenyl)-3-(1-oxo-6-(1-(tetrahydro-2H-pyran-2-yl)-1H-pyrazol-4-yl)isoindolin-2-yl)propanoate and methyl 3-(3-methoxyphenyl)-3-(1-oxo-5-(1-(tetrahydro-2H-pyran-2-yl)-1H-pyrazol-4-yl)isoindolin-2-yl)propanoate as a yellow solid. LCMS: Calculated: for C27H29N3O5: 475, Measured: 476 [M+H]+.

### Step 4: Methyl 3-(3-methoxyphenyl)-3-(1-oxo-6-(1H-pyrazol-4-yl)isoindolin-2-yl)propanoate and Methyl 3-(3-methoxyphenyl)-3-(1-oxo-5-(1H-pyrazol-4-yl)isoindolin-2-yl)propanoate

A solution of methyl 3-(3-methoxyphenyl)-3-(1-oxo-6-(1-(tetrahydro-2H-pyran-2-yl)-1H-pyrazol-4-yl)isoindolin-2-yl)propanoate and methyl 3-(3-methoxyphenyl)-3-(1-oxo-5-(1-(tetrahydro-2H-pyran-2-yl)-1H-pyrazol-4-yl)isoindolin-2-yl)propanoate (220 mg, 0.463 mmol) in DCM (1 mL) was treated with TFA (1 mL). The resulting mixture was stirred for 2 h at room temperature. The resulting mixture was concentrated in vacuo. Purification of the resulting residue by C18 reverse column chromatography (80 g, 0-100% ACN/H₂O (containing 0.05% TFA)) yielded a mixture of methyl 3-(3-methoxyphenyl)-3-(1-oxo-6-(1H-pyrazol-4-yl)isoindolin-2-yl)propanoate and methyl 3-(3-methoxyphenyl)-3-(1-oxo-5-(1H-pyrazol-4-yl)isoindolin-2-yl)propanoate as a white solid. LCMS: Calculated: for C22H21N3O4: 391, Measured: 392 [M+H]+.

### Step 5: 3-(3-Methoxyphenyl)-3-(1-oxo-6-(1H-pyrazol-4-yl)isoindolin-2-yl)propanoic acid

A solution of methyl 3-(3-methoxyphenyl)-3-(1-oxo-6-(1H-pyrazol-4-yl)isoindolin-2-yl)propanoate (150 mg, 0.383 mmol) in THE (2 mL), MeOH (2 mL) added 2M NaOH (2 mL, 4.00 mmol) was stirred at 25° C. After 12 h, the resulting solution was concentrated in vacuo and the residue was treated with 1N HCl (pH=5). 3-(3-Methoxyphenyl)-3-(1-oxo-6-(1H-pyrazol-4-yl)isoindolin-2-yl)propanoic acid was collected by filtration as an off-white solid.

¹H NMR (300 MHz, DMSO-d6) δ 12.69-12.72 (m, 2H), 8.13 (s, 2H), 7.70-7.75 (m, 2H), 7.62 (d, J=7.9 Hz, 1H), 7.23-7.26 (m, 1H), 6.83-6.94 (m, 3H), 5.69 (t, J=7.8 Hz, 1H), 4.50 (d, J=17.5 Hz, 1H), 4.14 (d, J=17.6 Hz, 1H), 3.72 (s, 3H), 3.07-3.13 (m, 2H); LC/MS: Calculated: for C21H19N3O4: 377, Measured: 378 [M+H]+.

### Step 6: 3-(3-Methoxyphenyl)-3-(1-oxo-5-(1H-pyrazol-4-yl)isoindolin-2-yl)propanoic acid

3-(3-Methoxyphenyl)-3-(1-oxo-5-(1H-pyrazol-4-yl)isoindolin-2-yl)propanoic acid was prepared according to the procedure described in STEP 5 above, substituting methyl 3-(3-methoxyphenyl)-3-(1-oxo-5-(1H-pyrazol-4-yl)isoindolin-2-yl)propanoate for methyl 3-(3-methoxyphenyl)-3-(1-oxo-6-(1H-pyrazol-4-yl)isoindolin-2-yl)propanoate.

¹H NMR (300 MHz, DMSO-d6) δ 8.16 (s, 2H), 7.85 (d, J=1.6 Hz, 1H), 7.82 (dd, J=7.9, 1.7 Hz, 1H), 7.50 (d, J=7.9 Hz, 1H), 7.27 (dd, J=8.7, 7.3 Hz, 1H), 6.93 (dd, J=7.8, 1.6 Hz, 2H), 6.84-6.87 (m, 1H), 5.72 (t, J=7.9 Hz, 1H), 4.48 (d, J=17.6 Hz, 1H), 4.11 (d, J=17.6 Hz, 1H), 3.72 (s, 3H), 3.03-3.21 (m, 2H); LCMS: Calculated: for C21H19N3O4: 377, Measured: 378 [M+H]+.

### Example 28

### 4-(3-methoxyphenyl)-4-(1-oxo-6-(1H-pyrazol-4-yl)isoindolin-2-yl)butanoic acid

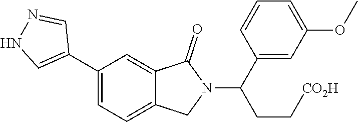 [SMILES: COc1cccc(C(CCC(=O)O)N2Cc3ccc(-c4cn[nH]c4)cc3C2=O)c1]

### And Example 29

### 4-(3-methoxyphenyl)-4-(1-oxo-5-(1H-pyrazol-4-yl)isoindolin-2-yl)butanoic acid

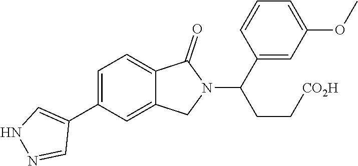 [SMILES: COc1cccc(C(CCC(=O)O)N2Cc3cc(-c4cn[nH]c4)ccc3C2=O)c1]

### Step 1: 3-(Bromo-1-oxoisoindolin-2-yl)-3-(3-methoxyphenyl)propanoate and methyl 3-(5-bromo-1-oxoisoindolin-2-yl)-3-(3-methoxyphenyl)propanoate

A solution of methyl 3-amino-3-(3-methoxyphenyl)propanoate (1.8 g, 7.33 mmol), AcOH (6.60 g, 110 mmol) in DCM (30 mL) was treated with 4-bromophthalaldehyde (1.56 g, 7.33 mmol) and warmed to 40° C. After 3 h, the reaction was quenched with water, extracted with EtOAc (2×25 mL), washed with brine, dried (Na₂SO₄), and concentrated in vacuo. Purification of the resulting residue by silica gel chromatography (40 g column, 0-100% EtOAc/Heptane) to yield a mixture of 3-(bromo-1-oxoisoindolin-2-yl)-3-(3-methoxyphenyl)propanoate and methyl 3-(5-bromo-1-oxoisoindolin-2-yl)-3-(3-methoxyphenyl)propanoate as a yellow oil. LCMS: Calculated: for C19H18BrNO4: 404.26, Measured: 406.2 [M+2H]+.

### Step 2: 6-Bromo-2-(3-hydroxy-1-(3-methoxyphenyl)propyl)isoindolin-1-one and 5-bromo-2-(3-hydroxy-1-(3-methoxyphenyl)propyl)isoindolin-1-one

A solution of 3-(bromo-1-oxoisoindolin-2-yl)-3-(3-methoxyphenyl)propanoate and methyl 3-(5-bromo-1-oxoisoindolin-2-yl)-3-(3-methoxyphenyl)propanoate (1.5 g, 3.711 mmol) in THE (20 mL) was treated with LiBH₄ (0.242 g, 11.13 mmol) and the resulting mixture was stirred at room temperature. After 12 h, the reaction was quenched with water, extracted with EtOAc (2×25 mL), washed with brine, dried (Na₂SO₄), and concentrated in vacuo. Purification of the resulting residue by silica gel chromatography (40 g column, 0-100% EtOAc/Heptane) yielded a mixture of 6-bromo-2-(3-hydroxy-1-(3-methoxyphenyl)propyl)isoindolin-1-one and 5-bromo-2-(3-hydroxy-1-(3-methoxyphenyl)propyl)isoindolin-1-one as a yellow oil. LCMS: Calculated: for C18H18BrNO3: 376, Measured: 378 [M+2H]+.

### Step 3: 6-Bromo-2-(3-hydroxy-1-(3-methoxyphenyl)propyl)isoindolin-1-one and 5-bromo-2-(3-hydroxy-1-(3-methoxyphenyl)propyl)isoindolin-1-one

A solution of 6-Bromo-2-(3-hydroxy-1-(3-methoxyphenyl)propyl)isoindolin-1-one and 5-bromo-2-(3-hydroxy-1-(3-methoxyphenyl)propyl)isoindolin-1-one (1 g, 2.66 mmol), TEA (0.808 g, 7.97 mmol) in DCM (50 mL) was treated with MsCl (0.396 g, 3.46 mmol) and was stirred at room temperature. After 3 h, the reaction was quenched with water, extracted with EtOAc (2×25 mL), washed with brine, dried (Na₂SO₄), and concentrated in vacuo. Purification of the resulting residue by silica gel chromatography (40 g column, 0-100% EtOAc/Heptane) yielded a mixture of 6-bromo-2-(3-hydroxy-1-(3-methoxyphenyl)propyl)isoindolin-1-one and 5-bromo-2-(3-hydroxy-1-(3-methoxyphenyl)propyl)isoindolin-1-one as a yellow oil. LCMS: Calculated: for C19H20BrNO5S: 454, Measured: 456 [M+H]+.

### Step 4: 4-(6-Bromo-1-oxoisoindolin-2-yl)-4-(3-methoxyphenyl)butanenitrile and 4-(5-bromo-1-oxoisoindolin-2-yl)-4-(3-methoxyphenyl)butanenitrile

A solution of 6-Bromo-2-(3-hydroxy-1-(3-methoxyphenyl)propyl)isoindolin-1-one and 5-bromo-2-(3-hydroxy-1-(3-methoxyphenyl)propyl)isoindolin-1-one (1 g, 2.201 mmol) in DMSO (20 mL) was treated with KCN (0.215 g, 3.302 mmol) and warmed to 40° C. After 12 h, the reaction was quenched with water, extracted with EtOAc (2×25 mL), washed with brine, dried (Na₂SO₄), and concentrated in vacuo. Purification of the resulting residue by silica gel chromatography (40 g column, 0-100% EtOAc/Heptane) yielded a mixture of 4-(6-bromo-1-oxoisoindolin-2-yl)-4-(3-methoxyphenyl)butanenitrile and 4-(5-bromo-1-oxoisoindolin-2-yl)-4-(3-methoxyphenyl)butanenitrile. LCMS: Calculated: for C19H17BrN2O2: 385, Measured: 407 [M+Na]+.

### Step 5: 4-(6-Bromo-1-oxoisoindolin-2-yl)-4-(3-methoxyphenyl)butanoic acid and 4-(5-bromo-1-oxoisoindolin-2-yl)-4-(3-methoxyphenyl)butanoic acid

A solution of 4-(6-bromo-1-oxoisoindolin-2-yl)-4-(3-methoxyphenyl)butanenitrile and 4-(5-bromo-1-oxoisoindolin-2-yl)-4-(3-methoxyphenyl)butanenitrile (200 mg, 0.519 mmol) in EtOH (4 mL) was treated with 1M NaOH (2 mL) and warmed to 90° C. After 12 h, the resulting mixture was concentrated in vacuo, treated with 1N HCl (pH=5), extracted with EtOAc (2×25 mL), washed with brine, dried (Na₂SO₄), and concentrated in vacuo to yield a mixture of 4-(6-bromo-1-oxoisoindolin-2-yl)-4-(3-methoxyphenyl)butanoic acid and 4-(5-bromo-1-oxoisoindolin-2-yl)-4-(3-methoxyphenyl)butanoic acid as a yellow oil. LCMS: Calculated: for C19H18BrNO4: 404, Measured: 406 [M+2H]+.

### Step 6: 4-(3-Methoxyphenyl)-4-(1-oxo-6-(1H-pyrazol-4-yl)isoindolin-2-yl)butanoic acid and 4-(3-methoxyphenyl)-4-(1-oxo-5-(1H-pyrazol-4-yl)isoindolin-2-yl)butanoic acid

4-(3-Methoxyphenyl)-4-(1-oxo-6-(1H-pyrazol-4-yl)isoindolin-2-yl)butanoic acid and 4-(3-methoxyphenyl)-4-(1-oxo-5-(1H-pyrazol-4-yl)isoindolin-2-yl)butanoic acid were prepared according to the procedure described in Example 1, Step 5, substituting a mixture of 4-(6-bromo-1-oxoisoindolin-2-yl)-4-(3-methoxyphenyl)butanoic acid and 4-(5-bromo-1-oxoisoindolin-2-yl)-4-(3-methoxyphenyl)butanoic acid for 6-bromo-2-(3-methoxybenzyl)isoindolin-1-one. Purification of the resulting residue by C18 reverse column chromatography (80 g, 0-100% ACN/H₂O (containing 0.05% TFA)) yielded 4-(3-methoxyphenyl)-4-(1-oxo-6-(1H-pyrazol-4-yl)isoindolin-2-yl)butanoic acid as a white solid and 4-(3-methoxyphenyl)-4-(1-oxo-5-(1H-pyrazol-4-yl)isoindolin-2-yl)butanoic acid as a white solid.

4-(3-Methoxyphenyl)-4-(1-oxo-6-(1H-pyrazol-4-yl)isoindolin-2-yl)butanoic acid ¹H NMR (300 MHz, DMSO-d6) δ 8.13 (s, 2H), 7.71-7.74 (m, 2H), 7.63 (d, J=7.9 Hz, 1H), 7.28 (t, J=7.8 Hz, 1H), 6.84-6.93 (m, 3H), 5.34 (dd, J=9.1, 5.6 Hz, 1H), 4.51 (d, J=17.5 Hz, 1H), 4.04 (d, J=17.6 Hz, 1H), 3.72 (s, 3H), 2.06-2.40 (m, 4H); LCMS: Calculated: for C22H21N3O4: 391, Measured: 392 [M+H]+

and 4-(3-methoxyphenyl)-4-(1-oxo-5-(1H-pyrazol-4-yl)isoindolin-2-yl)butanoic acid: ¹H NMR (300 MHz, DMSO-d6) δ 8.16 (s, 2H), 7.89 (d, J=1.6 Hz, 1H), 7.79 (dd, J=7.9, 1.7 Hz, 1H), 7.49 (d, J=7.8 Hz, 1H), 7.28 (t, J=7.7 Hz, 1H), 6.84-6.93 (m, 3H), 5.37 (dd, J=9.1, 5.6 Hz, 1H), 4.48 (d, J=17.7 Hz, 1H), 4.02 (d, J=17.7 Hz, 1H), 3.72 (s, 3H), 2.24-2.32 (m, 3H); LCMS: Calculated: for C22H21N3O4: 391, Measured: 392 [M+H]+

### Example 30

### 2-(2-hydroxy-1-(3-methoxyphenyl)ethyl)-6-(pyridin-3-yl)isoindolin-1-one

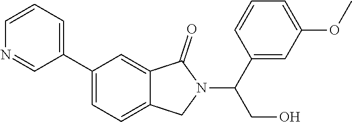 [SMILES: COc1cccc(C(CO)N2Cc3ccc(-c4cccnc4)cc3C2=O)c1]

2-(2-Hydroxy-1-(3-methoxyphenyl)ethyl)-6-(pyridin-3-yl)isoindolin-1-one was prepared according to the procedure described in Example 32, Step 2, substituting pyridin-3-yl boronic acid for tert-butyl 4-(4,4,5,5-tetramethyl-1,3,2-dioxaborolan-2-yl)-1H-pyrazole-1-carboxylate.

¹H NMR (400 MHz, DMSO-d6) δ 8.84-8.76 (m, 2H), 8.20-8.07 (m, 4H), 7.77 (d, J=8.0 Hz, 1H), 7.26 (t, J=7.8 Hz, 1H), 6.93-6.82 (m, 3H), 5.36 (m, 1H), 4.71 (d, J=18.3 Hz, 1H), 4.41 (d, J=18.2 Hz, 1H), 4.08-3.93 (m, 2H), 3.72 (s, 3H); LCMS: Calculated: for C22H20N2O3: 360, Measured: 361 [M+H]+.

### Example 31

### 6-(2-aminopyrimidin-4-yl)-2-(2-hydroxy-1-(3-methoxyphenyl)ethyl)isoindolin-1-one

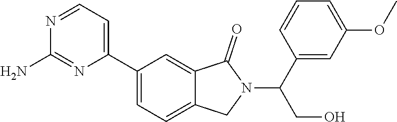 [SMILES: COc1cccc(C(CO)N2Cc3ccc(-c4ccnc(N)n4)cc3C2=O)c1]

6-(2-Aminopyrimidin-4-yl)-2-(2-hydroxy-1-(3-methoxyphenyl)ethyl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.61 (s, 1H), 8.45 (d, J=8.08 Hz, 1H), 8.34 (d, J=6.57 Hz, 1H), 7.74 (d, J=8.08 Hz, 1H), 7.57 (d, J=7.07 Hz, 1H), 7.24-7.33 (m, 1H), 6.92-7.00 (m, 2H), 6.83-6.92 (m, 1H), 5.52 (dd, J=5.05, 9.09 Hz, 1H), 4.78 (d, J=18.69 Hz, 1H), 4.46 (d, J=18.19 Hz, 1H), 4.18-4.30 (m, 1H), 4.05-4.18 (m, 1H), 3.71-3.83 (m, 3H); LCMS: Calculated: for C21H20N4O3: 375, Measured: 377 [M+2H]+.

### Example 32

### 2-(2-hydroxy-1-(3-methoxyphenyl)ethyl)-6-(1H-pyrazol-4-yl)-4-(pyridin-3-yl)isoindolin-1-one

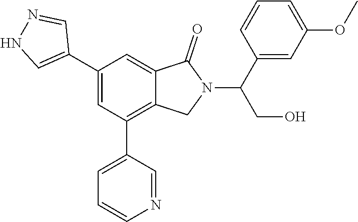 [SMILES: COc1cccc(C(CO)N2Cc3c(cc(-c4cn[nH]c4)cc3-c3cccnc3)C2=O)c1]

### Step 1: 4-Bromo-2-(2-hydroxy-1-(3-methoxyphenyl)ethyl)-6-iodoisoindolin-1-one

4-Bromo-2-(2-hydroxy-1-(3-methoxyphenyl)ethyl)-6-iodoisoindolin-1-one was prepared according to the procedure described in Example 14, Step 1 substituting methyl 3-bromo-2-(bromomethyl)-5-iodobenzoate for methyl 5-bromo-2-(bromomethyl)benzoate. LCMS: Calculated: for C17H15BrINO3: 488, Measured: 490 [M+2H]+.

### Step 2: 4-Bromo-2-(2-hydroxy-1-(3-methoxyphenyl)ethyl)-6-(1H-pyrazol-4-yl)isoindolin-1-one

Under a nitrogen atmosphere, a mixture of 4-Bromo-2-(2-hydroxy-1-(3-methoxyphenyl)ethyl)-6-iodoisoindolin-1-one (200 mg, 0.410 mmol), tert-butyl 4-(4,4,5,5-tetramethyl-1,3,2-dioxaborolan-2-yl)-1H-pyrazole-1-carboxylate (120.5 mg, 0.410 mmol), K₂CO₃ (113 mg, 0.818 mmol) and Pd(PPh₃)₄ (23.6 mg, 0.020 mmol) in 1,4-dioxane (10 mL) and H₂O (1 mL) was warmed to 95° C. After 12 h, the reaction mixture was allowed to cool to room temperature, diluted with EtOAc (50 mL), washed with brine (3×15 mL), dried (Na₂SO₄), and concentrated in vacuo. Purification of the resulting residue by silica gel chromatography (20 g column, 0-20% MeOH/DCM) yielded 4-bromo-2-(2-hydroxy-1-(3-methoxyphenyl)ethyl)-6-(1H-pyrazol-4-yl)isoindolin-1-one as a yellow solid. LCMS: Calculated: for C20H18BrN3O3: 428, Measured: 430 [M+2H]+.

### Step 3: 2-(2-Hydroxy-1-(3-methoxyphenyl)ethyl)-6-(1H-pyrazol-4-yl)-4-(pyridin-3-yl)isoindolin-1-one

2-(2-Hydroxy-1-(3-methoxyphenyl)ethyl)-6-(1H-pyrazol-4-yl)-4-(pyridin-3-yl)isoindolin-1-one was prepared according to the procedure described in Example 40, Step 3, substituting 4-bromo-2-(2-hydroxy-1-(3-methoxyphenyl)ethyl)-6-(1H-pyrazol-4-yl)isoindolin-1-one for (R)-4-bromo-2-(1-(3-methoxyphenyl)ethyl)-6-(1H-pyrazol-4-yl)isoindolin-1-one and pyridine-3-boronic acid for (2-tert-butyl-4-(4,4,5,5-tetramethyl-1,3,2-dioxaborolan-2-yl)pyridine.

¹H NMR (400 MHz, DMSO-d6) δ 8.87 (d, J=1.6 Hz, 1H), 8.61-8.68 (m, 1H), 8.29 (s, 1H), 8.10 (d, J=8.1 Hz, 1H), 7.94-8.01 (m, 2H), 7.51-7.65 (m, 2H), 7.23 (t, J=7.9 Hz, 1H), 6.86-6.93 (m, 2H), 6.79-6.86 (m, 1H), 5.33 (dd, J=8.9, 5.2 Hz, 1H), 5.05 (s, 1H), 4.74 (d, J=17.6 Hz, 1H), 4.45 (d, J=17.6 Hz, 1H), 4.03 (dd, J=11.6, 9.1 Hz, 1H), 3.92 (dd, J=11.6, 5.3 Hz, 1H), 3.71 (s, 3H); LCMS: Calculated: for C25H22N4O3: 426, Measured: 427 [M+H]+.

### Example 33

### 2-(2-hydroxy-1-(3-methoxyphenyl)ethyl)-4-(3-hydroxyphenyl)-6-(1H-pyrazol-4-yl)isoindolin-1-one

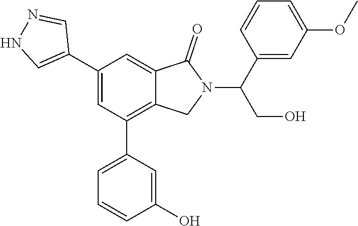 [SMILES: COc1cccc(C(CO)N2Cc3c(cc(-c4cn[nH]c4)cc3-c3cccc(O)c3)C2=O)c1]

2-(2-Hydroxy-1-(3-methoxyphenyl)ethyl)-4-(3-hydroxyphenyl)-6-(1H-pyrazol-4-yl)isoindolin-1-one was prepared according to the procedure described in Example 32, Step 3, substituting 3-(tetramethyl-1,3,2-dioxaborolan-2-yl)phenol for pyridine-3-boronic acid.

¹H NMR (300 MHz, DMSO-d6) δ 9.63 (s, 1H), 8.27 (s, 2H), 7.92 (d, J=1.6 Hz, 1H), 7.85 (d, J=1.6 Hz, 1H), 7.19-7.35 (m, 2H), 6.93-7.06 (m, 2H), 6.77-6.93 (m, 4H), 5.35 (dd, J=8.7, 5.4 Hz, 1H), 4.68 (d, J=17.6 Hz, 1H), 4.34 (d, J=17.6 Hz, 1H), 3.89-4.10 (m, 2H), 3.72 (s, 3H); LCMS: Calculated: for C26H23N3O4: 441, Measured: 442 [M+H]+.

### Example 34

### 4-(5-fluoropyridin-3-yl)-2-(2-hydroxy-1-(3-methoxyphenyl)ethyl)-6-(1H-pyrazol-4-yl)isoindolin-1-one

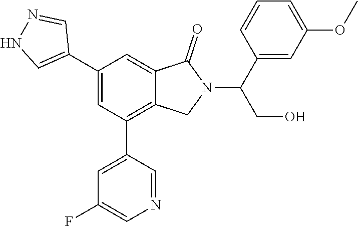 [SMILES: COc1cccc(C(CO)N2Cc3c(cc(-c4cn[nH]c4)cc3-c3cncc(F)c3)C2=O)c1]

4-(5-Fluoropyridin-3-yl)-2-(2-hydroxy-1-(3-methoxyphenyl)ethyl)-6-(1H-pyrazol-4-yl)isoindolin-1-one was prepared according to the procedure described in Example 32, Step 3, substituting (5-fluoropyridin-3-yl)boronic acid for pyridine-3-boronic acid.

¹H NMR (300 MHz, DMSO-d6) δ 13.00 (s, 1H), 8.74 (t, J=1.8 Hz, 1H), 8.64 (d, J=2.6 Hz, 1H), 8.43 (s, 1H), 8.03-8.15 (m, 2H), 7.98 (q, J=1.5 Hz, 2H), 7.21 (t, J=7.8 Hz, 1H), 6.76-6.93 (m, 3H), 5.31 (dd, J=9.0, 5.4 Hz, 1H), 5.03 (t, J=5.5 Hz, 1H), 4.75 (d, J=17.6 Hz, 1H), 4.49 (d, J=17.7 Hz, 1H), 4.02 (t, J=13.2 Hz, 1H), 3.81-3.96 (m, 1H), 3.69 (s, 3H); 19F NMR (376 MHz, DMSO) d −126.96; LCMS: Calculated: for C25H21FN4O3: 444, Measured: 445 [M+H]+.

### Example 35

### 2-(2-Hydroxy-1-(3-methoxyphenyl)ethyl)-4-(4-hydroxyphenyl)-6-(1H-pyrazol-4-yl)isoindolin-1-one

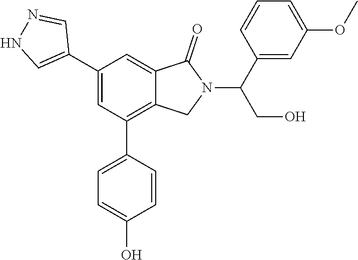 [SMILES: COc1cccc(C(CO)N2Cc3c(cc(-c4cn[nH]c4)cc3-c3ccc(O)cc3)C2=O)c1]

2-(2-Hydroxy-1-(3-methoxyphenyl)ethyl)-4-(4-hydroxyphenyl)-6-(1H-pyrazol-4-yl)isoindolin-1-one was prepared according to the procedure described in Example 32, Step 3, substituting 4-(tetramethyl-1,3,2-dioxaborolan-2-yl)phenol for pyridine-3-boronic acid.

¹H NMR (300 MHz, DMSO-d6) δ 9.63 (s, 1H), 8.21 (s, 2H), 7.81 (dd, J=12.3, 1.5 Hz, 2H), 7.37-7.47 (m, 2H), 7.21 (t, J=7.9 Hz, 1H), 6.76-6.90 (m, 5H), 5.31 (dd, J=8.8, 5.4 Hz, 1H), 4.67 (d, J=17.5 Hz, 1H), 4.32 (d, J=17.6 Hz, 1H), 3.85-4.08 (m, 3H), 3.69 (s, 3H); LCMS: Calculated: for C26H23N3O4: 441, Measured: 442 [M+H]+.

### Example 36

### 2-(2-Hydroxy-1-(3-methoxyphenyl)ethyl)-6-(1H-pyrazol-4-yl)-4-(pyridin-4-yl)isoindolin-1-one

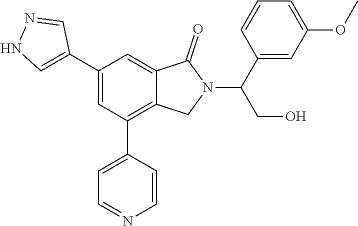 [SMILES: COc1cccc(C(CO)N2Cc3c(cc(-c4cn[nH]c4)cc3-c3ccncc3)C2=O)c1]

2-(2-Hydroxy-1-(3-methoxyphenyl)ethyl)-6-(1H-pyrazol-4-yl)-4-(pyridin-4-yl)isoindolin-1-one was prepared according to the procedure described in Example 32, Step 3, substituting pyridine-4-boronic acid for pyridine-3-boronic acid.

¹H NMR (400 MHz, DMSO-d6) δ 8.90 (brs, 2H), 8.33 (s, 2H), 8.08-8.15 (m, 4H), 7.26 (t, J=7.9 Hz, 1H), 6.82-6.96 (m, 3H), 5.37 (dd, J=9.1, 5.3 Hz, 1H), 4.84 (d, J=17.9 Hz, 1H), 4.58 (d, J=17.8 Hz, 1H), 3.92-4.10 (m, 2H), 3.73 (s, 3H); LCMS: Calculated: for C25H22N4O3: 426, Measured: 427 [M+H]+.

### Example 37

### 2-(2-hydroxy-1-(3-methoxyphenyl)ethyl)-6-(1H-pyrazol-4-yl)-4-(pyrimidin-5-yl)isoindolin-1-one

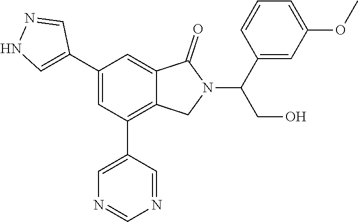 [SMILES: COc1cccc(C(CO)N2Cc3c(cc(-c4cn[nH]c4)cc3-c3cncnc3)C2=O)c1]

### Step 1: 4-Bromo-2-(2-hydroxy-1-(3-methoxyphenyl)ethyl)-6-(1-(tetrahydro-2H-pyran-2-yl)-1H-pyrazol-4-yl)isoindolin-1-one

4-Bromo-2-(2-hydroxy-1-(3-methoxyphenyl)ethyl)-6-(1-(tetrahydro-2H-pyran-2-yl)-1H-pyrazol-4-yl)isoindolin-1-one was prepared according to the procedure described in Example 83, Step 1, substituting 4-bromo-2-(2-hydroxy-1-(3-methoxyphenyl)ethyl)-6-iodoisoindolin-1-one for 5′-bromo-2′-[(3-methoxyphenyl)methyl]-2′,3′-dihydrospiro[cyclopentane-1,1′-isoindol]-3-en-3′-one. LCMS: Calculated: for C25H26BrN3O4: 512, Measured: 512 [M]+.

### Step 2: 4-Bromo-2-(2-(tert-butyldimethylsilyloxy)-1-(3-methoxyphenyl)ethyl)-6-(1-(tetrahydro-2H-pyran-2-yl)-1H-pyrazol-4-yl)isoindolin-1-one

A mixture of 4-Bromo-2-(2-hydroxy-1-(3-methoxyphenyl)ethyl)-6-(1-(tetrahydro-2H-pyran-2-yl)-1H-pyrazol-4-yl)isoindolin-1-one (700 mg, 1.37 mmol) and 2,6-lutidine (586 mg, 5.47 mmol) in DCM (20 mL) was treated with TBSOTf (1.08 g, 4.09 mmol) at 0° C. The resulting mixture was allowed to warm to room temperature. After 12 h, the reaction mixture was diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded 4-bromo-2-(2-(tert-butyldimethylsilyloxy)-1-(3-methoxyphenyl)ethyl)-6-(1-(tetrahydro-2H-pyran-2-yl)-1H-pyrazol-4-yl)isoindolin-1-one as a yellow oil. LC/MS: Calculated: for C31H40BrN3O4Si: 625, Measured: 626, 628 [M+H, M+3H]+.

### Step 3: 2-(2-Hydroxy-1-(3-methoxyphenyl)ethyl)-4-(pyrimidin-5-yl)-6-(1-(tetrahydro-2H-pyran-2-yl)-1H-pyrazol-4-yl)isoindolin-1-one

2-(2-Hydroxy-1-(3-methoxyphenyl)ethyl)-4-(pyrimidin-5-yl)-6-(1-(tetrahydro-2H-pyran-2-yl)-1H-pyrazol-4-yl)isoindolin-1-one was prepared according to the procedure described in Example 32, Step 3. substituting 4-(tetramethyl-1,3,2-dioxaborolan-2-yl)phenol for (pyrimidin-5-yl)boronic acid. LCMS: Calculated: for C29H29N5O4: 511, Measured: 512 [M+H]+

### Step 4: 2-(2-Hydroxy-1-(3-methoxyphenyl)ethyl)-6-(1H-pyrazol-4-yl)-4-(pyrimidin-5-yl)isoindolin-1-one

2-(2-Hydroxy-1-(3-methoxyphenyl)ethyl)-6-(1H-pyrazol-4-yl)-4-(pyrimidin-5-yl)isoindolin-1-one was prepared according to the procedure described in Example 60, Step 3, substituting 2-(2-Hydroxy-1-(3-methoxyphenyl)ethyl)-4-(pyrimidin-5-yl)-6-(1-(tetrahydro-2H-pyran-2-yl)-1H-pyrazol-4-yl)isoindolin-1-one for 4-bromo-2-((R)-1-(3-methoxyphenyl)ethyl)-6-(1-(tetrahydro-2H-pyran-2-yl)-1H-pyrazol-4-yl)isoindolin-1-one

¹H NMR (300 MHz, DMSO-d6) δ 12.99 (s, 1H), 9.26 (s, 1H), 9.15 (s, 2H), 8.27 (s, 2H), 8.04 (dd, J=13.3, 1.6 Hz, 2H), 7.23 (t, J=7.9 Hz, 1H), 6.79-6.96 (m, 3H), 5.34 (dd, J=8.9, 5.2 Hz, 1H), 5.05 (t, J=5.5 Hz, 1H), 4.81 (d, J=17.6 Hz, 1H), 4.54 (d, J=17.7 Hz, 1H), 4.05 (dt, J=15.2, 7.5 Hz, 1H), 3.92 (dt, J=11.2, 5.4 Hz, 1H), 3.71 (d, J=2.0 Hz, 3H); LCMS: Calculated: for C24H21N5O3: 427, Measured: 428 [M+H]+.

### Example 38

### 2-(2-hydroxy-1-(3-methoxyphenyl)ethyl)-6-(1H-pyrazol-4-yl)-4-(pyrimidin-4-yl)isoindolin-1-one

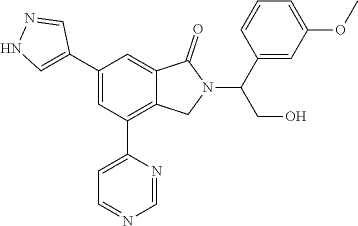 [SMILES: COc1cccc(C(CO)N2Cc3c(cc(-c4cn[nH]c4)cc3-c3ccncn3)C2=O)c1]

### Step 1: 2-(2-(tert-Butyldimethylsilyloxy)-1-(3-methoxyphenyl)ethyl)-4-(pyrimidin-4-yl)-6-(1-(tetrahydro-2H-pyran-2-yl)-1H-pyrazol-4-yl)isoindolin-1-one

Under a nitrogen atmosphere, a mixture of 4-bromo-2-(2-(tert-butyldimethylsilyloxy)-1-(3-methoxyphenyl)ethyl)-6-(1-(tetrahydro-2H-pyran-2-yl)-1H-pyrazol-4-yl)isoindolin-1-one (200 mg, 0.32 mmol), 4-(tributylstannyl)pyrimidine (354 mg, 0.96 mmol), CuI (60.8 mg, 0.32 mmol), LiCl (13.5 mg, 0.32 mmol), PPh₃ (84 mg, 0.32 mmol) and Pd₂(dba)₃ (146 mg, 0.16 mmol) in 1,4-dioxane (15 mL) was stirred at 120° C. After 12 h, the reaction mixture was allowed to cool to room temperature, diluted with EtOAc (50 mL), washed with brine (3×15 mL), dried (Na₂SO₄), and concentrated in vacuo.

Purification of the resulting residue by silica gel chromatography (24 g column, 0-100% EtOAc/Heptane) yielded 2-(2-(tert-butyldimethylsilyloxy)-1-(3-methoxyphenyl)ethyl)-4-(pyrimidin-4-yl)-6-(1-(tetrahydro-2H-pyran-2-yl)-1H-pyrazol-4-yl)isoindolin-1-one as a yellow oil. LCMS: Calculated: for C35H43N5O4Si: 625, Measured: 626 [M+H]+.

### Step 2: 2-(2-Hydroxy-1-(3-methoxyphenyl)ethyl)-6-(1H-pyrazol-4-yl)-4-(pyrimidin-4-yl)isoindolin-1-one

A mixture of 2-(2-(tert-butyldimethylsilyloxy)-1-(3-methoxyphenyl)ethyl)-4-(pyrimidin-4-yl)-6-(1-(tetrahydro-2H-pyran-2-yl)-1H-pyrazol-4-yl)isoindolin-1-one (110 mg, 0.176 mmol) in THE (3 mL) was treated with HCl (3 mL, 4M) at 0° C. The resulting mixture was allowed to warm to room temperature. After 1 h, the reaction mixture was treated with satd. aq. NaHCO₃ (20 mL), extracted with EtOAc (3×30 mL), dried (Na₂SO₄), and concentrated in vacuo. Purification of the resulting residue by C18 reverse column chromatography (80 g, 5%-35% ACN/H₂O (containing 0.05% TFA)) yielded 2-(2-hydroxy-1-(3-methoxyphenyl)ethyl)-6-(1H-pyrazol-4-yl)-4-(pyrimidin-4-yl)isoindolin-1-one as a yellow solid.

¹H NMR (400 MHz, DMSO-d6) δ 9.31 (s, 1H), 8.98 (d, J=5.2 Hz, 1H), 8.52 (d, J=1.6 Hz, 1H), 8.38 (s, 2H), 8.33 (dd, J=5.5, 1.3 Hz, 1H), 8.13 (d, J=1.5 Hz, 1H), 7.27 (t, J=7.8 Hz, 1H), 6.82-6.94 (m, 3H), 5.41 (dd, J=8.8, 5.5 Hz, 1H), 5.05 (d, J=19.0 Hz, 1H), 4.76 (d, J=18.9 Hz, 1H), 3.98-4.13 (m, 2H), 3.73 (s, 3H). 19F NMR (376 MHz, DMSO-d6) d −74.42. LCMS: Calculated: for C24H21N5O3: 427, Measured: 428 [M+H]+.

### Example 39

### 2-(2-hydroxy-1-(3-methoxyphenyl)ethyl)-6-(1H-pyrazol-4-yl)-4-(pyridin-2-yl)isoindolin-1-one

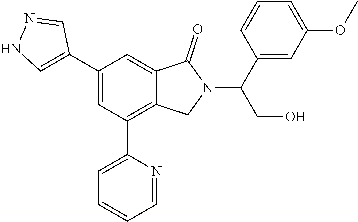 [SMILES: COc1cccc(C(CO)N2Cc3c(cc(-c4cn[nH]c4)cc3-c3ccccn3)C2=O)c1]

2-(2-Hydroxy-1-(3-methoxyphenyl)ethyl)-6-(1H-pyrazol-4-yl)-4-(pyridin-2-yl)isoindolin-1-one was prepared according to the procedure described in Example 38, Step 2, substituting 2-(tributylstannyl)pyridine for 4-(tributylstannyl)pyrimidine.

¹H NMR (400 MHz, DMSO-d6) δ 8.73 (d, J=4.8 Hz, 1H), 8.30-8.50 (m, 3H), 8.16 (d, J=8.0 Hz, 1H), 7.95-8.07 (m, 2H), 7.44 (dd, J=7.5, 4.8 Hz, 1H), 7.27 (t, J=7.9 Hz, 1H), 6.82-6.95 (m, 3H), 5.40 (dd, J=8.6, 5.5 Hz, 1H), 4.96 (d, J=18.7 Hz, 1H), 4.68 (d, J=18.7 Hz, 1H), 3.97-4.13 (m, 2H), 3.73 (s, 3H). 19F NMR (376 MHz, DMSO-d6) d −74.84; LCMS: Calculated: for C25H22N4O3: 426, Measured: 427 [M+H]+.

### Example 40

### (R)-4-(2-(tert-butyl)pyridin-4-yl)-2-(1-(3-methoxyphenyl)ethyl)-6-(1H-pyrazol-4-yl)isoindolin-1-one

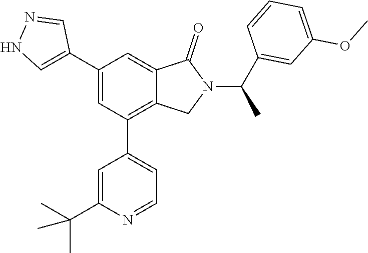 [SMILES: COc1cccc([C@@H](C)N2Cc3c(cc(-c4cn[nH]c4)cc3-c3ccnc(C(C)(C)C)c3)C2=O)c1]

### Step 1: (R)-4-Bromo-6-iodo-2-(1-(3-methoxyphenyl)ethyl)isoindolin-1-one

To a mixture of methyl 3-bromo-2-(bromomethyl)-5-iodobenzoate (1.98 g, 4.60 mmol) and (R)-1-(3-methoxyphenyl)ethanamine (760 mg, 5.0 mmol) in ACN (60 mL) was added K₂CO₃ (1.89 g, 13.6 mmol). The resulting mixture was warmed to 80° C. under nitrogen. After 16 h, the reaction mixture was diluted with 1N HCl (15 mL) and the organic layer was separated. The aqueous layer was extracted with EtOAc (3×25 mL). The combined organic layer was washed with brine (1×50 mL), dried (Na₂SO₄), filtered and concentrated in vacuo. Purification of the resulting residue by silica gel column chromatography (80 g, 0-100% EtOAc/heptane) yielded (R)-4-bromo-6-iodo-2-(1-(3-methoxyphenyl)ethyl)isoindolin-1-one as a colorless oil. LCMS: Calculated: for C17H15BrINO2: 472, Measured: 474 [M+2H]+.

### Step 2: (R)-4-Bromo-2-(1-(3-methoxyphenyl)ethyl)-6-(1H-pyrazol-4-yl)isoindolin-1-one

Under a nitrogen atmosphere, a mixture of (R)-4-bromo-6-iodo-2-(1-(3-methoxyphenyl)ethyl)isoindolin-1-one (1 g, 2.12 mmol), tert-butyl 4-(4,4,5,5-tetramethyl-1,3,2-dioxaborolan-2-yl)-1H-pyrazole-1-carboxylate (623 mg, 2.12 mmol), K₂CO₃ (585 mg, 4.23 mmol) and Pd(PPh₃)₄ (122 mg, 0.106 mmol) in 1,4-dioxane (20 mL) and H₂O (2 mL) was stirred at 95° C. After 12 h, the reaction mixture was allowed to cool to room temperature, diluted with EtOAc (80 mL), washed with brine (2×30 mL), dried (Na₂SO₄), filtered and concentrated in vacuo. Purification of the resulting residue by silica gel column chromatography (80 g, 0-15% MeOH/DCM) yielded (R)-4-bromo-2-(1-(3-methoxyphenyl)ethyl)-6-(1H-pyrazol-4-yl)isoindolin-1-one as a light yellow solid. LCMS: Calculated: for C20H18BrN3O2: 412, Measured: 414 [M+2H]+.

### Step 3: (R)-4-(2-tert-Butylpyridin-4-yl)-2-(1-(3-methoxyphenyl)ethyl)-6-(1H-pyrazol-4-yl)isoindolin-1-one

Under a nitrogen atmosphere, a mixture of (R)-4-bromo-2-(1-(3-methoxyphenyl)ethyl)-6-(1H-pyrazol-4-yl)isoindolin-1-one (150 mg, 0.364 mmol), 2-tert-butyl-4-(4,4,5,5-tetramethyl-1,3,2-dioxaborolan-2-yl)pyridine (190 mg, 0.728 mmol), K₂CO₃ (151 mg, 1.09 mmol) and Pd(PPh₃)₄ (21 mg, 0.018 mmol) in 1,4-dioxane (15 mL) and H₂O (1.5 mL) was stirred at 95° C. After 12 h, the reaction mixture was allowed to cool to room temperature, diluted with EtOAc (20 mL), washed with brine (2×30 mL), dried (Na₂SO₄), filtered and concentrated in vacuo. Purification of the resulting residue by C18 reverse column chromatography (80 g, 5%-35% ACN/H₂O (containing 0.05% TFA)) yielded (R)-4-(2-tert-butylpyridin-4-yl)-2-(1-(3-methoxyphenyl)ethyl)-6-(1H-pyrazol-4-yl)isoindolin-1-one as a light yellow solid.

¹H NMR (400 MHz, DMSO-d6) δ 8.73 (d, J=5.5 Hz, 1H), 8.33 (s, 2H), 8.07 (dd, J=7.2, 1.6 Hz, 2H), 7.79-7.91 (m, 2H), 7.26 (t, J=7.9 Hz, 1H), 6.88-6.95 (m, 2H), 6.85 (dd, J=8.2, 2.5 Hz, 1H), 5.52 (q, J=6.9 Hz, 1H), 4.78 (d, J=17.6 Hz, 1H), 4.22 (d, J=17.6 Hz, 1H), 3.73 (s, 3H), 1.64 (d, J=7.1 Hz, 3H), 1.42 (s, 9H); LCMS: Calculated: for C29H30N4O2: 466, Measured: 467 [M+H]+

### Example 41

### (R)-4-(5-(tert-butyl)pyridin-3-yl)-2-(1-(3-methoxyphenyl)ethyl)-6-(1H-pyrazol-4-yl)isoindolin-1-one

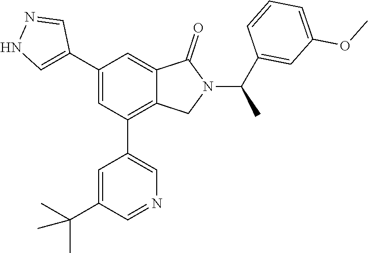 [SMILES: COc1cccc([C@@H](C)N2Cc3c(cc(-c4cn[nH]c4)cc3-c3cncc(C(C)(C)C)c3)C2=O)c1]

(R)-4-(5-(tert-butyl)pyridin-3-yl)-2-(1-(3-methoxyphenyl)ethyl)-6-(1H-pyrazol-4-yl)isoindolin-1-one was prepared according to the procedure described in Example 40, Step 3, substituting 5-tert-butylpyridin-3-ylboronic acid instead of 2-tert-butyl-4-(4,4,5,5-tetramethyl-1,3,2-dioxaborolan-2-yl)pyridine.

¹H NMR (300 MHz, DMSO-d6) δ 8.88 (d, J=2.0 Hz, 1H), 8.80 (d, J=2.1 Hz, 1H), 8.41 (t, J=2.1 Hz, 1H), 8.31 (s, 2H), 8.00-8.07 (m, 2H), 7.25 (t, J=7.9 Hz, 1H), 6.88-6.95 (m, 2H), 6.81-6.88 (m, 1H), 5.52 (q, J=7.0 Hz, 1H), 4.74 (d, J=17.5 Hz, 1H), 4.17 (d, J=17.5 Hz, 1H), 3.72 (s, 3H), 1.62 (d, J=7.1 Hz, 3H), 1.39 (s, 9H). 19F NMR (282 MHz, DMSO-d6) d −74.63; LCMS: Calculated: for C29H30N4O2: 466, Measured: 467 [M+H]+.

### Example 42

### (R)-2-(1-(3-methoxyphenyl)ethyl)-6-(1H-pyrazol-4-yl)-4-(2-(pyridin-3-yl)pyrimidin-5-yl)isoindolin-1-one

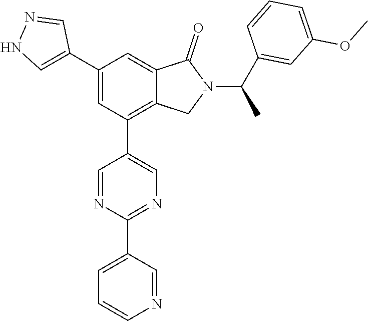 [SMILES: COc1cccc([C@@H](C)N2Cc3c(cc(-c4cn[nH]c4)cc3-c3cnc(-c4cccnc4)nc3)C2=O)c1]

(R)-2-(1-(3-Methoxyphenyl)ethyl)-6-(1H-pyrazol-4-yl)-4-(2-(pyridin-3-yl)pyrimidin-5-yl)isoindolin-1-one was prepared according to the procedure described in Example 40, Step 3, substituting 2-(pyridin-3-yl)pyrimidin-5-ylboronic acid instead of 2-tert-butyl-4-(4,4,5,5-tetramethyl-1,3,2-dioxaborolan-2-yl)pyridine.

¹H NMR (400 MHz, DMSO-d6) δ 9.63 (s, 1H), 9.32 (s, 2H), 8.77-8.95 (m, 2H), 8.33 (s, 2H), 8.15 (d, J=1.6 Hz, 1H), 8.06 (d, J=1.5 Hz, 1H), 7.75 (d, J=5.7 Hz, 1H), 7.26 (t, J=7.9 Hz, 1H), 6.89-7.00 (m, 2H), 6.84 (dd, J=8.2, 2.5 Hz, 1H), 5.54 (q, J=7.2 Hz, 1H), 4.90 (d, J=17.7 Hz, 1H), 4.36 (d, J=17.7 Hz, 1H), 3.73 (s, 3H), 1.66 (d, J=7.2 Hz, 3H); 19F NMR (376 MHz, DMSO-d6) d −74.86; Calculated: for C29H24N6O2: 488, Measured: 489 [M+H]+

### Example 43

### (R)-2-(1-(3-methoxyphenyl)ethyl)-6-(1H-pyrazol-4-yl)-4-(pyridin-3-yl)isoindolin-1-one

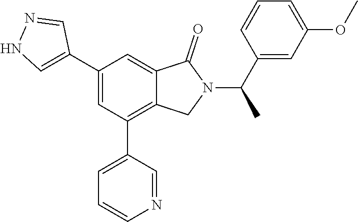 [SMILES: COc1cccc([C@@H](C)N2Cc3c(cc(-c4cn[nH]c4)cc3-c3cccnc3)C2=O)c1]

(R)-2-(1-(3-Methoxyphenyl)ethyl)-6-(1H-pyrazol-4-yl)-4-(pyridin-3-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.65 (br d, J=6.57 Hz, 1H), 8.18 (br s, 1H), 8.12 (d, J=1.52 Hz, 1H), 8.01 (d, J=1.01 Hz, 1H), 7.26 (t, J=8.08 Hz, 1H), 6.88-7.00 (m, 1H), 6.84 (dd, J=2.53, 8.08 Hz, 1H), 5.68 (d, J=7.07 Hz, 1H), 4.75 (d, J=17.68 Hz, 1H), 4.22 (d, J=18.19 Hz, 1H), 3.75 (s, 3H), 1.72 (d, J=7.07 Hz, 3H); LCMS: Calculated: for C25H22N4O2: 410, Measured: 411 [M+H]+

### Example 44

### (R)-4-(5-fluoropyridin-3-yl)-2-(1-(3-methoxyphenyl)ethyl)-6-(1H-pyrazol-4-yl)isoindolin-1-one

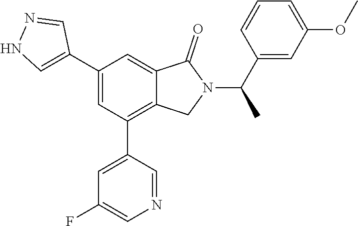 [SMILES: COc1cccc([C@@H](C)N2Cc3c(cc(-c4cn[nH]c4)cc3-c3cncc(F)c3)C2=O)c1]

(R)-4-(5-Fluoropyridin-3-yl)-2-(1-(3-methoxyphenyl)ethyl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.50-8.69 (m, 1H), 8.18 (s, 1H), 8.07 (d, J=1.52 Hz, 1H), 7.90-7.99 (m, 1H), 7.22-7.30 (m, 1H), 6.90-6.98 (m, 1H), 6.81-6.87 (m, 1H), 5.66 (d, J=7.07 Hz, 1H), 4.72 (d, J=17.68 Hz, 1H), 4.17 (d, J=17.68 Hz, 1H), 3.76 (s, 3H), 1.72 (d, J=7.07 Hz, 3H); LCMS: Calculated: for C25H21 FN4O2: 428, Measured: 429 [M+H]+.

### Example 45

### (R)-2-(1-(3-methoxyphenyl)ethyl)-6-(1H-pyrazol-4-yl)-4-(pyridin-4-yl)isoindolin-1-one

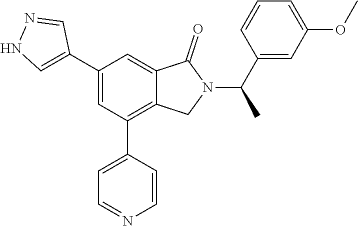 [SMILES: COc1cccc([C@@H](C)N2Cc3c(cc(-c4cn[nH]c4)cc3-c3ccncc3)C2=O)c1]

(R)-2-(1-(3-Methoxyphenyl)ethyl)-6-(1H-pyrazol-4-yl)-4-(pyridin-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.78-8.99 (m, 1H), 8.07-8.28 (m, 3H), 7.26 (t, J=8.08 Hz, 1H), 6.88-7.00 (m, 1H), 6.84 (dd, J=2.53, 8.08 Hz, 1H), 5.69 (d, J=7.07 Hz, 1H), 4.81 (s, 1H), 4.29 (d, J=17.68 Hz, 1H), 3.76 (s, 3H), 1.73 (d, J=7.07 Hz, 3H); LCMS: Calculated: for C25H22N4O2: 410, Measured: 411 [M+H]+

### Example 46

### (R)-4-(2-aminopyridin-3-yl)-2-(1-(3-methoxyphenyl)ethyl)-6-(1H-pyrazol-4-yl)isoindolin-1-one

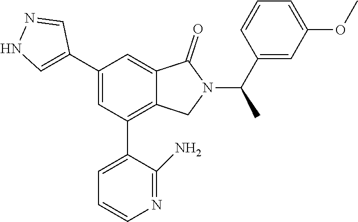 [SMILES: COc1cccc([C@@H](C)N2Cc3c(cc(-c4cn[nH]c4)cc3-c3cccnc3N)C2=O)c1]

(R)-4-(2-Aminopyridin-3-yl)-2-(1-(3-methoxyphenyl)ethyl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.12 (d, J=1.01 Hz, 1H), 7.95 (dd, J=1.52, 6.57 Hz, 1H), 7.85 (d, J=1.52 Hz, 1H), 7.24 (d, J=8.08 Hz, 1H), 7.00-7.06 (m, 1H), 6.91 (s, 1H), 6.79-6.86 (m, 1H), 5.61-5.74 (m, 1H), 4.34-4.50 (m, 1H), 4.01 (d, J=18.19 Hz, 1H), 3.75 (s, 3H), 1.68 (d, J=7.07 Hz, 3H); LCMS: Calculated: for C25H23N5O2: 425, Measured: 426 [M+H]+

### Example 47

### (R)-2-(1-(3-methoxyphenyl)ethyl)-4-(5-methoxypyridin-3-yl)-6-(1H-pyrazol-4-yl)isoindolin-1-one

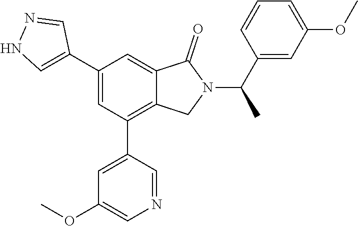 [SMILES: COc1cncc(-c2cc(-c3cn[nH]c3)cc3c2CN([C@H](C)c2cccc(OC)c2)C3=O)c1]

(R)-2-(1-(3-Methoxyphenyl)ethyl)-4-(5-methoxypyridin-3-yl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.17 (s, 1H), 8.09 (s, 1H), 7.97 (s, 1H), 7.20-7.33 (m, 1H), 6.90-7.00 (m, 1H), 6.79-6.90 (m, 1H), 5.61-5.71 (m, 1H), 4.67-4.76 (m, 1H), 4.13-4.20 (m, 1H), 3.92-4.03 (m, 3H), 3.76 (s, 3H), 1.72 (d, J=7.07 Hz, 3H); LCMS Calculated: for C26H24N4O3: 440, Measured: 441 [M+H]+.

### Example 48

### (R)-2-(1-(3-methoxyphenyl)ethyl)-4-(6-methoxypyridin-3-yl)-6-(1H-pyrazol-4-yl)isoindolin-1-one

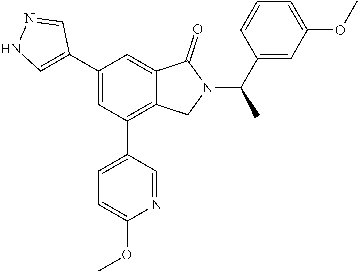 [SMILES: COc1cccc([C@@H](C)N2Cc3c(cc(-c4cn[nH]c4)cc3-c3ccc(OC)nc3)C2=O)c1]

(R)-2-(1-(3-Methoxyphenyl)ethyl)-4-(6-methoxypyridin-3-yl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.32 (br s, 1H), 7.99 (d, J=1.52 Hz, 1H), 7.93 (dd, J=2.53, 8.59 Hz, 1H), 7.85 (d, J=1.52 Hz, 1H), 7.26 (t, J=7.83 Hz, 1H), 6.89-6.98 (m, 1H), 6.84 (dd, J=2.53, 8.08 Hz, 1H), 5.66 (d, J=7.07 Hz, 1H), 4.65 (d, J=17.68 Hz, 1H), 4.12 (d, J=17.68 Hz, 1H), 3.91-4.02 (m, 3H), 3.76 (s, 3H), 1.71 (d, J=7.07 Hz, 3H); LCMS Calculated: for C26H24N4O3: 440, Measured: 441 [M+H]+.

### Example 49

### (R)-4-(4-hydroxyphenyl)-2-(1-(3-methoxyphenyl)ethyl)-6-(1H-pyrazol-4-yl)isoindolin-1-one

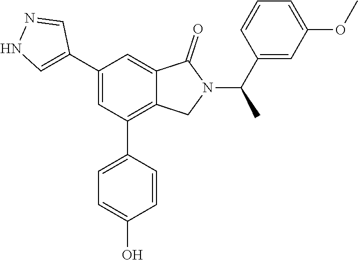 [SMILES: COc1cccc([C@@H](C)N2Cc3c(cc(-c4cn[nH]c4)cc3-c3ccc(O)cc3)C2=O)c1]

(R)-4-(4-Hydroxyphenyl)-2-(1-(3-methoxyphenyl)ethyl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.05-8.19 (m, 1H), 7.86-7.94 (m, 1H), 7.74-7.80 (m, 1H), 7.36 (d, J=8.59 Hz, 1H), 7.21-7.31 (m, 1H), 6.88 (d, J=8.59 Hz, 3H), 5.59-5.72 (m, 1H), 4.54-4.72 (m, 1H), 4.03-4.16 (m, 1H), 3.76 (s, 3H), 1.71 (d, J=7.07 Hz, 3H); LCMS Calculated: for C26H23N3O3: 425, Measured: 426 [M+H]+.

### Example 50

### ((R)-2-(1-(3-methoxyphenyl)ethyl)-6-(1H-pyrazol-4-yl)-4-(pyrimidin-5-yl)isoindolin-1-one

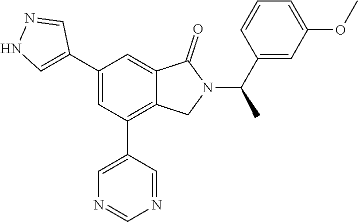 [SMILES: COc1cccc([C@@H](C)N2Cc3c(cc(-c4cn[nH]c4)cc3-c3cncnc3)C2=O)c1]

((R)-2-(1-(3-methoxyphenyl)ethyl)-6-(1H-pyrazol-4-yl)-4-(pyrimidin-5-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 9.20 (s, 1H), 9.04 (s, 2H), 8.15-8.28 (m, 2H), 8.09 (d, J=1.52 Hz, 1H), 7.98 (d, J=1.52 Hz, 1H), 7.26 (t, J=7.83 Hz, 1H), 6.88-7.01 (m, 2H), 6.85 (s, 1H), 5.67 (d, J=7.07 Hz, 1H), 4.75 (d, J=17.68 Hz, 1H), 4.21 (d, J=17.68 Hz, 1H), 3.76 (s, 3H), 1.72 (d, J=7.07 Hz, 3H); LCMS Calculated: for C24H21N5O2: 411, Measured: 412 [M+H]+.

### Example 51

### (R)-4-(2,6-dimethylpyridin-4-yl)-2-(1-(3-methoxyphenyl)ethyl)-6-(1H-pyrazol-4-yl)isoindolin-1-one

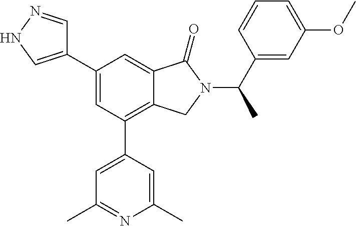 [SMILES: COc1cccc([C@@H](C)N2Cc3c(cc(-c4cn[nH]c4)cc3-c3cc(C)nc(C)c3)C2=O)c1]

(R)-4-(2,6-Dimethylpyridin-4-yl)-2-(1-(3-methoxyphenyl)ethyl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.13-8.26 (m, 1H), 8.07 (d, J=1.52 Hz, 1H), 7.96 (s, 1H), 7.26 (t, J=8.08 Hz, 1H), 6.89-6.99 (m, 1H), 6.77-6.89 (m, 1H), 5.61-5.73 (m, 1H), 4.86 (d, J=18.19 Hz, 1H), 4.25 (d, J=18.19 Hz, 1H), 3.75 (s, 3H), 2.79 (s, 6H), 1.75 (d, J=7.07 Hz, 3H); LCMS Calculated: for C27H26N4O2: 438, Measured: 439 [M+H]+.

### Example 52

### (R)-4-(3-hydroxyphenyl)-2-(1-(3-methoxyphenyl)ethyl)-6-(1H-pyrazol-4-yl)isoindolin-1-one

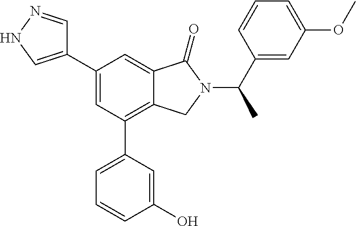 [SMILES: COc1cccc([C@@H](C)N2Cc3c(cc(-c4cn[nH]c4)cc3-c3cccc(O)c3)C2=O)c1]

(R)-4-(3-Hydroxyphenyl)-2-(1-(3-methoxyphenyl)ethyl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.09-8.27 (m, 1H), 7.97 (d, J=1.52 Hz, 1H), 7.81 (d, J=1.52 Hz, 1H), 7.28 (dt, J=6.57, 7.83 Hz, 1H), 6.87-7.01 (m, 2H), 6.77-6.87 (m, 1H), 5.66 (d, J=7.07 Hz, 1H), 4.59 (d, J=18.19 Hz, 1H), 4.13 (d, J=18.19 Hz, 1H), 3.76 (s, 3H), 1.63-1.74 (m, 3H); LCMS Calculated: for C26H23N3O3: 425, Measured: 426 [M+H]+.

### Example 53

### (R)-4-(3-methoxyphenyl)-2-(1-(3-methoxyphenyl)ethyl)-6-(1H-pyrazol-4-yl)isoindolin-1-one

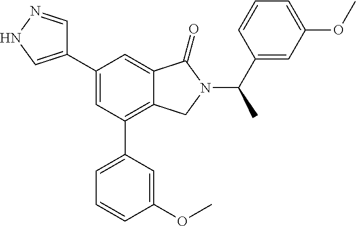 [SMILES: COc1cccc(-c2cc(-c3cn[nH]c3)cc3c2CN([C@H](C)c2cccc(OC)c2)C3=O)c1]

(R)-4-(3-Methoxyphenyl)-2-(1-(3-methoxyphenyl)ethyl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.20 (br d, J=1.52 Hz, 1H), 7.97 (d, J=1.01 Hz, 1H), 7.82 (d, J=1.52 Hz, 1H), 7.37 (t, J=7.83 Hz, 1H), 7.19-7.30 (m, 1H), 7.00-7.11 (m, 1H), 6.88-7.00 (m, 2H), 6.83 (dd, J=2.02, 8.08 Hz, 1H), 5.65 (d, J=7.07 Hz, 1H), 4.59 (d, J=17.68 Hz, 1H), 4.09 (d, J=17.68 Hz, 1H), 3.81 (s, 3H), 3.75 (s, 3H), 1.69 (d, J=7.07 Hz, 3H); LCMS Calculated: for C27H25N3O3: 439, Measured: 440 [M+H]+.

### Example 54

### (R)-4-(6-(2-hydroxypropan-2-yl)pyridin-3-yl)-2-(1-(3-methoxyphenyl)ethyl)-6-(1H-pyrazol-4-yl)isoindolin-1-one

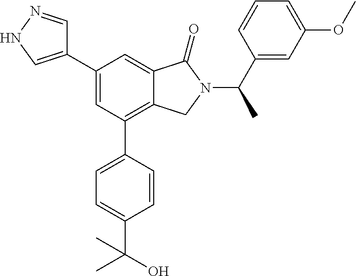 [SMILES: COc1cccc([C@@H](C)N2Cc3c(cc(-c4cn[nH]c4)cc3-c3ccc(C(C)(C)O)cc3)C2=O)c1]

(R)-4-(6-(2-Hydroxypropan-2-yl)pyridin-3-yl)-2-(1-(3-methoxyphenyl)ethyl)-6-(1H-pyrazol-4-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.81 (d, J=2.02 Hz, 1H), 8.60-8.68 (m, 1H), 8.07-8.21 (m, 4H), 8.00 (d, J=1.52 Hz, 1H), 7.26 (t, J=7.83 Hz, 1H), 6.89-7.01 (m, 2H), 6.84 (dd, J=2.02, 8.08 Hz, 1H), 5.58-5.75 (m, 1H), 4.73 (d, J=17.68 Hz, 1H), 4.22 (d, J=17.68 Hz, 1H), 3.75 (s, 3H), 1.59-1.81 (m, 9H); LCMS Calculated: for C28H28N4O3: 468, Measured: 469 [M+H]+.

### Example 55

### (R)-2-(1-(3-methoxyphenyl)ethyl)-6-(1H-pyrazol-4-yl)-4-(6-(trifluoromethyl)pyridin-3-yl)isoindolin-1-one

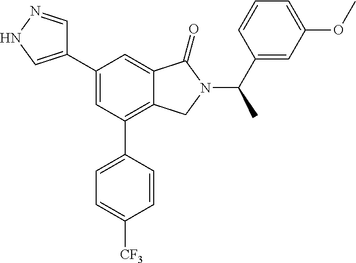 [SMILES: COc1cccc([C@@H](C)N2Cc3c(cc(-c4cn[nH]c4)cc3-c3ccc(C(F)(F)F)cc3)C2=O)c1]

(R)-2-(1-(3-Methoxyphenyl)ethyl)-6-(1H-pyrazol-4-yl)-4-(6-(trifluoromethyl)pyridin-3-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.83-8.99 (m, 1H), 8.19-8.41 (m, 1H), 8.06-8.13 (m, 1H), 7.84-8.06 (m, 2H), 7.41-7.63 (m, 2H), 7.22-7.31 (m, 1H), 6.88-6.99 (m, 3H), 6.78-6.88 (m, 1H), 5.58-5.72 (m, 1H), 4.66-4.76 (m, 1H), 4.14-4.24 (m, 1H), 3.76 (s, 3H), 1.71 (d, J=7.07 Hz, 3H); LCMS Calculated: for C26H21F3N4O2: 478, Measured: 479 [M+H]+.

### Example 56

### (R)-2-(1-(3-methoxyphenyl)ethyl)-6-(1H-pyrazol-4-yl)-4-(tetrahydro-2H-pyran-4-yl)isoindolin-1-one

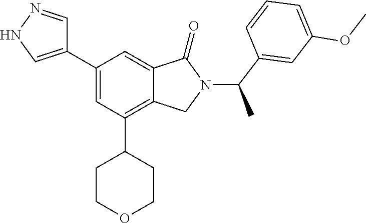 [SMILES: COc1cccc([C@@H](C)N2Cc3c(cc(-c4cn[nH]c4)cc3C3CCOCC3)C2=O)c1]

### Step 1: (R)-4-(3,6-Dihydro-2H-pyran-4-yl)-2-(1-(3-methoxyphenyl)ethyl)-6-(1H-pyrazol-4-yl)isoindolin-1-one

(R)-4-(3,6-Dihydro-2H-pyran-4-yl)-2-(1-(3-methoxyphenyl)ethyl)-6-(1H-pyrazol-4-yl)isoindolin-1-one was prepared according to the procedure described in Example 40, Step 3, substituting 2-(3,6-dihydro-2H-pyran-4-yl)-4,4,5,5-tetramethyl-1,3,2-dioxaborolane for 2-tert-butyl-4-(4,4,5,5-tetramethyl-1,3,2-dioxaborolan-2-yl)pyridine. LCMS: Calculated: for C25H25N3O3: 415, Measured: 416 [M+H]+

### Step 2: (R)-2-(1-(3-Methoxyphenyl)ethyl)-6-(1H-pyrazol-4-yl)-4-(tetrahydro-2H-pyran-4-yl)isoindolin-1-one

A mixture of (R)-4-(3,6-dihydro-2H-pyran-4-yl)-2-(1-(3-methoxyphenyl)ethyl)-6-(1H-pyrazol-4-yl)isoindolin-1-one (80 mg, 0.19 mmol) and 5% Pd/C (205 mg, 0.096 mmol) in MeOH (15 mL) was stirred under a hydrogen atmosphere at 50° C. After 12 h, the reaction mixture was allowed to cool to room temperature, filtered and filtrate was concentrated in vacuo. Purification of the resulting residue by C18 reverse column chromatography (80 g, 5%-35% ACN/H₂O (containing 0.05% TFA)) yielded (R)-2-(1-(3-methoxyphenyl)ethyl)-6-(1H-pyrazol-4-yl)-4-(tetrahydro-2H-pyran-4-yl)isoindolin-1-one as a white solid.

¹H NMR (300 MHz, DMSO-d6) δ 8.23 (s, 2H), 7.76 (dd, J=7.4, 1.6 Hz, 2H), 7.28 (t, J=7.8 Hz, 1H), 6.85-6.93 (m, 3H), 5.52 (q, J=7.3 Hz, 1H), 4.59 (d, J=17.7 Hz, 1H), 4.17 (d, J=17.6 Hz, 1H), 3.88-4.08 (m, 2H), 3.74 (s, 3H), 3.36-3.55 (m, 2H), 2.78-2.97 (m, 1H), 1.57-1.99 (m, 7H). LCMS: Calculated: for C25H27N3O3: 417, Measured: 418 [M+H]+

### Example 57

### 2-((R)-1-(3-methoxyphenyl)ethyl)-6-(1H-pyrazol-4-yl)-4-(pyrrolidin-3-yl)isoindolin-1-one

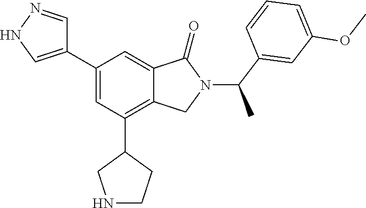 [SMILES: COc1cccc([C@@H](C)N2Cc3c(cc(-c4cn[nH]c4)cc3C3CCNC3)C2=O)c1]

### Step 1: tert-Butyl 3-(2-((R)-1-(3-methoxyphenyl)ethyl)-1-oxo-6-(1H-pyrazol-4-yl)isoindolin-4-yl)pyrrolidine-1-carboxylate

tert-Butyl 3-(2-((R)-1-(3-methoxyphenyl)ethyl)-1-oxo-6-(1H-pyrazol-4-yl)isoindolin-4-yl)pyrrolidine-1-carboxylate was prepared according to the procedure described in Example 56, Step 2, substituting tert-butyl 3-(4,4,5,5-tetramethyl-1,3,2-dioxaborolan-2-yl)-2,5-dihydro-1H-pyrrole-1-carboxylate for 2-(3,6-dihydro-2H-pyran-4-yl)-4,4,5,5-tetramethyl-1,3,2-dioxaborolane.

### Step 2: 2-((R)-1-(3-Methoxyphenyl)ethyl)-6-(1H-pyrazol-4-yl)-4-(pyrrolidin-3-yl)isoindolin-1-one

To a mixture of tert-butyl 3-(2-((R)-1-(3-methoxyphenyl)ethyl)-1-oxo-6-(1H-pyrazol-4-yl)isoindolin-4-yl)pyrrolidine-1-carboxylate (140 mg, 0.279 mmol) in DCM (2 mL) was added TFA (2 mL). The reaction was stirred at room temperature. After 2 h, the reaction mixture was concentrated. Purification of the resulting residue by C18 reverse column chromatography (80 g, 5%-45% ACN/H₂O (containing 0.05% TFA)) yielded 2-((R)-1-(3-methoxyphenyl)ethyl)-6-(1H-pyrazol-4-yl)-4-(pyrrolidin-3-yl)isoindolin-1-one as a white solid.

¹H NMR (400 MHz, DMSO-d6) δ 9.07 (brs, 1H), 8.89 (brs, 1H), 8.22 (s, 2H), 7.84 (dd, J=8.7, 1.5 Hz, 2H), 7.25-7.34 (m, 1H), 6.85-6.96 (m, 3H), 5.54 (dd, J=7.1, 2.4 Hz, 1H), 4.60 (dd, J=17.5, 11.3 Hz, 1H), 4.17 (t, J=16.8 Hz, 1H), 3.75 (d, J=1.1 Hz, 3H), 3.56-3.71 (m, 1H), 3.48 (q, J=8.2 Hz, 2H), 3.26 (ddt, J=20.3, 14.7, 8.4 Hz, 2H), 2.30-2.48 (m, 1H), 2.07 (ddt, J=27.6, 12.6, 9.3 Hz, 1H), 1.65 (d, J=7.2 Hz, 3H)./19F NMR (376 MHz, DMSO-d6) d −74.14; LCMS: Calculated: for C24H26N4O2: 402, Measured: 403 [M+H]+.

### Example 58

### (R)-2-(1-(3-methoxyphenyl)ethyl)-4-(piperidin-4-yl)-6-(1H-pyrazol-4-yl)isoindolin-1-one

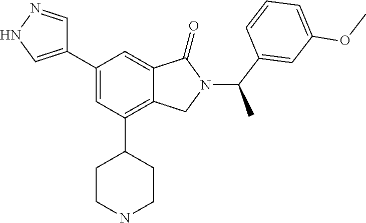 [SMILES: COc1cccc([C@@H](C)N2Cc3c(cc(-c4cn[nH]c4)cc3C3CCNCC3)C2=O)c1]

### Step 1: (R)-Benzyl4-(2-(1-(3-methoxyphenyl)ethyl)-1-oxo-6-(1H-pyrazol-4-yl)isoindolin-4-yl)-5,6-dihydropyridine-1(2H)-carboxylate

(R)-Benzyl4-(2-(1-(3-methoxyphenyl)ethyl)-1-oxo-6-(1H-pyrazol-4-yl)isoindolin-4-yl)-5,6-dihydropyridine-1(2H)-carboxylate was prepared according to the procedure described in Example 57, Step 1, substituting benzyl 4-(4,4,5,5-tetramethyl-1,3,2-dioxaborolan-2-yl)-5,6-dihydropyridine-1(2H)-carboxylate for tert-butyl 3-(4,4,5,5-tetramethyl-1,3,2-dioxaborolan-2-yl)-2,5-dihydro-1H-pyrrole-1-carboxylate. LCMS: Calculated: for C33H32N4O4: 548, Measured: 549 [M+H]+.

### Step 2: (R)-2-(1-(3-methoxyphenyl)ethyl)-4-(piperidin-4-yl)-6-(1H-pyrazol-4-yl)isoindolin-1-one

(R)-2-(1-(3-methoxyphenyl)ethyl)-4-(piperidin-4-yl)-6-(1H-pyrazol-4-yl)isoindolin-1-one was prepared according to the procedure described in Example 56, Step 2.

¹H NMR (400 MHz, DMSO-d6) δ 8.19 (brs, 2H), 7.72 (d, J=23.8 Hz, 2H), 7.28 (t, J=7.9 Hz, 1H), 6.85-6.93 (m, 3H), 5.52 (q, J=7.3 Hz, 1H), 4.56 (d, J=17.6 Hz, 1H), 4.14 (d, J=17.3 Hz, 1H), 3.74 (s, 3H), 3.03 (d, J=11.1 Hz, 2H), 2.55-2.88 (m, 3H), 1.67 (p, J=11.4, 10.6 Hz, 7H): LCMS: Calculated: C25H28N4O2: 416, Measured: 417 [M+H]+.

### Example 59

### 2-((R)-1-(3-methoxyphenyl)ethyl)-4-(piperidin-3-yl)-6-(1H-pyrazol-4-yl)isoindolin-1-one

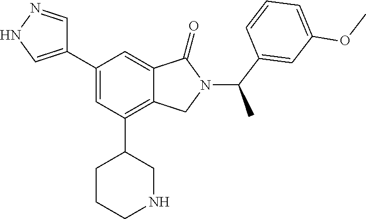 [SMILES: COc1cccc([C@@H](C)N2Cc3c(cc(-c4cn[nH]c4)cc3C3CCCNC3)C2=O)c1]

2-((R)-1-(3-Methoxyphenyl)ethyl)-4-(piperidin-3-yl)-6-(1H-pyrazol-4-yl)isoindolin-1-one was prepared according to the procedure described in Example 58, Step 2, substituting benzyl 3-(4,4,5,5-tetramethyl-1,3,2-dioxaborolan-2-yl)-5,6-dihydropyridine-1(2H)-carboxylate for benzyl 4-(4,4,5,5-tetramethyl-1,3,2-dioxaborolan-2-yl)-5,6-dihydropyridine-1 (2H)-carboxylate.

¹H NMR (400 MHz, DMSO-d6) δ 8.92 (d, J=11.4 Hz, 1H), 8.46 (dd, J=49.3, 11.9 Hz, 1H), 8.19 (s, 2H), 7.82 (dd, J=7.7, 1.9 Hz, 2H), 7.28 (tt, J=8.7, 1.1 Hz, 1H), 6.82-6.98 (m, 3H), 5.53 (p, J=7.0 Hz, 1H), 4.55 (dd, J=33.9, 17.3 Hz, 1H), 4.11 (dd, J=52.2, 17.4 Hz, 1H), 3.72 (d, J=2.1 Hz, 3H), 3.33 (q, J=10.8, 9.8 Hz, 2H), 3.00 (ddq, J=59.3, 35.8, 11.6 Hz, 3H), 1.67-1.98 (m, 4H), 1.62 (d, J=7.1 Hz, 3H); 19F NMR (376 MHz, DMSO-d6) d −74.54; LCMS: Calculated: C25H28N4O2: 416, Measured: 417 [M+H]+.

### Example 60

### (R)-2-(1-(3-methoxyphenyl)ethyl)-4-(4-methylpiperazin-1-yl)-6-(1H-pyrazol-4-yl)isoindolin-1-one

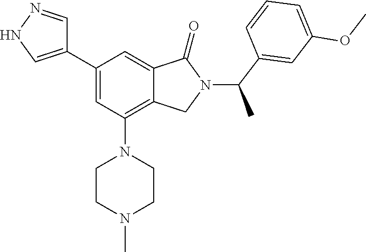 [SMILES: COc1cccc([C@@H](C)N2Cc3c(cc(-c4cn[nH]c4)cc3N3CCN(C)CC3)C2=O)c1]

### Step 1: 4-Bromo-2-((R)-1-(3-methoxyphenyl)ethyl)-6-(1-(tetrahydro-2H-pyran-2-yl)-1H-pyrazol-4-yl)isoindolin-1-one

Under a nitrogen atmosphere, a mixture of (R)-4-bromo-6-iodo-2-(1-(3-methoxyphenyl)ethyl)isoindolin-1-one (1.20 g, 2.54 mmol), 1-(tetrahydro-2H-pyran-2-yl)-4-(4,4,5,5-tetramethyl-1,3,2-dioxaborolan-2-yl)-1H-pyrazole (707 mg, 2.54 mmol), K₂CO₃ (703 mg, 5.087 mmol) and Pd(PPh₃)₄ (59 mg, 0.051 mmol) in 1,4-dioxane (20 mL) and H₂O (2 mL) was warmed to 95° C. After 12 h, the reaction mixture was allowed to cool to room temperature, diluted with EtOAc (100 mL), washed with brine (2×30 mL), dried (Na₂SO₄), filtered and concentrated in vacuo. Purification of the resulting residue by silica gel column chromatography (80 g, 0-100% EtOAc/heptane) yielded 4-bromo-2-((R)-1-(3-methoxyphenyl)ethyl)-6-(1-(tetrahydro-2H-pyran-2-yl)-1H-pyrazol-4-yl)isoindolin-1-one as a yellow oil. LC/MS: Calculated: for C25H26BrN3O3: 495.12, Measured: 496.1, 498.1 [M+H, M+2H]+.

### Step 2: (2-((R)-1-(3-Methoxyphenyl)ethyl)-4-(4-methylpiperazin-1-yl)-6-(1-(tetrahydro-2H-pyran-2-yl)-1H-pyrazol-4-yl)isoindolin-1-one

Under a nitrogen atmosphere, a mixture of 4-bromo-2-((R)-1-(3-methoxyphenyl)ethyl)-6-(1-(tetrahydro-2H-pyran-2-yl)-1H-pyrazol-4-yl)isoindolin-1-one (250 mg, 0.50 mmol), 1-methylpiperazine (151 mg, 1.51 mmol), Cs₂CO₃ (492 mg, 1.51 mmol), RuPhos (23.5 mg, 0.050 mmol) and Pd RuPhos (39 mg, 0.050 mmol) in toluene (15 mL) was stirred at 110° C. After 12 h, the reaction mixture was allowed to cool to room temperature, diluted with EtOAc (100 mL), washed with brine (2×30 mL), dried (Na₂SO₄), filtered and concentrated in vacuo. Purification of the resulting residue by silica gel column chromatography (20 g, 0-100% EtOAc/heptane) yielded (2-((R)-1-(3-methoxyphenyl)ethyl)-4-(4-methylpiperazin-1-yl)-6-(1-(tetrahydro-2H-pyran-2-yl)-1H-pyrazol-4-yl)isoindolin-1-one as a yellow solid. LC/MS: Calculated: for C30H37N5O3: 515, Measured: 516 [M+H]+.

### Step 3: (R)-2-(1-(3-Methoxyphenyl)ethyl)-4-(4-methylpiperazin-1-yl)-6-(1H-pyrazol-4-yl)isoindolin-1-one

To a mixture of (2-((R)-1-(3-methoxyphenyl)ethyl)-4-(4-methylpiperazin-1-yl)-6-(1-(tetrahydro-2H-pyran-2-yl)-1H-pyrazol-4-yl)isoindolin-1-one (150 mg, 0.291 mmol) in DCM (5 mL) was added TFA (1 mL). The reaction was stirred at room temperature for 1 h and concentrated in vacuo. Purification of the resulting residue by C18 reverse column chromatography (80 g, 5%-35% ACN/H₂O (containing 0.05% TFA)) yielded (R)-2-(1-(3-methoxyphenyl)ethyl)-4-(4-methylpiperazin-1-yl)-6-(1H-pyrazol-4-yl)isoindolin-1-one as a light yellow solid.

¹H NMR (400 MHz, DMSO-d6) δ 9.83 (brs, 1H), 8.22 (s, 2H), 7.60 (d, J=1.3 Hz, 1H), 7.37 (d, J=1.4 Hz, 1H), 7.23-7.33 (m, 1H), 6.82-6.94 (m, 3H), 5.52 (q, J=7.1 Hz, 1H), 4.58 (d, J=17.7 Hz, 1H), 4.09 (d, J=17.8 Hz, 1H), 3.74 (s, 3H), 3.67 (d, J=13.2 Hz, 1H), 3.54 (dd, J=20.4, 9.7 Hz, 3H), 3.19 (h, J=11.5, 10.8 Hz, 3H), 3.05 (t, J=12.7 Hz, 1H), 2.89 (d, J=3.7 Hz, 3H), 1.67 (d, J=7.1 Hz, 3H). 19F NMR (376 MHz, DMSO-d6) d −74.34. LC/MS: Calculated: for C25H29N5O2 431, Measured: 432. [M+H]+.

### Example 61

### (R)-2-(1-(3-methoxyphenyl)ethyl)-4-(piperazin-1-yl)-6-(1H-pyrazol-4-yl)isoindolin-1-one

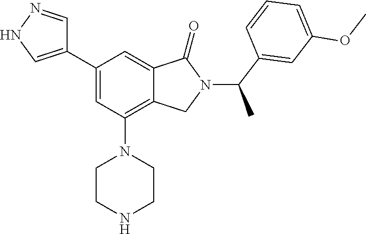 [SMILES: COc1cccc([C@@H](C)N2Cc3c(cc(-c4cn[nH]c4)cc3N3CCNCC3)C2=O)c1]

(R)-2-(1-(3-Methoxyphenyl)ethyl)-4-(piperazin-1-yl)-6-(1H-pyrazol-4-yl)isoindolin-1-one was prepared according to the procedure described in Example 60, Step 3, substituting tert-butyl piperazine-1-carboxylate for 1-methylpiperazine.

¹H NMR (400 MHz, DMSO-d6) δ 8.78 (brs, 2H), 8.22 (s, 2H), 7.60 (d, J=1.3 Hz, 1H), 7.36 (d, J=1.4 Hz, 1H), 7.24-7.32 (m, 1H), 6.83-6.94 (m, 3H), 5.51 (q, J=7.3 Hz, 1H), 4.58 (d, J=17.8 Hz, 1H), 4.12 (d, J=17.8 Hz, 1H), 3.74 (s, 3H), 3.34 (td, J=7.5, 3.3 Hz, 2H), 3.18-3.30 (m, 6H), 1.67 (d, J=7.2 Hz, 3H). 19F NMR (376 MHz, DMSO-d6) d −74.25. Calculated: for C24H27N5O2 417, Measured: 418. [M+H]+.

### Example 62

### 2-((R)-1-(3-methoxyphenyl)ethyl)-6-(1H-pyrazol-4-yl)-4-(pyrrolidin-3-yl)isoindolin-1-one

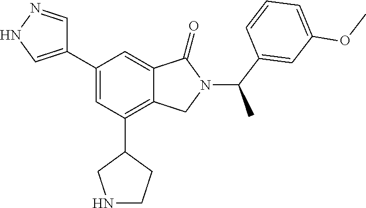 [SMILES: COc1cccc([C@@H](C)N2Cc3c(cc(-c4cn[nH]c4)cc3C3CCNC3)C2=O)c1]

2-((R)-1-(3-Methoxyphenyl)ethyl)-6-(1H-pyrazol-4-yl)-4-(pyrrolidin-3-yl)isoindolin-1-one was similarly prepared according to the procedures described herein, selecting and substituting suitable reagents and starting materials, as would be recognized by those skilled in the art.

¹H NMR (400 MHz, METHANOL-d4) δ 8.03-8.20 (m, 1H), 7.91 (d, J=1.01 Hz, 1H), 7.81 (s, 1H), 7.29 (s, 1H), 6.96 (br d, J=18.69 Hz, 1H), 6.82-6.91 (m, 1H), 5.63-5.73 (m, 1H), 4.60 (s, 1H), 4.18 (s, 1H), 3.49-3.71 (m, 1H), 3.30 (m, 1H), 2.38-2.60 (m, 1H), 2.09-2.26 (m, 1H), 1.74 (d, J=7.58 Hz, 2H); LCMS Calculated: for C24H26N4O2: 402, Measured: 403 [M+H]+.

### Example 63

### (R)-2-(1-(3-methoxyphenyl)ethyl)-6-(1H-pyrazol-4-yl)-4-(2,2,6,6-tetramethylpiperidin-4-yl)isoindolin-1-one

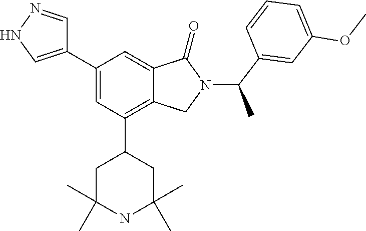 [SMILES: COc1cccc([C@@H](C)N2Cc3c(cc(-c4cn[nH]c4)cc3C3CC(C)(C)NC(C)(C)C3)C2=O)c1]

### Step 1: 4-Bromo-2-((R)-1-(3-methoxyphenyl)ethyl)-6-(1-(tetrahydro-2H-pyran-2-yl)-1H-pyrazol-4-yl)isoindolin-1-one

4-Bromo-2-((R)-1-(3-methoxyphenyl)ethyl)-6-(1-(tetrahydro-2H-pyran-2-yl)-1H-pyrazol-4-yl)isoindolin-1-one was prepared according to the procedure described in Example 83, Step 1, substituting (R)-4-bromo-6-iodo-2-(1-(3-methoxyphenyl)ethyl)isoindolin-1-one for 5′-bromo-2′-[(3-methoxyphenyl)methyl]-2′,3′-dihydrospiro[cyclopentane-1,1′-isoindol]-3-en-3′-one. LCMS: Calculated: for C25H26BrN₃O₃: 495, Measured: 496, 498 [M+H, M+H+2]+.

Step 2: 2-((R)-1-(3-Methoxyphenyl)ethyl)-6-(1-(tetrahydro-2H-pyran-2-yl)-1H-pyrazol-4-yl)-4-(2,2,6,6-tetramethyl-1,2,3,6-tetrahydropyridin-4-yl)isoindolin-1-one

Under a nitrogen atmosphere, a mixture of 4-bromo-2-((R)-1-(3-methoxyphenyl)ethyl)-6-(1-(tetrahydro-2H-pyran-2-yl)-1H-pyrazol-4-yl)isoindolin-1-one (150 mg, 0.302 mmol), 2,2,6,6-tetramethyl-4-(4,4,5,5-tetramethyl-1,3,2-dioxaborolan-2-yl)-1,2,3,6-tetrahydropyridine (80 mg, 0.302 mmol), K₂CO₃ (167 mg, 1.208 mmol) and Pd(PPh₃)₄ (349 mg, 0.302 mmol) in DMF (10 mL) and H₂O (1 mL) was warmed to 110° C. After 12 h, the resulting mixture was allowed to cool to room temperature, diluted with EtOAc (100 mL), washed with brine (4×30 mL), dried (Na₂SO₄), filtered and concentrated in vacuo. Purification of the resulting residue by silica gel column chromatography (40 g, 0-20% MeOH/DCM) yielded 2-((R)-1-(3-methoxyphenyl)ethyl)-6-(1-(tetrahydro-2H-pyran-2-yl)-1H-pyrazol-4-yl)-4-(2,2,6,6-tetramethyl-1,2,3,6-tetrahydropyridin-4-yl)isoindolin-1-one as a yellow solid. LCMS: Calculated: for C34H42N4O3: 554, Measured: 555 [M+H]+.

### Step 3: 2-((R)-1-(3-methoxyphenyl)ethyl)-6-(1-(tetrahydro-2H-pyran-2-yl)-1H-pyrazol-4-yl)-4-(2,2,6,6-tetramethylpiperidin-4-yl)isoindolin-1-one

2-((R)-1-(3-methoxyphenyl)ethyl)-6-(1-(tetrahydro-2H-pyran-2-yl)-1H-pyrazol-4-yl)-4-(2,2,6,6-tetramethylpiperidin-4-yl)isoindolin-1-one was prepared according to the procedure described in Example 56, Step 1. LCMS: Calculated: for C34H44N4O3: 556, Measured: 557 [M+H]+.

### Step 4: (R)-2-(1-(3-Methoxyphenyl)ethyl)-6-(1H-pyrazol-4-yl)-4-(2,2,6,6-tetramethylpiperidin-4-yl)isoindolin-1-one

(R)-2-(1-(3-Methoxyphenyl)ethyl)-6-(1H-pyrazol-4-yl)-4-(2,2,6,6-tetramethylpiperidin-4-yl)isoindolin-1-one was prepared according to the procedure described in Example 83, Step 3.

¹H NMR (300 MHz, DMSO-d6) δ 8.66 (d, J=12.6 Hz, 1H), 8.19 (s, 2H), 7.76-7.82 (m, 2H), 7.67 (s, 1H), 7.28 (t, J=7.8 Hz, 1H), 6.81-6.97 (m, 3H), 5.52 (q, J=7.0 Hz, 1H), 4.67 (d, J=17.9 Hz, 1H), 4.34 (d, J=17.8 Hz, 1H), 3.73 (s, 3H), 3.26 (d, J=12.8 Hz, 1H), 1.84 (dq, J=26.7, 13.4 Hz, 4H), 1.68 (d, J=7.2 Hz, 3H), 1.49 (d, J=10.3 Hz, 6H), 1.40 (s, 6H). 19F NMR (282 MHz, DMSO-d6) d −74.10; LCMS Calculated: for C29H36N4O2: 472, Measured: 473 [M+H]+.

### Example 64

### 2-(3-methoxybenzyl)-6-(5-oxo-1,5-dihydro-4H-1,2,4-triazol-4-yl)isoindolin-1-one

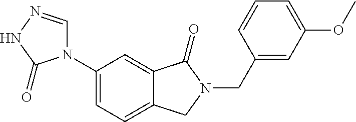 [SMILES: COc1cccc(CN2Cc3ccc(-n4cn[nH]c4=O)cc3C2=O)c1]

### Step 1: 6-(Diphenylmethyleneamino)-2-(3-methoxybenzyl)isoindolin-1-one

Under a nitrogen atmosphere, solution of 6-bromo-2-(3-methoxybenzyl)isoindolin-1-one (200 mg, 0.602 mmol) in toluene (5 ml) was successively treated with diphenylmethanimine (0.20 ml, 1.204 mmol), BINAP (37.5 mg, 0.060 mmol), Pd₂(dba)₃CHCl3 (55.1 mg, 0.060 mmol, 0.1 equiv) and NaOt-Bu (116 mg, 1.20 mmol). The resulting mixture was warmed to 100° C. After 12 h, the reaction mixture was allowed to cool to room temperature, diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded 6-(diphenylmethyleneamino)-2-(3-methoxybenzyl)isoindolin-1-one as a yellow solid. LCMS: Calculated: for C29H24N2O2: 432, Measured: 433 [M+H]+.

### Step 2: 6-Amino-2-(3-methoxybenzyl)isoindolin-1-one

To a solution of 6-(diphenylmethyleneamino)-2-(3-methoxybenzyl)isoindolin-1-one (200 mg, 0.462 mmol) in THE (5 ml) was treated with HCl (6 M) (5 ml). After 30 min, the reaction mixture was diluted with water, washed with ethyl acetate (3×15 mL), the aqueous layer pH was adjusted to pH 7 with aqueous satd. NaHCO₃ and the desired product was extracted from the aqueous layer with ethyl acetate (3×15 mL). The combined extracts were washed with brine (3×15 mL), dried (Na₂SO₄), and concentrated in vacuo to yield 6-amino-2-(3-methoxybenzyl)isoindolin-1-one as a yellow solid. LCMS: Calculated: for C16H16N2O2: 269, Measured: 559 [2M+Na]+.

### Step 3: 2-(3-Methoxybenzyl)-6-(5-oxo-1,5-dihydro-1,2,4-triazol-4-yl)isoindolin-1-one

A solution of 6-amino-2-(3-methoxybenzyl)isoindolin-1-one (100 mg, 0.373 mmol) in MeOH (10 ml) was treated with methyl hydrazinecarboxylate (67.147 mg, 0.745 mmol), followed by trimethoxymethane (79.103 mg, 0.745 mmol) and reaction mixture was warmed to 60° C. After 12 h, the resulting solution was treated with NaOCH₃ (40.270 mg, 0.745 mmol). After 2 h, the reaction mixture was allowed to cool to room temperature, diluted with EtOAc (50 mL), washed with brine (3×15 mL), dried (Na₂SO₄), and concentrated in vacuo. Purification of the resulting residue by silica gel chromatography (24 g column, 0-10% MeOH/DCM) yielded 2-(3-methoxybenzyl)-6-(5-oxo-1,5-dihydro-1,2,4-triazol-4-yl)isoindolin-1-one as a white solid.

¹H NMR (400 MHz, DMSO-d6) δ 12.04 (s, 1H), 8.50 (d, J=1.4 Hz, 1H), 8.06 (d, J=2.0 Hz, 1H), 7.92 (dd, J=8.2, 2.1 Hz, 1H), 7.66 (d, 1H), 7.21-7.31 (m, 1H), 6.78-6.88 (m, 3H), 4.70 (s, 2H), 4.39 (s, 2H), 3.71 (s, 3H); LCMS: Calculated: for C18H16N4O3: 336, Measured: 337 [M+H]+.

### Example 65

### (R)-2-(1-(3-methoxyphenyl)ethyl)-6-(5-oxo-1,5-dihydro-4H-1,2,4-triazol-4-yl)isoindolin-1-one

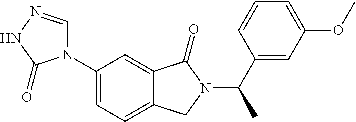 [SMILES: COc1cccc([C@@H](C)N2Cc3ccc(-n4cn[nH]c4=O)cc3C2=O)c1]

### Step 1: (R)-2-(1-(3-Methoxyphenyl)ethyl)-6-nitroisoindolin-1-one

(R)-2-(1-(3-Methoxyphenyl)ethyl)-6-nitroisoindolin-1-one was prepared according to the procedure described in Example 2, Step 1, substituting methyl 2-(bromomethyl)-5-nitrobenzoate for methyl 5-bromo-2-(bromomethyl)benzoate. LCMS: Calculated: for C17H16N2O4: 312, Measured: 647.25 [2M+Na]+.

### Step 2: (R)-6-Amino-2-(1-(3-methoxyphenyl)ethyl)isoindolin-1-one

A solution of (R)-2-(1-(3-Methoxyphenyl)ethyl)-6-nitroisoindolin-1-one (390 mg, 1.249 mmol) in MeOH (5 ml) was treated with Pd(OH)₂/C (39 mg) and placed under a hydrogen atmosphere at room temperature. After 30 min, the reaction mixture was filtered and concentrated in vacuo to yield (R)-6-amino-2-(1-(3-methoxyphenyl)ethyl)isoindolin-1-one as a yellow oil. LCMS: Calculated: for C17H18N2O2: 282, Measured: 587 [2M+Na]+.

### Step 3: (R)-2-(1-(3-Methoxyphenyl)ethyl)-6-(5-oxo-1,5-dihydro-4H-1,2,4-triazol-4-yl)isoindolin-1-one

(R)-2-(1-(3-Methoxyphenyl)ethyl)-6-(5-oxo-1,5-dihydro-4H-1,2,4-triazol-4-yl)isoindolin-1-one was prepared according to the procedure described in Example 64, Step 3, substituting (R)-6-amino-2-(1-(3-methoxyphenyl)ethyl)isoindolin-1-one for 6-amino-2-(3-methoxybenzyl)isoindolin-1-one.

¹H NMR (400 MHz, DMSO-d6) δ 12.03 (s, 1H), 8.49 (d, J=1.4 Hz, 1H), 8.03 (d, J=2.0 Hz, 1H), 7.91 (dd, J=8.2, 2.1 Hz, 1H), 7.66 (d, J=8.2 Hz, 1H), 7.26 (td, J=7.6, 1.2 Hz, 1H), 6.81-6.92 (m, 3H), 5.49 (q, J=7.1 Hz, 1 H), 4.56 (d, J=18.0 Hz, 1H), 4.14 (d, J=18.0 Hz, 1H), 3.72 (s, 3H), 1.62 (d, J=7.1 Hz, 3H); LCMS: Calculated: for C19H18N4O3: 350, Measured: 351 [M+H]+.

### Example 66

### 2-(3-ethoxybenzyl)-3,3-dimethyl-6-(5-oxo-1,5-dihydro-4H-1,2,4-triazol-4-yl)isoindolin-1-one

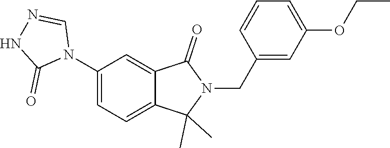 [SMILES: CCOc1cccc(CN2C(=O)c3cc(-n4cn[nH]c4=O)ccc3C2(C)C)c1]

### Step 1: 6-Bromo-2-(3-ethoxybenzyl)isoindolin-1-one

6-Bromo-2-(3-ethoxybenzyl)isoindolin-1-one was prepared according to the procedure described in Example 1, Step 1, substituting 1-(chloromethyl)-3-ethoxybenzene for 1-(chloromethyl)-3-methoxybenzene. LCMS: Calculated: for C17H16BrNO2346, Measured: 348 [M+2H]+.

### Step 2: 6-Bromo-2-(3-ethoxybenzyl)-3,3-dimethylisoindolin-1-one

6-Bromo-2-(3-ethoxybenzyl)-3,3-dimethylisoindolin-1-one was prepared according to the procedure described in Example 73, Step 1, substituting 6-bromo-2-(3-ethoxybenzyl)isoindolin-1-one for 6-bromo-2-(3-methoxybenzyl)isoindolin-1-one. LCMS: Calculated: for C19H20BrNO2: 374, Measured: 374 [M]+.

### Step 3: 2-(3-Ethoxybenzyl)-3,3-dimethyl-6-(5-oxo-1,5-dihydro-4H-1,2,4-triazol-4-yl)isoindolin-1-one

2-(3-Ethoxybenzyl)-3,3-dimethyl-6-(5-oxo-1,5-dihydro-4H-1,2,4-triazol-4-yl)isoindolin-1-one was prepared according to the procedure described in Example 64, Step 3, substituting (R)-2-(1-(3-methoxyphenyl)ethyl)-6-(5-oxo-1,5-dihydro-4H-1,2,4-triazol-4-yl)isoindolin-1-one for 6-amino-2-(3-methoxybenzyl)isoindolin-1-one.

¹H NMR (400 MHz, DMSO-d6) δ 12.03 (s, 1H), 8.48 (d, J=1.4 Hz, 1H), 8.03 (d, J=2.0 Hz, 1H), 7.93 (dd, J=8.2, 2.1 Hz, 1H), 7.80 (d, J=8.2 Hz, 1H), 7.19 (t, J=8.1 Hz, 1H), 6.90 (dt, J=3.8, 1.4 Hz, 2H), 6.81-6.73 (m, 1H), 4.65 (s, 2H), 3.96 (q, J=7.0 Hz, 2H), 1.37 (s, 6H), 1.28 (t, J=6.9 Hz, 3H); LCMS: Calculated: for C21H22N4O3: 378, Measured: 379 [M+H]+.

### Example 67

### 2-(3-methoxybenzyl)-6-(1H-1,2,3-triazol-4-yl)isoindolin-1-one

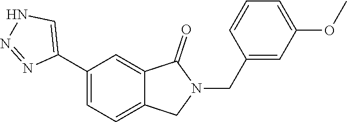 [SMILES: COc1cccc(CN2Cc3ccc(-c4c[nH]nn4)cc3C2=O)c1]

### Step 1: 2-(3-Methoxybenzyl)-6-((trimethylsilyl)ethynyl)isoindolin-1-one

Under a nitrogen atmosphere, a mixture of 6-bromo-2-(3-methoxybenzyl)isoindolin-1-one (730 mg, 2.20 mmol), ethynyltrimethylsilane (1.30 g, 13.24 mmol), Et₃N (2.89 g, 28.6 mmol), Pd(PPh₃)₂Cl₂ (77 mg, 0.110 mmol) and CuI (75 mg, 0.394 mmol) in DMF (20 mL) was warmed to 100° C.

After 12 h, the reaction mixture was allowed to cool to room temperature, diluted with EtOAc (50 mL), washed with brine (3×15 mL), dried (Na₂SO₄), and concentrated in vacuo. Purification of the resulting residue by silica gel chromatography (40 g column, 0-100% EtOAc/Heptane) yielded 2-(3-methoxybenzyl)-6-((trimethylsilyl)ethynyl)isoindolin-1-one as a yellow solid. LCMS: Calculated: for C21H23NO2Si: 349, Measured: 350 [M+H]+.

### Step 2: 6-Ethynyl-2-(3-methoxybenzyl)isoindolin-1-one

A mixture of 2-(3-methoxybenzyl)-6-((trimethylsilyl)ethynyl)isoindolin-1-one (600 mg, 1.717 mmol) in MeOH (20 mL) with K₂CO₃ (475 mg, 3.437 mmol) was stirred at room temperature. After 3 h, the reaction mixture was concentrated in vacuo. Purification of the resulting residue by silica gel chromatography (40 g column, 0-100% EtOAc/Heptane yielded 6-ethynyl-2-(3-methoxybenzyl)isoindolin-1-one as a yellow solid. LCMS: Calculated: for C18H15NO2: 277, Measured: 278 [M+H]+.

### Step 3: 2-(3-Methoxybenzyl)-6-(1H-1,2,3-triazol-4-yl)isoindolin-1-one

Under a nitrogen atmosphere, a mixture of 6-ethynyl-2-(3-methoxybenzyl)isoindolin-1-one (110 mg, 0.397 mmol) in TMSN₃ (2 mL) was warmed to 130° C. After 12 h, the reaction mixture was allowed to cool to room temperature, diluted with EtOAc (80 mL), washed with brine (2×30 mL), dried (Na₂SO₄), filtered and concentrated in vacuo. Purification of the resulting residue by recrystallization (EtOAc/PE: 1/10) yielded 2-(3-Methoxybenzyl)-6-(1H-1,2,3-triazol-4-yl)isoindolin-1-one as a light yellow solid.

¹H NMR (400 MHz, DMSO-d6) δ 15.18 (brs, 1H), 8.52 (brs, 1H), 8.20 (s, 1H), 8.12 (d, J=7.9 Hz, 1H), 7.65 (d, J=7.9 Hz, 1H), 7.26-7.30 (m, 1H), 6.84-6.88 (m, 3H), 4.72 (s, 2H), 4.41 (s, 2H), 3.74 (s, 3H); LCMS: Calculated: for C18H16N4O2: 320, Measured: 697 [2M+H]+.

### Example 68

### 2-(3-methoxybenzyl)-3,3-dimethyl-6-(1H-1,2,3-triazol-4-yl)isoindolin-1-one

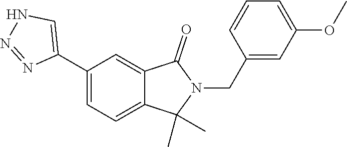 [SMILES: COc1cccc(CN2C(=O)c3cc(-c4c[nH]nn4)ccc3C2(C)C)c1]

2-(3-Methoxybenzyl)-3,3-dimethyl-6-(1H-1,2,3-triazol-4-yl)isoindolin-1-one was prepared according to the procedure described in Example 67, Step 3, substituting 6-bromo-2-(3-methoxybenzyl)-3,3-dimethylisoindolin-1-one for 6-bromo-2-(3-methoxybenzyl)isoindolin-1-one.

¹H NMR (400 MHz, DMSO-d6) δ 15.19 (brs, 1H), 8.52 (brs, 1H), 8.19 (s, 1H), 8.15 (dd, J=8.1, 1.7 Hz, 1H), 7.76 (d, J=7.9 Hz, 1H), 7.23 (dd, J=8.7, 7.3 Hz, 1H), 6.93-6.95 (m, 2H), 6.81 (dt, J=7.7, 2.0 Hz, 1H), 4.68 (s, 2H), 3.72 (s, 3H), 1.39 (s, 6H); LCMS: Calculated: for C20H20N4O2: 348, Measured: 697 [2M+H]+.

### Example 69

### 2-(3-Ethoxybenzyl)-3,3-dimethyl-6-(1H-1,2,3-triazol-4-yl)isoindolin-1-one

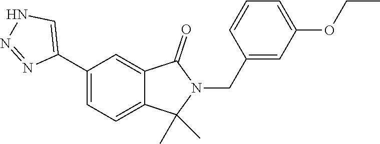 [SMILES: CCOc1cccc(CN2C(=O)c3cc(-c4c[nH]nn4)ccc3C2(C)C)c1]

2-(3-Ethoxybenzyl)-3,3-dimethyl-6-(1H-1,2,3-triazol-4-yl)isoindolin-1-one was prepared according to the procedure described in Example 67, Step 3, substituting 6-bromo-2-(3-ethoxybenzyl)-3,3-dimethylisoindolin-1-one for 6-bromo-2-(3-methoxybenzyl)isoindolin-1-one.

¹H NMR (300 MHz, DMSO-d6) δ 15.19 (brs, 1H), 8.50 (brs, 1H), 8.12-8.18 (m, 2H), 7.75 (d, J=8.0 Hz, 1H), 7.20 (t, J=8.1 Hz, 1H), 6.92 (dd, J=4.3, 2.3 Hz, 2H), 6.77-6.84 (m, 1H), 4.67 (s, 2H), 3.97 (q, J=7.0 Hz, 2H), 1.38 (s, 6H), 1.29 (t, J=6.9 Hz, 3H); LCMS: Calculated: for C20H20N4O2: 362, Measured: 363 [M+H]+.

### Example 70

### 5-(2-(3-methoxybenzyl)-3-oxoisoindolin-5-yl)-N-methylpicolinamide

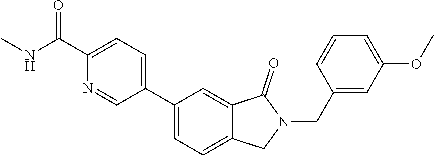

### Step 1: 2-(3-Methoxybenzyl)-6-(4,4,5,5-tetramethyl-1,3,2-dioxaborolan-2-yl)isoindolin-1-one

Under a nitrogen atmosphere, a mixture of 6-bromo-2-(3-methoxybenzyl)isoindolin-1-one (1.2 g, 3.61 mmol), bis(pinacolato)diboron (1.83 g, 7.21 mmol), KOAc (1.06 g, 10.8 mmol) and Pd(dppf)Cl₂ (147 mg, 0.180 mmol, 0.05 equiv) in DMF (30 mL) was warmed to 100° C. After 3 h, the reaction mixture was allowed to cool to room temperature, diluted with EtOAc (50 mL), washed with brine (3×15 mL), dried (Na₂SO₄), and concentrated in vacuo. Purification of the resulting residue by silica gel chromatography (80 g column, 0-100% EtOAc/Heptane) yielded 2-(3-methoxybenzyl)-6-(4,4,5,5-tetramethyl-1,3,2-dioxaborolan-2-yl)isoindolin-1-one as a light yellow solid. LCMS: Calculated: for C22H26BNO4: 379, Measured: 380 [M+H]+.

### Step 2: 5-(2-(3-Methoxybenzyl)-3-oxoisoindolin-5-yl)-N-methylpicolinamide

Under a nitrogen atmosphere, a mixture of 2-(3-methoxybenzyl)-6-(4,4,5,5-tetramethyl-1,3,2-dioxaborolan-2-yl)isoindolin-1-one (282 mg, 0.744 mmol), 5-bromo-N-methylpicolinamide (80 mg, 0.372 mmol), K₂CO₃ (154 mg, 1.114 mmol) and Pd(PPh₃)₄ (21.5 mg, 0.019 mmol) in DMF (15 mL) and H₂O (1 mL) was warmed to 100° C. After 3 h, the reaction mixture was allowed to cool to room temperature, diluted with EtOAc (50 mL), washed with brine (3×15 mL), dried (Na₂SO₄), and concentrated in vacuo. Purification of the resulting residue by silica gel chromatography (24 g column, 0-100% EtOAc/Heptane) yielded 5-(2-(3-methoxybenzyl)-3-oxoisoindolin-5-yl)-N-methylpicolinamide as a white solid.

¹H NMR (400 MHz, DMSO-d6) δ 9.01 (dd, J=2.3, 0.9 Hz, 1H), 8.83 (q, J=4.6 Hz, 1H), 8.37 (dd, J=8.2, 2.4 Hz, 1H), 8.10-8.12 (m, 2H), 8.04 (dd, J=7.9, 1.8 Hz, 1H), 7.73 (d, J=7.6 Hz, 1H), 7.28 (ddt, J=8.5, 7.0, 1.3 Hz, 1H), 6.85-6.89 (m, 3H), 4.74 (s, 2H), 4.45 (s, 2H), 3.74 (s, 3H), 2.85 (d, J=4.8 Hz, 3H); LCMS: Calculated: for C23H21N3O3: 387, Measured: 388 [M+H]+.

### Example 71

### 4-(2-(3-methoxybenzyl)-3-oxoisoindolin-5-yl)-N-methylpicolinamide

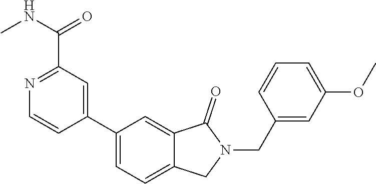

### Step 1: Ethyl 4-(2-(3-methoxybenzyl)-3-oxoisoindolin-5-yl)picolinate

Under a nitrogen atmosphere, a mixture of 2-(3-methoxybenzyl)-6-(4,4,5,5-tetramethyl-1,3,2-dioxaborolan-2-yl)isoindolin-1-one (150 mg, 0.396 mmol), ethyl 4-bromopicolinate (109 mg, 0.474 mmol), K₂CO₃ (164 mg, 1.187 mmol) and Pd(PPh₃)₄ (23 mg, 0.020 mmol) in DMF (10 mL) and H₂O (1 mL) was warmed to 100° C. After 2 h, the reaction mixture was allowed to cool to room temperature, diluted with EtOAc (25 mL), washed with brine (1×50 mL), dried (Na₂SO₄), filtered and concentrated in vacuo. Purification of the resulting residue by silica gel column chromatography (40 g, 0-100% EtOAc/heptane) yielded ethyl 4-(2-(3-methoxybenzyl)-3-oxoisoindolin-5-yl)picolinate as a yellow oil. LCMS: Calculated: for C24H22N2O4: 402, Measured: 403 [M+H]+.

### Step 2: 4-(2-(3-methoxybenzyl)-3-oxoisoindolin-5-yl)-N-methylpicolinamide

Under a nitrogen atmosphere, a mixture of ethyl 4-(2-(3-methoxybenzyl)-3-oxoisoindolin-5-yl)picolinate (100 mg, 0.248 mmol) in methanamine (2 mL, 4.00 mmol, 2M in THF) was warmed to 100° C. After 12 h, the reaction mixture was allowed to cool to room temperature and concentrated in vacuo. Purification of the resulting residue by C18 reverse column chromatography (80 g, 5%-35% ACN/H₂O (containing 0.05% TFA)) yielded 4-(2-(3-methoxybenzyl)-3-oxoisoindolin-5-yl)-N-methylpicolinamide as a light yellow solid.

¹H NMR (400 MHz, DMSO-d6) δ 8.85 (q, J=4.8 Hz, 1H), 8.72 (d, J=5.1 Hz, 1H), 8.33 (d, J=1.9 Hz, 1H), 8.13 (d, J=1.6 Hz, 1H), 8.09 (dd, J=7.9, 1.8 Hz, 1H), 8.03 (dd, J=5.1, 2.0 Hz, 1H), 7.74 (d, J=7.8 Hz, 1H), 7.26-7.30 (m, 1H), 6.80-6.95 (m, 3H), 4.74 (s, 2H), 4.46 (s, 2H), 3.74 (s, 3H), 2.86 (d, J=4.8 Hz, 3H). 19F NMR (376 MHz, DMSO-d6) d −74.59. LCMS: Calculated: for C23H21N3O3: 387, Measured: 388 [M+H]+.

### Example 72

### 6-(2-aminopyrimidin-4-yl)-2-(3-methoxybenzyl)-3,3-dimethylisoindolin-1-one

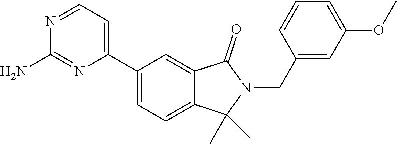 [SMILES: COc1cccc(CN2C(=O)c3cc(-c4ccnc(N)n4)ccc3C2(C)C)c1]

6-(2-aminopyrimidin-4-yl)-2-(3-methoxybenzyl)-3,3-dimethylisoindolin-1-one was prepared according to the procedure described in Example 70, Step 2, substituting 6-bromo-2-(3-ethoxybenzyl)-3,3-dimethylisoindolin-1-one for 6-bromo-2-(3-methoxybenzyl)isoindolin-1-one.

¹H NMR (400 MHz, DMSO-d6) δ 8.43 (s, 1H), 8.31-8.35 (m, 2H), 7.79 (d, J=8.0 Hz, 1H), 7.21-7.26 (m, 2H), 6.94 (dd, J=7.3, 1.5 Hz, 2H), 6.80-6.82 (m, 1H), 6.76 (s, 2H), 4.69 (s, 2H), 3.72 (s, 3H), 1.40 (s, 6H); LCMS: Calculated: for C22H22N4O2: 374, Measured: 375 [M+H]+.

### Example 73

### 2-(3-methoxybenzyl)-3,3-dimethyl-6-(1H-pyrazol-4-yl)isoindolin-1-one

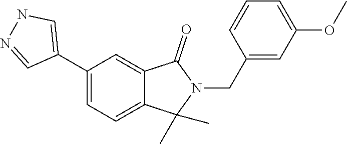 [SMILES: COc1cccc(CN2C(=O)c3cc(-c4cn[nH]c4)ccc3C2(C)C)c1]

### Step 1: 6-Bromo-2-(3-methoxybenzyl)-3,3-dimethylisoindolin-1-one

6-Bromo-2-(3-methoxybenzyl)-3,3-dimethylisoindolin-1-one was prepared according to the procedure described in Example 81, Step 1, substituting iodomethane for allyl bromide. LCMS: Calculated: for C18H18BrNO2: 360, Measured: 361 [M+H]+.

### Step 2: 2-(3-Methoxybenzyl)-3,3-dimethyl-6-(1H-pyrazol-4-yl)isoindolin-1-one

2-(3-Methoxybenzyl)-3,3-dimethyl-6-(1H-pyrazol-4-yl)isoindolin-1-one was prepared according to the procedure described in Example 1, Step 5, substituting 6-bromo-2-(3-methoxybenzyl)-3,3-dimethylisoindolin-1-one for 6-bromo-2-(3-methoxybenzyl)isoindolin-1-one.

¹H NMR (400 MHz, DMSO-d6) δ 13.00 (s, 1H), 8.34 (s, 1H), 8.03 (s, 1H), 7.92 (dd, J=1.6, 0.7 Hz, 1H), 7.87 (dd, J=7.9, 1.7 Hz, 1H), 7.63 (dd, J=7.9, 0.7 Hz, 1H), 7.22 (t, J=8.0 Hz, 1H), 6.93 (dd, J=7.3, 1.5 Hz, 2H), 6.77-6.84 (m, 1H), 4.66 (s, 2H), 3.71 (s, 3H), 1.37 (s, 6H). LCMS: Calculated: for C21H21N3O2: 347, Measured: 348 [M+H]+.

### Example 74

### 3,3-bis(2-hydroxyethyl)-2-(3-methoxybenzyl)-6-(1H-pyrazol-4-yl)isoindolin-1-one

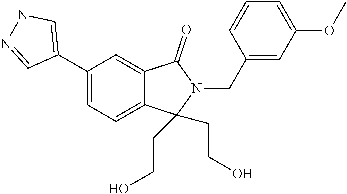 [SMILES: COc1cccc(CN2C(=O)c3cc(-c4cn[nH]c4)ccc3C2(CCO)CCO)c1]

### Step 1: 6-Bromo-3,3-bis(2-(tert-butyldimethylsilyloxy)ethyl)-2-(3-methoxybenzyl)isoindolin-1-one

6-Bromo-3,3-bis(2-(tert-butyldimethylsilyloxy)ethyl)-2-(3-methoxybenzyl)isoindolin-1-one was prepared according to the procedure described in Example 81, Step 1, substituting (2-bromoethoxy)(tert-butyl)dimethylsilane for allyl bromide. LCMS: Calculated: for C32H50BrNO4Si2: 648, Measured: 672.3[M+Na]+.

### Step 2: 3,3-Bis(2-(tert-butyldimethylsilyloxy)ethyl)-2-(3-methoxybenzyl)-6-(1H-pyrazol-4-yl)isoindolin-1-one

3,3-Bis(2-(tert-butyldimethylsilyloxy)ethyl)-2-(3-methoxybenzyl)-6-(1H-pyrazol-4-yl)isoindolin-1-one was prepared according to the procedure described in Example 1, Step 5, substituting 6-bromo-3,3-bis(2-(tert-butyldimethylsilyloxy)ethyl)-2-(3-methoxybenzyl)isoindolin-1-one for 6-bromo-2-(3-methoxybenzyl)isoindolin-1-one. LCMS: Calculated: for C35H53N3O4Si2: 635, Measured: 636 [M+H]+.

### Step 3: 3,3-Bis(2-hydroxyethyl)-2-(3-methoxybenzyl)-6-(1H-pyrazol-4-yl)isoindolin-1-one

A mixture of 3,3-bis(2-(tert-butyldimethylsilyloxy)ethyl)-2-(3-methoxybenzyl)-6-(1H-pyrazol-4-yl)isoindolin-1-one (160 mg, 0.252 mmol) in THE (10 mL) was treated with TBAF (1.01 mL, 1.01 mmol, 1M in THF) and the reaction was stirred at room temperature. After 2 h, diluted with EtOAc (50 mL), washed with brine (3×15 mL), dried (Na₂SO₄), filtered and concentrated in vacuo. Purification of the resulting residue by C18 reverse column chromatography (40 g, 0%-100% ACN/H₂O (containing 0.05% TFA)) yielded 3,3-bis(2-hydroxyethyl)-2-(3-methoxybenzyl)-6-(1H-pyrazol-4-yl)isoindolin-1-one as a white solid.

¹H NMR (400 MHz, DMSO-d6) δ 8.12 (s, 2H), 7.78-7.82 (m, 2H), 7.50 (d, J=7.9 Hz, 1H), 7.17 (t, J=7.8 Hz, 1H), 6.99-7.02 (m, 2H), 6.75 (dd, J=8.2, 2.6 Hz, 1H), 4.48 (s, 2H), 3.66 (s, 3H), 2.68 (td, J=9.5, 6.1 Hz, 2H), 2.57 (td, J=9.5, 6.2 Hz, 2H), 2.05 (tq, J=14.1, 7.1, 6.0 Hz, 4H); 19F NMR (376 MHz, DMSO-d6) d −74.31; LCMS: Calculated: for C23H25N3O4: 407, Measured: 408 [M+H]+.

### Example 75

### 3-(2-hydroxyethyl)-2-((R)-1-(3-methoxyphenyl)ethyl)-6-(1H-pyrazol-4-yl)isoindolin-1-one

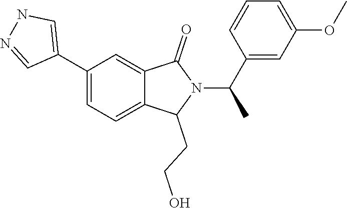 [SMILES: COc1cccc([C@@H](C)N2C(=O)c3cc(-c4cn[nH]c4)ccc3C2CCO)c1]

### Step 1: 6-Bromo-3-(2,3-dihydroxypropyl)-2-((R)-1-(3-methoxyphenyl)ethyl)isoindolin-1-one

A mixture of 3-allyl-6-bromo-2-((R)-1-(3-methoxyphenyl)ethyl)isoindolin-1-one (340 mg, 0.880 mmol) in acetone (15 mL) and H₂O (1.5 mL) with NMO (309 mg, 2.638 mmol) was treated OsO₄ (2.5% in t-BuOH, 447 mg, 0.044 mmol) and the reaction mixture was stirred at room temperature. After 4 h, the reaction mixture was diluted with EtOAc (30 mL), washed with brine (3×15 mL), dried (Na₂SO₄), filtered and concentrated in vacuo. Purification of the resulting residue by a silica gel chromatography (40 g, 0-100% EtOAc/heptane) yielded 6-bromo-3-(2,3-dihydroxypropyl)-2-((R)-1-(3-methoxyphenyl)ethyl)isoindolin-1-one as a white solid. LCMS: Calculated: for C20H22BrNO4: 420, Measured: 444 [M+Na]+.

### Step 2: 2-(5-bromo-2-((R)-1-(3-methoxyphenyl)ethyl)-3-oxoisoindolin-1-yl)acetaldehyde

A mixture of 6-bromo-3-(2,3-dihydroxypropyl)-2-((R)-1-(3-methoxyphenyl)ethyl)isoindolin-1-one (340 mg, 0.81 mmol) in MeOH (15 mL) and H₂O (1.5 mL) was added NaIO₄ (260 mg, 1.22 mmol). The reaction was stirred at room temperature for an overnight. After 12 h, the reaction mixture was diluted with EtOAc (30 mL), washed with brine (3×15 mL), dried (Na₂SO₄), filtered and concentrated in vacuo. Purification of the resulting residue by a silica gel chromatography (40 g, 0-100% EtOAc/heptane) yielded 2-(5-bromo-2-((R)-1-(3-methoxyphenyl)ethyl)-3-oxoisoindolin-1-yl)acetaldehyde as a colorless oil. LCMS: Calculated: for C19H18BrNO3: 388, Measured: 388 [M]+.

### Step 3: 6-Bromo-3-(2-hydroxyethyl)-2-((R)-1-(3-methoxyphenyl)ethyl)isoindolin-1-one

A mixture of 2-(5-bromo-2-((R)-1-(3-methoxyphenyl)ethyl)-3-oxoisoindolin-1-yl)acetaldehyde (250 mg, 0.64 mmol) in EtOH (15 mL) was treated with NaBH₄ (97 mg, 2.56 mmol) at 0° C. and the resulting mixture was allowed to warm to room temperature. After 2 h, the reaction mixture was poured into ice/water (30 mL) and extracted with EtOAc (3×15 mL), washed with brine (3×15 mL), dried (Na₂SO₄), filtered and concentrated in vacuo. Purification of the resulting residue by a silica gel chromatography (40 g, 0-100% EtOAc/heptane) yielded 6-bromo-3-(2-hydroxyethyl)-2-((R)-1-(3-methoxyphenyl)ethyl)isoindolin-1-one as colorless oil. LCMS: Calculated: for C19H20BrNO3: 390, Measured: 392 [M+2H]+.

### Step 4: 3-(2-Hydroxyethyl)-2-((R)-1-(3-methoxyphenyl)ethyl)-6-(1H-pyrazol-4-yl)isoindolin-1-one

3-(2-Hydroxyethyl)-2-((R)-1-(3-methoxyphenyl)ethyl)-6-(1H-pyrazol-4-yl)isoindolin-1-one was prepared according to the procedure described in Example 1, Step 5, substituting 6-bromo-3-(2-hydroxyethyl)-2-((R)-1-(3-methoxyphenyl)ethyl)isoindolin-1-one for 6-bromo-2-(3-methoxybenzyl)isoindolin-1-one.

¹H NMR (300 MHz, DMSO-d6) δ 12.98 (brs, 1H), 8.32 (brs, 1H), 8.02 (brs, 1H), 7.77-7.90 (m, 2H), 7.53 (dd, J=11.8, 7.9 Hz, 1H), 7.26 (dt, J=10.0, 8.0 Hz, 1H), 6.90-7.04 (m, 2H), 6.78-6.89 (m, 1H), 5.27 (dd, J=18.6, 7.3 Hz, 1H), 4.40-4.84 (m, 2H), 3.72 (d, J=3.2 Hz, 3H), 3.25 (d, J=27.7 Hz, 2H), 2.09 (d, J=46.6 Hz, 1H), 1.88 (dt, J=13.6, 6.6 Hz, 1H), 1.79 (d, J=7.3 Hz, 1H), 1.72 (d, J=7.2 Hz, 2H); LCMS: Calculated: for C22H23N3O3: 377, Measured: 378 [M+H]+.

### Example 76

### 22-(2-(3-methoxybenzyl)-3-oxo-5-(1H-pyrazol-4-yl)isoindolin-1-yl)acetic acid

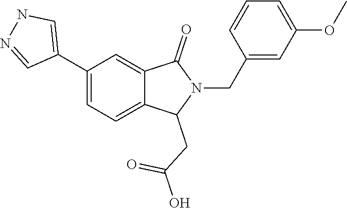 [SMILES: COc1cccc(CN2C(=O)c3cc(-c4cn[nH]c4)ccc3C2CC(=O)O)c1]

### Step 1: 2-(5-Bromo-2-(3-methoxybenzyl)-3-oxoisoindolin-1-yl)acetic acid

A mixture of 2-(5-bromo-2-((R)-1-(3-methoxyphenyl)ethyl)-3-oxoisoindolin-1-yl)acetaldehyde (200 mg, 0.534 mmol) in ACN (15 mL) and H₂O (3 mL) with NaH₂PO₄ (19.2 mg, 0.160 mmol) was treated with H₂O₂ (30% w/w, 60.6 mg, 0.53 mmol,). and NaClO₂ (87 mg, 0.96 mmol) at 0° C. After addition, the resulting mixture was allowed to warm to room temperature. After 4 h, the reaction mixture was diluted with water (20 mL), pH was adjusted to 6 with HCl (1N), the resulting mixture was extracted with EtOAc (3×15 mL), washed with brine (3×15 mL), dried (Na₂SO₄), filtered and concentrated in vacuo to yield 2-(5-bromo-2-(3-methoxybenzyl)-3-oxoisoindolin-1-yl)acetic acid as a light yellow solid. LCMS Calculated: for C18H16BrNO4: 390, Measured: 414 [M+Na]+.

### Step 2: 2-(2-(3-Methoxybenzyl)-3-oxo-5-(1H-pyrazol-4-yl)isoindolin-1-yl)acetic acid

2-(2-(3-Methoxybenzyl)-3-oxo-5-(1H-pyrazol-4-yl)isoindolin-1-yl)acetic acid was prepared according to the procedure described in Example 1, Step 5, substituting 2-(5-bromo-2-(3-methoxybenzyl)-3-oxoisoindolin-1-yl)acetic acid for 6-bromo-2-(3-methoxybenzyl)isoindolin-1-one.

¹H NMR (400 MHz, DMSO-d6) δ 12.72 (brs, 2H), 8.18 (brs, 2H), 7.93 (d, J=1.6 Hz, 1H), 7.84 (dd, J=7.9, 1.7 Hz, 1H), 7.52 (d, J=8.0 Hz, 1H), 7.23 (t, J=7.8 Hz, 1H), 6.80-6.84 (m, 3H), 5.03 (d, J=15.5 Hz, 1H), 4.68 (dd, J=7.2, 4.7 Hz, 1H), 4.36 (d, J=15.5 Hz, 1H), 3.71 (s, 3H), 3.00 (dd, J=16.3, 4.7 Hz, 1H), 2.63 (dd, J=16.3, 7.2 Hz, 1H); LCMS Calculated: for C21H19N3O4: 377, Measured: 378 [M+H]+.

### Example 77

### 2-((S)-2-((R)-1-(3-methoxyphenyl)ethyl)-3-oxo-5-(1H-pyrazol-4-yl)isoindolin-1-yl)acetic acid

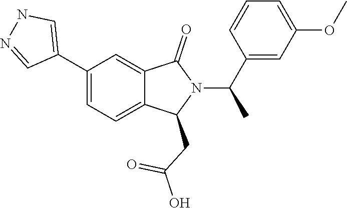 [SMILES: COc1cccc([C@@H](C)N2C(=O)c3cc(-c4cn[nH]c4)ccc3[C@@H]2CC(=O)O)c1]

### And Example 78

### 2-((R)-2-((R)-1-(3-methoxyphenyl)ethyl)-3-oxo-5-(1H-pyrazol-4-yl)isoindolin-1-yl)acetic acid

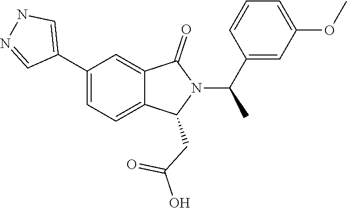 [SMILES: COc1cccc([C@@H](C)N2C(=O)c3cc(-c4cn[nH]c4)ccc3[C@H]2CC(=O)O)c1]

2-((S)-2-((R)-1-(3-Methoxyphenyl)ethyl)-3-oxo-5-(1H-pyrazol-4-yl)isoindolin-1-yl)acetic acid and 2-((R)-2-((R)-1-(3-Methoxyphenyl)ethyl)-3-oxo-5-(1H-pyrazol-4-yl)isoindolin-1-yl)acetic acid were prepared according to the procedure described in Example 76, Step 1, substituting (R)-6-bromo-2-(1-(3-methoxyphenyl)ethyl)isoindolin-1-one for 6-bromo-2-(3-methoxybenzyl)isoindolin-1-one.

2-((S)-2-((R)-1-(3-Methoxyphenyl)ethyl)-3-oxo-5-(1H-pyrazol-4-yl)isoindolin-1-yl)acetic acid: ¹H NMR (300 MHz, DMSO-d6) δ 8.75 (d, J=11.2 Hz, 2H), 7.87 (d, J=7.5 Hz, 2H), 7.47 (d, J=8.0 Hz, 1H), 7.12 (t, J=7.9 Hz, 1H), 6.76-6.98 (m, 2H), 6.70 (dd, J=8.1, 2.6 Hz, 1H), 5.15 (t, J=7.4 Hz, 1H), 4.87 (dd, J=6.6, 3.7 Hz, 1H), 3.57 (s, 3H), 2.74 (dd, J=16.7, 3.9 Hz, 1H), 2.29 (dd, J=16.8, 7.3 Hz, 1H), 1.63 (d, J=7.1 Hz, 3H) and

2-((R)-2-((R)-1-(3-Methoxyphenyl)ethyl)-3-oxo-5-(1H-pyrazol-4-yl)isoindolin-1-yl)acetic acid (Anti75): ¹H NMR (300 MHz, DMSO-d6) δ 8.63 (d, J=25.1 Hz, 3H), 7.75-8.00 (m, 2H), 7.45 (d, J=8.0 Hz, 2H), 7.19 (ddd, J=10.7, 7.2, 3.0 Hz, 2H), 6.71-6.97 (m, 6H), 5.23 (dd, J=7.1, 3.1 Hz, 2H), 4.58 (dd, J=6.8, 3.7 Hz, 1H), 3.62 (d, J=2.3 Hz, 3H), 3.00 (dd, J=16.8, 3.8 Hz, 1H), 2.51-2.63 (m, 1H), 1.48-1.70 (m, 3H); LCMS Calculated: for C22H21N3O4: 391, Measured: 392 [M+H]+.

### Example 79

### 3-((2H-tetrazol-5-yl)methyl)-2-(3-methoxybenzyl)-6-(1H-pyrazol-4-yl)isoindolin-1-one

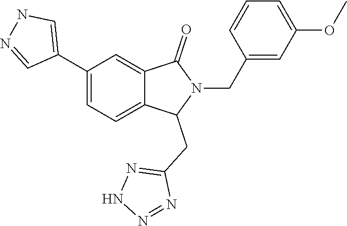 [SMILES: COc1cccc(CN2C(=O)c3cc(-c4cn[nH]c4)ccc3C2Cc2nn[nH]n2)c1]

### Step 1: 2-(5-Bromo-2-(3-methoxybenzyl)-3-oxoisoindolin-1-yl)acetaldehyde

2-(5-Bromo-2-(3-methoxybenzyl)-3-oxoisoindolin-1-yl)acetaldehyde was prepared according to the procedure described in Example 75, Step 2, substituting 3-allyl-6-bromo-2-(3-methoxybenzyl)isoindolin-1-one for 6-bromo-3-(2,3-dihydroxypropyl)-2-((R)-1-(3-methoxyphenyl)ethyl)isoindolin-1-one.

### Step 2: 2-(5-Bromo-2-(3-methoxybenzyl)-3-oxoisoindolin-1-yl)acetonitrile

Under a nitrogen atmosphere, a mixture of 2-(5-bromo-2-(3-methoxybenzyl)-3-oxoisoindolin-1-yl)acetaldehyde (300 mg, 0.80 mmol) in DMSO (10 mL) with NH₂OH.HCl (334 mg, 4.81 mmol) was stirred at 100° C.

After 12 h, the reaction mixture was allowed to cool to room temperature, diluted with EtOAc (3×15 mL), washed with brine (3×15 mL), dried (Na₂SO₄), filtered and concentrated in vacuo. Purification of the resulting residue by a silica gel chromatography (40 g, 0-100% EtOAc/heptane) yielded 2-(5-bromo-2-(3-methoxybenzyl)-3-oxoisoindolin-1-yl)acetonitrile as a yellow solid.

### Step 3: 2-(2-(3-Methoxybenzyl)-3-oxo-5-(1-(tetrahydro-2H-pyran-2-yl)-1H-pyrazol-4-yl)isoindolin-1-yl)acetonitrile

Under a nitrogen atmosphere, a mixture of 2-(5-bromo-2-(3-methoxybenzyl)-3-oxoisoindolin-1-yl)acetonitrile (130 mg, 0.350 mmol), 1-(tetrahydro-2H-pyran-2-yl)-4-(4,4,5,5-tetramethyl-1,3,2-dioxaborolan-2-yl)-1H-pyrazole (146 mg, 0.525 mmol), K₂CO₃ (145 mg, 1.049 mmol) and Pd(PPh₃)₄ (20 mg, 0.017 mmol) in DMF (15 mL) and H₂O (1.5 mL) was stirred at 100° C. After 3 h, the reaction mixture was allowed to cool to room temperature, diluted with EtOAc (3×15 mL), washed with brine (3×15 mL), dried (Na₂SO₄), filtered and concentrated in vacuo. Purification of the resulting residue by a silica gel chromatography (40 g, 0-100% EtOAc/heptane) yielded 2-(2-(3-methoxybenzyl)-3-oxo-5-(1-(tetrahydro-2H-pyran-2-yl)-1H-pyrazol-4-yl)isoindolin-1-yl)acetonitrile as a yellow solid.

### Step 4: 3-((2H-Tetrazol-5-yl)methyl)-2-(3-methoxybenzyl)-6-(1-(tetrahydro-2H-pyran-2-yl)-1H-pyrazol-4-yl)isoindolin-1-one

Under a nitrogen atmosphere, a mixture of 2-(2-(3-methoxybenzyl)-3-oxo-5-(1-(tetrahydro-2H-pyran-2-yl)-1H-pyrazol-4-yl)isoindolin-1-yl)acetonitrile (100 mg, 0.226 mmol), TMSN₃ (130 mg, 1.128 mmol) and dibutyltin oxide (5.6 mg, 0.022 mmol) in toluene (10 mL) was stirred at 110° C. After 2 h, the reaction mixture was allowed to cool to room temperature, diluted with EtOAc (3×15 mL), washed with brine (3×15 mL), dried (Na₂SO₄), filtered and concentrated in vacuo. Purification of the resulting residue by a silica gel chromatography (40 g, 0-100% EtOAc/heptane) yielded 3-((2H-tetrazol-5-yl)methyl)-2-(3-methoxybenzyl)-6-(1-(tetrahydro-2H-pyran-2-yl)-1H-pyrazol-4-yl)isoindolin-1-one as a yellow solid.

### Step 5: 3-((2H-Tetrazol-5-yl)methyl)-2-(3-methoxybenzyl)-6-(1H-pyrazol-4-yl)isoindolin-1-one

A mixture of 3-((2H-tetrazol-5-yl)methyl)-2-(3-methoxybenzyl)-6-(1-(tetrahydro-2H-pyran-2-yl)-1H-pyrazol-4-yl)isoindolin-1-one (40 mg, 0.082 mmol) in DCM (5 mL) was treated with TFA (140 mg, 1.23 mmol) and allowed to stir at room temperature. After 2 h, diluted with EtOAc (50 mL), washed with brine (3×15 mL), dried (Na₂SO₄), filtered and concentrated in vacuo. Purification of the resulting residue by C18 reverse column chromatography (40 g, 0%-100% ACN/H₂O (containing 0.05% TFA)) yielded 3-((2H-tetrazol-5-yl)methyl)-2-(3-methoxybenzyl)-6-(1H-pyrazol-4-yl)isoindolin-1-one as a white solid.

¹H NMR (400 MHz, DMSO-d6) δ 8.17 (brs, 2H), 7.85 (s, 1H), 7.81 (dd, J=7.9, 1.7 Hz, 1H), 7.32 (d, J=8.0 Hz, 1H), 7.26 (t, J=7.7 Hz, 1H), 6.81-6.87 (m, 3H), 5.17 (d, J=15.5 Hz, 1H), 4.84 (dd, J=6.0, 4.4 Hz, 1H), 4.41 (d, J=15.5 Hz, 1H), 3.73 (s, 3H), 3.68 (d, J=4.1 Hz, 1H), 3.54-3.63 (m, 1H); LCMS Calculated: for C21H19N7O2: 401, Measured: 402 [M+H]+.

### Example 80

### 2-(2-(3-ethoxybenzyl)-3-oxo-5-(1H-pyrazol-4-yl)isoindolin-1-yl)acetonitrile

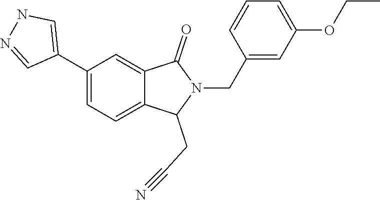 [SMILES: CCOc1cccc(CN2C(=O)c3cc(-c4cn[nH]c4)ccc3C2CC#N)c1]

2-(2-(3-Ethoxybenzyl)-3-oxo-5-(1H-pyrazol-4-yl)isoindolin-1-yl)acetonitrile was prepared according to the procedure described in Example 79, Step 3, substituting tert-butyl 4-(4,4,5,5-tetramethyl-1,3,2-dioxaborolan-2-yl)-1H-pyrazole-1-carboxylate for 1-(tetrahydro-2H-pyran-2-yl)-4-(4,4,5,5-tetramethyl-1,3,2-dioxaborolan-2-yl)-1H-pyrazole and 6-bromo-2-(3-ethoxybenzyl)isoindolin-1-one for 6-bromo-2-(3-methoxybenzyl)isoindolin-1-one.

¹H NMR (400 MHz, DMSO-d6) δ 8.23 (brs, 2H), 8.00 (s, 1H), 7.93 (dd, J=8.0, 1.7 Hz, 1H), 7.67 (d, J=7.9 Hz, 1H), 7.24 (t, J=7.8 Hz, 1H), 6.82-6.89 (m, 3H), 5.05 (d, J=15.4 Hz, 1H), 4.68 (t, J=4.1 Hz, 1H), 4.41 (d, J=15.4 Hz, 1H), 3.93-4.01 (m, 2H), 3.43 (s, 2H), 1.29 (t, J=7.0 Hz, 3H)./19F NMR (376 MHz, DMSO-d6) d −74.13; LCMS Calculated: for C21H19N7O2: 472, Measured: 473 [M+H]+.

### Example 81

### 2′-(3-methoxybenzyl)-5′-(1H-pyrazol-4-yl)spiro[cyclopentane-1,1′-isoindolin]-3-en-3′-one

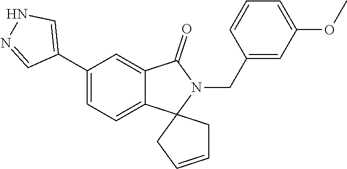 [SMILES: COc1cccc(CN2C(=O)c3cc(-c4cn[nH]c4)ccc3C23CC=CC3)c1]

### Step 1: 3,3-Diallyl-6-bromo-2-(3-methoxybenzyl)isoindolin-1-one

To a mixture of 6-bromo-2-(3-methoxybenzyl)isoindolin-1-one (3 g, 9.031 mmol) in THE (100 mL) was added NaH (60%, 1.8 g, 45.0 mmol,) and stirred at room temperature for 1 h. The reaction mixture was treated with 3-bromoprop-1-ene (6.56 g, 54.3 mmol), the resulting mixture was warmed to 80° C. After 12 h, the resulting reaction mixture was poured into Ice/water (150 mL) and extracted with EtOAc (3×150 mL), washed with water (2×150 mL) and brine (2×150 mL), dried (Na₂SO₄) and concentrated in vacuo. Purification of the resulting residue by silica gel chromatography (120 g, 0-100% EtOAc/Heptane) yielded 3,3-diallyl-6-bromo-2-(3-methoxybenzyl)isoindolin-1-one as a yellow oil. LCMS: Calculated: for C22H22BrNO2: 412, Measured: MH+: 412 [M]+

### Step 2: 3-Allyl-6-bromo-2-(3-methoxybenzyl)isoindolin-1-one

To a mixture of 6-bromo-2-(3-methoxybenzyl)isoindolin-1-one (1.1 g, 3.31 mmol) in THE (35 mL) was added NaH (60%, 530 mg, 13.3 mmol), and the resulting mixture was stirred at room temperature for 20 min then treated with 3-bromoprop-1-ene (1.60 g, 13.3 mmol). The reaction mixture was stirred at 65° C. for 4 h. The resulting solution was poured into ice/water (50 mL) and extracted with EtOAc (3×50 mL), washed with brine (3×50 mL), dried (Na₂SO₄), and concentrated in vacuo. Purification of the resulting residue by silica gel chromatography (80 g, 0-100% EtOAc/Heptane) yielded 3-allyl-6-bromo-2-(3-methoxybenzyl)isoindolin-1-one as a yellow oil. LCMS: Calculated: for 372, Measured: 372 [M]+.

### Step 3: 3-Allyl-6-bromo-2-((R)-1-(3-methoxyphenyl)ethyl)isoindolin-1-one

3-Allyl-6-bromo-2-((R)-1-(3-methoxyphenyl)ethyl)isoindolin-1-one was prepared according to the procedure described in Example 81, Step 2, substituting (R)-6-bromo-2-(1-(3-methoxyphenyl)ethyl)isoindolin-1-one for 6-bromo-2-(3-methoxybenzyl)isoindolin-1-one. LCMS: Calculated: for C20H20BrNO2: 386, Measured: 408 [M+Na]+.

### Step 4: 5′-Bromo-2′-[(3-methoxyphenyl)methyl]-2′,3′-dihydrospiro[cyclopentane-1,1′-isoindol]-3-en-3′-one

To a solution of 3,3-diallyl-6-bromo-2-(3-methoxybenzyl)isoindolin-1-one (8.6 g, 0.021 mol) in DCM (150 mL) added [1,3-bis-(2,4,6-trimethylphenyl)-2-imidazolidinylidene]dichloro(phenylmethylene)(tricyclohexylphosphino)ruthenium (0.887 g, 1.04 mmol). The resulting mixture was stirred at room temperature. After 12 h, the resulting solution was concentrated in vacuo. Purification of the resulting residue by silica gel chromatography (0-100% EtOAc/Heptane) yielded 5′-bromo-2′-[(3-methoxyphenyl)methyl]-2′,3′-dihydrospiro[cyclopentane-1,1′-isoindol]-3-en-3′-one as a yellow oil. LCMS: Calculated: for C20H18BrNO2: 384, Measured: 386[M+2H]+.

### Step 5: 2′-(3-Methoxybenzyl)-5′-(1H-pyrazol-4-yl)spiro[cyclopentane-1,1′-isoindolin]-3-en-3′-one

Under a nitrogen atmosphere, a mixture of 5′-bromo-2′-[(3-methoxyphenyl)methyl]-2′,3′-dihydrospiro[cyclopentane-1,1′-isoindol]-3-en-3′-one (301 mg, 0.783 mmol), tert-butyl 4-(4,4,5,5-tetramethyl-1,3,2-dioxaborolan-2-yl)-1H-pyrazole-1-carboxylate (691.231 mg, 2.35 mmol) and Pd(PPh₃)₄ (45 mg, 0.0392 mmol) was added a solution of K₂CO₃ (325 mg, 2.35 mmol) in water (2 mL). The reaction mixture was stirred for an overnight at 110° C. The resulting mixture was allowed to cool to room temperature, diluted with EtOAc (50 mL), washed with brine (3×15 mL), dried (Na₂SO₄), filtered and concentrated in vacuo. Purification of the resulting residue by a silica gel chromatography (40 g, 0-100% EtOAc/heptane) yielded 2′-(3-methoxybenzyl)-5′-(1H-pyrazol-4-yl)spiro[cyclopentane-1,1′-isoindolin]-3-en-3′-one as white solid/residue. Further Purification of the resulting residue by HPLC (0-100% ACN/water containing 5% TFA) yielded 2′-(3-methoxybenzyl)-5′-(1H-pyrazol-4-yl)spiro[cyclopentane-1,1′-isoindolin]-3-en-3′-one as a white solid.

¹H NMR (300 MHz, METHANOL-d4) 8.2-8.1 (bs, 2H), 7.96 (s, 1H), 7.86 (d, J=1.8 Hz, 1H), 7.54 (d, J=1.8 Hz, 1H), 7.20 (t, J=7.8 Hz, 1H), 6.90-6.86 (m, 2H), 6.8 (d, J=7.9 Hz, 1H), 5.85 (s, 2H), 4.65 (s, 2H), 3.73 (s, 3H), 2.83-2.75 (m, 2H), 2.68-2.60 (m, 2H); LCMS: Calculated: for C23H21N3O2: 372, Measured: 372 [M]+.

### Example 82

### 2′-(2-hydroxy-1-(3-methoxyphenyl)ethyl)-5′-(1H-pyrazol-4-yl)spiro[cyclopentane-1,1′-isoindolin]-3-en-3′-one

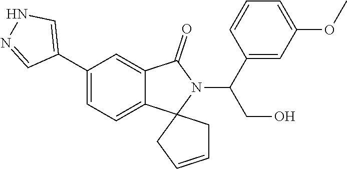 [SMILES: COc1cccc(C(CO)N2C(=O)c3cc(-c4cn[nH]c4)ccc3C23CC=CC3)c1]

2′-(2-hydroxy-1-(3-methoxyphenyl)ethyl)-5′-(1H-pyrazol-4-yl)spiro[cyclopentane-1,1′-isoindolin]-3-en-3′-one was prepared according to the procedure described in Example 81, Step 1, substituting 6-Bromo-2-(2-hydroxy-1-(3-methoxyphenyl)ethyl)isoindolin-1-one for 6-bromo-2-(3-methoxybenzyl)isoindolin-1-one.

¹H NMR (400 MHz, METHANOL-d4) δ 8.06 (s, 1H), 7.89-7.94 (m, 1H), 7.81-7.89 (m, 1H), 7.42-7.53 (m, 1H), 7.18-7.28 (m, 1H), 7.08-7.17 (m, 1H), 6.98-7.08 (m, 1H), 6.77-6.89 (m, 1H), 5.94-6.02 (m, 1H), 5.77-5.89 (m, 1H), 4.75-4.83 (m, 2H), 4.48-4.60 (m, 1H), 3.85-3.96 (m, 1H), 3.76 (s, 3H), 3.10-3.23 (m, 1H), 2.86-3.01 (m, 2H), 2.50-2.75 (m, 2H); LCMS: Calculated: for C24H23N3O3: 401; Measured: 402 [M+H]+.

### Example 83

### 2′-(3-methoxybenzyl)-5′-(1H-pyrazol-4-yl)spiro[cyclopentane-1,1′-isoindolin]-3′-one

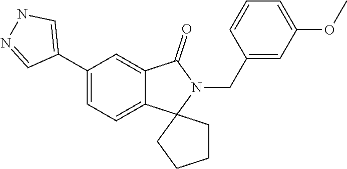 [SMILES: COc1cccc(CN2C(=O)c3cc(-c4cn[nH]c4)ccc3C23CCCC3)c1]

### Step 1: 2′-[(3-Methoxyphenyl)methyl]-5′-[1-(oxan-2-yl)-1H-pyrazol-4-yl]-2′,3′-dihydrospiro[cyclopentane-1,1′-isoindol]-3-en-3′-one

Under a nitrogen atmosphere, a mixture of 5′-bromo-2′-[(3-methoxyphenyl)methyl]-2′,3′-dihydrospiro[cyclopentane-1,1′-isoindol]-3-en-3′-one (200 mg, 0.520 mmol), 1-(tetrahydro-2H-pyran-2-yl)-4-(4,4,5,5-tetramethyl-1,3,2-dioxaborolan-2-yl)-1H-pyrazole (434 mg, 1.56 mmol), K₂CO₃ (216 mg, 1.56 mmol) and Pd(PPh₃)₄ (30 mg, 0.026 mmol) in DMF (15 mL) and H₂O (1 mL) was stirred at 100° C. After 3 h, the reaction mixture was allowed to cool, diluted with EtOAc (60 mL), washed with water (1×30 mL), brine (1×30 mL), dried (Na₂SO₄), filtered and concentrated in vacuo. Purification of the resulting residue by silica gel column chromatography (40 g, 0-20% MeOH/DCM) yielded 2′-[(3-methoxyphenyl)methyl]-5′-[1-(oxan-2-yl)-1H-pyrazol-4-yl]-2′,3′-dihydrospiro[cyclopentane-1,1′-isoindol]-3-en-3′-one as a yellow oil. LCMS: Calculated: for C28H29N3O3: 455, Measured: 456 [M+H]+

### Step 2: 2′-[(3-methoxyphenyl)methyl]-5′-[1-(oxan-2-yl)-1H-pyrazol-4-yl]-2′,3′-dihydrospiro[cyclopentane-1,1′-isoindole]-3′-one

A mixture of 2′-[(3-methoxyphenyl)methyl]-5′-[1-(oxan-2-yl)-1H-pyrazol-4-yl]-2′,3′-dihydrospiro[cyclopentane-1,1′-isoindol]-3-en-3′-one (180 mg, 0.395 mmol, 1.00 equiv) in MeOH (15 mL) with Pd(OH)₂/C (180 mg) was stirred under hydrogen for 2 h at room temperature. It was filtrated, the filtrate was concentrated. The residue was applied onto a silica gel column (40 g, MeOH/DCM: 1/10) to give 150 mg (83.0%) of 2′-[(3-methoxyphenyl)methyl]-5′-[1-(oxan-2-yl)-1H-pyrazol-4-yl]-2′,3′-dihydrospiro[cyclopentane-1,1′-isoindole]-3′-one as yellow oil. LCMS: Calculated: for C28H31N3O3: 457, Measured: 458 [M+H]+

### Step 3: 2′-[(3-Methoxyphenyl)methyl]-5′-(1H-pyrazol-4-yl)-2′,3′-dihydrospiro[cyclopentane-1,1′-isoindole]-3′-one

To a mixture of 2′-[(3-methoxyphenyl)methyl]-5′-[1-(oxan-2-yl)-1H-pyrazol-4-yl]-2′,3′-dihydrospiro[cyclopentane-1,1′-isoindole]-3′-one (140 mg, 0.306 mmol) in DCM (10 mL) was added TFA (349 mg, 3.061 mmol) and the resulting mixture was stirred at room temperature. After 2 h, the reaction mixture was concentrated in vacuo. Purification of the resulting residue by C18 reverse phase column chromatography (80 g, 0-50% ACN/H₂O (containing 0.05% TFA)) yielded 2′-[(3-methoxyphenyl)methyl]-5′-(1H-pyrazol-4-yl)-2′,3′-dihydrospiro[cyclopentane-1,1′-isoindole]-3′-one as a colorless solid.

¹H NMR (400 MHz, DMSO-d6) δ 8.20 (s, 2H), 7.91 (d, J=1.2 Hz, 1H), 7.87 (dd, J=8.0, 1.8 Hz, 1H), 7.56 (d, J=7.9 Hz, 1H), 7.23 (t, J=8.0 Hz, 1H), 6.84-6.89 (m, 2H), 6.78-6.84 (m, 1H), 4.67 (s, 2H), 3.71 (s, 3H), 1.98 (dt, J=12.2, 7.0 Hz, 2H), 1.89 (q, J=5.5, 3.7 Hz, 4H), 1.77 (dt, J=11.6, 5.2 Hz, 2H); 19F NMR (376 MHz, DMSO-d6) d −74.56; LCMS: Calculated: for C23H23N3O2: 373, Measured: 374 [M+H]+.

### Example 84

### 2′-(2-hydroxy-1-(3-methoxyphenyl)ethyl)-5′-(1H-pyrazol-4-yl)spiro[cyclopentane-1,1′-isoindolin]-3′-one

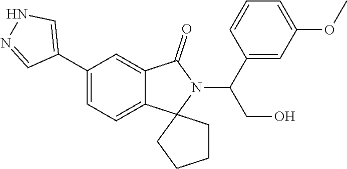 [SMILES: COc1cccc(C(CO)N2C(=O)c3cc(-c4cn[nH]c4)ccc3C23CCCC3)c1]

2′-(2-hydroxy-1-(3-methoxyphenyl)ethyl)-5′-(1H-pyrazol-4-yl)spiro[cyclopentane-1,1′-isoindolin]-3′-one was prepared according to the procedure described in Example 83, Step 1.

¹H NMR (400 MHz, METHANOL-d4) δ 8.10 (br s, 2H), 7.82-7.94 (m, 2H), 7.53 (d, J=8.08 Hz, 1H), 7.19-7.28 (m, 1H), 7.10-7.16 (m, 1H), 7.04 (d, J=7.58 Hz, 1H), 6.84 (s, 1H), 4.73 (d, J=11.12 Hz, 1H), 4.59-4.68 (m, 1H), 4.02 (dd, J=4.80, 11.37 Hz, 1H), 3.76 (s, 3H), 2.42-2.61 (m, 1H), 1.86-2.14 (m, 6H), 1.69-1.81 (m, 1H); LCMS: Calculated: for C24H25N3O3: 403; Measured: 404 [M+H]+.

### Example 85

### (R)-2′-(1-(3-methoxyphenyl)ethyl)-5′-(1H-pyrazol-4-yl)spiro[cyclopentane-1,1′-isoindolin]-3′-one

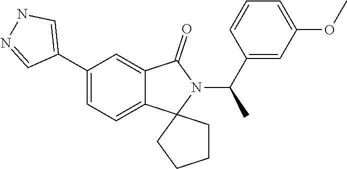 [SMILES: COc1cccc([C@@H](C)N2C(=O)c3cc(-c4cn[nH]c4)ccc3C23CCCC3)c1]

(R)-2′-(1-(3-Methoxyphenyl)ethyl)-5′-(1H-pyrazol-4-yl)spiro[cyclopentane-1,1′-isoindolin]-3′-one was prepared according to the procedure described in Example 83, Step 3.

¹H NMR (300 MHz, DMSO-d6) δ 8.12 (s, 2H), 7.84-7.75 (m, 2H), 7.49 (d, J=7.9 Hz, 1H), 7.18 (t, J=7.9 Hz, 1H), 7.01-6.92 (m, 2H), 6.76 (dd, J=8.3, 2.3 Hz, 1H), 4.67 (q, J=6.9 Hz, 1H), 3.67 (s, 3H), 2.22 (dt, J=12.5, 7.5 Hz, 1H), 2.02-1.86 (m, 5H), 1.81 (d, J=7.0 Hz, 4H), 1.67 (t, J=6.4 Hz, 1H); LCMS: Calculated: for C24H25N3O2: 387, Measured: 388 [M+H]+.

### Example 86

### 2′-(3-methoxybenzyl)-5′-(1H-pyrazol-4-yl)spiro[cyclohexane-1,1′-isoindolin]-3′-one

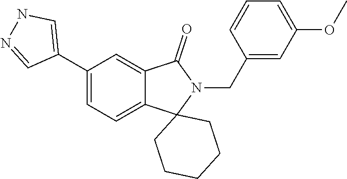 [SMILES: COc1cccc(CN2C(=O)c3cc(-c4cn[nH]c4)ccc3C23CCCCC3)c1]

2′-(3-methoxybenzyl)-5′-(1H-pyrazol-4-yl)spiro[cyclohexane-1,1′-isoindolin]-3′-one was prepared according to the procedure described in Example 88, Step 1, substituting cyclohexone for tert-butyl 4-oxopiperidine-1-carboxylate.

¹H NMR (400 MHz, CHLOROFORM-d) δ 8.13 (d, J=1.52 Hz, 1H), 7.80-7.89 (m, 1H), 7.64-7.74 (m, 1H), 7.16-7.25 (m, 1H), 6.82-6.93 (m, 1H), 6.74-6.82 (m, 1H), 5.03-5.38 (br s, 2H), 4.79 (s, 2H), 3.77 (s, 3H), 1.90 (br d, J=3.54 Hz, 9H), 1.17-1.53 (m, 4H); LCMS: Calculated: for C24H25N3O2: 387; Measured: 388 [M+H]+.

### Example 87

### 4-hydroxy-2′-(3-methoxybenzyl)-5′-(1H-pyrazol-4-yl)-2,3-dihydrospiro[indene-1,1′-isoindolin]-3′-one

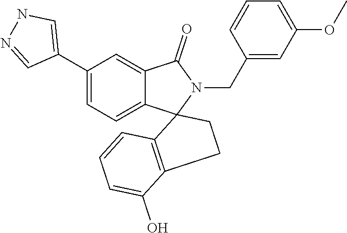 [SMILES: COc1cccc(CN2C(=O)c3cc(-c4cn[nH]c4)ccc3C23CCc2c(O)cccc23)c1]

4-hydroxy-2′-(3-methoxybenzyl)-5′-(1H-pyrazol-4-yl)-2,3-dihydrospiro[indene-1,1′-isoindolin]-3′-one was prepared according to the procedure described in Example 88, Step 1, substituting 4-(benzyloxy)-2,3-dihydro-1H-inden-1-one for tert-butyl 4-oxopiperidine-1-carboxylate.

¹H NMR (400 MHz, METHANOL-d4) δ 7.96-8.14 (m, 2H), 7.76-7.85 (m, 1H), 7.20-7.30 (m, 1H), 7.08-7.15 (m, 1H), 6.85-6.95 (m, 3H), 6.64-6.80 (m, 2H), 5.95-6.07 (m, 1H), 4.73-4.86 (m, 1H), 3.96-4.13 (m, 1H), 3.72 (s, 3H), 2.92-3.16 (m, 3H), 2.41-2.56 (m, 1H), 2.27-2.41 (m, 1H); LCMS: Calculated: for C27H23N3O3: 437; Measured: 438 [M+H]+.

### Example 88

### 2-(3-methoxybenzyl)-5-(1H-pyrazol-4-yl)spiro[isoindoline-1,4′-piperidin]-3-one

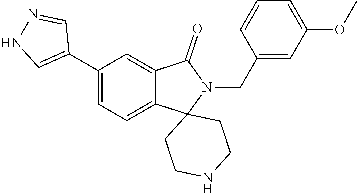 [SMILES: COc1cccc(CN2C(=O)c3cc(-c4cn[nH]c4)ccc3C23CCNCC3)c1]

### Step 1: tert-Butyl 4-(3-methoxybenzylimino)piperidine-1-carboxylate

tert-Butyl 4-(3-methoxybenzylimino)piperidine-1-carboxylate was prepared according to the procedure described in Example 98/99, Step 1, substituting tert-butyl 4-oxopiperidine-1-carboxylate for cyclopentanone. LCMS: Calculated: for C18H26N2O3: 318, Measured: 319 [M+H]+.

### Step 2: tert-Butyl 4-(5-bromo-2-iodo-N-(3-methoxybenzyl)benzamido)-3,6 dihydropyridine-1(2H)-carboxylate

tert-Butyl 4-(5-bromo-2-iodo-N-(3-methoxybenzyl)benzamido)-3,6 dihydropyridine-1(2H)-carboxylate was prepared according to the procedure described in Example 98/99, Step 2, substituting tert-butyl 4-(3-methoxybenzylimino)piperidine-1-carboxylate for N-(3-methoxybenzyl)cyclopentanimine. LCMS: Calculated: for C25H28BrIN2O4: 627, Measured: 650 [M+Na]+.

### Step 3: tert-Butyl 5-bromo-2-(3-methoxybenzyl)-3-oxo-2′,3′-dihydro-1′H-spiro[isoindoline-1,4′-pyridine]-1′-carboxylate

tert-Butyl 5-bromo-2-(3-methoxybenzyl)-3-oxo-2′,3′-dihydro-1′H-spiro[isoindoline-1,4′-pyridine]-1′-carboxylate was prepared according to the procedure described in Example 98/99, Step 3, substituting tert-Butyl 4-(5-bromo-2-iodo-N-(3-methoxybenzyl)benzamido)-3,6 dihydropyridine-1(2H)-carboxyl for 5-bromo-N-(cyclopent-1-en-1-yl)-2-iodo-N-(3-methoxybenzyl)benzamide. LCMS: Calculated: for C25H27BrN₂O₄: 499, Measured: 500 [M+H]+.

### Step 4: tert-Butyl 2-[(3-methoxyphenyl)methyl]-5-[1-(oxan-2-yl)-1H-pyrazol-4-yl]-3-oxo-2,2′,3,3′-tetrahydro-1′H-spiro[isoindole-1,4′-pyridine]-1′-carboxylate

tert-Butyl 2-[(3-methoxyphenyl)methyl]-5-[1-(oxan-2-yl)-1H-pyrazol-4-yl]-3-oxo-2,2′,3,3′-tetrahydro-1′H-spiro[isoindole-1,4′-pyridine]-1′-carboxylate was prepared according to the procedure described in Example 98/99, Step 4, substituting tert-butyl 5-bromo-2-(3-methoxybenzyl)-3-oxo-2′,3′-dihydro-1′H-spiro[isoindoline-1,4′-pyridine]-1′-carboxylate for 5′-bromo-2′-(3-methoxybenzyl)spiro[cyclopentane-1,1′-isoindolin]-2-en-3′-one. LCMS: Calculated: for C33H38N4O5: 570, Measured: 571 [M+H]+.

### Step 5: tert-Butyl 2-[(3-methoxyphenyl)methyl]-5-[1-(oxan-2-yl)-1H-pyrazol-4-yl]-3-oxo-2,3-dihydrospiro-[isoindole-1,4′-piperidine]-1′-carboxylate

A mixture of tert-butyl 2-[(3-methoxyphenyl)methyl]-5-[1-(oxan-2-yl)-1H-pyrazol-4-yl]-3-oxo-2,2′,3,3′-tetrahydro-1′H-spiro[isoindole-1,4′-pyridine]-1′-carboxylate (200 mg, 0.350 mmol) in MeOH (30 mL) was treated with Pd(OH)₂/C (400 mg, 10%) and placed under a hydrogen atmosphere at room temperature. After 12 h, the reaction mixture was filtered, and the filtrate was concentrated in vacuo to yield tert-butyl 2-[(3-methoxyphenyl)methyl]-5-[1-(oxan-2-yl)-1H-pyrazol-4-yl]-3-oxo-2,3-dihydrospiro-[isoindole-1,4′-piperidine]-1′-carboxylate as a yellow oil. LCMS: Calculated: for C33H40N4O5: 572, Measured: 573 [M+H]+.

### Step 6: 2-(3-methoxybenzyl)-5-(1H-pyrazol-4-yl)spiro[isoindoline-1,4′-piperidin]-3-one

To a mixture of tert-butyl 2-[(3-methoxyphenyl)methyl]-5-[1-(oxan-2-yl)-1H-pyrazol-4-yl]-3-oxo-2,3-dihydrospiro-[isoindole-1,4′-piperidine]-1′-carboxylate (140 mg, 0.244 mmol) in DCM (15 mL) was treated with TFA (139 mg, 1.219 mmol) at room temperature. After 2 h, the reaction mixture was diluted with DCM, washed with aq. NaHCO₃, dried (Na₂SO₄), filtered and concentrated in vacuo. Purification of the resulting residue by HPLC (0-100% ACN/H₂O containing 0.05% NH₃H₂O) yielded 2-(3-methoxybenzyl)-5-(1H-pyrazol-4-yl)spiro[isoindoline-1,4′-piperidin]-3-one as a white solid.

¹H NMR (400 MHz, DMSO-d6) δ 13.05 (brs, 1H), 8.20 (brs, 2H), 7.99-8.00 (m, 2H), 7.83 (dd, J=8.1, 1.8 Hz, 1H), 7.21 (t, J=8.1 Hz, 1H), 6.84-6.86 (m, 2H), 6.78-6.81 (m, 1H), 4.71 (s, 2H), 3.71 (s, 3H), 3.04-3.10 (m, 2H), 2.93-2.95 (m, 2H), 2.00 (td, J=12.6, 5.0 Hz, 2H), 1.20 (d, J=12.6 Hz, 2H); LCMS: Calculated: for C23H24N4O2: 388, Measured: 389 [M+H]+.

### Example 89

### 2-(3-methoxybenzyl)-5-(1H-pyrazol-4-yl)-2′,3′,5′,6′-tetrahydrospiro[isoindoline-1,4′-pyran]-3-one

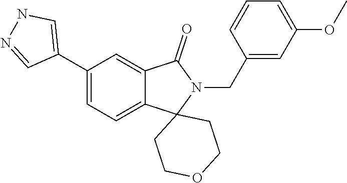 [SMILES: COc1cccc(CN2C(=O)c3cc(-c4cn[nH]c4)ccc3C23CCOCC3)c1]

2-(3-Methoxybenzyl)-5-(1H-pyrazol-4-yl)-2′,3′,5′,6′-tetrahydrospiro[isoindoline-1,4′-pyran]-3-one was prepared according to the procedure described in Example 88, Step 1, substituting tetrahydro-4H-pyran-4-one for tert-butyl 4-oxopiperidine-1-carboxylate.

¹H NMR (400 MHz, CHLOROFORM-d) δ 8.12 (d, J=1.52 Hz, 1H), 7.89-8.05 (m, 2H), 7.67-7.79 (m, 1H), 7.15-7.27 (m, 1H), 6.84-6.96 (m, 2H), 6.72-6.80 (m, 1H), 4.75-4.87 (m, 1H), 4.81 (s, 2H), 3.98-4.13 (m, 4H), 3.77 (s, 3H), 2.20-2.39 (m, 2H), 1.41 (br d, J=14.15 Hz, 2H), 1.25 (s, 1H); LCMS: Calculated: for C23H23N3O3: 389; Measured: 390 [M+H]+.

### Example 90

### 2′-(3-methoxybenzyl)-5′-(1H-pyrazol-4-yl)-4,5-dihydro-2H-spiro[furan-3,1′-isoindolin]-3′-one

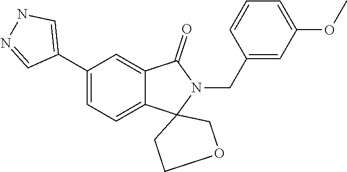 [SMILES: COc1cccc(CN2C(=O)c3cc(-c4cn[nH]c4)ccc3C23CCOC3)c1]

2′-(3-Methoxybenzyl)-5′-(1H-pyrazol-4-yl)-4,5-dihydro-2H-spiro[furan-3,1′-isoindolin]-3′-one was prepared according to the procedure described in Example 88, Step 1, substituting dihydrofuran-3(2H)-one for tert-butyl 4-oxopiperidine-1-carboxylate.

¹H NMR (400 MHz, METHANOL-d4) δ 8.13 (br s, 2H), 8.00 (d, J=1.52 Hz, 1H), 7.92 (dd, J=2.02, 8.08 Hz, 1H), 7.62 (d, J=8.08 Hz, 1H), 7.19-7.28 (m, 1H), 6.88-6.97 (m, 2H), 6.83 (dd, J=2.02, 9.09 Hz, 1H), 4.72-4.86 (m, 2H), 4.02-4.22 (m, 2H), 3.90-4.02 (m, 1H), 3.80-3.87 (m, 1H), 3.76 (s, 3H), 2.25-2.40 (m, 1H); LCMS: Calculated: for C22H21N3O3: 375; Measured: 376 [M+H]+.

### Example 91

### N-(2,6-dimethylbenzyl)-3-((3′-oxo-5′-(1H-pyrazol-4-yl)spiro[cyclopentane-1,1′-isoindolin]-2′-yl)methyl)benzamide

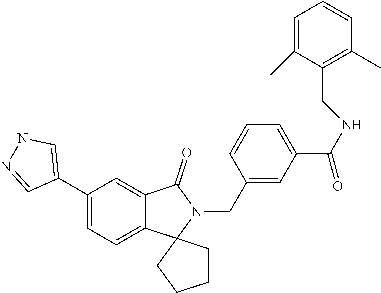 [SMILES: Cc1cccc(C)c1CNC(=O)c1cccc(CN2C(=O)c3cc(-c4cn[nH]c4)ccc3C23CCCC3)c1]

### Step 1: Methyl 3-((6-bromo-1-oxoisoindolin-2-yl)methyl)benzoate

To a solution of 6-bromoisoindolin-1-one (5 g, 23.580 mmol, 1.00 equiv.) in N,N-dimethylformamide (30 mL) and THE (30 ml) was added sodium hydride (1.04 g, 26.002 mmol, 1.1 equiv.) and TBAI (1.7 g, 4.595 mmol, 0.2 equiv.) at 0° C. The solution was stirred for 30 min at 25° C. To the resulting mixture was then added methyl 3-(bromomethyl)benzoate (5.9 g, 25.756 mmol, 1.1 equiv.) at 0° C. The solution was stirred for 2 h at 25° C. The reaction was then quenched by the addition of water. The resulting solution was extracted with ethyl acetate and washed by water and saturated salt water, then dried with Na₂SO₄. The organic layer was concentrated under vacuum to yield methyl 3-((6-bromo-1-oxoisoindolin-2-yl)methyl)benzoate as a yellow solid. LC/MS: Calculated: for C17H14BrNO3: 360.20, Measured 360.9 [M+H]+

### Step 2: Methyl 3-((1,1-diallyl-5-bromo-3-oxoisoindolin-2-yl)methyl)benzoate

Methyl 3-((1,1-diallyl-5-bromo-3-oxoisoindolin-2-yl)methyl)benzoate was prepared according to the procedure described in Example 81, Step 1 except substituting methyl 3-((6-bromo-1-oxoisoindolin-2-yl)methyl)benzoate for 6-bromo-2-(3-methoxybenzyl)isoindolin-1-one.

### Step 3: Methyl 3-((5′-bromo-3′-oxospiro[cyclopentane-1,1′-isoindolin]-3-en-2′-yl)methyl)benzoate

Methyl 3-((5′-bromo-3′-oxospiro[cyclopentane-1,1′-isoindolin]-3-en-2′-yl)methyl)benzoate was prepared according to the procedure described in Example 81, Step 4, substituting methyl 3-((1,1-diallyl-5-bromo-3-oxoisoindolin-2-yl)methyl)benzoate for 3-diallyl-6-bromo-2-(3-methoxybenzyl)isoindolin-1-one.

### Step 4: 3-((3′-oxo-5′-(1H-pyrazol-4-yl)spiro[cyclopentane-1,1′-isoindolin]-3-en-2′-yl)methyl)benzoic acid

Potassium carbonate (5.4 g, 38.809 mmol, 8.0 equiv.) was added to methyl 3-{5′-bromo-3′-oxo-2′,3′-dihydrospiro[cyclopentane-1,1′-isoindol]-3-en-2′-ylmethyl}benzoate (2 g, 4.851 mmol, 1 equiv.) in DMF (25 ml) and H₂O (5 ml). tert-Butyl 4-(4,4,5,5-tetramethyl-1,3,2-dioxaborolan-2-yl)-1H-pyrazole-1-carboxylate (1.7 g, 5.821 mmol, 1.2 equiv.) was added, followed by addition of Pd(PPh₃)₄ (560 mg, 0.485 mmol, 0.1 equiv.) under protection of N₂ and the resulting mixture was stirred overnight at 85° C. Water was added, and the resulting solution was combined and concentrated at room temperature. The resulting residue was applied onto a silica gel column with methanol/dichloromethane (0-5%) to yield 3-((3′-oxo-5′-(1H-pyrazol-4-yl)spiro[cyclopentane-1,1′-isoindolin]-3-en-2′-yl)methyl)benzoic acid as a light green solid. LCMS: Calculated: for C23H19N3O3: 385, Measured: 386 [M+H]+.

### Step 5: N-(2,6-Dimethylbenzyl)-3-((3′-oxo-5′-(1H-pyrazol-4-yl)spiro[cyclopentane-1,1′-isoindolin]-3-en-2′-yl)methyl)benzamide

A mixture of 3-((3′-oxo-5′-(1H-pyrazol-4-yl)spiro[cyclopentane-1,1′-isoindolin]-3-en-2′-yl)methyl)benzoic acid (220 mg, 0.571 mmol), (2,6-dimethylphenyl)methanamine (93 mg, 0.685 mmol) in DMF (5 mL), N,N-diisopropylethylamine (0.3 mL, 1.712 mmol), was treated with HATU (260 mg, 0.685 mmol) and the reaction mixture was stirred at room temperature. After 12 h, the resulting solution was diluted with water (1 mL) and concentrated in vacuo. Purification of the resulting residue by silica gel column chromatography (12 g, 0-100% EtOAc/Heptane) yielded N-(2,6-dimethylbenzyl)-3-((3′-oxo-5′-(1H-pyrazol-4-yl)spiro[cyclopentane-1,1′-isoindolin]-3-en-2′-yl)methyl)benzamide as a light yellow solid. LCMS: Calculated: for C32H30N4O2: 502, Measured: 503 [M+H]+.

### Step 6: N-(2,6-Dimethylbenzyl)-3-((3′-oxo-5′-(1H-pyrazol-4-yl)spiro[cyclopentane-1,1′-isoindolin]-2′-yl)methyl)benzamide

N-(2,6-dimethylbenzyl)-3-((3′-oxo-5′-(1H-pyrazol-4-yl)spiro[cyclopentane-1,1′-isoindolin]-3-en-2′-yl)methyl)benzamide (80 mg, 0.159 mmol), Pd(OH)₂ (45 mg, 0.318 mmol) were mixed in methanol (5 mL) and placed under a hydrogen atmosphere at room temperature. After 2 h, the reaction mixture was filtered and concentrated in vacuo to yield N-(2,6-dimethylbenzyl)-3-((3′-oxo-5′-(1H-pyrazol-4-yl)spiro[cyclopentane-1,1′-isoindolin]-2′-yl)methyl)benzamide as a white solid.

¹H NMR (400 MHz, DMSO-d6) δ 13.01 (s, 1H), 8.47 (t, J=4.9 Hz, 1H), 8.35 (s, 1H), 8.04 (s, 1H), 7.84-7.94 (m, 2H), 7.80 (d, J=2.0 Hz, 1H), 7.70 (m, J=6.9, 1.8 Hz, 1H), 7.57 (d, J=8.0 Hz, 1H), 7.31-7.42 (m, 2H), 7.08 (dd, J=8.5, 6.2 Hz, 1H), 7.02 (d, J=6.8 Hz, 2H), 4.74 (s, 2H), 4.46 (d, J=4.8 Hz, 2H), 2.35 (s, 6H), 1.66-1.98 (m, 8H); LCMS: Calculated: for C32H32N4O2: 504, Measured: 505 [M+H]+.

### Example 92

### N-((3-methylpyridin-4-yl)methyl)-3-((3′-oxo-5′-(1H-pyrazol-4-yl)spiro[cyclopentane-1,1′-isoindolin]-2′-yl)methyl)benzamide

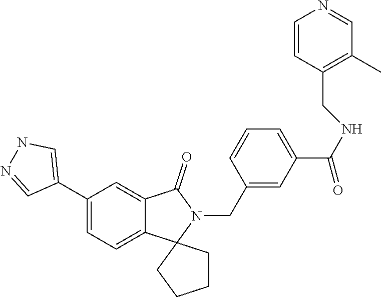 [SMILES: Cc1cnccc1CNC(=O)c1cccc(CN2C(=O)c3cc(-c4cn[nH]c4)ccc3C23CCCC3)c1]

N-((3-Methylpyridin-4-yl)methyl)-3-((3′-oxo-5′-(1H-pyrazol-4-yl)spiro[cyclopentane-1,1′-isoindolin]-2′-yl)methyl)benzamide was prepared according to the procedure described in Example 91, Step 3, substituting (4-methylpyridin-3-yl)methanamine for (2,6-dimethylphenyl)methanamine.

¹H NMR (400 MHz, DMSO-d6) δ 12.98-13.04 (m, 1H), 8.94 (t, J=5.4 Hz, 1H), 8.40 (s, 1H), 8.29-8.37 (m, 2H), 8.01-8.06 (m, 1H), 7.80-7.97 (m, 3H), 7.71-7.78 (m, 1H), 7.54-7.67 (m, 1H), 7.42 (d, J=7.1 Hz, 2H), 7.19 (d, J=4.9 Hz, 1H), 4.76 (s, 2H), 4.47 (d, J=5.5 Hz, 2H), 2.34 (s, 3H), 1.93 (m, J=32.1, 12.2, 6.6 Hz, 6H), 1.79 (dd, J=11.3, 5.5 Hz, 2H); LCMS: Calculated: for C30H29N5O2: 491, Measured: 492 [M+H]+.

### Example 93

### 3-hydroxy-2′-(3-methoxybenzyl)-5′-(1H-pyrazol-4-yl)spiro[cyclopentane-1,1′-isoindolin]-3′-one

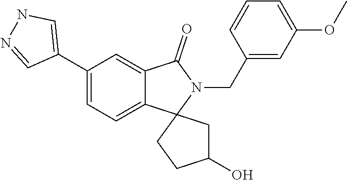 [SMILES: COc1cccc(CN2C(=O)c3cc(-c4cn[nH]c4)ccc3C23CCC(O)C3)c1]

### Step 1: 5′-Bromo-3-hydroxy-2′-(3-methoxybenzyl)spiro[cyclopentane-1,1′-isoindolin]-3′-one

To a mixture of 5′-bromo-2′-[(3-methoxyphenyl)methyl]-2′,3′-dihydrospiro[cyclopentane-1,1′-isoindol]-3-en-3′-one (300 mg, 0.781 mmol) in THE (20 mL) was added BH₃ (1 M in THF, 2.34 mL, 2.34 mmol) and the reaction mixture was stirred at room temperature. After 12 h, the resulting mixture was treated with NaOH (3M, 5.2 mL, 15.6 mmol) and H₂O₂ (30% w/w, 2.21 g, 19.5 mmol) was stirred at room temperature. After 3 h, EtOAc (100 mL) was added and the resulting solution was washed with water (1×10 mL), brine (1×10 mL), dried (Na₂SO₄), filtered and concentrated in vacuo. Purification of the resulting residue by silica gel column chromatography (40 g, 0-50% EtOAc/heptane) yielded 5′-bromo-3-hydroxy-2′-(3-methoxybenzyl)spiro[cyclopentane-1,1′-isoindolin]-3′-one as a light yellow solid. LCMS: Calculated: for C20H20BrNO3: 402, Measured: 402 [M]+

### Step 2: 3-Hydroxy-2′-(3-methoxybenzyl)-5′-(1H-pyrazol-4-yl)spiro[cyclopentane-1,1′-isoindolin]-3′-one

3-Hydroxy-2′-(3-methoxybenzyl)-5′-(1H-pyrazol-4-yl)spiro[cyclopentane-1,1′-isoindolin]-3′-one was prepared according to the procedure described in Example 81, Step 5.

¹H NMR (400 MHz, DMSO-d6) δ 8.19 (s, 2H), 7.90 (d, J=7.2 Hz, 2H), 7.73-7.75 (m, 1H), 7.23 (t, J=7.8 Hz, 1H), 6.84-6.92 (m, 2H), 6.78-6.84 (m, 1H), 4.65 (q, J=16.4 Hz, 2H), 4.38 (dq, J=7.6, 4.1 Hz, 1H), 3.71 (s, 3H), 2.21 (dd, J=14.5, 5.8 Hz, 1H), 2.05 (ddtd, J=16.1, 12.2, 9.5, 6.0 Hz, 2H), 1.72-1.91 (m, 3H)./19F NMR (376 MHz, DMSO-d6) d −74.80. LCMS: Calculated: for C23H23N3O3: 389, Measured: MH+: 390 [M+H]+.

### Example 94

### 2′-(3-methoxybenzyl)-3-(methylamino)-5′-(1H-pyrazol-4-yl)spiro[cyclopentane-1,1′-isoindolin]-3′-one

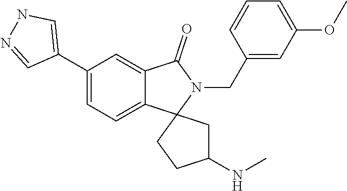 [SMILES: CNC1CCC2(C1)c1ccc(-c3cn[nH]c3)cc1C(=O)N2Cc1cccc(OC)c1]

2′-(3-methoxybenzyl)-3-(methylamino)-5′-(1H-pyrazol-4-yl)spiro[cyclopentane-1,1′-isoindolin]-3′-one was similarly prepared according to the procedures described herein, selecting and substituting suitable reagents and starting materials, as would be recognized by those skilled in the art.

¹H NMR (400 MHz, METHANOL-d4) δ 8.10 (s, 2H), 7.90-8.07 (m, 2H), 7.56-7.65 (m, 1H), 7.28 (s, 1H), 6.79-7.00 (m, 2H), 4.81-4.95 (m, 1H), 4.68-4.80 (m, 1H), 3.96-4.11 (m, 1H), 3.76 (d, J=5.56 Hz, 3H), 2.64 (s, 3H), 2.37-2.55 (m, 2H), 2.09-2.34 (m, 4H), 1.88-2.09 (m, 2H); LCMS: Calculated: for C24H26N4O2: 402; Measured: 403 [M+H]+.

### Example 95

### methyl (2′-(3-methoxybenzyl)-3′-oxo-5′-(1H-pyrazol-4-yl)spiro[cyclopentane-1,1′-isoindolin]-3-yl)carbamate

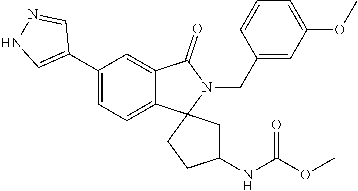 [SMILES: COC(=O)NC1CCC2(C1)c1ccc(-c3cn[nH]c3)cc1C(=O)N2Cc1cccc(OC)c1]

### Step 1: 5′-Bromo-2′-[(3-methoxyphenyl)methyl]-3′-oxo-2′,3′-dihydrospiro[cyclopentane-1,1′-isoindole]-3-yl methanesulfonate

To a solution of 5′-bromo-3-hydroxy-2′-(3-methoxybenzyl)spiro[cyclopentane-1,1′-isoindolin]-3′-one (4 g, 9.94 mmol) in DCM (80 mL) was added triethylamine (3.02 g, 29.8 mmol), MsCl (1.48 g, 12.9 mmol). The resulting mixture was stirred for 3 h at 25° C. The reaction was quenched with H₂O. The resulting mixture was extracted with EtOAc (3×50 mL). The organic layers were combined, dried (Na₂SO₄), filtered and concentrated in vacuo. Purification of the resulting residue by silica gel chromatography (0-50% EtOAc/heptane) yielded 5′-bromo-2′-[(3-methoxyphenyl)methyl]-3′-oxo-2′,3′-dihydrospiro[cyclopentane-1,1′-isoindole]-3-yl methanesulfonate as a yellow oil. LCMS: Calculated: for C21H22BrNO5S: 480, Measured: 504 [M+Na]+.

### Step 2: 3-Azido-5′-bromo-2′-[(3-methoxyphenyl)methyl]-2′,3′-dihydrospiro[cyclopentane-1,1′-isoindole]-3′-one

To a solution of 5′-bromo-2′-[(3-methoxyphenyl)methyl]-3′-oxo-2′,3′-dihydrospiro[cyclopentane-1,1′-isoindole]-3-yl methanesulfonate (800 mg, 1.67 mmol) in DMF (10 mL) was added NaN₃ (162 mg, 2.49 mmol). The resulting mixture was stirred at 25° C. overnight. The reaction was quenched with H₂O. The resulting mixture was extracted with EtOAc (25 mL). The organic layers were combined, dried (Na₂SO₄), filtered and concentrated in vacuo. The resulting mixture containing 3-azido-5′-bromo-2′-[(3-methoxyphenyl)methyl]-2′,3′-dihydrospiro[cyclopentane-1,1′-isoindole]-3′-one was used in the next strep without further purification (650 mg). LCMS: Calculated: for C20H19BrN₄O₂: 427.29, Measured: 449.2 [M+Na]+.

### Step 3: 3-Amino-5′-bromo-2′-[(3-methoxyphenyl)methyl]-2′,3′-dihydrospiro[cyclopentane-1,1′-isoindole]-3′-one

To a solution of 3-azido-5′-bromo-2′-[(3-methoxyphenyl)methyl]-2′,3′-dihydrospiro[cyclopentane-1,1′-isoindole]-3′-one (650 mg, 1.52 mmol) in THE (5 mL), H₂O (5 mL), was added triphenylphosphine (1.197 g, 4.56 mmol). The resulting mixture was stirred at 25° C. overnight. The reaction was quenched with H₂O. The resulting mixture was extracted with ethyl acetate. The organic layers were combined, dried (Na₂SO₄), filtered and concentrated in vacuo. Purification of the resulting residue by silica gel chromatography (0-100% EtOAc/heptane) yielded 3-amino-5′-bromo-2′-[(3-methoxyphenyl)methyl]-2′,3′-dihydrospiro[cyclopentane-1,1′-isoindole]-3′-one as a yellow oil. LCMS: Calculated: for C20H21BrN2O₂: 401, Measured: 403 [M+2H]+.

### Step 4: Methyl N-{5′-Bromo-2′-[(3-methoxyphenyl)methyl]-3′-oxo-2′,3′-dihydrospiro[cyclopentane-1,1′-isoindole]-3-yl}carbamate

To a solution of 3-amino-5′-bromo-2′-[(3-methoxyphenyl)methyl]-2′,3′-dihydrospiro[cyclopentane-1,1′-isoindole]-3′-one (450 mg, 1.12 mmol) in DCM (10 mL) was added pyridine (0.266 g, 3.36 mmol), methyl chloroformate (0.159 g, 1.68 mmol). The resulting mixture was stirred at room temperature. After 12 h, the reaction was treated with H₂O (5 mL) and the resulting mixture was extracted with DCM (3×10 mL). The organic layers were combined, dried (Na₂SO₄), filtered and concentrated in vacuo. Purification of the resulting residue by silica gel chromatography (0-100% EtOAc/heptane) yielded Methyl N-{5′-Bromo-2′-[(3-methoxyphenyl)methyl]-3′-oxo-2′,3′-dihydrospiro[cyclopentane-1,1′-isoindole]-3-yl}carbamate as a yellow oil. LCMS: Calculated: for C22H23BrN2O₄: 459, Measured: 458 [M−H]+.

### Step 5: Methyl (2′-(3-methoxybenzyl)-3′-oxo-5′-(1H-pyrazol-4-yl)spiro[cyclopentane-1,1′-isoindolin]-3-yl)carbamate

Methyl (2′-(3-methoxybenzyl)-3′-oxo-5′-(1H-pyrazol-4-yl)spiro[cyclopentane-1,1′-isoindolin]-3-yl)carbamate was prepared according to the procedure described in Example 81, Step 5.

¹H NMR (300 MHz, DMSO-d6) δ 8.18 (d, J=1.7 Hz, 2H), 7.89 (d, J=7.7 Hz, 2H), 7.57-7.63 (m, 1H), 7.44 (d, J=7.7 Hz, 1H), 7.22 (dd, J=8.8, 7.5 Hz, 1H), 6.79-6.88 (m, 3H), 4.56-4.81 (m, 2H), 4.23 (q, J=7.7 Hz, 1H), 3.70 (d, J=1.8 Hz, 3H), 3.48 (d, J=1.7 Hz, 3H), 1.56-2.26 (m, 6H); LCMS: Calculated: for C25H26N4O4: 446, Measured: 447 [M+H]+.

### Example 96

### 2′-(3-methoxybenzyl)-3′-oxo-5′-(1H-pyrazol-4-yl)spiro[cyclopentane-1,1′-isoindoline]-3-carboxylic acid

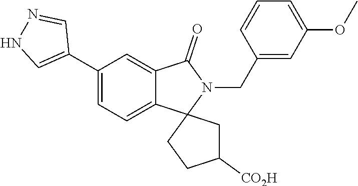 [SMILES: COc1cccc(CN2C(=O)c3cc(-c4cn[nH]c4)ccc3C23CCC(C(=O)O)C3)c1]

### Step 1: 5′-Bromo-2′-[(3-methoxyphenyl)methyl]-3′-oxo-2′,3′-dihydrospiro[cyclopentane-1,1′-isoindole]-3-yl methanesulfonate

A solution of 5′-bromo-3-hydroxy-2′-(3-methoxybenzyl)spiro[cyclopentane-1,1′-isoindolin]-3′-one (4 g, 9.94 mmol), TEA (3.022 g, 29.8 mmol) in DCM (80 mL) was treated with MsCl (1.48 g, 12.9 mmol) at 25° C. After 3 h, the reaction was treated with H₂O (5 mL) and the resulting mixture was extracted with EtOAc (3×10 mL). The organic layers were combined, dried (Na₂SO₄), filtered and concentrated in vacuo. Purification of the resulting residue by silica gel chromatography (0-100% EtOAc/heptane) yielded 5′-bromo-2′-[(3-methoxyphenyl)methyl]-3′-oxo-2′,3′-dihydrospiro[cyclopentane-1,1′-isoindole]-3-yl methanesulfonate as a yellow oil. LC/MS: Calculated: for C21H22BrNO5S: 480.37, Measured: 504.2 [M+Na]+.

### Step 2: 5′-Bromo-2′-[(3-methoxyphenyl)methyl]-3′-oxo-2′,3′-dihydrospiro[cyclopentane-1,1′-isoindole]-3-carbonitrile

A solution of 5′-bromo-2′-[(3-methoxyphenyl)methyl]-3′-oxo-2′,3′-dihydrospiro[cyclopentane-1,1′-isoindole]-3-yl methanesulfonate (1.5 g, 3.12 mmol) in DMSO (20 mL) was treated with KCN (0.305 g, 4.65 mmol) was warmed to 40° C. After 12 h, the reaction was treated with H₂O (5 mL) and the resulting mixture was extracted with EtOAc (3×10 mL). The organic layers were combined, dried (Na₂SO₄), filtered and concentrated in vacuo. Purification of the resulting residue by silica gel chromatography (0-100% EtOAc/heptane) yielded 5′-bromo-2′-[(3-methoxyphenyl)methyl]-3′-oxo-2′,3′-dihydrospiro[cyclopentane-1,1′-isoindole]-3-carbonitrile as a yellow oil. LCMS: Calculated: for C21H19BrN2O2: 411.29, Measured: 411.2 [M+H]+.

### Step 3: 5′-Bromo-2′-[(3-methoxyphenyl)methyl]-3′-oxo-2′,3′-dihydrospiro[cyclopentane-1,1′-isoindole]-3-carboxylic acid

A solution of 5′-bromo-2′-[(3-methoxyphenyl)methyl]-3′-oxo-2′,3′-dihydrospiro[cyclopentane-1,1′-isoindole]-3-carbonitrile (530 mg, 1.289 mmol) in EtOH (4 mL) was treated with 1M NaOH (2 mL). The resulting mixture was warmed at 90° C. After 12 h, the resulting solution was concentrated in vacuo, the residue was treated with 1N HCl (pH=5) and product was collected by filtration to yield 5′-bromo-2′-[(3-methoxyphenyl)methyl]-3′-oxo-2′,3′-dihydrospiro[cyclopentane-1,1′-isoindole]-3-carboxylic acid as an off-white solid. LCMS: Calculated: for C21H20BrNO4: 430.29, Measured: 452.2 [M+Na]+.

### Step 4: 5′-Bromo-2′-[(3-methoxyphenyl)methyl]-3′-oxo-2′,3′-dihydrospiro[cyclopentane-1,1′-isoindole]-3-carboxamide

A solution of 5′-bromo-2′-[(3-methoxyphenyl)methyl]-3′-oxo-2′,3′-dihydrospiro[cyclopentane-1,1′-isoindole]-3-carboxylic acid (400 mg, 0.930 mmol) in DMF (10 mL), DIEA (0.721 g, 5.58 mmol), and NH₄Cl (0.249 mg, 4.65 mmol) was treated with HATU (0.530 g, 1.40 mmol) and the resulting mixture was allowed to stir at room temperature. After 12 h, the reaction was treated with H₂O (5 mL) and the resulting mixture was extracted with EtOAc (3×10 mL). The organic layers were combined, dried (Na₂SO₄), filtered and concentrated in vacuo. Purification of the resulting residue by silica gel chromatography (0-100% EtOAc/heptane) yielded 5′-bromo-2′-[(3-methoxyphenyl)methyl]-3′-oxo-2′,3′-dihydrospiro[cyclopentane-1,1′-isoindole]-3-carboxamide as a yellow solid. LCMS: Calculated: for C21H21 BrN2O3: 429.31, Measured: 431.2 [M+H]+.

### Step 5: 2′-(3-Methoxybenzyl)-3′-oxo-5′-(1H-pyrazol-4-yl)spiro[cyclopentane-1,1′-isoindoline]-3-carboxylic acid

A solution of 5′-bromo-2′-[(3-methoxyphenyl)methyl]-3′-oxo-2′,3′-dihydrospiro[cyclopentane-1,1′-isoindole]-3-carboxamide (0.15 g, 0.349 mmol) H₂O (0.3 mL), K₂CO₃ (0.144 g, 1.046 mmol), Pd(PPh₃)₄ (0.020 g, 0.017 mmol) in DMF (3 mL) was treated with tert-butyl 4-(4,4,5,5-tetramethyl-1,3,2-dioxaborolan-2-yl)-1H-pyrazole-1-carboxylate (0.205 g, 0.697 mmol) and the resulting solution was warmed to 100° C. After 5 h, the resulting mixture was allowed to cool to room temperature, diluted with EtOAc (100 mL), washed with brine (4×30 mL), dried (Na₂SO₄), filtered and concentrated in vacuo. Purification of the resulting residue by C18 reverse phase column chromatography (80 g, 0-50% ACN/H₂O (containing 0.05% TFA)) yielded 2′-(3-methoxybenzyl)-3′-oxo-5′-(1H-pyrazol-4-yl)spiro[cyclopentane-1,1′-isoindoline]-3-carboxylic acid as a white solid.

¹H NMR (300 MHz, DMSO-d6) δ 8.18 (d, J=2.1 Hz, 2H), 7.85-7.90 (m, 2H), 7.61 (dd, J=27.0, 7.9 Hz, 1H), 7.22 (td, J=7.8, 4.2 Hz, 1H), 6.78-6.88 (m, 3H), 4.61-4.77 (m, 2H), 3.70 (s, 3H), 3.18 (dp, J=25.8, 8.5 Hz, 1H), 1.70-2.34 (m, 6H); LCMS: Calculated: for C24H23N3O4: 417, Measured: 418 [M+H]+.

### Example 97

### 2′-(3-methoxybenzyl)-3′-oxo-5′-(1H-pyrazol-4-yl)spiro[cyclopentane-1,1′-isoindoline]-3-carboxamide

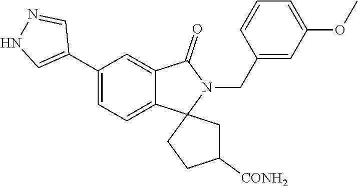 [SMILES: COc1cccc(CN2C(=O)c3cc(-c4cn[nH]c4)ccc3C23CCC(C(N)=O)C3)c1]

2′-(3-Methoxybenzyl)-3′-oxo-5′-(1H-pyrazol-4-yl)spiro[cyclopentane-1,1′-isoindoline]-3-carboxamide was prepared according to the procedure described in Example 96, Step 5, substituting 5′-bromo-2′-[(3-methoxyphenyl)methyl]-3′-oxo-2′,3′-dihydrospiro[cyclopentane-1,1′-isoindole]-3-carboxamide for 5′-bromo-2′-[(3-methoxyphenyl)methyl]-3′-oxo-2′,3′-dihydrospiro[cyclopentane-1,1′-isoindole]-3-carboxylic acid.

¹H NMR (300 MHz, DMSO-d6) δ 8.18 (d, J=3.2 Hz, 2H), 7.91 (ddd, J=8.6, 4.7, 1.6 Hz, 2H), 7.64 (dd, J=20.0, 8.2 Hz, 1H), 7.38 (s, 1H), 7.24 (q, J=7.8 Hz, 1H), 6.79-6.89 (m, 4H), 4.57-4.81 (m, 2H), 3.04 (dt, J=28.0, 8.2 Hz, 1H), 1.71-2.33 (m, 6H); LCMS: Calculated: for C24H24N4O3: 416, Measured: 417 [M+H]+.

### Example 98

### 2′-(3-methoxybenzyl)-5′-(1H-pyrazol-4-yl)-3a,4,5,6a-tetrahydrospiro[cyclopenta[d]oxazole-6,1′-isoindoline]-2,3′(3H)-dione

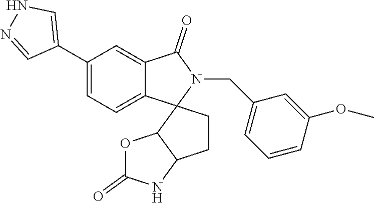 [SMILES: COc1cccc(CN2C(=O)c3cc(-c4cn[nH]c4)ccc3C23CCC2NC(=O)OC23)c1]

### And Example 99

### 2′-(3-methoxybenzyl)-5′-(1H-pyrazol-4-yl)-3a,5,6,6a-tetrahydrospiro[cyclopenta[d]oxazole-4,1′-isoindoline]-2,3′(3H)-dione

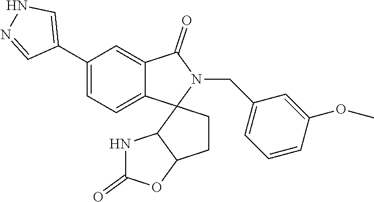 [SMILES: COc1cccc(CN2C(=O)c3cc(-c4cn[nH]c4)ccc3C23CCC2OC(=O)NC23)c1]

### Step 1: N-(3-Methoxybenzyl)cyclopentanimine

A mixture of cyclopentanone (1 g, 11.9 mmol) and (3-methoxyphenyl)methanamine (1.63 g, 11.9 mmol) in toluene (30 mL) was stirred at 80° C. After 3 h, the resulting solution was concentrated in vacuo to yield N-(3-methoxybenzyl)cyclopentanimine as a brown oil. LCMS: Calculated: for C13H17NO: 203, Measured: 204 [M+H]+.

### Step 2: 5-Bromo-N-(cyclopent-1-en-1-yl)-2-iodo-N-(3-methoxybenzyl)benzamide

To a mixture of N-(3-methoxybenzyl)cyclopentanimine (2.4 g, 11.8 mmol) in toluene (60 mL) with 5-bromo-2-iodobenzoyl chloride (3.67 g, 10.6 mmol) was added Et₃N (1.79 g, 17.7 mmol). The reaction mixture was warmed to 80° C. After 12 h, the reaction mixture was allowed to cool to room temperature, diluted with EtOAc (100 mL), washed with water (2×40 mL), brine (2×40 mL), dried (Na₂SO₄), filtered and concentrated in vacuo. Purification of the resulting residue by silica gel column chromatography (120 g, 0-25% EtOAc/Heptane) yielded 5-bromo-N-(cyclopent-1-en-1-yl)-2-iodo-N-(3-methoxybenzyl)benzamide as a brown oil. LCMS: Calculated: for C20H19BrINO2: 512, Measured: 512 [M+]+.

### Step 3: 5′-Bromo-2′-(3-methoxybenzyl)spiro[cyclopentane-1,1′-isoindolin]-2-en-3′-one

Under a nitrogen atmosphere, a mixture of 5-bromo-N-(cyclopent-1-en-1-yl)-2-iodo-N-(3-methoxybenzyl)benzamide (2 g, 3.91 mmol), K₂CO₃ (901 mg, 6.52 mmol), TBAB (1.26 g, 3.91 mmol), PPh₃ (164 mg, 0.625 mmol) and Pd(OAc)₂ (175 mg, 0.779 mmol) in acetonitrile (50 mL) was stirred at 80° C.

After 12 h, the reaction mixture was allowed to cool to room temperature, diluted with EtOAc (200 mL), washed with brine (3×50 mL), dried (Na₂SO₄), filtered and concentrated in vacuo. Purification of the resulting residue by silica gel column chromatography (80 g, 0-20% EtOAc/Heptane) yielded 5′-bromo-2′-(3-methoxybenzyl)spiro[cyclopentane-1,1′-isoindolin]-2-en-3′-one as a yellow oil. LCMS: Calculated: for C20H18BrNO2: 384, Measured: 384 [M]+.

### Step 4: 2′-(3-Methoxybenzyl)-5′-(1-(tetrahydro-2H-pyran-2-yl)-1H-pyrazol-4-yl)spiro[cyclopentane-1,1′-isoindolin]-2-en-3′-one

To a solution of 5′-bromo-2′-(3-methoxybenzyl)spiro[cyclopentane-1,1′-isoindolin]-2-en-3′-one (3.01 g, 7.83 mmol) in DMF (30 mL) and H₂O (3 mL) was added 1-(tetrahydro-2H-pyran-2-yl)-4-(4,4,5,5-tetramethyl-1,3,2-dioxaborolan-2-yl)-1H-pyrazole (3.28 g, 11.8 mmol), potassium carbonate (3.25 g, 23.5 mmol) and Pd(PPh₃)₄ (0.18 g, 0.156 mmol). The resulting mixture was stirred at 100° C. under nitrogen for 3 h. The reaction was quenched with H₂O (10 mL). The resulting mixture was extracted with EtOAc (5×150 mL). The organic layers were combined, dried (Na₂SO₄), filtered and concentrated in vacuo. Purification of the resulting residue by silica gel chromatography (120 g, 0-80% EtOAc/heptane) yielded 2′-(3-methoxybenzyl)-5′-(1-(tetrahydro-2H-pyran-2-yl)-1H-pyrazol-4-yl)spiro[cyclopentane-1,1′-isoindolin]-2-en-3′-one as a light brown oil. LCMS: Calculated: for C28H29N3O3: 455, Measured: 456 [M+H]+.

### Step 5: 2′-[(3-Methoxyphenyl)methyl]-5′-[1-(oxan-2-yl)-1H-pyrazol-4-yl]-2′,3′-dihydro-6-oxaspiro[bicyclo[3.1.0]hexane-2,1′-isoindole]-3′-one

To a mixture of 2′-(3-Methoxybenzyl)-5′-(1-(tetrahydro-2H-pyran-2-yl)-1H-pyrazol-4-yl)spiro[cyclopentane-1,1′-isoindolin]-2-en-3′-one (2 g, 4.390 mmol) in acetone (50 mL) and H₂O (50 mL) was added NaHCO₃ (922 mg, 10.9 mmol) followed by oxone (1.11 g, 6.59 mmol). The reaction was stirred at room temperature for 12 h, diluted with DCM, washed with brine, dried (Na₂SO₄), filtered and concentrated in vacuo. Purification of the resulting residue by silica gel chromatography (0-100% EtOAc/Heptane) yielded 2′-[(3-methoxyphenyl)methyl]-5′-[1-(oxan-2-yl)-1H-pyrazol-4-yl]-2′,3′-dihydro-6-oxaspiro[bicyclo[3.1.0]hexane-2,1′-isoindole]-3′-one as a yellow oil. LCMS: Calculated: for C28H29N3O4:471, Measured: 493 [M+Na]+.

### Step 6: 3-Azido-2-hydroxy-2′-(3-methoxybenzyl)-5′-(1-(tetrahydro-2H-pyran-2-yl)-1H-pyrazol-4-yl)spiro[cyclopentane-1,1′-isoindolin]-3′-one and 2-azido-3-hydroxy-2′-(3-methoxybenzyl)-5′-(1-(tetrahydro-2H-pyran-2-yl)-1H-pyrazol-4-yl)spiro[cyclopentane-1,1′-isoindolin]-3′-one

To a mixture of 2′-[(3-methoxyphenyl)methyl]-5′-[1-(oxan-2-yl)-1H-pyrazol-4-yl]-2′,3′-dihydro-6-oxaspiro[bicyclo[3.1.0]hexane-2,1′-isoindole]-3′-one (600 mg, 1.28 mmol) in DMF (10 mL) was added NaN₃ (91 mg, 1.40 mmol). The reaction mixture was warmed to 80° C. under nitrogen. After 4 d, the resulting mixture was allowed to cool room temperature, treated with H₂O (3 mL), washed with EtOAc (3×10 mL) and the organic layers was combined. The combined organic layer was washed with brine, dried (Na₂SO₄), filtered and concentrated in vacuo. Purification of the resulting residue by silica gel column chromatography (20 g, 0-50% EtOAc/heptane) yielded a mixture of 3-azido-2-hydroxy-2′-(3-methoxybenzyl)-5′-(1-(tetrahydro-2H-pyran-2-yl)-1H-pyrazol-4-yl)spiro[cyclopentane-1,1′-isoindolin]-3′-one and 2-azido-3-hydroxy-2′-(3-methoxybenzyl)-5′-(1-(tetrahydro-2H-pyran-2-yl)-1H-pyrazol-4-yl)spiro[cyclopentane-1,1′-isoindolin]-3′-one as a yellow solid. LCMS: Calculated: for C28H30N6O4:514, Measured: 515 [M+H]+.

### Step 7: Ethyl N-{5-hydroxy-2′-[(3-methoxyphenyl)methyl]-5′-[1-(oxan-2-yl)-1H-pyrazol-4-yl]-3′-oxo-2′,3′-dihydrospiro[cyclopentane-1,1′-isoindole]-4-yl}carbamate and ethyl N-{4-hydroxy-2′-[(3-methoxyphenyl)methyl]-5′-[1-(oxan-2-yl)-1H-pyrazol-4-yl]-3′-oxo-2′,3′-dihydrospiro[cyclopentane-1,1′-isoindole]-5-yl}carbamate

To a mixture of 3-azido-2-hydroxy-2′-(3-methoxybenzyl)-5′-(1-(tetrahydro-2H-pyran-2-yl)-1H-pyrazol-4-yl)spiro[cyclopentane-1,1′-isoindolin]-3′-one and 2-azido-3-hydroxy-2′-(3-methoxybenzyl)-5′-(1-(tetrahydro-2H-pyran-2-yl)-1H-pyrazol-4-yl)spiro[cyclopentane-1,1′-isoindolin]-3′-one (200 mg, 0.389 mmol) in THE (5 mL) was added DEPC (126.039 mg, 0.777 mmol) followed by Pd(OH)₂/C (10% w/w). The reaction was stirred at room temperature under H₂ for 1 h, filtered and concentrated to yield a mixture ethyl N-{5-hydroxy-2′-[(3-methoxyphenyl)methyl]-5′-[1-(oxan-2-yl)-1H-pyrazol-4-yl]-3′-oxo-2′,3′-dihydrospiro[cyclopentane-1,1′-isoindole]-4-yl}carbamate and ethyl N-{4-hydroxy-2′-[(3-methoxyphenyl)methyl]-5′-[1-(oxan-2-yl)-1H-pyrazol-4-yl]-3′-oxo-2′,3′-dihydrospiro[cyclopentane-1,1′-isoindole]-5-yl}carbamate as an off-white solid. The solids were used in the next step without any further purification

### Step 8: 2′-[(3-methoxyphenyl)methyl]-5′-[1-(oxan-2-yl)-1H-pyrazol-4-yl]-2,2′,3,3′,3a,4,5,6a-octahydrospiro[cyclopenta[d][1,3]oxazole-1,1′-isoindole]-3′,5-dione and 2′-[(3-methoxyphenyl)methyl]-5′-[1-(oxan-2-yl)-1H-pyrazol-4-yl]-2,2′,3,3′,3a,5,6,6a-octahydrospiro[cyclopenta[d][1,3]oxazole-1,1′-isoindole]-3′,5-dione

To a mixture of ethyl N-{5-hydroxy-2′-[(3-methoxyphenyl)methyl]-5′-[1-(oxan-2-yl)-1H-pyrazol-4-yl]-3′-oxo-2′,3′-dihydrospiro[cyclopentane-1,1′-isoindole]-4-yl}carbamate and ethyl N-{4-hydroxy-2′-[(3-methoxyphenyl)methyl]-5′-[1-(oxan-2-yl)-1H-pyrazol-4-yl]-3′-oxo-2′,3′-dihydrospiro[cyclopentane-1,1′-isoindole]-5-yl}carbamate (200 mg, 0.36 mmol) in THE (1 mL) was added NaOCH₃ (38.5 mg, 0.71 mmol). The reaction was stirred at room temperature. After 12 h, the reaction mixture was filtered and concentrated in vacuo to yield a mixture of 2′-[(3-methoxyphenyl)methyl]-5′-[1-(oxan-2-yl)-1H-pyrazol-4-yl]-2,2′,3,3′,3a,4,5,6a-octahydrospiro[cyclopenta[d][1,3]oxazole-1,1′-isoindole]-3′,5-dione and 2′-[(3-methoxyphenyl)methyl]-5′-[1-(oxan-2-yl)-1H-pyrazol-4-yl]-2,2′,3,3′,3a,5,6,6a-octahydrospiro[cyclopenta[d][1,3]oxazole-1,1′-isoindole]-3′,5-dione as a brown solid. LCMS: Calculated: for C29H30N4O5:514, Measured: 515 [M+H]+.

### Step 9: 2′-[(3-Methoxyphenyl)methyl]-5′-(1H-pyrazol-4-yl)-2,2′,3,3′,3a,4,5,6a-octahydrospiro[cyclopenta[d][1,3]oxazole-1,1′-isoindole]-3′,5-dione and 2′-[(3-methoxyphenyl)methyl]-5′-(1H-pyrazol-4-yl)-2,2′,3,3′,3a,5,6,6a-octahydrospiro[cyclopenta[d][1,3]oxazole-1,1′-isoindole]-3′,5-dione

To a mixture of 2′-[(3-methoxyphenyl)methyl]-5′-[1-(oxan-2-yl)-1H-pyrazol-4-yl]-2,2′,3,3′,3a,4,5,6a-octahydrospiro[cyclopenta[d][1,3]oxazole-1,1′-isoindole]-3′,5-dione and 2′-[(3-methoxyphenyl)methyl]-5′-[1-(oxan-2-yl)-1H-pyrazol-4-yl]-2,2′,3,3′,3a,5,6,6a-octahydrospiro[cyclopenta[d][1,3]oxazole-1,1′-isoindole]-3′,5-dione (180 mg, 0.350 mmol) in DCM (5 mL) was added TFA (2 mL). The reaction was stirred at room temperature for 2 h, diluted with DCM, washed with aq. NaHCO₃, dried (Na₂SO₄), filtered and concentrated in vacuo. Purification of the resulting residue by HPLC (0-100% ACN/H₂O containing 5% TFA) yielded 2′-[(3-methoxyphenyl)methyl]-5′-(1H-pyrazol-4-yl)-2,2′,3,3′,3a,4,5,6a-octahydrospiro[cyclopenta[d][1,3]oxazole-1,1′-isoindole]-3′,5-dione and 2′-[(3-methoxyphenyl)methyl]-5′-(1H-pyrazol-4-yl)-2,2′,3,3′,3a,5,6,6a-octahydrospiro[cyclopenta[d][1,3]oxazole-1,1′-isoindole]-3′,5-dione as a white solid.

2′-[(3-Methoxyphenyl)methyl]-5′-(1H-pyrazol-4-yl)-2,2′,3,3′,3a,4,5,6a-octahydrospiro[cyclopenta[d][1,3]oxazole-1,1′-isoindole]-3′,5-dione: ¹H NMR (300 MHz, DMSO-d6) δ 8.21 (s, 2H), 7.85-8.07 (m, 3H), 7.43 (d, J=8.1 Hz, 1H), 7.23 (t, J=8.2 Hz, 1H), 6.76-6.86 (m, 3H), 4.83-4.95 (m, 2H), 4.39-4.51 (m, 2H), 3.70 (s, 3H), 2.09 (dt, J=8.3, 4.3 Hz, 2H), 1.75-1.97 (m, 2H).

2′-[(3-Methoxyphenyl)methyl]-5′-(1H-pyrazol-4-yl)-2,2′,3,3′,3a,5,6,6a-octahydrospiro[cyclopenta[d][1,3]oxazole-1,1′-isoindole]-3′,5-dione ¹H NMR (300 MHz, DMSO-d6) δ 8.21 (s, 2H), 8.11 (s, 1H), 7.98 (d, J=1.6 Hz, 1H), 7.85 (dd, J=8.0, 1.8 Hz, 1H), 7.51 (d, J=8.0 Hz, 1H), 7.19 (t, J=7.9 Hz, 1H), 6.68-6.82 (m, 3H), 5.10 (d, J=16.4 Hz, 1H), 4.51-4.77 (m, 3H), 3.68 (s, 3H), 2.26-2.45 (m, 2H), 1.76-1.87 (m, 1H), 1.52 (d, J=5.7 Hz, 1H). LCMS: Calculated: for C24H22N4O4: 430, Measured: 431 [M+H]+.

### Example 100

### 2,3-dihydroxy-2′-(3-methoxybenzyl)-5′-(1H-pyrazol-4-yl)spiro[cyclopentane-1,1′-isoindolin]-3′-one

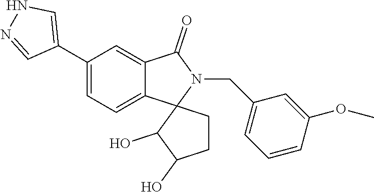 [SMILES: COc1cccc(CN2C(=O)c3cc(-c4cn[nH]c4)ccc3C23CCC(O)C3O)c1]

### Step 1: 5′-Bromo-2,3-dihydroxy-2′-[(3-methoxyphenyl)methyl]-2′,3′-dihydrospiro[cyclopentane-1,1′-isoindole]-3′-one

To a mixture of 5′-bromo-2′-(3-methoxybenzyl)spiro[cyclopentane-1,1′-isoindolin]-2-en-3′-one (500 mg, 1.30 mmol) in acetone (15 mL) and H₂O (1.5 mL) with NMO (457 mg, 3.90 mmol) was added OsO₄ (2.5% in t-BuOH, 661 mg, 0.065 mmol) and the reaction mixture was stirred at room temperature. After 12 h, the reaction mixture was allowed to cool to room temperature, diluted with EtOAc (50 mL), washed with brine (3×15 mL), dried (Na₂SO₄), filtered and concentrated in vacuo. Purification of the resulting residue by silica gel column chromatography (40 g, 0-15% MeOH/DCM) yielded 5′-bromo-2,3-dihydroxy-2′-[(3-methoxyphenyl)methyl]-2′,3′-dihydrospiro[cyclopentane-1,1′-isoindole]-3′-one as a light yellow solid, which was used in the next step without further purification.

### Step 2: 2,3-Dihydroxy-2′-[(3-methoxyphenyl)methyl]-5′-(1H-pyrazol-4-yl)-2′,3′-dihydrospiro[cyclopentane-1,1′-isoindole]-3′-one

2,3-Dihydroxy-2′-[(3-methoxyphenyl)methyl]-5′-(1H-pyrazol-4-yl)-2′,3′-dihydrospiro[cyclopentane-1,1′-isoindole]-3′-one was prepared according to the procedure described in Example 1, Step 5.

¹H NMR (300 MHz, DMSO-d6) δ 8.16 (s, 2H), 7.89-7.83 (m, 2H), 7.82-7.77 (m, 1H), 7.28-7.15 (m, 1H), 6.94-6.74 (m, 3H), 4.79-4.57 (m, 2H), 4.19 (d, J=4.7 Hz, 1H), 4.11 (d, J=4.9 Hz, 1H), 3.70 (s, 3H), 2.29-1.91 (m, 2H), 1.90-1.71 (m, 2H); LCMS: Calculated: for C23H23N3O4: 405, Measured: 406 [M+H]+.

### Example 101

### 3,4-dihydroxy-2′-(3-methoxybenzyl)-5′-(1H-pyrazol-4-yl)spiro[cyclopentane-1,1′-isoindolin]-3′-one

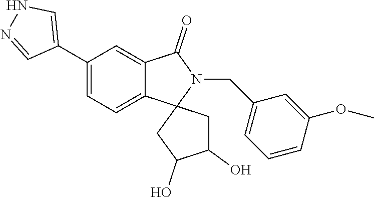 [SMILES: COc1cccc(CN2C(=O)c3cc(-c4cn[nH]c4)ccc3C23CC(O)C(O)C3)c1]

3,4-dihydroxy-2′-(3-methoxybenzyl)-5′-(1H-pyrazol-4-yl)spiro[cyclopentane-1,1′-isoindolin]-3′-one was similarly prepared according to the procedures described herein, selecting and substituting suitable reagents and starting materials, as would be recognized by those skilled in the art.

¹H NMR (400 MHz, METHANOL-d4) δ 7.80-8.20 (m, 5H), 7.48-7.56 (m, 1H), 7.24 (s, 1H), 6.87-6.99 (m, 1H), 6.72-6.87 (m, 1H), 5.49 (s, 1H), 4.75 (s, 1H), 4.18-4.40 (m, 2H), 3.65-3.85 (m, 3H), 2.24-2.36 (m, 1H), 2.16 (br dd, J=6.06, 7.58 Hz, 4H), LCMS: Calculated: for C23H23N3O4: 405, Measured: 406 [M+H]+.

### Example 102

### 2′-(3-methoxybenzyl)-3′-oxo-5′-(1H-pyrazol-4-yl)spiro[cyclopentane-1,1′-isoindoline]-2-carboxylic acid

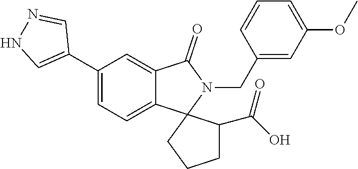 [SMILES: COc1cccc(CN2C(=O)c3cc(-c4cn[nH]c4)ccc3C23CCCC3C(=O)O)c1]

### Step 1: Ethyl (Z)-2-((3-methoxybenzyl)imino)cyclopentane-1-carboxylate

Ethyl (Z)-2-((3-methoxybenzyl)imino)cyclopentane-1-carboxylate was prepared according to the procedure describe din Example 98/99, Step 1, substituting ethyl 2-oxocyclopentanecarboxylate for cyclopentanone. LCMS: Calculated: for C16H21 NO3: 275, Measured: 276 [M+H]+.

### Step 2: Ethyl 2-(5-bromo-2-iodo-N-(3-methoxybenzyl)benzamido)cyclopent-1-enecarboxylate

Ethyl 2-(5-bromo-2-iodo-N-(3-methoxybenzyl)benzamido)cyclopent-1-enecarboxylate was prepared according to the procedure described in Example 98/99, Step 1, substituting ethyl (Z)-2-((3-methoxybenzyl)imino)cyclopentane-1-carboxylate for N-(3-methoxybenzyl)cyclopentanimine. LCMS: Calculated: for C23H23BrINO4: 584, found 607 [M+Na]+.

### Step 3: Ethyl 5′-bromo-2′-[(3-methoxyphenyl)methyl]-3′-oxo-2′,3′-dihydrospiro[cyclopentane-1,1′-isoindol]-2-ene-2-carboxylate

Ethyl 5′-bromo-2′-[(3-methoxyphenyl)methyl]-3′-oxo-2′,3′-dihydrospiro[cyclopentane-1,1′-isoindol]-2-ene-2-carboxylate was prepared according to the procedure described in Example 98/99, Step 3, substituting ethyl 2-(5-bromo-2-iodo-N-(3-methoxybenzyl)benzamido)cyclopent-1-enecarboxylate for 5-bromo-N-(cyclopent-1-en-1-yl)-2-iodo-N-(3-methoxybenzyl)benzamide. LCMS: Calculated: for C23H22BrNO4: 456.3, found 456.2[M]+.

### Step 4: Ethyl 2′-[(3-methoxyphenyl)methyl]-3′-oxo-5′-(1H-pyrazol-4-yl)-2′,3′-dihydrospiro[cyclopentane-1,1′-isoindol]-2-ene-2-carboxylate

Ethyl 2′-[(3-methoxyphenyl)methyl]-3′-oxo-5′-(1H-pyrazol-4-yl)-2′,3′-dihydrospiro[cyclopentane-1,1′-isoindol]-2-ene-2-carboxylate was prepared according to the procedure described in Example 2, Step 2, substituting Ethyl 5′-bromo-2′-[(3-methoxyphenyl)methyl]-3′-oxo-2′,3′-dihydrospiro[cyclopentane-1,1′-isoindol]-2-ene-2-carboxylate for (R)-6-bromo-2-(1-(3-methoxyphenyl)ethyl)isoindolin-1-one. LCMS: Calculated: for C26H25N3O4: 443.5, found 444.3[M+H]+.

### Step 5: Ethyl 2′-[(3-methoxyphenyl)methyl]-3′-oxo-5′-(1H-pyrazol-4-yl)-2′,3′-dihydrospiro[cyclopentane-1,1′-isoindole]-2-carboxylate

To a solution of ethyl 2′-[(3-methoxyphenyl)methyl]-3′-oxo-5′-(1H-pyrazol-4-yl)-2′,3′-dihydrospiro[cyclopentane-1,1′-isoindol]-2-ene-2-carboxylate (640 mg, 1.44 mmol) in glacial acetic acid (10 mL) was added Pd/C (300 mg) and the reaction mixture was warmed to 130° C. After 12 h, the reaction mixture was filtered, and the filtrate was concentrated in vacuo. The residue was dissolved in EtOAc (10 mL) washed with satd. aq. NaHCO₃ (1×5 mL), brine (1×5 mL), dried (Na₂SO₄), filtered and concentrated in vacuo to yield ethyl 2′-[(3-methoxyphenyl)methyl]-3′-oxo-5′-(1H-pyrazol-4-yl)-2′,3′-dihydrospiro[cyclopentane-1,1′-isoindole]-2-carboxylate as a white solid. LCMS: Calculated: for C26H27N3O4: 445, Measured: 445 [M]+.

### Step 6: 2′-[(3-Methoxyphenyl)methyl]-3′-oxo-5′-(1H-pyrazol-4-yl)-2′,3′-dihydrospiro[cyclopentane-1,1′-isoindole]-2-carboxylic acid

To a mixture of ethyl 2′-[(3-methoxyphenyl)methyl]-3′-oxo-5′-(1H-pyrazol-4-yl)-2′,3′-dihydrospiro[cyclopentane-1,1′-isoindole]-2-carboxylate (100 mg, 0.224 mmol) in THE (1 mL) was added LiOH (5.0 M, 0.23 mL). The mixture was stirred overnight at room temperature. The reaction was concentrated and diluted with H₂O. The resulting mixture was treated with acetic acid to adjust pH (pH=5) using acetic acid and concentrated. Purification of the resulting residue by reverse phase chromatography (40 g, 0-45% ACN/H₂O (containing 0.05% TFA) yielded 2′-[(3-methoxyphenyl)methyl]-3′-oxo-5′-(1H-pyrazol-4-yl)-2′,3′-dihydrospiro[cyclopentane-1,1′-isoindole]-2-carboxylic acid as a white solid.

¹H NMR (300 MHz, DMSO) δ 8.11-8.20 (m, 2H), 7.85 (t d, J=3.6, 1.5 Hz, 1H), 7.77 (d d, J=8.0, 1.7 Hz, 1H), 7.52 (d d, J=59.3, 8.3 Hz, 1H), 7.11-7.23 (m, 1H), 6.70-6.94 (m, 3H), 4.93 (dd, J=39.8, 16.5 Hz, 1H), 4.38 (dd, J=99.2, 16.6 Hz, 1H), 3.66 (s, 3H), 3.38 (td, J=10.9, 8.7 Hz, 1H), 1.84-2.36 (m, 4H), 1.60-1.84 (m, 2H); LCMS: Calculated: for C24H23N3O4: 417, Measured: 418 [M+H]+.

### Example 103

### 2′-(3-methoxybenzyl)-3′-oxo-5′-(1H-pyrazol-4-yl)spiro[cyclopentane-1,1′-isoindoline]-2-carboxamide

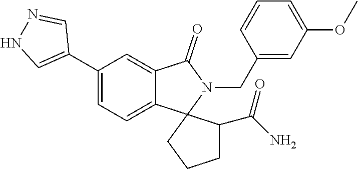 [SMILES: COc1cccc(CN2C(=O)c3cc(-c4cn[nH]c4)ccc3C23CCCC3C(N)=O)c1]

A mixture of 2′-[(3-methoxyphenyl)methyl]-3′-oxo-5′-(1H-pyrazol-4-yl)-2′,3′-dihydrospiro[cyclopentane-1,1′-isoindole]-2-carboxylic acid (100 mg, 0.240 mmol) and NH₄Cl (128 mg, 2.34 mmol) in DMF (2 mL) was treated with HATU (182 mg, 0.479 mmol) and triethylamine (363 mg, 3.60 mmol) and allowed to stir at room temperature. After 12 h, the reaction mixture was diluted with EtOAc (100 mL), washed with water (2×40 mL), brine (2×40 mL), dried (Na₂SO₄), filtered and concentrated in vacuo. Purification of the resulting residue by reverse phase chromatography (40 g, 0-100% ACN/H₂O (containing 0.05% TFA) yielded 2′-[(3-methoxyphenyl)methyl]-3′-oxo-5′-(1H-pyrazol-4-yl)-2′,3′-dihydrospiro[cyclopentane-1,1′-isoindole]-2-carboxamide as a white solid.

¹H NMR (300 MHz, DMSO) δ 8.15 (d, J=10.2 Hz, 2H), 7.69-7.90 (m, 2H), 7.48 (dd, J=49.0, 8.2 Hz, 1H), 7.17 (q, J=7.7 Hz, 1H), 6.67-6.91 (m, 3H), 6.44-6.56 (m, 2H), 5.00 (dd, J=16.7, 8.4 Hz, 1H), 4.44 (t, J=16.3 Hz, 1H), 3.66 (d, J=3.5 Hz, 3H), 3.06-3.19 (m, 1H), 2.14-2.39 (m, 1H), 1.56-2.11 (m, 5H); LCMS: Calculated: for C24H24N4O3: 416, Measured: 417 [M+H]+.

### Example 104

### Methyl (2′-(3-methoxybenzyl)-3′-oxo-5′-(1H-pyrazol-4-yl)spiro[cyclopentane-1,1′-isoindolin]-2-yl)carbamate

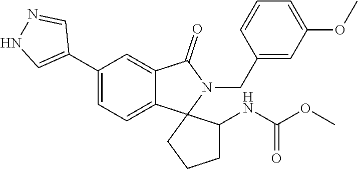 [SMILES: COC(=O)NC1CCCC12c1ccc(-c3cn[nH]c3)cc1C(=O)N2Cc1cccc(OC)c1]

A mixture of 2′-[(3-methoxyphenyl)methyl]-3′-oxo-5′-(1H-pyrazol-4-yl)-2′,3′-dihydrospiro[cyclopentane-1,1′-isoindole]-2-carboxylic acid (150 mg, 0.359 mmol) and triethylamine (109 mg, 1.08 mmol) in toluene (6 mL) was treated with diphenylphosphoryl azide (296 mg, 1.08 mmol) and the resulting mixture was warmed to 110° C. After 12 h, the resulting solution was concentrated, and the residue was dissolved in methanol (6 mL). Sodium methanolate (116 mg, 2.16 mmol) was added and the reaction mixture was warmed to 80° C. for 1 h. The mixture was concentrated in vacuo. Purification of the resulting residue by reverse phase chromatography (40 g, 0-100% ACN/H₂O (containing 0.05% TFA) yielded methyl (2′-(3-methoxybenzyl)-3′-oxo-5′-(1H-pyrazol-4-yl)spiro[cyclopentane-1,1′-isoindolin]-2-yl)carbamate as a white solid.

¹H NMR (300 MHz, DMSO-d6) δ 8.15-8.29 (m, 2H), 7.81-7.95 (m, 2H), 7.45-7.66 (m, 1H), 7.03-7.31 (m, 1H), 6.58-6.89 (m, 3H), 4.78-5.16 (m, 1H), 4.34-4.61 (m, 2H), 3.70 (s, 3H), 3.28-3.44 (m, 3H), 1.75-2.23 (m, 5H), 1.50-1.70 (m, 1H); LCMS: Calculated: for C25H26N4O4: 446, Measured: 447 [M+H]+.

Table PD-1 below lists ¹H NMR and LCMS values measured for a representative sample of representative compounds of the present invention, wherein the sample was prepared as described in the schemes and examples above.

TABLE PD-1
Measured ¹H NMR and MS

| | Example | |
|---|---|---|
| ID No. | No. | NMR Data |
| 1 | 1 | 1H NMR (300 MHz, METHANOL-d₄) 8.35 (bs, 1H), 8.05 |
| | | (bs, 1H), 7.96 (s, 1H), 7.85 (d, J = 1.8 Hz, 1H), 7.68-7.62 |
| | | (m, 1H), 7.56-7.52 (m, 1H), 7.48 (d, J = 7.9 Hz, 1H), 7.28 |
| | | (t, J = 7.8 Hz, 1H), 6.94-6.82 (m, 2H), 4.76 (s, 2H), 4.35 |
| | | (s, 2H), 3.75 (s, 3H); LCMS: Calculated: for |
| | | C19H17N3O2: 319, Measured: 320 [M + H]+. |
| 2 | 3 | 1H NMR (400 MHz, CHLOROFORM-d) Shift 7.91-8.08 |
| | | (m, 3H), 7.60-7.79 (m, 1H), 7.40 (d, J = 7.58 Hz, 1H), |
| | | 6.77-6.95 (m, 2H), 4.79 (s, 2H), 4.29 (s, 2H), 4.00 (q, |
| | | J = 7.07 Hz, 2H), 1.39 (t, J = 6.82 Hz, 3H); LCMS: |
| | | Calculated: for C20H19N3O2: 333, Measured: 334 |
| | | [M + H]+. |
| 3 | | ¹H NMR (400 MHz, METHANOL-d₄) δ 8.82-8.97 (m, 1H), |
| | | 8.45 (br d, J = 3.03 Hz, 1H), 8.07 (s, 1H), 8.00 (s, 1H), |
| | | 7.91 (dd, J = 1.26, 7.83 Hz, 1H), 7.56-7.70 (m, 2H), 7.17- |
| | | 7.34 (m, 1H), 6.76-6.94 (m, 3H), 4.79 (s, 2H), 4.41 (s, |
| | | 2H), 3.76 (s, 3H); LCMS: Calculated: for C23H19N3O2: |
| | | 369, Measured: 370 [M + H]+. |
| 4 | | ¹H NMR (400 MHz, METHANOL-d₄) δ 8.62-8.70 (m, 1H), |
| | | 8.43-8.50 (m, 1H), 8.27-8.38 (m, 1H), 7.74 (s, 1H), 7.57 |
| | | (d, J = 6.06 Hz, 1H), 7.28 (t, J = 8.08 Hz, 1H), 6.83-6.98 (m, |
| | | 3H), 4.81 (s, 2H), 4.51 (s, 2H), 3.77 (s, 3H); LCMS: |
| | | Calculated: for C20H18N4O2: 346, Measured: 347 |
| | | [M + H]+. |
| 6 | 81 | 1H NMR (300 MHz, METHANOL-d₄) 8.2-8.1 (bs, 2H), |
| | | 7.96 (s, 1H), 7.86 (d, J = 1.8 Hz, 1H), 7.54 (d, J = 1.8 Hz, |
| | | 1H), 7.20 (t, J = 7.8 Hz, 1H), 6.90-6.86 (m, 2H), 6.8 (d, |
| | | J = 7.9 Hz, 1H), 5.85 (s, 2H), 4.65 (s, 2H), 3.73 (s, 3H), |
| | | 2.83-2.75 (m, 2H), 2.68-2.60 (m, 2H); LCMS: Calculated: |
| | | for C23H21N3O2: 372, Measured: 372 [M]+. |
| 7 | 73 | 1H NMR (400 MHz, DMSO-d6) d 13.00 (s, 1H), 8.34 (s, |
| | | 1H), 8.03 (s, 1H), 7.92 (dd, J = 1.6, 0.7 Hz, 1H), 7.87 (dd, |
| | | J = 7.9, 1.7 Hz, 1H), 7.63 (dd, J = 7.9, 0.7 Hz, 1H), 7.22 |
| | | (t, J = 8.0 Hz, 1H), 6.93 (dd, J = 7.3, 1.5 Hz, 2H), 6.77- |
| | | 6.84 (m, 1H), 4.66 (s, 2H), 3.71 (s, 3H), 1.37 (s, 6H). |
| | | LCMS: Calculated: for C21H21N3O2: 347, Measured: |
| | | 348 [M + H]+. |
| 8 | 101 | 1H NMR (400 MHz, METHANOL-d4) δ 7.80-8.20 (m, |
| | | 5H), 7.48-7.56 (m, 1H), 7.24 (s, 1H), 6.87-6.99 (m, 1H), |
| | | 6.72-6.87 (m, 1H), 5.49 (s, 1H), 4.75 (s, 1H), 4.18-4.40 |
| | | (m, 2H), 3.65-3.85 (m, 3H), 2.24-2.36 (m, 1H), 2.16 (br |
| | | dd, J = 6.06, 7.58 Hz, 4H); LCMS: Calculated: for |
| | | C23H23N3O4: 405, Measured: 406 [M + H]+. |
| 9 | | ¹H NMR (400 MHz, METHANOL-d₄) δ 8.11-8.29 (m, 2H), |
| | | 7.96-8.00 (m, 1H), 7.81-7.87 (m, 1H), 7.50-7.56 (m, 1H), |
| | | 7.30-7.39 (m, 1H), 7.21 (br s, 2H), 7.08-7.14 (m, 1H), |
| | | 4.81 (s, 2H), 4.41 (s, 2H), 2.94 (s, 3H); LCMS: |
| | | Calculated: for C19H18N4O3S: 382, Measured: 383 |
| | | [M+HJ+. |
| 10 | | ¹H NMR (400 MHz, METHANOL-d₄) 5 8.43-8.57 (m, 1H), |
| | | 8.30 (s, 1H), 8.12 (d, J = 8.08 Hz, 1H), 7.82 (d, J = 8.08 Hz, |
| | | 1H), 7.78 (d, J = 3.54 Hz, 1H), 7.73 (br d, J = 6.06 Hz, 1H), |
| | | 7.31-7.40 (m, 1H), 7.24 (s, 1H), 7.20 (brd, J = 8.08 Hz, |
| | | 1H), 7.14 (d, J = 7.58 Hz, 1H), 7.03 (d, J = 3.54 Hz, 1H), |
| | | 4.87 (s, 2H), 4.57 (s, 2H), 2.95 (s, 3H); LCMS: |
| | | Calculated: for C23H20N4O3S: 432, Measured: 433 |
| | | [M + H]+. |
| 11 | | ¹H NMR (400 MHz, METHANOL-d₄) δ 8.66-8.71 (m, 1H), |
| | | 8.46-8.53 (m, 1H), 8.33-8.39 (m, 1H), 7.73-7.78 (m, 1H), |
| | | 7.59-7.65 (m, 1H), 7.26 (s, 1H), 6.82-6.94 (m, 3H), 4.81 |
| | | (s, 2H), 4.51 (s, 2H), 4.01 (d, J = 7.07 Hz, 2H), 1.36 (t, |
| | | J = 7.07 Hz, 3H); LCMS: Calculated: for C21H20N4O2: |
| | | 360, Measured: 361 [M + H]+. |
| 12 | | ¹H NMR (400 MHz, METHANOL-d₄) δ 7.98-8.06 (m, 2H), |
| | | 7.75-7.80 (m, 1H), 7.61 (dd, J = 1.52, 8.08 Hz, 1H), 7.48- |
| | | 7.54 (m, 2H), 7.42-7.48 (m, 1H), 7.36-7.41 (m, 2H), 7.27 |
| | | (t, J = 7.83 Hz, 1H), 6.82-6.90 (m, 3H), 5.63 (d, J = 14.65 |
| | | Hz, 1H), 5.08 (s, 1H), 4.26-4.35 (m, 2H), 4.11 (br d, |
| | | J = 5.56 Hz, 1H), 3.96-4.04 (m, 2H), 3.58 (br d, J = 10.11 |
| | | Hz, 1H), 1.34 (t, J = 7.07 Hz, 3H); LCMS: Calculated: for |
| | | C30H26N4O4: 506, Measured: 507 [M + H]+. |
| 13 | 2 | 1H NMR (400 MHz, DMSO-d6) d 13.01 (s, 1H), 8.34 (s, |
| | | 1H), 8.03 (s, 1H), 7.93 (d, J = 1.8 Hz, 1H), 7.84 (m, 1H), |
| | | 7.52 (d, J = 7.9 Hz, 1H), 7.28 (t, J = 7.8 Hz, 1H), 6.94- |
| | | 6.82 (m, 3H), 5.52 (m, 1H), 4.51 (d, J = 17.7 Hz, 1H), |
| | | 4.10 (d, J = 17.6 Hz, 1H), 3.35 (m, 1H), 1.63 (d, J = 7.2 |
| | | Hz, 3H); LCMS: Calculated: for C20H19N3O2: 333.384, |
| | | Measured: MH+: 334.1 [M + H]+. |
| 14 | 88 | 1H NMR (400 MHz, DMSO-d6) d 13.05 (brs, 1H), 8.20 |
| | | (brs, 2H), 7.99-8.00 (m, 2H), 7.83 (dd, J = 8.1, 1.8 Hz, |
| | | 1H), 7.21 (t, J = 8.1 Hz, 1H), 6.84-6.86 (m, 2H), 6.78- |
| | | 6.81 (m, 1H), 4.71 (s, 2H), 3.71 (s, 3H), 3.04-3.10 (m, |
| | | 2H), 2.93-2.95 (m, 2H), 2.00 (td, J = 12.6, 5.0 Hz, 2H), |
| | | 1.20 (d, J = 12.6 Hz, 2H); LCMS: Calculated: for |
| | | C23H24N4O2: 388, Measured: 389 [M + H]+. |
| 15 | | ¹H NMR (400 MHz, METHANOL-d₄) δ 8.03-8.14 (m, 2H), |
| | | 7.85-7.95 (m, 1H), 7.64 (br d, J = 7.58 Hz, 2H), 7.26 (s, |
| | | 1H), 6.89-6.97 (m, 2H), 6.83-6.88 (m, 1H), 5.06-5.17 (m, |
| | | 1H), 4.75 (s, 1H), 4.55 (d, J = 15.66 Hz, 1H), 3.76 (s, 3H), |
| | | 3.23 (t, J = 4.55 Hz, 2H); LCMS: Calculated: for |
| | | C21H18N4O2: 358, Measured: 359 [M + H]+. |
| 16 | 6 | 1H NMR (400 MHz, METHANOL-d₄) δ 8.04-8.23 (m, 1H), |
| | | 7.99 (s, 1H), 7.85 (dd, J = 1.52, 8.08 Hz, 1H), 7.53 (d, |
| | | J = 8.08 Hz, 1H), 7.31-7.40 (m, 1H), 6.95-7.16 (m, 2H), |
| | | 4.83 (s, 1H), 4.42 (s, 1H); LCMS: Calculated: for |
| | | C18H14FN3O: 307, Measured: 308 [M + H]+. |
| 17 | 72 | 1H NMR (400 MHz, DMSO-d6) d 8.43 (s, 1H), 8.31- |
| | | 8.35 (m, 2H), 7.79 (d, J = 8.0 Hz, 1H), 7.21-7.26 (m, |
| | | 2H), 6.94 (dd, J = 7.3, 1.5 Hz, 2H), 6.80-6.82 (m, 1H), |
| | | 6.76 (s, 2H), 4.69 (s, 2H), 3.72 (s, 3H), 1.40 (s, 6H); |
| | | LCMS: Calculated: for C22H22N4O2: 374, Measured: |
| | | 375 [M + H]+. |
| 18 | | ¹H NMR (400 MHz, METHANOL-d₄) δ 8.06 (s, 2H), 7.94- |
| | | 8.02 (m, 3H), 7.82-7.89 (m, 1H), 7.44-7.61 (m, 3H), 4.89 |
| | | (s, 2H), 4.42 (s, 2H); LCMS: Calculated: for |
| | | C22H22N4O2: 333, Measured: 334 [M + H]+. |
| 19 | | ¹H NMR (400 MHz, METHANOL-d₄) δ 8.12-8.25 (m, 2H), |
| | | 8.00 (s, 1H), 7.83-7.88 (m, 1H), 7.70-7.79 (m, 2H), 7.43- |
| | | 7.56 (m, 3H), 4.88 (s, 2H), 4.43 (s, 2H), 4.18 (td, J = 6.76, |
| | | 13.26 Hz, 1H), 1.20-1.26 (m, 6H); LCMS: Calculated: for |
| | | C22H22N4O2: 374, Measured: 375 [M + H]+. |
| 20 | 67 | 1H NMR (400 MHz, DMSO-d6) d 15.18 (brs, 1H), 8.52 |
| | | (brs, 1H), 8.20 (s, 1H), 8.12 (d, J = 7.9 Hz, 1H), 7.65 (d, |
| | | J = 7.9 Hz, 1H), 7.26-7.30 (m, 1H), 6.84-6.88 (m, 3H), |
| | | 4.72 (s, 2H), 4.41 (s, 2H), 3.74 (s, 3H); LCMS: |
| | | Calculated: for C18H16N4O2: 320, Measured: |
| | | 697[2M + H]+. |
| 21 | 68 | 1H NMR (400 MHz, DMSO-d6) d 15.19 (brs, 1H), 8.52 |
| | | (brs, 1H), 8.19 (s, 1H), 8.15 (dd, J = 8.1, 1.7 Hz, 1H), |
| | | 7.76 (d, J = 7.9 Hz, 1H), 7.23 (dd, J = 8.7, 7.3 Hz, 1H), |
| | | 6.93-6.95 (m, 2H), 6.81 (dt, J = 7.7, 2.0 Hz, 1H), 4.68 (s, |
| | | 2H), 3.72 (s, 3H), 1.39 (s, 6H); LCMS: Calculated: for |
| | | C20H20N4O2: 348, Measured: 697 [2M + H]+. |
| 22 | 7 | 1H NMR (400 MHz, METHANOL-d₄) δ 8.10 (br s, 1H), |
| | | 7.98 (s, 1H), 7.84 (d, J = 7.58 Hz, 1H), 7.34-7.56 (m, 3H), |
| | | 4.86 (s, 1H), 4.42 (s, 1H), 4.10 (s, 1H), 2.61-2.77 (m, |
| | | 2H); LCMS: Calculated: for C19H18N4O: 318, |
| | | Measured: 319 [M + H]+. |
| 23 | 70 | 1H NMR (400 MHz, DMSO-d6) d 9.01 (dd, J = 2.3, 0.9 |
| | | Hz, 1H), 8.83 (q, J = 4.6 Hz, 1H), 8.37 (dd, J = 8.2, 2.4 |
| | | Hz, 1H), 8.10-8.12 (m, 2H), 8.04 (dd, J = 7.9, 1.8 Hz, |
| | | 1H), 7.73 (d, J = 7.6 Hz, 1H), 7.28 (ddt, J = 8.5, 7.0, 1.3 |
| | | Hz, 1H), 6.85-6.89 (m, 3H), 4.74 (s, 2H), 4.45 (s, 2H), |
| | | 3.74 (s, 3H), 2.85 (d, J = 4.8 Hz, 3H); LCMS: Calculated: |
| | | for C23H21N3O3: 387, Measured: 388 [M + H]+. |
| 24 | | ¹H NMR (400 MHz, METHANOL-d₄) δ 8.04-8.15 (m, 3H), |
| | | 7.93 (s, 1H), 7.86 (d, J = 8.59 Hz, 2H), 7.67 (d, J = 7.58 Hz, |
| | | 1H), 7.62 (d, J = 8.08 Hz, 1H), 7.48-7.56 (m, 1H), 7.38- |
| | | 7.46 (m, 2H), 7.09-7.20 (m, 1H), 6.32 (q, J = 6.57 Hz, 1H), |
| | | 4.26 (d, J = 17.68 Hz, 1H), 3.28-3.42 (m, 2H), 1.77 (d, |
| | | J = 7.07 Hz, 3H); LCMS: Calculated: for C23H19N3O: |
| | | 353, Measured: 354 [M + H]+. |
| 25 | | ¹H NMR (400 MHz, METHANOL-d₄) δ 8.36-8.46 (m, 1H), |
| | | 8.10 (s, 2H), 7.85-7.92 (m, 3H), 7.79-7.84 (m, 1H), 7.57- |
| | | 7.64 (m, 1H), 5.57-5.75 (m, 1H), 4.61-4.81 (m, 2H), 2.82 |
| | | (s, 3H), 1.89 (d, J = 7.58 Hz, 3H); LCMS: Calculated: for |
| | | C19H18N4O: 318, Measured: 319 [M + H]+. |
| 26 | | ¹H NMR (400 MHz, METHANOL-d₄) δ 8.20 (s, 1H), 8.09- |
| | | 8.18 (m, 2H), 7.82 (d, J = 7.58 Hz, 1H), 7.72 (d, J = 8.08 |
| | | Hz, 1H), 7.63 (d, J = 7.58 Hz, 1H), 7.49-7.59 (m, 2H), 7.26 |
| | | (d, J = 8.08 Hz, 1H), 5.35 (d, J = 16.67 Hz, 1H), 5.13 (d, |
| | | J = 16.67 Hz, 1H), 4.61 (q, J = 6.57 Hz, 1H), 1.45 (d, J = 6.57 |
| | | Hz, 3H); LCMS: Calculated: for C20H17N3O3: 347, |
| | | Measured: 348 [M + H]+. |
| 27 | 8 | 1H NMR (400 MHz, METHANOL-d4) δ 8.10 (br s, 1H), |
| | | 7.95 (d, J = 1.01 Hz, 1H), 7.83 (dd, J = 1.52, 7.58 Hz, 1H), |
| | | 7.53 (d, J = 8.08 Hz, 1H), 6.87 (d, J = 3.03 Hz, 1H), 6.49- |
| | | 6.75 (m, 1H), 4.43 (s, 1H), 2.41 (s, 3H); LCMS: |
| | | Calculated: for C17H15N3OS: 309, Measured: 310 |
| | | [M + H]+. |
| 28 | 5 | 1H NMR (400 MHz, METHANOL-d4) δ 8.10 (br s, 1H), |
| | | 7.96 (s, 1H), 7.84 (d, J = 8.08 Hz, 1H), 7.53 (d, J = 7.58 Hz, |
| | | 1H), 7.35 (d, J = 5.05 Hz, 1H), 7.11 (d, J = 3.03 Hz, 1H), |
| | | 6.99 (dd, J = 3.28, 5.31 Hz, 1H), 5.00 (s, 1H), 4.45 (s, 1H); |
| | | LCMS: Calculated: for C16H13N3OS: 295, Measured: |
| | | 296 [M + H]+. |
| 29 | | ¹H NMR (400 MHz, METHANOL-d₄) δ 8.08 (br s, 2H), |
| | | 7.95 (s, 1H), 7.78-7.83 (m, 1H), 7.49 (d, J = 8.08 Hz, 1H), |
| | | 7.26 (t, J = 8.08 Hz, 1H), 6.93 (d, J = 7.58 Hz, 1H), 6.90 (s, |
| | | 1H), 6.78-6.85 (m, 1H), 5.59-5.69 (m, 1H), 4.47-4.56 (m, |
| | | 1H), 4.06-4.15 (m, 1H), 4.00 (d, J = 7.07 Hz, 2H), 1.70 (d, |
| | | J = 7.07 Hz, 3H), 1.35 (t, J = 7.07 Hz, 3H); LCMS: |
| | | Calculated: for C21H21N3O2: 347, Measured: 348 |
| | | [M + H]+. |
| 30 | | ¹H NMR (400 MHz, METHANOL-d4) δ 8.16 (br s, 1H), |
| | | 7.88 (dd, J = 6.82, 7.83 Hz, 1H), 7.50-7.69 (m, 1H), 7.24- |
| | | 7.36 (m, 2H), 6.91-7.01 (m, 2H), 6.86 (dd, J = 2.02, 8.08 |
| | | Hz, 1H), 5.61 (q, J = 7.24 Hz, 1H), 4.53 (d, J = 18.19 Hz, |
| | | 1H), 4.10 (d, J = 18.19 Hz, 1H), 3.77 (s, 3H), 1.69 (d, |
| | | J = 7.58 Hz, 3H); LCMS: Calculated: for C20H18FN3O2: |
| | | 351, Measured: 352 [M + H]+. |
| 31 | | ¹H NMR (400 MHz, METHANOL-d₄) δ 8.11 (br s, 2H), |
| | | 7.80 (d, J = 7.58 Hz, 1H), 7.58-7.67 (m, 3H), 7.48-7.56 (m, |
| | | 2H), 7.33 (d, J = 8.08 Hz, 1H), 5.31 (d, J = 15.66 Hz, 1H), |
| | | 4.60 (d, J = 15.66 Hz, 1H), 4.51 (q, J = 6.57 Hz, 1H), 4.06- |
| | | 4.17 (m, 1H), 1.44 (d, J = 6.57 Hz, 3H), 1.24 (d, J = 6.57 |
| | | Hz, 3H), 1.15 (d, J = 6.57 Hz, 3H); LCMS: Calculated: for |
| | | C23H24N4O2: 388, Measured: 389 [M + H]+. |
| 32 | | ¹H NMR (400 MHz, METHANOL-d₄) δ 8.09 (br s, 2H), |
| | | 7.77 (s, 1H), 7.55-7.75 (m, 4H), 7.40-7.53 (m, 3H), 7.23- |
| | | 7.32 (m, 1H), 7.17 (q, J = 9.09 Hz, 1H), 5.38 (d, J = 15.66 |
| | | Hz, 1H), 4.62 (d, J = 15.66 Hz, 1H), 4.54 (d, J = 6.57 Hz, |
| | | 1H), 1.43 (d, J = 7.07 Hz, 2H); LCMS: Calculated: for |
| | | C26H20F2N4O2: 458, Measured: 459 [M + H]+. |
| 33 | | ¹H NMR (400 MHz, METHANOL-d₄) δ 8.65-8.71 (m, 1H), |
| | | 8.49-8.57 (m, 1H), 8.32-8.40 (m, 1H), 7.82-7.89 (m, 1H), |
| | | 7.50-7.59 (m, 1H), 7.23-7.33 (m, 1H), 6.94 (s, 2H), 6.84- |
| | | 6.89 (m, 1H), 5.13 (d, J = 15.66 Hz, 1H), 4.59 (d, J = 15.16 |
| | | Hz, 1H), 3.77 (s, 3H); LCMS: Calculated: for |
| | | C22H19N5O2: 385, Measured: 386 [M + H]+. |
| 34 | | ¹H NMR (400 MHz, METHANOL-d₄) δ 8.06-8.14 (m, 1H), |
| | | 7.99-8.02 (m, 1H), 7.76-7.91 (m, 3H), 7.61-7.68 (m, 1H), |
| | | 7.50-7.59 (m, 4H), 7.37-7.44 (m, 1H), 7.24 (d, J = 10.61 |
| | | Hz, 1H), 4.92 (s, 2H), 4.46 (s, 2H); LCMS: Calculated: for |
| | | C25H18F2N4O2: 444, Measured: 445 [M + H]+. |
| 35 | | ¹H NMR (400 MHz, METHANOL-d₄) δ 8.12-8.20 (m, 1H), |
| | | 8.07-8.10 (m, 1H), 8.01-8.05 (m, 1H), 7.33-7.39 (m, 1H), |
| | | 7.22-7.31 (m, 1H), 6.82-6.93 (m, 3H), 4.77 (s, 2H), 4.39 |
| | | (s, 2H), 3.77 (s, 3H); LCMS: Calculated: for |
| | | C19H16FN3O2: 337, Measured: 338 [M + H]+. |
| 36 | | ¹H NMR (400 MHz, METHANOL-d₄) δ 8.54-8.64 (m, 1H), |
| | | 8.34-8.43 (m, 1H), 7.45-7.59 (m, 2H), 7.22-7.34 (m, 1H), |
| | | 6.88 (br s, 3H), 4.78 (s, 2H), 4.50 (s, 2H), 3.77 (s, 3H); |
| | | LCMS: Calculated: for C20H17FN4O2: 364, Measured: |
| | | 365 [M + H]+. |
| 37 | 71 | 1H NMR (400 MHz, DMSO-d6) d 8.85 (q, J = 4.8 Hz, |
| | | 1H), 8.72 (d, J = 5.1 Hz, 1H), 8.33 (d, J = 1.9 Hz, 1H), |
| | | 8.13 (d, J = 1.6 Hz, 1H), 8.09 (dd, J = 7.9, 1.8 Hz, 1H), |
| | | 8.03 (dd, J = 5.1, 2.0 Hz, 1H), 7.74 (d, J = 7.8 Hz, 1H), |
| | | 7.26-7.30 (m, 1H), 6.80-6.95 (m, 3H), 4.74 (s, 2H), |
| | | 4.46 (s, 2H), 3.74 (s, 3H), 2.86 (d, J = 4.8 Hz, 3H). 19F |
| | | NMR (376 MHz, DMSO-d6) d −74.59. LCMS: Calculated: |
| | | for C23H21N3O3: 387, Measured: 388 [M + H]+. |
| 38 | 14 | 1H NMR (300 MHz, DMSO-d6) d 12.94 (s, 1 H), 8.29 (s, |
| | | 1 H), 8.00 (s, 1 H), 7.88 (d, J = 1.6 Hz, 1 H), 7.81 (dd, J = |
| | | 8.0, 1.7 Hz, 1 H), 7.51 (d, J = 7.9 Hz, 1 H), 7.23 (td, J = |
| | | 7.3, 2.0 Hz, 1 H), 6.78-6.89 (m, 3 H), 5.31 (dd, J = 8.3, |
| | | 5.8 Hz, 1 H), 5.06 (t, J = 5.5 Hz, 1 H), 4.57 (d, J = 17.7 |
| | | Hz, 1 H), 4.26 (d, J = 17.6 Hz, 1 H), 3.93 (dq, J = 11.5, |
| | | 6.5, 5.7 Hz, 2 H), 3.69 (s, 3 H); LCMS: Calculated: for |
| | | C20H19N3O3: 349, Measured: 350 [M + H]+. |
| 39 | 69 | 1H NMR (300 MHz, DMSO-d6) d 15.19 (brs, 1H), 8.50 |
| | | (brs, 1H), 8.12-8.18 (m, 2H), 7.75 (d, J = 8.0 Hz, 1H), |
| | | 7.20 (t, J = 8.1 Hz, 1H), 6.92 (dd, J = 4.3, 2.3 Hz, 2H), |
| | | 6.77-6.84 (m, 1H), 4.67 (s, 2H), 3.97 (q, J = 7.0 Hz, 2H), |
| | | 1.38 (s, 6H), 1.29 (t, J = 6.9 Hz, 3H); LCMS: Calculated: |
| | | for C20H20N4O2: 362, Measured: 363 [M + H]+. |
| 40 | 65 | 1H NMR (400 MHz, DMSO-d6) d 12.03 (s, 1 H), 8.49 (d, |
| | | J = 1.4 Hz, 1 H), 8.03 (d, J = 2.0 Hz, 1 H), 7.91 (dd, J = |
| | | 8.2, 2.1 Hz, 1 H), 7.66 (d, J = 8.2 Hz, 1 H), 7.26 (td, J = |
| | | 7.6, 1.2 Hz, 1 H), 6.81-6.92 (m, 3 H), 5.49 (q, J = 7.1 Hz, |
| | | 1 H), 4.56 (d, J = 18.0 Hz, 1 H), 4.14 (d, J = 18.0 Hz, 1 |
| | | H), 3.72 (s, 3 H), 1.62 (d, J = 7.1 Hz, 3 H); LCMS: |
| | | Calculated: for C19H18N4O3: 350, Measured: 351 |
| | | [M + H]+. |
| 41 | 74 | 1H NMR (400 MHz, DMSO-d6) d 8.12 (s, 2H), 7.78- |
| | | 7.82 (m, 2H), 7.50 (d, J = 7.9 Hz, 1H), 7.17 (t, J = 7.8 Hz, |
| | | 1H), 6.99-7.02 (m, 2H), 6.75 (dd, J = 8.2, 2.6 Hz, 1H), |
| | | 4.48 (s, 2H), 3.66 (s, 3H), 2.68 (td, J = 9.5, 6.1 Hz, 2H), |
| | | 2.57 (td, J = 9.5, 6.2 Hz, 2H), 2.05 (tq, J = 14.1, 7.1, 6.0 |
| | | Hz, 4H); 19F NMR (376 MHz, DMSO-d6) d −74.31; |
| | | LCMS: Calculated: for C23H25N3O4: 407, Measured: |
| | | 408 [M + H]+. |
| 42 | 83 | 1H NMR (400 MHz, DMSO-d6) d 8.20 (s, 2H), 7.91 (d, |
| | | J = 1.2 Hz, 1H), 7.87 (dd, J = 8.0, 1.8 Hz, 1H), 7.56 (d, J = |
| | | 7.9 Hz, 1H), 7.23 (t, J = 8.0 Hz, 1H), 6.84-6.89 (m, 2H), |
| | | 6.78-6.84 (m, 1H), 4.67 (s, 2H), 3.71 (s, 3H), 1.98 (dt, |
| | | J = 12.2, 7.0 Hz, 2H), 1.89 (q, J = 5.5, 3.7 Hz, 4H), 1.77 |
| | | (dt, J = 11.6, 5.2 Hz, 2H).; 19F NMR (376 MHz, DMSO- |
| | | d6) d −74.56; LCMS: Calculated: for C23H23N3O2: 373, |
| | | Measured: 374 [M + H]+. |
| 43 | 9 | 1H NMR (400 MHz, METHANOL-d₄) δ 8.10 (s, 1H), 7.96 |
| | | (d, J = 1.01 Hz, 1H), 7.85 (dd, J = 1.52, 8.08 Hz, 1H), 7.55 |
| | | (d, J = 8.08 Hz, 1H), 6.78 (t, J = 3.79 Hz, 1H), 6.42 (dd, |
| | | J = 1.77, 3.79 Hz, 1H), 4.85 (d, J = 2.53 Hz, 1H), 4.46 (s, |
| | | 1H); LCMS: Calculated: for C16H12FN3OS: 313, |
| | | Measured: 314 [M + H]+. |
| 44 | 64 | 1H NMR (400 MHz, DMSO-d6) d 12.04 (s, 1 H), 8.50 (d, |
| | | J = 1.4 Hz, 1 H), 8.06 (d, J = 2.0 Hz, 1 H), 7.92 (dd, J = |
| | | 8.2, 2.1 Hz, 1 H), 7.66 (d, 1 H), 7.21-7.31 (m, 1 H), 6.78- |
| | | 6.88 (m, 3 H), 4.70 (s, 2 H), 4.39 (s, 2 H), 3.71 (s, 3 H); |
| | | LCMS: Calculated: for C18H16N4O3: 336, Measured: |
| | | 337 [M + H]+. |
| 45 | 66 | 1H NMR (400 MHz, DMSO-d6) d 12.03 (s, 1 H), 8.48 (d, |
| | | J = 1.4 Hz, 1 H), 8.03 (d, J = 2.0 Hz, 1 H), 7.93 (dd, J = |
| | | 8.2, 2.1 Hz, 1 H), 7.80 (d, J = 8.2 Hz, 1 H), 7.19 (t, J = |
| | | 8.1 Hz, 1 H), 6.90 (dt, J = 3.8, 1.4 Hz, 2 H), 6.81-6.73 |
| | | (m, 1 H), 4.65 (s, 2 H), 3.96 (q, J = 7.0 Hz, 2 H), 1.37 (s, |
| | | 6 H), 1.28 (t, J = 6.9 Hz, 3 H); LCMS: Calculated: for |
| | | C21H22N4O3: 378, Measured: 379 [M + H]+.. |
| 46 | 93 | 1H NMR (400 MHz, DMSO-d6) d 8.19 (s, 2H), 7.90 (d, |
| | | J = 7.2 Hz, 2H), 7.73-7.75 (m, 1H), 7.23 (t, J = 7.8 Hz, |
| | | 1H), 6.84-6.92 (m, 2H), 6.78-6.84 (m, 1H), 4.65 (q, J = |
| | | 16.4 Hz, 2H), 4.38 (dq, J = 7.6, 4.1 Hz, 1H), 3.71 (s, 3H), |
| | | 2.21 (dd, J = 14.5, 5.8 Hz, 1H), 2.05 (ddtd, J = 16.1, 12.2, |
| | | 9.5, 6.0 Hz, 2H), 1.72-1.91 (m, 3H)./19F NMR (376 |
| | | MHz, DMSO-d6) d −74.80. LCMS: Calculated: for |
| | | C23H23N3O3: 389, Measured: MH+: 390 [M + H]+. |
| 47 | 13 | 1H NMR (300 MHz, DMSO-d6) d 8.98 (t, J = 4.4 Hz, 2H), |
| | | 8.69-8.53 (m, 2H), 8.33 (d, J = 5.4 Hz, 1H), 7.74 (d, J = |
| | | 8.1 Hz, 1H), 7.25 (t, J = 8.2 Hz, 1H), 6.83 (d, J = 6.4 Hz, |
| | | 3H), 4.71 (s, 2H), 4.45 (s, 2H), 3.70 (s, 3H), 2.84 (d, J = |
| | | 4.7 Hz, 3H); LCMS: Calculated: for C22H20N4O3: 388, |
| | | Measured: 389 [M + H]+. |
| 48 | | ¹H NMR (400 MHz, METHANOL-d₄) δ 8.13 (br s, 2H), |
| | | 7.82-7.92 (m, 1H), 7.19-7.35 (m, 2H), 6.94 (d, J = 7.58 Hz, |
| | | 1H), 6.90 (s, 1H), 6.84 (br d, J = 8.08 Hz, 1H), 5.60 (q, |
| | | J = 7.07 Hz, 1H), 4.52 (d, J = 18.19 Hz, 1H), 4.09 (d, |
| | | J = 18.19 Hz, 1H), 4.00 (q, J = 7.07 Hz, 2H), 1.69 (d, J = 7.07 |
| | | Hz, 3H), 1.35 (t, J = 7.07 Hz, 3H); LCMS: Calculated: for |
| | | C21H20FN3O2: 365, Measured: 366 [M + H]+. |
| 49 | 75 | 1H NMR (300 MHz, DMSO-d6) d 12.98 (brs, 1H), 8.32 |
| | | (brs, 1H), 8.02 (brs, 1H), 7.77-7.90 (m, 2H), 7.53 (dd, |
| | | J = 11.8, 7.9 Hz, 1H), 7.26 (dt, J = 10.0, 8.0 Hz, 1H), 6.90- |
| | | 7.04 (m, 2H), 6.78-6.89 (m, 1H), 5.27 (dd, J = 18.6, 7.3 |
| | | Hz, 1H), 4.40-4.84 (m, 2H), 3.72 (d, J = 3.2 Hz, 3H), |
| | | 3.25 (d, J = 27.7 Hz, 2H), 2.09 (d, J = 46.6 Hz, 1H), 1.88 |
| | | (dt, J = 13.6, 6.6 Hz, 1H), 1.79 (d, J = 7.3 Hz, 1H), 1.72 |
| | | (d, J = 7.2 Hz, 2H); LCMS: Calculated: for C22H23N3O3: |
| | | 377, Measured: 378 [M + H]+. |
| 50 | | ¹H NMR (400 MHz, METHANOL-d₄) δ 8.42-8.55 (m, 1H), |
| | | 8.21-8.32 (m, 1H), 8.03-8.13 (m, 1H), 7.75-7.83 (m, 2H), |
| | | 7.66-7.74 (m, 1H), 7.20-7.31 (m, 1H), 6.97-7.05 (m, 1H), |
| | | 6.81-6.93 (m, 3H), 4.82 (s, 2H), 4.51 (s, 2H), 3.77 (s, |
| | | 3H); LCMS: Calculated: for C23H19N3O2: 369, |
| | | Measured: 370 [M + H]+. |
| 51 | | ¹H NMR (400 MHz, METHANOL-d₄) δ 8.09 (s, 1H), 7.93 |
| | | (dd, J = 7.33, 10.86 Hz, 2H), 7.67 (d, J = 8.08 Hz, 1H), |
| | | 7.18-7.30 (m, 3H), 6.94 (d, J = 7.58 Hz, 1H), 6.90 (s, 1H), |
| | | 6.83 (br d, J = 8.59 Hz, 1H), 5.60-5.68 (m, 1H), 4.57-4.65 |
| | | (m, 1H), 4.18 (d, J = 18.19 Hz, 1H), 3.99 (q, J = 7.07 Hz, |
| | | 2H), 1.69-1.75 (m, 3H), 1.32-1.39 (m, 3H); LCMS: |
| | | Calculated: for C23H23N3O2: 373, Measured: 374 |
| | | [M + H]+. |
| 52 | 25 | 1H NMR (400 MHz, METHANOL-d₄) δ 8.01-8.25 (m, 1H), |
| | | 7.94 (s, 1H), 7.82 (dd, J = 1.26, 7.83 Hz, 1H), 7.28-7.61 |
| | | (m, 1H), 7.02-7.23 (m, 1H), 6.78-6.95 (m, 1H), 5.68 (dd, |
| | | J = 5.05, 8.08 Hz, 1H), 4.54-4.73 (m, 1H), 4.35 (d, J = 17.68 |
| | | Hz, 1H), 4.15-4.23 (m, 1H), 4.04-4.15 (m, 1H), 3.82-3.86 |
| | | (m, 1H), 3.76-3.79 (m, 1H), 3.72-3.75 (m, 1H), 3.30-3.40 |
| | | (m, 6H); LCMS: Calculated: for C21H21N3O4: 379, |
| | | Measured: 380 [M + H]+. |
| 53 | 16 | 1H NMR (400 MHz, DMSO-d6) d 12.98 (s, 1H), 8.33 (s, |
| | | 1H), 8.02 (s, 1H), 7.91 (s, 1H), 7.84 (dd, J = 7.9, 1.7 Hz, |
| | | 1H), 7.54 (d, J = 7.9 Hz, 1H), 7.18-7.30 (m, 1H), 6.76- |
| | | 6.91 (m, 3H), 5.34 (dd, J = 8.5, 5.7 Hz, 1H), 5.08 (t, J = |
| | | 5.5 Hz, 1H), 4.60 (d, J = 17.7 Hz, 1H), 4.29 (d, J = 17.6 |
| | | Hz, 1H), 3.89-4.08 (m, 4H), 1.30 (t, J = 6.9 Hz, 3H); |
| | | LCMS: Calculated: for C21H21N3O3: 363, Measured: |
| | | 364 [M + H]+. |
| 54 | | ¹H NMR (400 MHz, CHLOROFORM-d) δ 9.52 (br s, 1H), |
| | | 8.41 (d, J = 5.05 Hz, 1H), 8.24-8.31 (m, 1H), 7.89-7.96 (m, |
| | | 1H), 7.55 (d, J = 7.58 Hz, 1H), 7.38-7.44 (m, 1H), 7.29 (s, |
| | | 1H), 7.23 (d, J = 5.05 Hz, 1H), 6.98 (d, J = 7.58 Hz, 1H), |
| | | 6.87-6.92 (m, 1H), 6.81-6.85 (m, 1H), 6.71-6.75 (m, 1H), |
| | | 4.83 (s, 2H), 4.63 (s, 2H), 4.35-4.40 (m, 2H), 3.77 (s, |
| | | 3H); LCMS: Calculated: for C25H21N3O4: 427, |
| | | Measured: 428 [M + H]+. |
| 55 | | ¹H NMR (400 MHz, METHANOL-d₄) δ 8.08-8.22 (m, 2H), |
| | | 7.98 (s, 1H), 7.83 (d, J = 8.08 Hz, 1H), 7.52 (d, J = 8.08 Hz, |
| | | 1H), 7.28 (t, J = 7.83 Hz, 1H), 6.90-6.98 (m, 2H), 6.87 (br |
| | | d, J = 8.08 Hz, 1H), 4.79 (s, 2H), 4.65 (s, 2H), 4.38 (s, 2H); |
| | | LCMS: Calculated: for C20H17N3O4: 363, Measured: |
| | | 364 [M + H]+. |
| 56 | | ¹H NMR (400 MHz, CHLOROFORM-d) δ 8.03 (s, 1H), |
| | | 7.94 (s, 2H), 7.66-7.73 (m, 1H), 7.40 (d, J = 8.08 Hz, 1H), |
| | | 7.29 (m, 1H), 6.93-6.99 (m, 1H), 6.85-6.89 (m, 1H), 6.78- |
| | | 6.85 (m, 1H), 4.79 (s, 2H), 4.62 (s, 2H), 4.29 (s, 2H), 3.76 |
| | | (s, 3H); LCMS: Calculated: for C21H19N3O4: 377, |
| | | Measured: 378 [M + H]+. |
| 57 | | ¹H NMR (400 MHz, METHANOL-d₄) δ 8.41-8.51 (m, 1H), |
| | | 8.25-8.31 (m, 1H), 8.06-8.13 (m, 1H), 7.78-7.83 (m, 1H), |
| | | 7.72-7.76 (m, 1H), 7.63-7.69 (m, 1H), 7.27-7.35 (m, 1H), |
| | | 6.98 (s, 3H), 6.86-6.92 (m, 1H), 4.85 (s, 2H), 4.67 (s, |
| | | 2H), 4.54 (s, 2H); LCMS: Calculated: for C24H19N3O4: |
| | | 413, Measured: 414 [M + H]+. |
| 59 | 18 | 1H NMR (400 MHz, DMSO-d6) d 12.99 (brs, 1H), 8.34 |
| | | (brs, 1H), 8.03 (brs, 1H), 7.92 (s, 1H), 7.84-7.86 (m, |
| | | 1H), 7.54 (d, J = 7.8 Hz, 1H), 7.28 (t, J = 8.2 Hz, 1H), |
| | | 6.87-6.91 (m, 3H), 5.53 (dd, J = 8.9, 5.3 Hz, 1H), 4.53 |
| | | (d, J = 17.5 Hz, 1H), 4.31 (d, J = 17.5 Hz, 1H), 4.03 (t, |
| | | J = 9.7 Hz, 1H), 3.87 (dd, J = 10.6, 5.2 Hz, 1H), 3.74 (s, |
| | | 3H), 3.33 (s, 3H); LCMS: Calculated: for C21H21N3O3: |
| | | 363, Measured: 364 [M + H]+. |
| 60 | 20 | 1H NMR (400 MHz, DMSO-d6) d 12.79 (brs, 2H), 8.17 |
| | | (s, 2H), 7.91 (d, J = 1.6 Hz, 1H), 7.83 (dd, J = 7.9, 1.7 |
| | | Hz, 1H), 7.53 (d, J = 7.9 Hz, 1H), 7.26 (t, J = 7.9 Hz, 1H), |
| | | 6.84-6.92 (m, 3H), 5.53 (dd, J = 8.7, 5.0 Hz, 1H), 4.68 |
| | | (d, J = 17.7 Hz, 1H), 4.28 (d, J = 17.7 Hz, 1H), 4.14- |
| | | 4.22 (m, 1H), 4.11 (s, 2H), 4.00 (dd, J = 10.2, 5.1 Hz, |
| | | 1H), 3.72 (s, 3H); LCMS Calculated: for C22H21N3O5: |
| | | 407, Measured: 408 [M + H]+. |
| 61 | 76 | 1H NMR (400 MHz, DMSO-d6) d 12.72 (brs, 2H), 8.18 |
| | | (brs, 2H), 7.93 (d, J = 1.6 Hz, 1H), 7.84 (dd, J = 7.9, 1.7 |
| | | Hz, 1H), 7.52 (d, J = 8.0 Hz, 1H), 7.23 (t, J = 7.8 Hz, 1H), |
| | | 6.80-6.84 (m, 3H), 5.03 (d, J = 15.5 Hz, 1H), 4.68 (dd, |
| | | J = 7.2, 4.7 Hz, 1H), 4.36 (d, J = 15.5 Hz, 1H), 3.71 (s, 3H), |
| | | 3.00 (dd, J = 16.3, 4.7 Hz, 1H), 2.63 (dd, J = 16.3, 7.2 |
| | | Hz, 1H); LCMS Calculated: for C21H19N3O4: 377, |
| | | Measured: 378 [M + H]+. |
| 62 | 21 | 1H NMR (400 MHz, DMSO-d6) d 12.97 (brs, 1H), 8.31 |
| | | (brs, 1H), 8.03 (brs, 1H), 7.90 (d, J = 1.6 Hz, 1H), 7.84 |
| | | (dd, J = 7.9, 1.7 Hz, 1H), 7.53 (d, J = 7.9 Hz, 1H), 7.26 (t, |
| | | J = 8.1 Hz, 1H), 7.10-7.23 (m, 2H), 6.85-6.90 (m, 3H), |
| | | 5.56 (dd, J = 9.1, 5.0 Hz, 1H), 4.65 (d, J = 17.7 Hz, 1H), |
| | | 4.29 (d, J = 17.7 Hz, 1H), 4.17 (t, J = 9.7 Hz, 1H), 3.98 |
| | | (dd, J = 10.4, 5.0 Hz, 1H), 3.90 (s, 2H), 3.72 (s, 3H); |
| | | LCMS: Calculated: for C22H22N4O4: 406, Measured: |
| | | 407 [M + H]+ |
| 63 | 22 | 1H NMR (400 MHz, DMSO-d6) d 12.98 (brs, 1H), 8.33 |
| | | (brs, 1H), 8.02 (brs, 1H), 7.91 (d, J = 1.6 Hz, 1H), 7.85 |
| | | (dd, J = 7.9, 1.7 Hz, 1H), 7.54 (d, J = 7.9 Hz, 1H), 7.27 (t, |
| | | J = 8.2 Hz, 1H), 6.86-6.91 (m, 3H), 5.50 (dd, J = 8.8, 5.2 |
| | | Hz, 1H), 4.57-4.62 (m, 2H), 4.35 (d, J = 17.7 Hz, 1H), |
| | | 4.09 (dd, J = 10.6, 8.8 Hz, 1H), 3.96 (dd, J = 10.6, 5.2 |
| | | Hz, 1H), 3.74 (s, 3H), 3.49-3.57 (m, 4H); LCMS: |
| | | Calculated: for C22H23N3O4: 393, Measured: 394 |
| | | [M + H]+ |
| 64 | | 1H NMR (400 MHz, METHANOL-d₄) δ 8.61 (s, 1H), 8.45 |
| | | (d, J = 8.08 Hz, 1H), 8.34 (d, J = 6.57 Hz, 1H), 7.74 (d, |
| | | J = 8.08 Hz, 1H), 7.57 (d, J = 7.07 Hz, 1H), 7.24-7.33 (m, |
| | | 1H), 6.92-7.00 (m, 2H), 6.83-6.92 (m, 1H), 5.52 (dd, |
| | | J = 5.05, 9.09 Hz, 1H), 4.78 (d, J = 18.69 Hz, 1H), 4.46 (d, |
| | | J = 18.19 Hz, 1H), 4.18-4.30 (m, 1H), 4.05-4.18 (m, 1H), |
| | | 3.71-3.83 (m, 3H); LCMS: Calculated: for C21H20N4O3: |
| | | 375, Measured: 377 [M + 2H]+. |
| 65 | 80 | 1H NMR (400 MHz, DMSO-d6) d 8.23 (brs, 2H), 8.00 (s, |
| | | 1H), 7.93 (dd, J = 8.0, 1.7 Hz, 1H), 7.67 (d, J = 7.9 Hz, |
| | | 1H), 7.24 (t, J = 7.8 Hz, 1H), 6.82-6.89 (m, 3H), 5.05 (d, |
| | | J = 15.4 Hz, 1H), 4.68 (t, J = 4.1 Hz, 1H), 4.41 (d, J = |
| | | 15.4 Hz, 1H), 3.93-4.01 (m, 2H), 3.43 (s, 2H), 1.29 (t, |
| | | J = 7.0 Hz, 3H)./19F NMR (376 MHz, DMSO-d6) d −74.13; |
| | | LCMS Calculated: for C21H19N7O2: 472, Measured: |
| | | 473 [M + H]+. |
| 66 | | ¹H NMR (400 MHz, METHANOL-d₄) δ 8.86-8.96 (m, 2H), |
| | | 8.43-8.50 (m, 2H), 8.35-8.40 (m, 1H), 8.18-8.24 (m, 1H), |
| | | 7.78-7.85 (m, 1H), 7.23-7.32 (m, 1H), 6.90 (s, 3H), 4.83 |
| | | (s, 2H), 4.53 (s, 2H), 3.77 (s, 3H); LCMS Calculated: for |
| | | C21H18N2O2: 330, Measured: 331 [M + H]+. |
| 67 | | ¹H NMR (400 MHz, METHANOL-d₄) δ 8.02 (s, 2H), 7.96 |
| | | (s, 1H), 7.75-7.81 (m, 1H), 7.46 (d, J = 8.08 Hz, 1H), 7.26- |
| | | 7.33 (m, 1H), 6.99-7.06 (m, 2H), 6.89 (dd, J = 1.77, 8.34 |
| | | Hz, 1H), 6.03 (s, 1H), 3.93 (d, J = 17.68 Hz, 1H), 3.79 (s, |
| | | 3H); LCMS Calculated: for C20H17N3O4: 363, |
| | | Measured: 364 [M + H]+. |
| 68 | | ¹H NMR (400 MHz, METHANOL-d₄) δ 8.16 (br s, 2H), |
| | | 7.98 (s, 1H), 7.83 (d, J = 8.08 Hz, 1H), 7.51 (d, J = 8.08 Hz, |
| | | 1H), 7.34-7.45 (m, 2H), 7.27 (t, J = 7.83 Hz, 1H), 6.98 (d, |
| | | J = 7.58 Hz, 1H), 4.79 (s, 2H), 4.39 (s, 2H), 3.70 (s, 3H); |
| | | LCMS Calculated: for C20H18N4O3: 362, Measured: |
| | | 363 [M + H]+. |
| 69 | 4 | 1H NMR (400 MHz, METHANOL-d₄) δ 8.12 (br s, 2H), |
| | | 7.98 (d, J = 1.01 Hz, 1H), 7.83 (dd, J = 1.52, 8.08 Hz, 1H), |
| | | 7.52 (d, J = 8.08 Hz, 1H), 7.24 (t, J = 7.74 Hz, 1H), 6.81- |
| | | 6.87 (m, 3H), 4.77 (s, 2H), 4.57 (td, J = 6.06, 12.13 Hz, |
| | | 1H), 4.39 (s, 2H), 2.01-2.04 (m, 1H), 1.27 (d, J = 6.06 Hz, |
| | | 6H); LCMS: Calculated: for C21H21N3O2: 347, |
| | | Measured: 348 [M + H]+. |
| 70 | | ¹H NMR (400 MHz, METHANOL-d₄) δ 8.08-8.21 (m, 2H), |
| | | 7.98 (s, 1H), 7.82-7.89 (m, 1H), 7.48-7.55 (m, 1H), 7.34- |
| | | 7.41 (m, 1H), 6.97-7.02 (m, 1H), 6.92 (s, 2H), 6.14-6.16 |
| | | (m, 1H), 4.71-4.77 (m, 1H), 3.98-4.05 (m, 1H), 3.81 (s, |
| | | 6H); LCMS: Calculated: for C21H19N3O4: 377, |
| | | Measured: 378 [M + H]+. |
| 71 | | ¹H NMR (400 MHz, METHANOL-d₄) δ 8.10 (s, 2H), 7.98 |
| | | (s, 1H), 7.79-7.87 (m, 1H), 7.50 (d, J = 8.08 Hz, 1H), 7.37 |
| | | (s, 1H), 6.93-7.04 (m, 3H), 6.12 (s, 1H), 4.75 (d, J = 17.18 |
| | | Hz, 1H), 3.98 (d, J = 17.68 Hz, 1H), 3.81 (s, 3H); LCMS: |
| | | Calculated: for C20H17N3O4: 363, Measured: 364 |
| | | [M + H]+. |
| 72 | | ¹H NMR (400 MHz, METHANOL-d₄) δ 8.06-8.13 (m, 2H), |
| | | 7.95-7.99 (m, 1H), 7.81-7.85 (m, 1H), 7.48 (d, J = 7.58 Hz, |
| | | 1H), 7.33-7.40 (m, 1H), 6.98 (dd, J = 1.77, 8.34 Hz, 1H), |
| | | 6.90-6.95 (m, 2H), 6.14 (s, 1H), 4.72 (d, J = 17.68 Hz, 1H), |
| | | 3.99 (d, J = 17.18 Hz, 1H), 3.80 (d, J = 1.52 Hz, 6H); |
| | | LCMS: Calculated: for C21H19N3O4: 377, Measured: |
| | | 378 [M + H]+. |
| 73 | | ¹H NMR (400 MHz, METHANOL-d₄) δ 8.04-8.12 (m, 2H), |
| | | 7.95-8.01 (m, 1H), 7.81-7.87 (m, 1H), 7.47-7.53 (m, 1H), |
| | | 7.32-7.40 (m, 1H), 6.90 (s, 3H), 6.14 (s, 1H), 4.73 (d, |
| | | J = 17.18 Hz, 1H), 3.93-4.10 (m, 3H), 3.81 (s, 3H), 1.38 (t, |
| | | J = 6.82 Hz, 3H); LCMS: Calculated: for C22H21N3O4: |
| | | 301, Measured: 392 [M + H]+. |
| 74 | | ¹H NMR (400 MHz, METHANOL-d₄) δ 8.16 (br s, 2H), |
| | | 7.96-8.01 (m, 1H), 7.79-7.87 (m, 1H), 7.50 (s, 1H), 7.35 |
| | | (s, 1H), 6.91-7.03 (m, 3H), 6.11 (s, 1H), 4.74 (d, J = 17.18 |
| | | Hz, 1H), 4.02-4.09 (m, 2H), 3.98 (d, J = 17.18 Hz, 1H), |
| | | 1.38 (t, J = 7.07 Hz, 3H); LCMS: Calculated: for |
| | | C21H19N3O4: 377, Measured: 378 [M + H]+. |
| 75 | 90 | 1H NMR (400 MHz, METHANOL-d₄) δ 8.13 (br s, 2H), |
| | | 8.00 (d, J = 1.52 Hz, 1H), 7.92 (dd, J = 2.02, 8.08 Hz, 1H), |
| | | 7.62 (d, J = 8.08 Hz, 1H), 7.19-7.28 (m, 1H), 6.88-6.97 (m, |
| | | 2H), 6.83 (dd, J = 2.02, 9.09 Hz, 1H), 4.72-4.86 (m, 2H), |
| | | 4.02-4.22 (m, 2H), 3.90-4.02 (m, 1H), 3.80-3.87 (m, 1H), |
| | | 3.76 (s, 3H), 2.25-2.40 (m, 1H); LCMS: Calculated: for |
| | | C22H21N3O3: 375; Measured: 376 [M + H]+. |
| 76 | | ¹H NMR (400 MHz, METHANOL-d₄) δ 8.83-8.96 (m, 2H), |
| | | 8.40-8.47 (m, 2H), 8.33-8.39 (m, 1H), 8.15-8.23 (m, 1H), |
| | | 7.73-7.82 (m, 1H), 7.32-7.43 (m, 1H), 6.93-7.05 (m, 2H), |
| | | 6.92 (s, 1H), 6.17 (s, 1H), 4.85 (d, J = 18.19 Hz, 1H), |
| | | 4.15 (d, J = 18.19 Hz, 1H), 4.05 (qd, J = 3.41, 6.95 Hz, 2H), |
| | | 3.82 (s, 3H), 1.39 (t, J = 7.07 Hz, 3H); LCMS: Calculated: |
| | | for C24H22N2O4: 402, Measured: 403 [M + H]+. |
| 77 | | ¹H NMR (400 MHz, METHANOL-d₄) δ 8.84-8.97 (m, 2H), |
| | | 8.43 (br d, J = 4.55 Hz, 2H), 8.35 (s, 1H), 8.18 (d, J = 8.08 |
| | | Hz, 1H), 7.77 (d, J = 8.08 Hz, 1H), 7.36 (t, J = 7.83 Hz, 1H), |
| | | 6.93-7.03 (m, 3H), 6.10-6.19 (m, 1H), 4.88 (d, J = 18.69 |
| | | Hz, 1H), 4.14 (s, 1H), 4.05 (dq, J = 4.04, 6.74 Hz, 2H), |
| | | 1.34-1.44 (m, 3H); LCMS: Calculated: for C23H20N2O4: |
| | | 388, Measured: 389 [M + H]+. |
| 78 | 82 | 1H NMR (400 MHz, METHANOL-d₄) δ 8.06 (s, 1H), 7.89- |
| | | 7.94 (m, 1H), 7.81-7.89 (m, 1H), 7.42-7.53 (m, 1H), 7.18- |
| | | 7.28 (m, 1H), 7.08-7.17 (m, 1H), 6.98-7.08 (m, 1H), 6.77- |
| | | 6.89 (m, 1H), 5.94-6.02 (m, 1H), 5.77-5.89 (m, 1H), 4.75- |
| | | 4.83 (m, 2H), 4.48-4.60 (m, 1H), 3.85-3.96 (m, 1H), 3.76 |
| | | (s, 3H), 3.10-3.23 (m, 1H), 2.86-3.01 (m, 2H), 2.50-2.75 |
| | | (m, 2H); LCMS: Calculated: for C24H23N3O3: 401;, |
| | | Measured: 402 [M + H]+. |
| 79 | 84 | 1H NMR (400 MHz, METHANOL-d₄) δ 8.10 (br s, 2H), |
| | | 7.82-7.94 (m, 2H), 7.53 (d, J = 8.08 Hz, 1H), 7.19-7.28 (m, |
| | | 1H), 7.10-7.16 (m, 1H), 7.04 (d, J = 7.58 Hz, 1H), 6.84 (s, |
| | | 1H), 4.73 (d, J = 11.12 Hz, 1H), 4.59-4.68 (m, 1H), 4.02 |
| | | (dd, J = 4.80, 11.37 Hz, 1H), 3.76 (s, 3H), 2.42-2.61 (m, |
| | | 1H), 1.86-2.14 (m, 6H), 1.69-1.81 (m, 1H); LCMS: |
| | | Calculated: for C24H25N3O3: 403; Measured: 404 |
| | | [M + H]+. |
| 80 | 15 | 1H NMR (300 MHz, DMSO-d6) d 12.97 (s, 1H), 8.31 (s, |
| | | 1H), 8.01 (s, 1H), 7.92-7.77 (m, 2H), 7.51 (d, J = 7.9 |
| | | Hz, 1H), 7.26 (t, J = 7.8 Hz, 1H), 6.97-6.80 (m, 3H), |
| | | 5.49 (m, 1H), 4.54 (d, J = 17.6 Hz, 1H), 4.23 (d, J = 17.6 |
| | | Hz, 1H), 3.72 (s, 3H), 3.21-3.06 (m, 1H), 2.58 (m, 1H), |
| | | 2.21 (s, 6H); LCMS: Calculated: for C22H24N4O2: 376, |
| | | Measured: 377 [M + H]+. |
| 81 | 86 | 1H NMR (400 MHz, CHLOROFORM-d) δ 8.13 (d, J = 1.52 |
| | | Hz, 1H), 7.80-7.89 (m, 1H), 7.64-7.74 (m, 1H), 7.16-7.25 |
| | | (m, 1H), 6.82-6.93 (m, 1H), 6.74-6.82 (m, 1H), 5.03-5.38 |
| | | (br s, 2H), 4.79 (s, 2H), 3.77 (s, 3H), 1.90 (br d, J = 3.54 |
| | | Hz, 9H), 1.17-1.53 (m, 4H); LCMS: Calculated: for |
| | | C24H25N3O2: 387; Measured: 388 [M + H]+. |
| 82 | 89 | 1H NMR (400 MHz, CHLOROFORM-d) δ 8.12 (d, J = 1.52 |
| | | Hz, 1H), 7.89-8.05 (m, 2H), 7.67-7.79 (m, 1H), 7.15-7.27 |
| | | (m, 1H), 6.84-6.96 (m, 2H), 6.72-6.80 (m, 1H), 4.75-4.87 |
| | | (m, 1H), 4.81 (s, 2H), 3.98-4.13 (m, 4H), 3.77 (s, 3H), |
| | | 2.20-2.39 (m, 2H), 1.41 (br d, J = 14.15 Hz, 2H), 1.25 (s, |
| | | 1H); LCMS: Calculated: for C23H23N3O3: 389; |
| | | Measured: 390 [M + H]+. |
| 83 | 30 | 1H NMR (400 MHz, DMSO-d6) d 8.84-8.76 (m, 2H), |
| | | 8.20-8.07 (m, 4H), 7.77 (d, J = 8.0 Hz, 1H), 7.26 (t, J = |
| | | 7.8 Hz, 1H), 6.93-6.82 (m, 3H), 5.36 (m, 1H), 4.71 (d, |
| | | J = 18.3 Hz, 1H), 4.41 (d, J = 18.2 Hz, 1H), 4.08-3.93 (m, |
| | | 2H), 3.72 (s, 3H); LCMS: Calculated: for C22H20N2O3: |
| | | 360, Measured: 361 [M + H]+. |
| 84 | | 1H NMR (400 MHz, DMSO-d6) d 8.17 (brs, 2H), 7.85 (s, |
| | | 1H), 7.81 (dd, J = 7.9, 1.7 Hz, 1H), 7.32 (d, J = 8.0 Hz, |
| | | 1H), 7.26 (t, J = 7.7 Hz, 1H), 6.81-6.87 (m, 3H), 5.17 (d, |
| | | J = 15.5 Hz, 1H), 4.84 (dd, J = 6.0, 4.4 Hz, 1H), 4.41 (d, |
| | | J = 15.5 Hz, 1H), 3.73 (s, 3H), 3.68 (d, J = 4.1 Hz, 1H), |
| | | 3.54-3.63 (m, 1H); LCMS Calculated: for C21H19N7O2: |
| | | 401, Measured: 402 [M + H]+. |
| 85 | | ¹H NMR (400 MHz, METHANOL-d₄) δ 8.11-8.31 (m, 2H), |
| | | 7.99 (s, 1H), 7.85 (d, J = 8.08 Hz, 1H), 7.51 (d, J = 7.58 Hz, |
| | | 1H), 7.34-7.39 (m, 2H), 7.18-7.31 (m, 4H), 6.86-6.97 (m, |
| | | 3H), 5.07 (s, 2H), 4.77 (s, 2H), 4.30 (s, 2H); LCMS |
| | | Calculated: for C25H21N3O2: 395, Measured: 396 |
| | | [M + H]+. |
| 86 | 19 | 1H NMR (400 MHz, DMSO-d6) d 12.99 (s, 1H), 8.33 (s, |
| | | 1H), 8.04 (d, J = 17.5 Hz, 1H), 7.93 (s, 1H), 7.90-7.80 |
| | | (m, 1H), 7.53 (d, J = 7.9 Hz, 1H), 7.28 m, 3H), 7.02- |
| | | 6.85 (m, 6H), 5.71 (t, J = 6.9 Hz, 1H), 4.60 (m, 3H), 4.33 |
| | | (d, J = 17.6 Hz, 1H), 3.73 (s, 3H), 2.48 (s, 2H); LCMS: |
| | | Calculated: for C26H23N3O3: 425, Measured: 426 |
| | | [M + H]+. |
| 87 | 10 | 1H NMR (300 MHz, DMSO-d6) d 12.97 (s, 1H), 8.32 (s, |
| | | 1H), 7.98-8.06 (m, 1H), 7.93 (d, J = 1.7 Hz, 1H), 7.83 |
| | | (dd, J = 7.9, 1.7 Hz, 1H), 7.51 (d, J = 8.0 Hz, 1H), 7.27 (t, |
| | | J = 7.9 Hz, 1H), 6.85-6.91 (m, 3H), 4.70-4.76 (m, 4H), |
| | | 4.34 (s, 2H); LCMS: Calculated: for C21H17N3O2: 343, |
| | | Measured: 344 [M + H]+. |
| 88 | 11 | 1H NMR (400 MHz, DMSO-d6) d 12.97 (s, 1H), 8.05- |
| | | 8.37 (m, 2H), 7.96 (s, 1H), 7.86 (d, J = 7.9 Hz, 1H), 7.54 |
| | | (d, J = 7.9 Hz, 1H), 7.33 (t, J = 7.8 Hz, 1H), 6.91-7.01 |
| | | (m, 3H), 5.49 (s, 2H), 4.73 (s, 2H), 4.37 (s, 2H); LCMS: |
| | | Calculated: for C20H17N7O2 387, Measured: 388 |
| | | [M + H]+. |
| 89 | 87 | 1H NMR (400 MHz, METHANOL-d₄) δ 7.96-8.14 (m, 2H), |
| | | 7.76-7.85 (m, 1H), 7.20-7.30 (m, 1H), 7.08-7.15 (m, 1H), |
| | | 6.85-6.95 (m, 3H), 6.64-6.80 (m, 2H), 5.95-6.07 (m, 1H), |
| | | 4.73-4.86 (m, 1H), 3.96-4.13 (m, 1H), 3.72 (s, 3H), 2.92- |
| | | 3.16 (m, 3H), 2.41-2.56 (m, 1H), 2.27-2.41 (m, 1H); |
| | | LCMS: Calculated: for C27H23N3O3: 437; Measured: |
| | | 438 [M + H]+. |
| 90 | | ¹H NMR (400 MHz, METHANOL-d₄) δ 8.11-8.33 (m, 1H), |
| | | 7.98 (s, 1H), 7.82 (s, 1H), 7.52 (d, J = 7.58 Hz, 1H), 7.22- |
| | | 7.32 (m, 1H), 6.88-6.94 (m, 1H), 6.80-6.87 (m, 2H), 4.97- |
| | | 5.01 (m, 1H), 4.78 (s, 2H), 4.38 (s, 2H), 3.79-3.97 (m, |
| | | 4H), 2.15-2.28 (m, 1H), 2.00-2.12 (m, 1H); LCMS: |
| | | Calculated: for C22H21N3O3: 375; Measured: 376 |
| | | [M + H]+. |
| 91 | | ¹H NMR (400 MHz, METHANOL-d₄) δ 8.10-8.29 (m, 2H), |
| | | 7.99 (s, 1H), 7.81-7.87 (m, 1H), 7.53 (d, J = 8.08 Hz, 1H), |
| | | 7.27 (t, J = 7.83 Hz, 1H), 6.93 (d, J = 7.58 Hz, 1H), 6.63- |
| | | 6.76 (m, 2H), 5.24 (t, J = 5.56 Hz, 1H), 4.97 (t, J = 6.57 Hz, |
| | | 2H), 4.78 (s, 2H), 4.62-4.68 (m, 2H), 4.39 (s, 2H); |
| | | LCMS: Calculated: for C21H19N3O3: 361; Measured: |
| | | 362 [M + H]+. |
| 93 | 24 | 1H NMR (400 MHz, DMSO-d6) d 12.98 (s, 1 H), 8.34 (s, |
| | | 1 H), 7.71-8.18 (m, 4 H), 7.20-7.70 (m, 6 H), 6.99-7.07 |
| | | (m, 2 H), 6.90-6.98 (m, 2 H), 4.61 (d, J = 17.4 Hz, 1 H), |
| | | 3.91 (d, J = 17.5 Hz, 1 H), 3.73 (s, 3 H); LCMS: |
| | | Calculated: for C26H21N3O3: 423, Measured: 424 |
| | | [M + H]+. |
| 94 | | 1H NMR (400 MHz, DMSO-d6) d 8.17 (brs, 2H), 7.85 (s, |
| | | 1H), 7.81 (dd, J = 7.9, 1.7 Hz, 1H), 7.32 (d, J = 8.0 Hz, |
| | | 1H), 7.26 (t, J = 7.7 Hz, 1H), 6.81-6.87 (m, 3H), 5.17 (d, |
| | | J = 15.5 Hz, 1H), 4.84 (dd, J = 6.0, 4.4 Hz, 1H), 4.41 (d, |
| | | J = 15.5 Hz, 1H), 3.73 (s, 3H), 3.68 (d, J = 4.1 Hz, 1H), |
| | | 3.54-3.63 (m, 1H); LCMS Calculated: for C21H19N7O2: |
| | | 401, Measured: 402 [M + H]+. |
| 95 | 23 | 1H NMR (300 MHz, DMSO-d6) d 12.98 (s, 1H), 8.28- |
| | | 8.36 (m, 1H), 8.02 (s, 1H), 7.94 (d, J = 1.6 Hz, 1H), 7.85 |
| | | (dd, J = 7.8, 1.7 Hz, 1H), 7.54 (d, J = 8.0 Hz, 1H), 7.30 (t, |
| | | J = 8.2 Hz, 1H), 6.87-6.99 (m, 2H), 6.74-6.82 (m, 2H), |
| | | 4.72 (d, J = 17.6 Hz, 1H), 4.17 (d, J = 17.7 Hz, 1H), 3.70 |
| | | (s, 3H); LCMS: Calculated: for C20H17N7O2: 387, |
| | | Measured: 388 [M + H]+. |
| 97 | | ¹H NMR (400 MHz, METHANOL-d₄) δ 8.17 (br s, 2H), |
| | | 7.98 (s, 1H), 7.85 (d, J = 8.08 Hz, 1H), 7.61-7.69 (m, 2H), |
| | | 7.52-7.60 (m, 2H), 7.28 (t, J = 8.08 Hz, 1H), 6.97-7.05 (m, |
| | | 2H), 6.81-6.89 (m, 1H), 4.60-4.74 (m, 2H), 4.22-4.33 (m, |
| | | 1H), 3.70-3.82 (m, 3H), 1.55-1.72 (m, 1H), 0.88 (br d, |
| | | J = 4.04 Hz, 1H), 0.63-0.73 (m, 1H), 0.42-0.60 (m, 2H); |
| | | LCMS: Calculated: for C22H21N3O2: 359, Measured: |
| | | 360 [M + H]+. |
| 98 | 77/78 | 1H NMR (300 MHz, DMSO-d6) d 8.75 (d, J = 11.2 Hz, |
| | | 2H), 7.87 (d, J = 7.5 Hz, 2H), 7.47 (d, J = 8.0 Hz, 1H), |
| | | 7.12 (t, J = 7.9 Hz, 1H), 6.76-6.98 (m, 2H), 6.70 (dd, J = |
| | | 8.1, 2.6 Hz, 1H), 5.15 (t, J = 7.4 Hz, 1H), 4.87 (dd, J = |
| | | 6.6, 3.7 Hz, 1H), 3.57 (s, 3H), 2.74 (dd, J = 16.7, 3.9 Hz, |
| | | 1H), 2.29 (dd, J = 16.8, 7.3 Hz, 1H), 1.63 (d, J = 7.1 Hz, |
| | | 3H); LCMS Calculated: for C22H21N3O4: 391, |
| | | Measured: 392 [M + H]+. |
| 99 | 77/78 | 1H NMR (300 MHz, DMSO-d6) d 8.63 (d, J = 25.1 Hz, |
| | | 3H), 7.75-8.00 (m, 2H), 7.45 (d, J = 8.0 Hz, 2H), 7.19 |
| | | (ddd, J = 10.7, 7.2, 3.0 Hz, 2H), 6.71-6.97 (m, 6H), 5.23 |
| | | (dd, J = 7.1, 3.1 Hz, 2H), 4.58 (dd, J = 6.8, 3.7 Hz, 1H), |
| | | 3.62 (d, J = 2.3 Hz, 3H), 3.00 (dd, J = 16.8, 3.8 Hz, 1H), |
| | | 2.51-2.63 (m, 1H), 1.48-1.70 (m, 3H); LCMS |
| | | Calculated: for C22H21N3O4: 391, Measured: 392 |
| | | [M + H]+. |
| 100 | | ¹H NMR (400 MHz, METHANOL-d₄) δ 8.08 (s, 2H), 7.94 |
| | | (s, 1H), 7.79 (d, J = 7.58 Hz, 1H), 7.47 (d, J = 7.58 Hz, 1H), |
| | | 7.27 (t, J = 7.83 Hz, 1H), 6.94 (d, J = 7.58 Hz, 1H), 6.91 (s, |
| | | 1H), 6.85 (dd, J = 2.02, 8.08 Hz, 1H), 5.64 (q, J = 7.07 Hz, |
| | | 1H), 4.50 (d, J = 18.19 Hz, 1H), 4.08 (d, J = 17.68 Hz, 1H), |
| | | 3.76 (s, 3H), 1.69 (d, J = 7.07 Hz, 3H); LC/MS: Calculated: |
| | | for C20H19N3O2: 333, Measured: 334 [M + H]+. |
| 101 | 26/27 | 1H NMR (300 MHz, DMSO-d6) d 12.69-12.72 (m, 2H), |
| | | 8.13 (s, 2H), 7.70-7.75 (m, 2H), 7.62 (d, J = 7.9 Hz, |
| | | 1H), 7.23-7.26 (m, 1H), 6.83-6.94 (m, 3H), 5.69 (t, J = |
| | | 7.8 Hz, 1H), 4.50 (d, J = 17.5 Hz, 1H), 4.14 (d, J = 17.6 |
| | | Hz, 1H), 3.72 (s, 3H), 3.07-3.13 (m, 2H); LC/MS: |
| | | Calculated: for C21H19N3O4: 377, Measured: 378 |
| | | [M + H]+. |
| 102 | 100 | ¹H NMR (300 MHz, DMSO-d6) d 8.16 (s, 2H), 7.89- |
| | | 7.83 (m, 2H), 7.82-7.77 (m, 1H), 7.28-7.15 (m, 1H), |
| | | 6.94-6.74 (m, 3H), 4.79-4.57 (m, 2H), 4.19 (d, J = 4.7 |
| | | Hz, 1H), 4.11 (d, J = 4.9 Hz, 1H), 3.70 (s, 3H), 2.29- |
| | | 1.91 (m, 2H), 1.90-1.71 (m, 2H); LCMS: Calculated: for |
| | | C23H23N3O4: 405, Measured: 406 [M + H]+. |
| 103 | | ¹H NMR (400 MHz, METHANOL-d₄) δ 8.08 (s, 2H), 7.96 |
| | | (s, 1H), 7.84 (d, J = 8.08 Hz, 1H), 7.52 (d, J = 7.58 Hz, 1H), |
| | | 7.32 (q, J = 7.92 Hz, 3H), 7.04-7.12 (m, 2H), 6.93-7.00 (m, |
| | | 3H), 6.90 (br d, J = 8.08 Hz, 1H), 4.79 (s, 2H), 4.39 (s, |
| | | 2H); LCMS: Calculated: for C24H19N3O2: 381, |
| | | Measured: 382 [M + H]+. |
| 104 | | ¹H NMR (400 MHz, METHANOL-d₄) δ 8.07 (s, 2H), 7.96 |
| | | (s, 1H), 7.80-7.86 (m, 1H), 7.50-7.56 (m, 1H), 7.30-7.42 |
| | | (m, 4H), 7.06-7.13 (m, 1H), 6.97 (br d, J = 8.08 Hz, 4H), |
| | | 5.63-5.73 (m, 1H), 4.54 (d, J = 18.19 Hz, 1H), 4.14 (d, |
| | | J = 17.68 Hz, 1H), 1.72 (d, J = 7.07 Hz, 3H); LCMS: |
| | | Calculated: for C25H21N3O2: 395, Measured: 396 |
| | | [M + H]+. |
| 105 | | ¹H NMR (400 MHz, CHLOROFORM-d) δ 8.36-8.44 (m, |
| | | 1H), 7.97 (s, 1H), 7.88 (s, 2H), 7.64 (s, 1H), 7.38 (d, |
| | | J = 7.58 Hz, 1H), 6.88 (d, J = 1.52 Hz, 1H), 6.77 (br d, |
| | | J = 5.56 Hz, 1H), 4.91-4.97 (m, 2H), 4.46 (s, 2H), 3.82- |
| | | 3.86 (m, 3H); LCMS: Calculated: for C18H16N4O2: 320, |
| | | Measured: 321 [M + H]+. |
| 106 | | ¹H NMR (400 MHz, METHANOL-d₄) δ 9.25 (br s, 1H), |
| | | 8.81-8.98 (m, 1H), 8.58 (s, 1H), 8.45 (d, J = 8.08 Hz, 1H), |
| | | 8.11 (d, J = 5.05 Hz, 1H), 7.71 (d, J = 8.08 Hz, 1H), 7.28- |
| | | 7.39 (m, 3H), 7.05-7.14 (m, 2H), 6.94-7.01 (m, 3H), 6.90 |
| | | (br d, J = 8.08 Hz, 1H), 4.82 (s, 2H), 4.49 (s, 2H); LCMS: |
| | | Calculated: for C25H19N3O2: 393, Measured: 394 |
| | | [M + H]+. |
| 107 | 96 | 1H NMR (300 MHz, DMSO-d6) d 8.18 (d, J = 2.1 Hz, |
| | | 2H), 7.85-7.90 (m, 2H), 7.61(dd, J = 27.0, 7.9 Hz, 1H), |
| | | 7.22 (td, J = 7.8, 4.2 Hz, 1H), 6.78-6.88 (m, 3H), 4.61- |
| | | 4.77 (m, 2H), 3.70 (s, 3H), 3.18 (dp, J = 25.8, 8.5 Hz, |
| | | 1H), 1.70-2.34 (m, 6H); LCMS: Calculated: for |
| | | C24H23N3O4: 417, Measured: 418 [M + H]+. |
| 108 | 97 | 1H NMR (300 MHz, DMSO-d6) d 8.18 (d, J = 3.2 Hz, |
| | | 2H), 7.91 (ddd, J = 8.6, 4.7, 1.6 Hz, 2H), 7.64 (dd, J = |
| | | 20.0, 8.2 Hz, 1H), 7.38 (s, 1H), 7.24 (q, J = 7.8 Hz, 1H), |
| | | 6.79-6.89 (m, 4H), 4.57-4.81 (m, 2H), 3.04 (dt, J = |
| | | 28.0, 8.2 Hz, 1H), 1.71-2.33 (m, 6H); LCMS: |
| | | Calculated: for C24H24N4O3: 416, Measured: 417 |
| | | [M + H]+. |
| 109 | 95 | 1H NMR (300 MHz, DMSO-d6) d 8.18 (d, J = 1.7 Hz, |
| | | 2H), 7.89 (d, J = 7.7 Hz, 2H), 7.57-7.63 (m, 1H), 7.44 (d, |
| | | J = 7.7 Hz, 1H), 7.22 (dd, J = 8.8, 7.5 Hz, 1H), 6.79- |
| | | 6.88 (m, 3H), 4.56-4.81 (m, 2H), 4.23 (q, J = 7.7 Hz, |
| | | 1H), 3.70 (d, J = 1.8 Hz, 3H), 3.48 (d, J = 1.7 Hz, 3H), |
| | | 1.56-2.26 (m, 6H); LCMS: Calculated: for |
| | | C25H26N4O4: 446, Measured: 447 [M + H]+. |
| 110 | 102 | 1H NMR (300 MHz, DMSO) d 8.11-8.20 (m, 2 H), 7.85 |
| | | (t d, J = 3.6, 1.5 Hz, 1 H), 7.77 (d d, J = 8.0, 1.7 Hz, 1 H), |
| | | 7.52 (d d, J = 59.3, 8.3 Hz, 1 H), 7.11-7.23 (m, 1 H), |
| | | 6.70-6.94 (m, 3 H), 4.93 (dd, J = 39.8, 16.5 Hz, 1 H), |
| | | 4.38 (dd, J = 99.2, 16.6 Hz, 1 H), 3.66 (s, 3 H), 3.38 (td, |
| | | J = 10.9, 8.7 Hz, 1 H), 1.84-2.36 (m, 4 H), 1.60-1.84 (m, |
| | | 2 H); LCMS: Calculated: for C24H23N3O4: 417, |
| | | Measured: 418 [M + H]+. |
| 111 | 103 | 1H NMR (300 MHz, DMSO) d 8.15 (d, J = 10.2 Hz, 2 H), |
| | | 7.69-7.90 (m, 2 H), 7.48 (dd, J = 49.0, 8.2 Hz, 1 H), |
| | | 7.17 (q, J = 7.7 Hz, 1 H), 6.67-6.91 (m, 3 H), 6.44- |
| | | 6.56 (m, 2 H), 5.00 (dd, J = 16.7, 8.4 Hz, 1 H), 4.44 (t, J = |
| | | 16.3 Hz, 1 H), 3.66 (d, J = 3.5 Hz, 3 H), 3.06-3.19 (m, 1 |
| | | H), 2.14-2.39 (m, 1 H), 1.56-2.11 (m, 5 H); LCMS: |
| | | Calculated: for C24H24N4O3: 416, Measured: 417 |
| | | [M + H]+. |
| 112 | | ¹H NMR (400 MHz, METHANOL-d₄) δ 8.13 (br s, 2H), |
| | | 7.97 (s, 1H), 7.83 (br d, J = 7.58 Hz, 1H), 7.52-7.58 (m, |
| | | 1H), 7.27 (br t, J = 7.83 Hz, 1H), 6.89-6.97 (m, 2H), 6.85 |
| | | (br d, J = 8.59 Hz, 1H), 5.44-5.56 (m, 1H), 4.66 (br d, |
| | | J = 17.68 Hz, 1H), 4.34 (br d, J = 17.68 Hz, 1H), 4.17-4.25 |
| | | (m, 1H), 4.08-4.16 (m, 1H), 4.01 (q, J = 7.07 Hz, 2H), |
| | | 1.31-1.43 (m, 3H); LCMS: Calculated: for C21H21N3O3: |
| | | 363, Measured: 364 [M + H]+. |
| 113 | | 1H NMR (300 MHz, DMSO-d6) d 12.94 (s, 1 H), 8.29 (s, |
| | | 1 H), 8.00 (s, 1 H), 7.88 (d, J = 1.6 Hz, 1 H), 7.81 (dd, J = |
| | | 8.0, 1.7 Hz, 1 H), 7.51 (d, J = 7.9 Hz, 1 H), 7.23 (td, J = |
| | | 7.3, 2.0 Hz, 1 H), 6.78-6.89 (m, 3 H), 5.31 (dd, J = 8.3, |
| | | 5.8 Hz, 1 H), 5.06 (t, J = 5.5 Hz, 1 H), 4.57 (d, J = 17.7 |
| | | Hz, 1 H), 4.26 (d, J = 17.6 Hz, 1 H), 3.93 (dq, J = 11.5, |
| | | 6.5, 5.7 Hz, 2 H), 3.69 (s, 3 H); LCMS: Calculated: for |
| | | C20H19N3O3: 349, Measured: 350 [M + H]+. |
| 114 | | ¹H NMR (400 MHz, METHANOL-d₄) δ 8.03-8.18 (m, 2H), |
| | | 7.96 (s, 1H), 7.80-7.86 (m, 1H), 7.53 (d, J = 8.08 Hz, 1H), |
| | | 7.27 (t, J = 8.08 Hz, 1H), 6.90-6.97 (m, 2H), 6.81-6.87 (m, |
| | | 1H), 5.50 (dd, J = 5.31, 8.84 Hz, 1H), 4.66 (d, J = 17.68 Hz, |
| | | 1H), 4.34 (d, J = 17.68 Hz, 1H), 4.17-4.26 (m, 1H), 4.09- |
| | | 4.15 (m, 1H), 4.01 (d, J = 6.57 Hz, 2H), 1.36 (t, J = 6.82 Hz, |
| | | 3H); LCMS: Calculated: for C21H21N3O3: 363, |
| | | Measured: 364 [M + H]+. |
| 115 | 104 | 1H NMR (300 MHz, DMSO-d6) d 8.15-8.29 (m, 2 H), |
| | | 7.81-7.95 (m, 2 H), 7.45-7.66 (m, 1 H), 7.03-7.31 |
| | | (m, 1 H), 6.58-6.89 (m, 3 H), 4.78-5.16 (m, 1 H), 4.34- |
| | | 4.61 (m, 2 H), 3.70 (s, 3 H), 3.28-3.44 (m, 3 H), 1.75- |
| | | 2.23 (m, 5 H), 1.50-1.70 (m, 1 H); LCMS: Calculated: |
| | | for C25H26N4O4: 446, Measured: 447 [M + H]+. |
| 117 | 28/29 | 1H NMR (300 MHz, DMSO-d6) d 8.13 (s, 2H), 7.71- |
| | | 7.74 (m, 2H), 7.63 (d, J = 7.9 Hz, 1H), 7.28 (t, J = 7.8 Hz, |
| | | 1H), 6.84-6.93 (m, 3H), 5.34 (dd, J = 9.1, 5.6 Hz, 1H), |
| | | 4.51 (d, J = 17.5 Hz, 1H), 4.04 (d, J = 17.6 Hz, 1H), 3.72 |
| | | (s, 3H), 2.06-2.40 (m, 4H); LCMS: Calculated: for |
| | | C22H21N3O4: 391, Measured: 392 [M + H]+ |
| 118 | 94 | 1H NMR (400 MHz, METHANOL-d₄) δ 8.10 (s, 2H), 7.90- |
| | | 8.07 (m, 2H), 7.56-7.65 (m, 1H), 7.28 (s, 1H), 6.79-7.00 |
| | | (m, 2H), 4.81-4.95 (m, 1H), 4.68-4.80 (m, 1H), 3.96-4.11 |
| | | (m, 1H), 3.76 (d, J = 5.56 Hz, 3H), 2.64 (s, 3H), 2.37-2.55 |
| | | (m, 2H), 2.09-2.34 (m, 4H), 1.88-2.09 (m, 2H); LCMS: |
| | | Calculated: for C24H26N4O2: 402; Measured: 403 |
| | | [M + H]+. |
| 119 | | ¹H NMR (400 MHz, METHANOL-d₄) δ 8.20-8.23 (m, 1H), |
| | | 7.98-8.01 (m, 1H), 7.82-7.85 (m, 1H), 7.74 (s, 3H), 7.70 |
| | | (s, 2H), 7.44-7.56 (m, 2H), 7.26-7.32 (m, 1H), 6.51 (s, |
| | | 1H), 4.90 (s, 2H), 4.45 (s, 2H); LCMS: Calculated: for |
| | | C21H17N5O: 355; Measured: 356 [M + H]+. |
| 120 | 14 | 1H NMR (400 MHz, METHANOL-d₄) δ 8.50-8.69 (m, 1 H), |
| | | 8.18 (s, 1H), 8.07 (d, J = 1.52 Hz, 1H), 7.90-7.99 (m, 1H), |
| | | 7.22-7.30 (m, 1H), 6.90-6.98 (m, 1H), 6.81-6.87 (m, 1H), |
| | | 5.66 (d, J = 7.07 Hz, 1H), 4.72 (d, J = 17.68 Hz, 1H), 4.17 |
| | | (d, J = 17.68 Hz, 1H), 3.76 (s, 3H), 1.72 (d, J = 7.07 Hz, |
| | | 3H); LCMS: Calculated: for C25H21FN4O2: 428, |
| | | Measured: 429 [M + H]+. |
| 121 | 45 | 1H NMR (400 MHz, METHANOL-d₄) δ 8.78-8.99 (m, 1H), |
| | | 8.07-8.28 (m, 3H), 7.26 (t, J = 8.08 Hz, 1H), 6.88-7.00 (m, |
| | | 1H), 6.84 (dd, J = 2.53, 8.08 Hz, 1H), 5.69 (d, J = 7.07 Hz, |
| | | 1H), 4.81 (s, 1H), 4.29 (d, J = 17.68 Hz, 1H), 3.76 (s, 3H), |
| | | 1.73 (d, J = 7.07 Hz, 3H); LCMS: Calculated: for |
| | | C25H22N4O2: 410, Measured: 411 [M + H]+ |
| 122 | 85 | 1H NMR (300 MHz, DMSO-d6) d 8.12 (s, 2H), 7.84- |
| | | 7.75 (m, 2H), 7.49 (d, J = 7.9 Hz, 1H), 7.18 (t, J = 7.9 Hz, |
| | | 1H), 7.01-6.92 (m, 2H), 6.76 (dd, J = 8.3, 2.3 Hz, 1H), |
| | | 4.67 (q, J = 6.9 Hz, 1H), 3.67 (s, 3H), 2.22 (dt, J = 12.5, |
| | | 7.5 Hz, 1H), 2.02-1.86 (m, 5H), 1.81 (d, J = 7.0 Hz, 4H), |
| | | 1.67 (t, J = 6.4 Hz, 1H); LCMS: Calculated: for |
| | | C24H25N3O2: 387, Measured: 388 [M + H]+. |
| 123 | 105 | 1H NMR (400 MHz, DMSO-d6) d 8.24 (d, J = 8.7 Hz, 2 |
| | | H), 8.02 (d, J = 1.7 Hz, 1 H), 7.90-8.00 (m, 1 H), 7.58- |
| | | 7.76 (m, 3 H), 7.02-7.32 (m, 2 H), 6.73-6.90 (m, 3 H), |
| | | 4.27-5.34 (m, 2 H), 3.99 (s, 1 H), 3.69 (d, J = 4.7 Hz, 3 |
| | | H), 1.56-2.37 (m, 6 H); LCMS: Calculated: for |
| | | C23H24N4O2: 388, Measured: 389 [M + H]+. |
| 124 | 43 | 1H NMR (400 MHz, METHANOL-d₄) δ 8.65 (br d, J = 6.57 |
| | | Hz, 1H), 8.18 (br s, 1H), 8.12 (d, J = 1.52 Hz, 1H), 8.01 (d, |
| | | J = 1.01 Hz, 1H), 7.26 (t, J = 8.08 Hz, 1H), 6.88-7.00 (m, |
| | | 1H), 6.84 (dd, J = 2.53, 8.08 Hz, 1H), 5.68 (d, J = 7.07 Hz, |
| | | 1H), 4.75 (d, J = 17.68 Hz, 1H), 4.22 (d, J = 18.19 Hz, 1H), |
| | | 3.75 (s, 3H), 1.72 (d, J = 7.07 Hz, 3H); LCMS: Calculated: |
| | | for C25H22N4O2: 410, Measured: 411 [M + H]+ |
| 125 | 46 | 1H NMR (400 MHz, METHANOL-d₄) δ 8.12 (d, J = 1.01 |
| | | Hz, 1H), 7.95 (dd, J = 1.52, 6.57 Hz, 1H), 7.85 (d, J = 1.52 |
| | | Hz, 1H), 7.24 (d, J = 8.08 Hz, 1H), 7.00-7.06 (m, 1H), 6.91 |
| | | (s, 1H), 6.79-6.86 (m, 1H), 5.61-5.74 (m, 1H), 4.34-4.50 |
| | | (m, 1H), 4.01 (d, J = 18.19 Hz, 1H), 3.75 (s, 3H), 1.68 (d, |
| | | J = 7.07 Hz, 3H); LCMS: Calculated: for C25H23N5O2: |
| | | 425, Measured: 426 [M + H]+ |
| 126 | 47 | 1H NMR (400 MHz, METHANOL-d₄) δ 8.17 (s, 1H), 8.09 |
| | | (s, 1H), 7.97 (s, 1H), 7.20-7.33 (m, 1H), 6.90-7.00 (m, |
| | | 1H), 6.79-6.90 (m, 1H), 5.61-5.71 (m, 1H), 4.67-4.76 (m, |
| | | 1H), 4.13-4.20 (m, 1H), 3.92-4.03 (m, 3H), 3.76 (s, 3H), |
| | | 1.72 (d, J = 7.07 Hz, 3H); LCMS Calculated: for |
| | | C26H24N4O3: 440, Measured: 441 [M + H]+. |
| 127 | 48 | 1H NMR (400 MHz, METHANOL-d₄) δ 8.32 (br s, 1H), |
| | | 7.99 (d, J = 1.52 Hz, 1H), 7.93 (dd, J = 2.53, 8.59 Hz, 1H), |
| | | 7.85 (d, J = 1.52 Hz, 1H), 7.26 (t, J = 7.83 Hz, 1H), 6.89- |
| | | 6.98 (m, 1H), 6.84 (dd, J = 2.53, 8.08 Hz, 1H), 5.66 (d, |
| | | J = 7.07 Hz, 1H), 4.65 (d, J = 17.68 Hz, 1H), 4.12 (d, |
| | | J = 17.68 Hz, 1H), 3.91-4.02 (m, 3H), 3.76 (s, 3H), 1.71 (d, |
| | | J = 7.07 Hz, 3H); LCMS Calculated: for C26H24N4O3: |
| | | 440, Measured: 441 [M + H]+. |
| 128 | | ¹H NMR (400 MHz, METHANOL-d₄) δ 8.05-8.12 (m, 2H), |
| | | 7.98-8.01 (m, 1H), 7.82-7.88 (m, 1H), 7.73-7.77 (m, 1H), |
| | | 7.69-7.73 (m, 1H), 7.54-7.68 (m, 1H), 7.43-7.54 (m, 3H), |
| | | 4.88 (s, 2H), 4.43 (s, 2H), 3.75-3.80 (m, 1H), 2.29 (br d, |
| | | J = 4.55 Hz, 2H), 1.72-1.81 (m, 1H), 1.45-1.59 (m, 4H), |
| | | 1.25-1.33 (m, 1H), 1.16-1.25 (m, 2H); LCMS Calculated: |
| | | for C26H26N4O2: 426, Measured: 427 [M + H]+. |
| 129 | 49 | 1H NMR (400 MHz, METHANOL-d₄) δ 8.05-8.19 (m, 1H), |
| | | 7.86-7.94 (m, 1H), 7.74-7.80 (m, 1H), 7.36 (d, J = 8.59 Hz, |
| | | 1H), 7.21-7.31 (m, 1H), 6.88 (d, J = 8.59 Hz, 3H), 5.59- |
| | | 5.72 (m, 1H), 4.54-4.72 (m, 1H), 4.03-4.16 (m, 1H), 3.76 |
| | | (s, 3H), 1.71 (d, J = 7.07 Hz, 3H); LCMS Calculated: for |
| | | C26H23N3O3: 425, Measured: 426 [M + H]+. |
| 130 | 50 | 1H NMR (400 MHz, METHANOL-d₄) δ 9.20 (s, 1H), 9.04 |
| | | (s, 2H), 8.15-8.28 (m, 2H), 8.09 (d, J = 1.52 Hz, 1H), 7.98 |
| | | (d, J = 1.52 Hz, 1H), 7.26 (t, J = 7.83 Hz, 1H), 6.88-7.01 (m, |
| | | 2H), 6.85 (s, 1H), 5.67 (d, J = 7.07 Hz, 1H), 4.75 (d, |
| | | J = 17.68 Hz, 1H), 4.21 (d, J = 17.68 Hz, 1H), 3.76 (s, 3H), |
| | | 1.72 (d, J = 7.07 Hz, 3H); LCMS Calculated: for |
| | | C24H21N5O2: 411, Measured: 412 [M + H]+. |
| 131 | 91 | 1H NMR (400 MHz, DMSO-d6) d 13.01 (s, 1H), 8.47 (t, |
| | | J = 4.9 Hz, 1H), 8.35 (s, 1H), 8.04 (s, 1H), 7.84-7.94 (m, |
| | | 2H), 7.80 (d, J = 2.0 Hz, 1H), 7.70 (m, J = 6.9, 1.8 Hz, |
| | | 1H), 7.57 (d, J = 8.0 Hz, 1H), 7.31-7.42 (m, 2H), 7.08 |
| | | (dd, J = 8.5, 6.2 Hz, 1H), 7.02 (d, J = 6.8 Hz, 2H), 4.74 |
| | | (s, 2H), 4.46 (d, J = 4.8 Hz, 2H), 2.35 (s, 6H), 1.66-1.98 |
| | | (m, 8H); LCMS: Calculated: for C32H32N4O2: 504, |
| | | Measured: 505 [M + H]+. |
| 132 | 51 | 1H NMR (400 MHz, METHANOL-d₄) δ 8.13-8.26 (m, 1H), |
| | | 8.07 (d, J = 1.52 Hz, 1H), 7.96 (s, 1H), 7.26 (t, J = 8.08 Hz, |
| | | 1H), 6.89-6.99 (m, 1H), 6.77-6.89 (m, 1H), 5.61-5.73 (m, |
| | | 1H), 4.86 (d, J = 18.19 Hz, 1H), 4.25 (d, J = 18.19 Hz, 1H), |
| | | 3.75 (s, 3H), 2.79 (s, 6H), 1.75 (d, J = 7.07 Hz, 3H); LCMS |
| | | Calculated: for C27H26N4O2: 438, Measured: 439 |
| | | [M + H]+. |
| 133 | 36 | 1H NMR (400 MHz, DMSO-d6) d 8.90 (brs, 2H), 8.33 (s, |
| | | 2H), 8.08-8.15 (m, 4H), 7.26 (t, J = 7.9 Hz, 1H), 6.82- |
| | | 6.96 (m, 3H), 5.37 (dd, J = 9.1, 5.3 Hz, 1H), 4.84 (d, J = |
| | | 17.9 Hz, 1H), 4.58 (d, J = 17.8 Hz, 1H), 3.92-4.10 (m, |
| | | 2H), 3.73 (s, 3H); LCMS: Calculated: for C25H22N4O3: |
| | | 426, Measured: 427 [M + H]+. |
| 134 | 92 | 1H NMR (400 MHz, DMSO-d6) d 12.98-13.04 (m, 1H), |
| | | 8.94 (t, J = 5.4 Hz, 1H), 8.40 (s, 1H), 8.29-8.37 (m, 2H), |
| | | 8.01-8.06 (m, 1H), 7.80-7.97 (m, 3H), 7.71-7.78 (m, |
| | | 1H), 7.54-7.67 (m, 1H), 7.42 (d, J = 7.1 Hz, 2H), 7.19 |
| | | (d, J = 4.9 Hz, 1H), 4.76 (s, 2H), 4.47 (d, J = 5.5 Hz, 2H), |
| | | 2.34 (s, 3H), 1.93 (m, J = 32.1, 12.2, 6.6 Hz, 6H), 1.79 |
| | | (dd, J = 11.3, 5.5 Hz, 2H); LCMS: Calculated: for |
| | | C30H29N5O2: 491, Measured: 492 [M + H]+. |
| 135 | | ¹H NMR (400 MHz, METHANOL-d₄) δ 8.07-8.21 (m, 2H), |
| | | 7.98 (s, 1H), 7.84 (dd, J = 1.52, 7.58 Hz, 1H), 7.50-7.56 |
| | | (m, 1H), 7.16 (t, J = 7.83 Hz, 1H), 6.76-6.80 (m, 1H), 6.68- |
| | | 6.74 (m, 2H), 4.75 (s, 2H), 4.39 (s, 2H); LCMS: |
| | | Calculated: for C18H15N3O2: 305, Measured: 306 |
| | | [M + H]+. |
| 136 | | ¹H NMR (400 MHz, METHANOL-d₄) δ 8.10-8.32 (m, 2H), |
| | | 7.96 (s, 1H), 7.81 (dd, J = 1.26, 7.83 Hz, 1H), 7.49 (d, |
| | | J = 7.58 Hz, 1H), 7.34-7.42 (m, 1H), 7.20 (d, J = 7.58 Hz, |
| | | 1H), 6.64-7.09 (m, 2H), 4.81 (s, 2H), 4.34-4.40 (m, 2H), |
| | | 2.24 (s, 3H); LCMS: Calculated: for C20H17N3O3: 347, |
| | | Measured: 348 [M + H]+. |
| 137 | | ¹H NMR (400 MHz, METHANOL-d₄) δ 8.23-8.64 (m, 1H), |
| | | 7.94-8.02 (m, 1H), 7.83 (d, J = 7.58 Hz, 1H), 7.48-7.68 (m, |
| | | 1H), 7.51 (d, J = 7.58 Hz, 1H), 7.34-7.43 (m, 1H), 7.21 (d, |
| | | J = 7.58 Hz, 1H), 6.93-7.10 (m, 2H), 4.82 (s, 2H), 4.39 (s, |
| | | 2H), 1.81-1.92 (m, 1H), 0.98-1.11 (m, 4H); LCMS: |
| | | Calculated: for C22H19N3O3: 373, Measured: 374 |
| | | [M + H]+. |
| 138 | | ¹H NMR (400 MHz, METHANOL-d₄) δ 8.10-8.29 (m, 1H), |
| | | 7.98 (s, 1H), 7.80-7.86 (m, 1H), 7.52 (d, J = 8.08 Hz, 1H), |
| | | 7.40 (d, J = 8.08 Hz, 1H), 7.24 (d, J = 7.58 Hz, 1H), 7.03- |
| | | 7.14 (m, 2H), 4.86 (s, 2H), 4.84 (s, 2H), 4.40 (s, 2H), 2.13 |
| | | (s, 3H); LCMS: Calculated: for C22H19N3O5: 405, |
| | | Measured: 406 [M + H]+. |
| 139 | 52 | 1H NMR (400 MHz, METHANOL-d₄) δ 8.09-8.27 (m, 1H), |
| | | 7.97 (d, J = 1.52 Hz, 1H), 7.81 (d, J = 1.52 Hz, 1H), 7.28 |
| | | (dt, J = 6.57, 7.83 Hz, 1H), 6.87-7.01 (m, 2H), 6.77-6.87 |
| | | (m, 1H), 5.66 (d, J = 7.07 Hz, 1H), 4.59 (d, J = 18.19 Hz, |
| | | 1H), 4.13 (d, J = 18.19 Hz, 1H), 3.76 (s, 3H), 1.63-1.74 (m, |
| | | 3H); LCMS Calculated: for C26H23N3O3: 425, |
| | | Measured: 426 [M + H]+. |
| 140 | 53 | 1H NMR (400 MHz, METHANOL-d₄) δ 8.20 (br d, J = 1.52 |
| | | Hz, 1H), 7.97 (d, J = 1.01 Hz, 1H), 7.82 (d, J = 1.52 Hz, |
| | | 1H), 7.37 (t, J = 7.83 Hz, 1H), 7.19-7.30 (m, 1H), 7.00-7.11 |
| | | (m, 1H), 6.88-7.00 (m, 2H), 6.83 (dd, J = 2.02, 8.08 Hz, |
| | | 1H), 5.65 (d, J = 7.07 Hz, 1H), 4.59 (d, J = 17.68 Hz, 1H), |
| | | 4.09 (d, J = 17.68 Hz, 1H), 3.81 (s, 3H), 3.75 (s, 3H), 1.69 |
| | | (d, J = 7.07 Hz, 3H); LCMS Calculated: for C27H25N3O3: |
| | | 439, Measured: 440 [M + H]+. |
| 141 | 54 | 1H NMR (400 MHz, METHANOL-d₄) δ 8.81 (d, J = 2.02 |
| | | Hz, 1H), 8.60-8.68 (m, 1H), 8.07-8.21 (m, 4H), 8.00 (d, |
| | | J = 1.52 Hz, 1H), 7.26 (t, J = 7.83 Hz, 1H), 6.89-7.01 (m, |
| | | 2H), 6.84 (dd, J = 2.02, 8.08 Hz, 1H), 5.58-5.75 (m, 1H), |
| | | 4.73 (d, J = 17.68 Hz, 1H), 4.22 (d, J = 17.68 Hz, 1H), 3.75 |
| | | (s, 3H), 1.59-1.81 (m, 9H); LCMS Calculated: for |
| | | C28H28N4O3: 468, Measured: 469 [M + H]+. |
| 142 | 32 | 1H NMR (400 MHz, DMSO-d6) d 8.87 (d, J = 1.6 Hz, |
| | | 1H), 8.61-8.68 (m, 1H), 8.29 (s, 1H), 8.10 (d, J = 8.1 |
| | | Hz, 1H), 7.94-8.01 (m, 2H), 7.51-7.65 (m, 2H), 7.23 (t, |
| | | J = 7.9 Hz, 1H), 6.86-6.93 (m, 2H), 6.79-6.86 (m, 1H), |
| | | 5.33 (dd, J = 8.9, 5.2 Hz, 1H), 5.05 (s, 1H), 4.74 (d, J = |
| | | 17.6 Hz, 1H), 4.45 (d, J = 17.6 Hz, 1H), 4.03 (dd, J = |
| | | 11.6, 9.1 Hz, 1H), 3.92 (dd, J = 11.6, 5.3 Hz, 1H), 3.71 (s, |
| | | 3H); LCMS: Calculated: for C25H22N4O3: 426, |
| | | Measured: 427 [M + H]+. |
| 143 | 33 | 1H NMR (300 MHz, DMSO-d6) d 9.63 (s, 1H), 8.27 (s, |
| | | 2H), 7.92 (d, J = 1.6 Hz, 1H), 7.85 (d, J = 1.6 Hz, 1H), |
| | | 7.19-7.35 (m, 2H), 6.93-7.06 (m, 2H), 6.77-6.93 (m, |
| | | 4H), 5.35 (dd, J = 8.7, 5.4 Hz, 1H), 4.68 (d, J = 17.6 Hz, |
| | | 1H), 4.34 (d, J = 17.6 Hz, 1H), 3.89-4.10 (m, 2H), 3.72 |
| | | (s, 3H); LCMS: Calculated: for C26H23N3O4: 441, |
| | | Measured: 442 [M + H]+. |
| 144 | 62 | 1H NMR (400 MHz, METHANOL-d₄) δ 8.03-8.20 (m, 1H), |
| | | 7.91 (d, J = 1.01 Hz, 1H), 7.81 (s, 1H), 7.29 (s, 1H), 6.96 |
| | | (br d, J = 18.69 Hz, 1H), 6.82-6.91 (m, 1H), 5.63-5.73 (m, |
| | | 1H), 4.60 (s, 1H), 4.18 (s, 1H), 3.49-3.71 (m, 1H), 3.30 |
| | | (m, 1H), 2.38-2.60 (m, 1H), 2.09-2.26 (m, 1H), 1.74 (d, |
| | | J = 7.58 Hz, 2H); LCMS Calculated: for C24H26N4O2: |
| | | 402, Measured: 403 [M + H]+. |
| 145 | 55 | 1H NMR (400 MHz, METHANOL-d₄) δ 8.83-8.99 (m, 1H), |
| | | 8.19-8.41 (m, 1H), 8.06-8.13 (m, 1H), 7.84-8.06 (m, 2H), |
| | | 7.41-7.63 (m, 2H), 7.22-7.31 (m, 1H), 6.88-6.99 (m, 3H), |
| | | 6.78-6.88 (m, 1H), 5.58-5.72 (m, 1H), 4.66-4.76 (m, 1H), |
| | | 4.14-4.24 (m, 1H), 3.76 (s, 3H), 1.71 (d, J = 7.07 Hz, 3H); |
| | | LCMS Calculated: for C26H21F3N4O2: 478, Measured: |
| | | 479 [M + H]+. |
| 146 | 59 | 1H NMR (400 MHz, DMSO-d6) d 8.92 (d, J = 11.4 Hz, |
| | | 1H), 8.46 (dd, J = 49.3, 11.9 Hz, 1H), 8.19 (s, 2H), 7.82 |
| | | (dd, J = 7.7, 1.9 Hz, 2H), 7.28 (tt, J = 8.7, 1.1 Hz, 1H), |
| | | 6.82-6.98 (m, 3H), 5.53 (p, J = 7.0 Hz, 1H), 4.55 (dd, |
| | | J = 33.9, 17.3 Hz, 1H), 4.11 (dd, J = 52.2, 17.4 Hz, 1H), |
| | | 3.72 (d, J = 2.1 Hz, 3H), 3.33 (q, J = 10.8, 9.8 Hz, 2H), |
| | | 3.00 (ddq, J = 59.3, 35.8, 11.6 Hz, 3H), 1.67-1.98 (m, |
| | | 4H), 1.62 (d, J = 7.1 Hz, 3H); 19F NMR (376 MHz, |
| | | DMSO-d6) d −74.54; LCMS: Calculated: C25H28N4O2: |
| | | 416, Measured: 417 [M + H]+.. |
| 147 | 58 | 1H NMR (400 MHz, DMSO-d6) d 8.19 (brs, 2H), 7.72 (d, |
| | | J = 23.8 Hz, 2H), 7.28 (t, J = 7.9 Hz, 1H), 6.85-6.93 (m, |
| | | 3H), 5.52 (q, J = 7.3 Hz, 1H), 4.56 (d, J = 17.6 Hz, 1H), |
| | | 4.14 (d, J = 17.3 Hz, 1H), 3.74 (s, 3H), 3.03 (d, J = 11.1 |
| | | Hz, 2H), 2.55-2.88 (m, 3H), 1.67 (p, J = 11.4, 10.6 Hz, |
| | | 7H): LCMS: Calculated: C25H28N4O2: 416, Measured: |
| | | 417 [M + H]+. |
| 148 | 56 | 1H NMR (300 MHz, DMSO-d6) d 8.23 (s, 2H), 7.76 (dd, |
| | | J = 7.4, 1.6 Hz, 2H), 7.28 (t, J = 7.8 Hz, 1H), 6.85-6.93 |
| | | (m, 3H), 5.52 (q, J = 7.3 Hz, 1H), 4.59 (d, J = 17.7 Hz, |
| | | 1H), 4.17 (d, J = 17.6 Hz, 1H), 3.88-4.08 (m, 2H), 3.74 |
| | | (s, 3H), 3.36-3.55 (m, 2H), 2.78-2.97 (m, 1H), 1.57- |
| | | 1.99 (m, 7H). LCMS: Calculated: for C25H27N3O3: 417, |
| | | Measured: 418 [M + H]+ |
| 149 | 57 | 1H NMR (400 MHz, DMSO-d6) d 9.07 (brs, 1H), 8.89 |
| | | (brs, 1H), 8.22 (s, 2H), 7.84 (dd, J = 8.7, 1.5 Hz, 2H), |
| | | 7.25-7.34 (m, 1H), 6.85-6.96 (m, 3H), 5.54 (dd, J = 7.1, |
| | | 2.4 Hz, 1H), 4.60 (dd, J = 17.5, 11.3 Hz, 1H), 4.17 (t, J = |
| | | 16.8 Hz, 1H), 3.75 (d, J = 1.1 Hz, 3H), 3.56-3.71 (m, |
| | | 1H), 3.48 (q, J = 8.2 Hz, 2H), 3.26 (ddt, J = 20.3, 14.7, |
| | | 8.4 Hz, 2H), 2.30-2.48 (m, 1H), 2.07 (ddt, J = 27.6, 12.6, |
| | | 9.3 Hz, 1H), 1.65 (d, J = 7.2 Hz, 3H)./19F NMR (376 |
| | | MHz, DMSO-d6) d −74.14; LCMS: Calculated: for |
| | | C24H26N4O2: 402, Measured: 403 [M + H]+. |
| 150 | 98/99 | 1H NMR (300 MHz, DMSO-d6) d 8.21 (s, 2 H), 8.11 (s, 1 |
| | | H), 7.98 (d, J = 1.6 Hz, 1 H), 7.85 (dd, J = 8.0, 1.8 Hz, 1 |
| | | H), 7.51 (d, J = 8.0 Hz, 1 H), 7.19 (t, J = 7.9 Hz, 1 H), |
| | | 6.68-6.82 (m, 3 H), 5.10 (d, J = 16.4 Hz, 1 H), 4.51-4.77 |
| | | (m, 3 H), 3.68 (s, 3 H), 2.26-2.45 (m, 2 H), 1.76-1.87 (m, |
| | | 1 H), 1.52 (d, J = 5.7 Hz, 1 H). LCMS: Calculated: for |
| | | C24H22N4O4: 430, Measured: 431 [M + H]+. |
| 151 | 98/99 | 1H NMR (300 MHz, DMSO-d6) d 8.21 (s, 2 H), 7.85-8.07 |
| | | (m, 3 H), 7.43 (d, J = 8.1 Hz, 1 H), 7.23 (t, J = 8.2 Hz, 1 |
| | | H), 6.76-6.86 (m, 3 H), 4.83-4.95 (m, 2 H), 4.39-4.51 (m, |
| | | 2 H), 3.70 (s, 3 H), 2.09 (dt, J = 8.3, 4.3 Hz, 2 H), 1.75- |
| | | 1.97 (m, 2 H). LCMS: Calculated: for C24H22N4O4: 430, |
| | | Measured: 431 [M + H]+. |
| 152 | 34 | 1H NMR (300 MHz, DMSO-d6) d 13.00 (s, 1H), 8.74 (t, |
| | | J = 1.8 Hz, 1H), 8.64 (d, J = 2.6 Hz, 1H), 8.43 (s, 1H), 8.03- |
| | | 8.15 (m, 2H), 7.98 (q, J = 1.5 Hz, 2H), 7.21 (t, J = 7.8 |
| | | Hz, 1H), 6.76-6.93 (m, 3H), 5.31 (dd, J = 9.0, 5.4 Hz, |
| | | 1H), 5.03 (t, J = 5.5 Hz, 1H), 4.75 (d, J = 17.6 Hz, 1H), |
| | | 4.49 (d, J = 17.7 Hz, 1H), 4.02 (t, J = 13.2 Hz, 1H), 3.81- |
| | | 3.96 (m, 1H), 3.69 (s, 3H); 19F NMR (376 MHz, |
| | | DMSO) d −126.96; LCMS: Calculated: for |
| | | C25H21FN4O3: 444, Measured: 445 [M + H]+. |
| 153 | 35 | 1H NMR (300 MHz, DMSO-d6) d 9.63 (s, 1H), 8.21 (s, |
| | | 2H), 7.81 (dd, J = 12.3, 1.5 Hz, 2H), 7.37-7.47 (m, 2H), |
| | | 7.21 (t, J = 7.9 Hz, 1H), 6.76-6.90 (m, 5H), 5.31 (dd, J = |
| | | 8.8, 5.4 Hz, 1H), 4.67 (d, J = 17.5 Hz, 1H), 4.32 (d, J = |
| | | 17.6 Hz, 1H), 3.85-4.08 (m, 3H), 3.69 (s, 3H); LCMS: |
| | | Calculated: for C26H23N3O4: 441, Measured: 442 |
| | | [M + H]+. |
| 154 | 40 | 1H NMR (400 MHz, DMSO-d6) d 8.73 (d, J = 5.5 Hz, |
| | | 1H), 8.33 (s, 2H), 8.07 (dd, J = 7.2, 1.6 Hz, 2H), 7.79- |
| | | 7.91 (m, 2H), 7.26 (t, J = 7.9 Hz, 1H), 6.88-6.95 (m, 2H), |
| | | 6.85 (dd, J = 8.2, 2.5 Hz, 1H), 5.52 (q, J = 6.9 Hz, 1H), |
| | | 4.78 (d, J = 17.6 Hz, 1H), 4.22 (d, J = 17.6 Hz, 1H), 3.73 |
| | | (s, 3H), 1.64 (d, J = 7.1 Hz, 3H), 1.42 (s, 9H); LCMS: |
| | | Calculated: for C29H30N4O2: 466, Measured: 467 |
| | | [M + H]+ |
| 155 | 61 | 1H NMR (400 MHz, DMSO-d6) d 8.78 (brs, 2H), 8.22 (s, |
| | | 2H), 7.60 (d, J = 1.3 Hz, 1H), 7.36 (d, J = 1.4 Hz, 1H), |
| | | 7.24-7.32 (m, 1H), 6.83-6.94 (m, 3H), 5.51 (q, J = 7.3 |
| | | Hz, 1H), 4.58 (d, J = 17.8 Hz, 1H), 4.12 (d, J = 17.8 Hz, |
| | | 1H), 3.74 (s, 3H), 3.34 (td, J = 7.5, 3.3 Hz, 2H), 3.18- |
| | | 3.30 (m, 6H), 1.67 (d, J = 7.2 Hz, 3H). 19F NMR (376 |
| | | MHz, DMSO-d6) d −74.25. Calculated: for C24H27N5O2 |
| | | 417, Measured: 418. [M + H]+. |
| 156 | 60 | 1H NMR (300 MHz, DMSO-d6) d 12.94 (s, 1 H), 8.29 (s, |
| | | 1 H), 8.00 (s, 1 H), 7.88 (d, J = 1.6 Hz, 1 H), 7.81 (dd, J = |
| | | 8.0, 1.7 Hz, 1 H), 7.51 (d, J = 7.9 Hz, 1 H), 7.23 (td, J = |
| | | 7.3, 2.0 Hz, 1 H), 6.78-6.89 (m, 3 H), 5.31 (dd, J = 8.3, |
| | | 5.8 Hz, 1 H), 5.06 (t, J = 5.5 Hz, 1 H), 4.57 (d, J = 17.7 |
| | | Hz, 1 H), 4.26 (d, J = 17.6 Hz, 1 H), 3.93 (dq, J = 11.5, |
| | | 6.5, 5.7 Hz, 2 H), 3.69 (s, 3 H); LCMS: Calculated: for |
| | | C20H19N3O3: 349, Measured: 350 [M + H]+. |
| 158 | 37 | 1H NMR (300 MHz, DMSO-d6) d 12.99 (s, 1H), 9.26 (s, |
| | | 1H), 9.15 (s, 2H), 8.27 (s, 2H), 8.04 (dd, J = 13.3, 1.6 Hz, |
| | | 2H), 7.23 (t, J = 7.9 Hz, 1H), 6.79-6.96 (m, 3H), 5.34 |
| | | (dd, J = 8.9, 5.2 Hz, 1H), 5.05 (t, J = 5.5 Hz, 1H), 4.81 (d, |
| | | J = 17.6 Hz, 1H), 4.54 (d, J = 17.7 Hz, 1H), 4.05 (dt, J = |
| | | 15.2, 7.5 Hz, 1H), 3.92 (dt, J = 11.2, 5.4 Hz, 1H), 3.71 (d, |
| | | J = 2.0 Hz, 3H); LCMS: Calculated: for C24H21N5O3: |
| | | 427, Measured: 428 [M + H]+. |
| 159 | 41 | 1H NMR (300 MHz, DMSO-d6) d 8.88 (d, J = 2.0 Hz, |
| | | 1H), 8.80 (d, J = 2.1 Hz, 1H), 8.41 (t, J = 2.1 Hz, 1H), |
| | | 8.31 (s, 2H), 8.00-8.07 (m, 2H), 7.25 (t, J = 7.9 Hz, 1H), |
| | | 6.88-6.95 (m, 2H), 6.81-6.88 (m, 1H), 5.52 (q, J = 7.0 |
| | | Hz, 1H), 4.74 (d, J = 17.5 Hz, 1H), 4.17 (d, J = 17.5 Hz, |
| | | 1H), 3.72 (s, 3H), 1.62 (d, J = 7.1 Hz, 3H), 1.39 (s, 9H). |
| | | 19F NMR (282 MHz, DMSO-d6) d −74.63; LCMS: |
| | | Calculated: for C29H30N4O2: 466, Measured: 467 |
| | | [M + H]+. |
| 160 | 63 | 1H NMR (300 MHz, DMSO-d6) d 8.66 (d, J = 12.6 Hz, |
| | | 1H), 8.19 (s, 2H), 7.76-7.82 (m, 2H), 7.67 (s, 1H), 7.28 |
| | | (t, J = 7.8 Hz, 1H), 6.81-6.97 (m, 3H), 5.52 (q, J = 7.0 |
| | | Hz, 1H), 4.67 (d, J = 17.9 Hz, 1H), 4.34 (d, J = 17.8 Hz, |
| | | 1H), 3.73 (s, 3H), 3.26 (d, J = 12.8 Hz, 1H), 1.84 (dq, J = |
| | | 26.7, 13.4 Hz, 4H), 1.68 (d, J = 7.2 Hz, 3H), 1.49 (d, J = |
| | | 10.3 Hz, 6H), 1.40 (s, 6H). 19F NMR (282 MHz, DMSO- |
| | | d6) d −74.10; LCMS Calculated: for C29H36N4O2: 472, |
| | | Measured: 473 [M + H]+. |
| 161 | 39 | 1H NMR (400 MHz, DMSO-d6) d 8.73 (d, J = 4.8 Hz, |
| | | 1H), 8.30-8.50 (m, 3H), 8.16 (d, J = 8.0 Hz, 1H), 7.95- |
| | | 8.07 (m, 2H), 7.44 (dd, J = 7.5, 4.8 Hz, 1H), 7.27 (t, J = |
| | | 7.9 Hz, 1H), 6.82-6.95 (m, 3H), 5.40 (dd, J = 8.6, 5.5 |
| | | Hz, 1H), 4.96 (d, J = 18.7 Hz, 1H), 4.68 (d, J = 18.7 Hz, |
| | | 1H), 3.97-4.13 (m, 2H), 3.73 (s, 3H). 19F NMR (376 |
| | | MHz, DMSO-d6) d −74.84; LCMS: Calculated: for |
| | | C25H22N4O3: 426, Measured: 427 [M + H]+. |
| 162 | 38 | 1H NMR (400 MHz, DMSO-d6) d 9.31 (s, 1H), 8.98 (d, |
| | | J = 5.2 Hz, 1H), 8.52 (d, J = 1.6 Hz, 1H), 8.38 (s, 2H), 8.33 |
| | | (dd, J = 5.5, 1.3 Hz, 1H), 8.13 (d, J = 1.5 Hz, 1H), 7.27 (t, |
| | | J = 7.8 Hz, 1H), 6.82-6.94 (m, 3H), 5.41 (dd, J = 8.8, 5.5 |
| | | Hz, 1H), 5.05 (d, J = 19.0 Hz, 1H), 4.76 (d, J = 18.9 Hz, |
| | | 1H), 3.98-4.13 (m, 2H), 3.73 (s, 3H). 19F NMR (376 |
| | | MHz, DMSO-d6) d −74.42. LCMS: Calculated: for |
| | | C24H21N5O3: 427, Measured: 428 [M + H]+. |

### Biological Example 1

### hGRK2 LANCE Ultra In Vitro Assay

G-protein coupled receptor kinases (GR Kinases) desensitize activated G-protein coupled receptors (GPCRs), by phosphorylation of cytoplasmic loops or carboxyl-terminal tails of GPCRs. GRK2 is one of the 6 different GR kinases and is implicated in heart failure and diabetes.

The purpose of the LANCE Ultra assay (http:/www.perkinelmer.com/Resources/TechnicalResources/ApplicationSupportKnowledgebase/LANCE/lance.xhtml) is used to test inhibitors against GRK2 in its inactive state. This assay is sensitive and requires as low as 10 nM enzyme, in a total volume of 10 μL. In addition, the ATP concentration can be varied over a broad range, without interfering with the assay or changing the assay condition. This property makes it easy to characterize very potent ATP-competitive inhibitors by increasing ATP concentrations. Testing inhibitors routinely at both high and low ATP concentrations also enables identification of potential non-ATP competitive inhibitors.

Paroxetine was used as the reference compound in this assay. The IC₅₀ value determined by the LANCE Ultra assay was 8.3 μM, which is in good agreement with the literature value (THAL, D. M., et al. “Paroxetine is a direct inhibitor of G protein-coupled receptor kinase 2 and increases myocardial contractility”, ACS Chemical Biology, 2012, pp 1830, Vol. 7).

This assay measures IC₅₀ values of test compounds (inhibitors) by monitoring GRK2 enzymatic activity at varying inhibitor concentrations.

Test compounds were dissolved in DMSO at 1 mM and were 3-fold serial diluted. The compound DMSO solutions were then added (100 nL) into a plate well using an acoustic dispenser. To each well was then added 20 nM GRK2 (5 μL) in assay buffer (20 mM HEPES, pH 7.5, 10 mM MgCl₂, 0.001% Tween-20®). The plate was sealed and centrifuged at 1000 rpm for 1 min. The plate and wells containing a mixture of GRK2 and test compound were incubated at ambient temperature for 30 min (prior to initializing the enzymatic reaction).

Enzyme reactions were initiated by the addition of 4.9 μL Substrates/Eu-Ab mix to each well. For assays at low ATP concentration (1×Kₘ value), the Substrate/Eu-Ab mix contains 60 μM ATP, 400 nM ULight-peptide (LANCE® Ultra ULight™-DNA Topoisomerase 2-alpha (Thr1342) Peptide), and 8 nM Eu-Ab (LANCE® Ultra Europium-anti-phospho-DNA Topoisomerase 2-alpha (Thr1342)) in the assay buffer. For assays at high ATP concentration (20×Kₘ value), the Substrate/Eu-Ab mix contains 1.2 mM ATP, 400 nM ULight-peptide, and 8 nM Eu-Ab in the assay buffer. Final concentrations of reagents in the assays were as follows: 20 mM HEPES, pH 7.5; 10 mM MgCl₂; 0.001% Tween-20® (w/v); 30 or 600 μM ATP; 200 nM ULight-peptide; 4 nM Eu-Ab; 10 nM GRK2; and 1% DMSO.

The plates were sealed and centrifuged at 1000 rpm for 1 min. For reactions at low ATP concentration (30 μM), reaction mixtures were incubated at ambient temperature for 120 min. For reactions at high ATP concentration (600 μM), reaction mixtures were incubated at ambient temperature for 60 min.

The enzyme reactions were quenched by addition of 10 μL of 12 mM EDTA in 1× LANCE detection solution to each well. The plates were then incubated at ambient temperature for 30 min. Time-resolved fluorescence signal of reactions were read on an EnVision or PHERAstar plate reader with the following parameters: Excitation wavelength=337 nm; emission wavelength (donor)=620 nm; emission wavelength (acceptor)=665 nm.

To calculate IC₅₀ values, compounds were serially diluted 3-fold and tested in 11-point dose responses. The raw HTRF data were converted to % active as follows:

% active=(sample−NC)/(PC−NC)*100

where NC is the mean of negative control (reactions without GRK2), and PC is the mean of positive control (reactions with GRK2 but without inhibitor). IC₅₀ values were determined from a 4-parameter fit, using the following equation:

Y=Bottom+(Top−Bottom)/(1+10^{((Log IC50-X)*Hill slope)})

where X=log₁₀ of the compound concentration.

### Biological Example 2

### GRK2 Transcreener® Assay

Test compounds were dissolved in 100% DMSO and then added into a 384-well Corning 3676 plate using an acoustic dispenser. Positive and negative control wells received an equal volume of DMSO. The final DMSO concentration in the assay is 1%.

15 μM ATP (6.5 uL) in assay buffer (10 mM HEPES, pH 7.5, 2 mM DTT, 5 mM MgCl₂, 0.005% Brij™-35) was added to each well, followed by the addition of 1.5 μL of 1 mM peptide substrate (amino acid sequence: MEFTEAESNMNDLVSEYQ). The plate was placed in centrifuge equipped with a spin-bucket rotor and spun for 1 min at 1000 rpm.

GRK2 enzymatic reactions were initiated with the addition of 2 μL/well of 50 nM GRK2 in assay buffer. Plates were centrifuged for 1 min at 1000 rpm. For negative control wells, the order of reagent addition was reversed: 10 μL/well ADP detection mix (see below) was added first, followed by the addition of 2 μL/well of the GRK2 solution. Reaction mixtures were incubated at ambient temperature for 2 hours. Final concentrations of reagents in the assays were as follows:

10 mM HEPES, pH 7.5

2 mM DTT

5 mM MgCl₂

0.005% Brij™-35 (w/v)

10 μM ATP

150 μM MEFTEAESNMNDLVSEYQ peptide

10 nM GRK2

1% DMSO

Following incubation, the reactions were quenched with 10 μL/well of the Transcreener ADP detection mix. The detection mix contains 4 nM Alexa633 tracer, 11.8 μg/mL anti-ADP antibody and 1× “stop & detect” buffer (BellBrook Labs, catalog number 3010-10K). The plates were then centrifuged for 1 min at 1000 rpm.

Fluorescence polarization values of the reaction mixtures were read on a Safire plate reader after a 60 min incubation at ambient temperature. Excitation wavelength=590 nm; emission wavelength=650 nm.

To calculate IC₅₀ values, compounds were serially diluted 2-fold and tested in 11-point dose responses. The fluorescence polarization data were converted to % activity as follows:

% activity=(sample−NC)/(PC−NC)*100

where NC is the mean of negative control (ADP detection mix added prior to GRK2 addition), and PC is the mean of positive control (GRK2 reaction without inhibitor). IC₅₀ values are determined from a 4-parameter fit, using the following equation:

Y=Bottom+(Top−Bottom)/(1+10^{((Log IC50-X)*Hill slope)})

where X=log₁₀ of the compound concentration.

Representative compounds of the present invention were tested according to the procedure described in Biological Example 1 and Biological Example 2, above, with results as listed in Table 4 below. Results are reported as IC₅₀ values; with multiple measurements listed individually. Variability for the functional assay was typically within 2-fold.

TABLE 4
Biological Activity, Compounds of Formula (I)

| | Biological Example 1 | Biological Example 2 |
|---|---|---|
| | GRK2 Lancer | GRK2 Transcreener |
| ID No. | IC₅₀ (μM) | IC₅₀ (μM) |
| 1 | | <0.005; 0.005 |
| 2 | | 0.009 |
| 3 | | 0.885 |
| 4 | | 0.080 |
| 6 | | 0.01; 0.01 |
| 7 | | 0.005 |
| 8 | | 0.009 |
| 9 | | 0.291 |
| 10 | | 0.719 |
| 11 | | 0.383 |
| 12 | | 1.615 |
| 13 | <0.005 | <0.005 |
| 14 | | <0.005 |
| 15 | 0.005 | <0.005 |
| 16 | | 0.017 |
| 17 | | 0.048 |
| 18 | | 0.139 |
| 19 | <0.005 | <0.005 |
| 20 | | 0.020 |
| 21 | | 0.054 |
| 22 | | 1.202 |
| 23 | | >50 |
| 24 | | 1.458 |
| 25 | | 0.546 |
| 26 | >20 | |
| 27 | 0.324 | |
| 28 | 0.091 | |
| 29 | 0.006 | |
| 30 | <0.005 | |
| 31 | 5.381 | |
| 32 | 0.261 | |
| 33 | 0.098 | |
| 34 | 0.006 | |
| 35 | 0.005 | |
| 36 | 0.241 | |
| 37 | >20 | |
| 38 | 0.006 | |
| 39 | 0.196 | |
| 40 | 0.115 | |
| 41 | 0.034 | |
| 42 | 0.006 | |
| 43 | 0.088 | |
| 44 | 0.382 | |
| 45 | 1.619 | |
| 46 | 0.007 | |
| 47 | ~10 | |
| 48 | 0.010 | |
| 49 | <0.005 | |
| 50 | 0.334 | |
| 51 | 0.526 | |
| 52 | 0.742 | |
| 53 | 0.008 | |
| 54 | 0.292 | |
| 55 | 0.011 | |
| 56 | <0.005 | |
| 57 | 2.560 | |
| 59 | 0.009 | |
| 60 | 0.017 | |
| 61 | 0.012 | |
| 62 | 0.007 | |
| 63 | 0.008 | |
| 64 | 0.047 | |
| 65 | 0.039 | |
| 66 | 0.023 | |
| 67 | 0.969 | |
| 68 | 0.135 | |
| 69 | 0.006 | |
| 70 | 0.237 | |
| 71 | 0.815 | |
| 72 | 0.130 | |
| 73 | 1.388 | |
| 74 | 4.205 | |
| 75 | 0.007 | |
| 76 | >20 | |
| 77 | >20 | |
| 78 | 0.060 | |
| 79 | 0.030 | |
| 80 | 0.044 | |
| 81 | 0.011 | |
| 82 | 0.007 | |
| 83 | 0.024 | |
| 84 | 0.167 | |
| 85 | 0.052 | |
| 86 | 0.037 | |
| 87 | <0.005 | |
| 88 | 0.029 | |
| 89 | 0.144 | |
| 90 | 0.137 | |
| 91 | 0.116 | |
| 92 | 5.010 | |
| 93 | 0.020 | |
| 94 | 0.652 | |
| 96 | 0.012 | |
| 97 | 0.012 | |
| 98 | 0.106 | |
| 99 | 0.359 | |
| 100 | 6.236 | |
| 102 | 0.006 | |
| 103 | 0.033 | |
| 104 | >11 | |
| 105 | 0.071 | |
| 106 | 0.419 | |
| 107 | 0.005 | |
| 108 | 0.004 | |
| 109 | 0.006 | |
| 110 | 0.307 | |
| 111 | 0.007 | |
| 112 | 0.306 | |
| 113 | 0.003 | |
| 114 | 0.006 | |
| 115 | 0.177 | |
| 117 | 0.404 | |
| 118 | <0.005 | |
| 119 | 0.113 | |
| 120 | 0.009 | |
| 121 | 0.010 | |
| 122 | 1.184 | |
| 123 | 0.015 | |
| 124 | 0.005 | |
| 125 | 0.022 | |
| 126 | 0.024 | |
| 127 | 0.047 | |
| 128 | <0.005 | |
| 129 | 0.011 | |
| 130 | <0.005 | |
| 131 | <0.005 | |
| 132 | 0.005 | |
| 133 | 0.021 | |
| 134 | 0.003 | |
| 135 | 0.005 | |
| 136 | 0.014 | |
| 137 | 0.034 | |
| 138 | 0.002 | |
| 139 | 0.033 | |
| 140 | 0.265 | |
| 141 | 0.020 | |
| 142 | 0.008 | |
| 143 | 0.005 | |
| 144 | 0.007 | |
| 145 | 0.285 | |
| 150 | 0.013 | |
| 151 | 0.011 | |
| 152 | 0.017 | |
| 153 | 0.019 | |
| 154 | 0.932 | |
| 155 | 0.007 | |
| 156 | 0.011 | |
| 158 | 0.027 | |
| 164 | | 0.305 |
| 165 | | >50 |
| 166 | 0.005 | |
| 167 | >20 | |
| 168 | 0.146 | |
| 169 | 0.542 | |
| 170 | 0.006 | |
| 171 | 0.104 | |
| 172 | 0.148 | |
| 173 | 0.092 | |
| 174 | 0.003 | |
| 175 | 0.046 | |

### Biological Example 3—Prophetic Example

### GLP-1 Mediated Beta-Arrestin Recruitment Assay

PathHunter® eXpress GLP1R CHO-K1 β-Arrestin cells are plated at 6000/well in a 384-well PDL white and opaque plate in F12 medium with 10% FBS, 0.3 mg/ml hygromycin, and 0.8 mg/ml G418. The plate is maintained in a humidified incubator at 37° C. and 5% CO₂ for 2 days before the experiment. On the day of the experiment, the cells are washed once with the Assay Buffer (HBSS with calcium and magnesium, 20 mM HEPES, and 0.1% fatty-acid free BSA). Test compound or vehicle (DMSO) is added to the cells at the indicated concentrations, 10 min prior to the addition of GLP-1. The final DMSO concentration is 0.1%. After 90 min incubation at 37° C., the detection reagent is added the cells, followed by 60 min incubation at the room temperature. The plate is read on MicroBeta LumiJet (PerkinElmer, Waltham, Mass.).

### Formulation Example 1

### Solid, Oral Dosage Form—Prophetic Example

As a specific embodiment of an oral composition, 100 mg of the Compound #13 (prepared as in Example 2) is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

We claim:

1. A compound of formula (I-P) 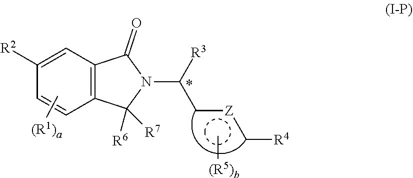
wherein
a is an integer from 0 to 3;
R¹ is selected from the group consisting of halogen, hydroxy, C₁₋₄alkyl, fluorinated C₁₋₂alkyl, C₁₋₄alkoxy, fluorinated C₁₋₂alkoxy, cyano, phenyl, pyridinyl, pyrimidinyl, pyrrolidinyl, piperidinyl, piperazinyl, and tetrahydro-pyranyl;
wherein the phenyl, pyridinyl, pyrimidinyl, pyrrolidinyl, piperidinyl, piperazinyl, and tetrahydro-pyranyl is optionally substituted with one or more substituents independently selected from the group halogen, hydroxy, C₁₋₄alkyl, fluorinated C₁₋₄alkyl, hydroxy substituted C₁₋₄alkyl, C₁₋₄alkoxy, fluorinated C₁₋₄alkoxy and NR^{J}R^{K}; wherein R^{d} and R^{K} are each independently selected from the group consisting of hydrogen, methyl and ethyl;
provided that when R¹ is selected from the group consisting of phenyl, pyridinyl, pyrimidinyl, pyrrolidinyl, piperidinyl, piperazinyl, and tetrahydropyranyl, wherein the phenyl, pyridinyl, pyrimidinyl, pyrrolidinyl, piperidinyl, piperazinyl, and tetrahydropyranyl are optionally substituted, then a is 1 and the R¹ group is bound at the 6-position of the isoindolin-2-one;
R² is selected from the group consisting of 5 to 10 membered heteroaryl and 5 to 10 membered heterocycloalkyl;
wherein the 5 to 10 membered heteroaryl or 5 to 10 membered heterocycloalkyl is optionally substituted with one or more substituents independently selected from the group consisting of oxo, —NR^{A}R^{B} and —C(O)—NR^{A}R^{B}; wherein R^{A} and R^{B} are each independently selected from the group consisting of hydrogen and C₁₋₂alkyl;
R³ is selected from the group consisting of hydrogen, —C₁₋₄alkyl, —C₁₋₄alkoxy, —(C₁₋₂alkyl)-OH, —(C₁₋₂alkyl)-CO₂H, —(C₁₋₂alkyl)-O—(C₁₋₂alkyl), —(C₁₋₂alkyl)-O—(C₁₋₂alkyl)-OH, —(C₁₋₂alkyl)-O—(C₁₋₂alkyl)-CO₂H, —(C₁₋₂alkyl)-O-phenyl, —(C₁₋₂alkyl)-O—(C₁₋₂alkyl)-phenyl, —(C₁₋₂alkyl)-NR^{P}R^{Q}, —(C₁₋₂alkyl)-O—(C₁₋₂alkyl)-C(O)—NR^{P}R^{Q}, —CO₂H, —C(O)O—(C₁₋₂alkyl), —C(O)—NR^{P}R^{Q}, —C(O)-phenyl, C₃₋₆cycloalkyl, 1,2,3,5-tetrazol-4-yl and −(C₁₋₂alkyl)-1,2,3,5-tetrazol-4-yl;
wherein R^{P} and R^{Q} are each independently selected from the group consisting of hydrogen, methyl and ethyl; 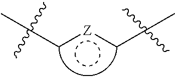
is selected from the group consisting of 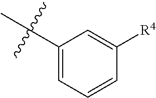
(wherein Z is CH), 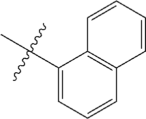 [SMILES: *c1cccc2ccccc12]
(wherein Z is CH and R⁴ is H), 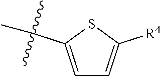
(wherein Z is S), 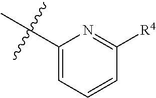
(wherein Z is N), 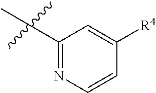
(wherein Z is CH), 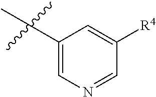
(wherein Z is CH) and 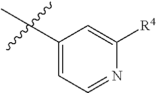
(wherein Z is CH);
R⁴ is selected from the group consisting of hydrogen, halogen, hydroxy, C₁₋₄alkyl, fluorinated C₁₋₂alkyl, —(C₁₋₂alkyl)-NR^{S}R^{T}, —(C₁₋₂alkyl)-CO₂H, —(C₁₋₂alkyl)-C(O)O—(C₁₋₄alkyl), C₁₋₄alkoxy, fluorinated C₁₋₂alkoxy, —O—(C₁₋₂alkyl)-CN, —O—(C₁₋₂alkyl)-CO₂H, —O—(C₁₋₂alkyl)-C(O)—O—(C₁₋₂alkyl), —O-phenyl, —O—(C₁₋₂alkyl)-phenyl, —O—(C₁₋₂alkyl)-(1,2,3,5-tetrazol-4-yl), —O-(oxetan-3-yl), —O-(tetrahydro-furan-3-yl), —O—C(O)—(C₁₋₂alkyl), —O—C(O)—C₃₋₆cycloalkyl, —O—C(O)—NR^{S}R^{T}, O—C(O)—(C₁₋₂alkyl)-O—C(O)—(C₁₋₂alkyl), —CO₂H, —C(O)—O—(C₁₋₄alkyl), —C(O)—NR^{S}R^{T}, —C(O)—NH-(phenyl), —C(O)—NH-(benzyl), —C(O)—NH-(pyridinyl), —C(O)—NH—(C₁₋₂alkyl)-(pyridinyl), —C(O)—NH-((1R,2S,4S)-bicyclo[2.2.1]heptan-2-yl), —C(O)—NH-((1R,2R,4R)-bicyclo[2.2.1]heptan-2-yl), —NH—SO₂—(C₁₋₂alkyl), —(C₁₋₂alkyl)-SO₂—NR^{S}R^{T}, and pyrazol-1-yl;
wherein the phenyl, benzyl or pyridinyl, whether alone or as part of a substituent group is optionally substituted with one to two substituents independently selected from the group consisting of halogen, C₁₋₄alkyl and C₁₋₄alkoxy; and wherein R^{S} and R^{T} are each independently selected from the group consisting of hydrogen and C₁₋₄alkyl;
b is an integer from 0 to 4;
each R⁵ is independently selected from the group consisting of halogen, C₁₋₄alkyl and C₁₋₄alkoxy;
R⁶ and R⁷ are the same and are selected from the group consisting of hydrogen, C₁₋₂alkyl and hydroxy substituted C₁₋₂alkyl;
alternatively, R⁶ is hydrogen and R⁷ is selected from the group consisting of —(C₁₋₂alkyl)-CN, —(C₁₋₂alkyl)-OH, —(C₁₋₂alkyl)-CO₂H, —(C₁₋₂alkyl)-(1,2,3,5-tetrazol-4-yl) and −(C₁₋₂alkyl)-(isoindolin-2-yl-1,3-dione);
alternatively, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3,3-diyl, tetrahydro-pyran-4′,4′-diyl, 2,3-dihydro-inden-1′1′-diyl, hexahydro-2H-cyclopenta[d]oxazol-4′,4′-diyl-2-one and hexahydro-2H-cyclopenta[d]oxazol-6′,6′-diyl-2-one;
wherein the cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3′,3′-diyl, tetrahydro-pyran-4′,4′-diyl or 2,3-dihydro-inden-1′1′-diyl is optionally substituted with one to two substituents independently selected from the group consisting of hydroxy, C₁₋₄alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen, methyl and ethyl;
or stereoisomer or pharmaceutically acceptable salt thereof.

2. The compound of claim 1, wherein
a is an integer from 0 to 2;
R¹ is selected from the group consisting of halogen, C₁₋₄alkyl, fluorinated C₁₋₂alkyl, C₁₋₄alkoxy, fluorinated C₁₋₂alkoxy, phenyl, pyridinyl, pyrimidinyl, pyrrolidinyl, piperidinyl, piperazinyl, and tetrahydro-pyranyl;
wherein the phenyl, pyridinyl, pyrimidinyl, pyrrolidinyl, piperidinyl, piperazinyl, and tetrahydro-pyranyl is optionally substituted with one or more substituents independently selected from the group halogen, hydroxy, C₁₋₄alkyl, fluorinated C₁₋₄alkyl, hydroxy substituted C₁₋₄alkyl, C₁₋₄alkoxy, fluorinated C₁₋₄alkoxy and NR^{J}R^{K}; wherein R^{d} and R^{K} are each independently selected from the group consisting of hydrogen, methyl and ethyl;
provided that when R¹ is selected from the group consisting of phenyl, pyridinyl, pyrimidinyl, pyrrolidinyl, piperidinyl, piperazinyl, and tetrahydropyranyl, wherein the phenyl, pyridinyl, pyrimidinyl, pyrrolidinyl, piperidinyl, piperazinyl, or tetrahydropyranyl are optionally substituted, then a is 1 and the R¹ group is bound at the 6-position of the isoindolin-2-one;
R² is selected from the group consisting of 5 to 10 membered heteroaryl and 5 to 10 membered heterocycloalkyl;
wherein the 5 to 10 membered heteroaryl or 5 to 10 membered heterocycloalkyl is optionally substituted with one to two substituents independently selected from the group consisting of oxo, —NR^{A}R^{B} and −(O)—NR^{A}R^{B}; wherein R^{A} and R^{B} are each independently selected from the group consisting of hydrogen and C₁₋₂alkyl;
R³ is selected from the group consisting of hydrogen, —C₁₋₄alkyl, —C₁₋₄alkoxy, —(C₁₋₂alkyl)-OH, —(C₁₋₂alkyl)-CO₂H, —(C₁₋₂alkyl)-O—(C₁₋₂alkyl), —(C₁₋₂alkyl)-0-(C₁₋₂alkyl)-OH, —(C₁₋₂alkyl)-O—(C₁₋₂alkyl)-CO₂H, —(C₁₋₂alkyl)-O-phenyl, —(C₁₋₂alkyl)-O—(C₁₋₂alkyl)-phenyl, —(C₁₋₂alkyl)-NR^{P}R^{Q}, —(C₁₋₂alkyl)-O—(C₁₋₂alkyl)-C(O)—NR^{P}R^{Q}, —CO₂H, —C(O)O—(C₁₋₂alkyl), —C(O)—NR^{P}R^{Q}, —C(O)-phenyl, C₃₋₆cycloalkyl, 1,2,3,5-tetrazol-4-yl and −(C₁₋₂alkyl)-1,2,3,5-tetrazol-4-yl;
wherein R^{P} and R^{Q} are each independently selected from the group consisting of hydrogen, methyl and ethyl; 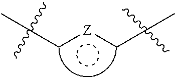
is selected from the group consisting of 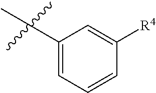
(wherein Z is CH), 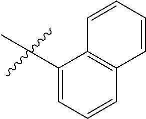 [SMILES: *c1cccc2ccccc12]
(wherein Z is CH and R⁴ is H), 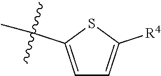
(wherein Z is S), 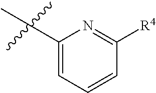
(wherein Z is N), 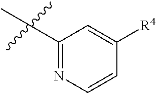
(wherein Z is CH), 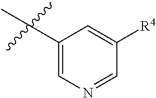
(wherein Z is CH) and 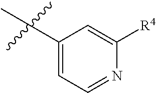
(wherein Z is CH);
R⁴ is selected from the group consisting of hydrogen, halogen, hydroxy, C₁₋₄alkyl, fluorinated C₁₋₂alkyl, —(C₁₋₂alkyl)-NR^{S}R^{T}, —(C₁₋₂alkyl)-CO₂H, —(C₁₋₂alkyl)-C(O)O—(C₁₋₄alkyl), C₁₋₄alkoxy, fluorinated C₁₋₂alkoxy, —O—(C₁₋₂alkyl)-CN, —O—(C₁₋₂alkyl)-CO₂H, —O—(C₁₋₂alkyl)-C(O)—O—(C₁₋₂alkyl), —O-phenyl, —O—(C₁₋₂alkyl)-phenyl, —O—(C₁₋₂alkyl)-(1,2,3,5-tetrazol-4-yl), —O-(oxetan-3-yl), —O-(tetrahydro-furan-3-yl), —O—C(O)—(C₁₋₂alkyl), —O—C(O)—C₃₋₆cycloalkyl, —O—C(O)—NR^{S}R^{T}, O—C(O)—(C₁₋₂alkyl)-O—C(O)—(C₁₋₂alkyl), —CO₂H, —C(O)—O—(C₁₋₄alkyl), —C(O)—NR^{S}R^{T}, —C(O)—NH-(phenyl), —C(O)—NH-(benzyl), —C(O)—NH-(pyridinyl), —C(O)—NH—(C₁₋₂alkyl)-(pyridinyl), —C(O)—NH-((1R,2S,4S)-bicyclo[2.2.1]heptan-2-yl), —C(O)—NH-((1R,2R,4R)-bicyclo[2.2.1]heptan-2-yl), —NH—SO₂—(C₁₋₂alkyl), —(C₁₋₂alkyl)-SO₂—NR^{S}R^{T}, and pyrazol-1-yl;
wherein the phenyl, benzyl or pyridinyl, whether alone or as part of a substituent group is optionally substituted with one to two substituents independently selected from the group consisting of halogen, C₁₋₄alkyl and C₁₋₄alkoxy; and wherein R^{S} and R^{T} are each independently selected from the group consisting of hydrogen and C₁₋₄alkyl;
b is an integer from 0 to 2;
each R⁵ is independently selected from the group consisting of halogen, C₁₋₄alkyl and C₁₋₄alkoxy;
R⁶ and R⁷ are the same and are selected from the group consisting of hydrogen, C₁₋₂alkyl and hydroxy substituted C₁₋₂alkyl;
alternatively, R⁶ is hydrogen and R⁷ is selected from the group consisting of —(C₁₋₂alkyl)-CN, —(C₁₋₂alkyl)-OH, —(C₁₋₂alkyl)-CO₂H, —(C₁₋₂alkyl)-(1,2,3,5-tetrazol-4-yl) and −(C₁₋₂alkyl)-(isoindolin-2-yl-1,3-dione);
alternatively, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3,3-diyl, tetrahydro-pyran-4′,4′-diyl, 2,3-dihydro-inden-1′1′-diyl, hexahydro-2H-cyclopenta[d]oxazol-4′,4′-diyl-2-one and hexahydro-2H-cyclopenta[d]oxazol-6′,6′-diyl-2-one;
wherein the cyclopent-1′1′-diyl, cyclohex-1′1′-diyl, cyclopent-3-en-1′1′-diyl, cyclohex-2-en-1′1′-diyl, cyclohex-3-en-1′1′-diyl, piperidin-4′,4′-diyl, tetrahydro-furan-3′,3′-diyl, tetrahydro-pyran-4′,4′-diyl or 2,3-dihydro-inden-1′1′-diyl is optionally substituted with one to two substituents independently selected from the group consisting of hydroxy, C₁₋₄alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen, methyl and ethyl;
or stereoisomer or pharmaceutically acceptable salt thereof.

3. The compound of claim 2, wherein
a is an integer from 0 to 1;
R¹ is selected from the group consisting of halogen, phenyl, pyridinyl, pyrimidinyl, pyrrolidinyl, piperidinyl, piperazinyl, and tetrahydro-pyranyl;
wherein the phenyl, pyridinyl, pyrimidinyl, pyrrolidinyl, piperidinyl, piperazinyl, and tetrahydro-pyranyl is optionally substituted with one to four substituents independently selected from the group halogen, hydroxy, C₁₋₄alkyl, fluorinated C₁₋₂alkyl, hydroxy substituted C₁₋₄alkyl, C₁₋₂alkoxy, and amino;
provided that when R¹ is selected from the group consisting of phenyl, pyridinyl, pyrimidinyl, pyrrolidinyl, piperidinyl, piperazinyl, and tetrahydro-pyranyl, wherein the phenyl, pyridinyl, pyrimidinyl, pyrrolidinyl, piperidinyl, piperazinyl, and tetrahydro-pyranyl is optionally substituted, then a is 1 and the R¹ group is bound at the 6-position of the isoindolin-2-one;
R² is selected from the group consisting of 5 to 6 membered heteroaryl, 9 to 10 membered heteroaryl, 5 to 6 membered heterocycloalkyl and 9 to 10 membered heterocycloalkyl;
wherein the of 5 to 6 membered heteroaryl, 9 to 10 membered heteroaryl, 5 to 6 membered heterocycloalkyl or 9 to 10 membered heterocycloalkyl is optionally substituted with a substituent selected from the group consisting of oxo, —NR^{A}R^{B} and —C(O)—NR^{A}R^{B}; wherein R^{A} and R^{B} are each independently selected from the group consisting of hydrogen and C₁₋₂alkyl;
R³ is selected from the group consisting of hydrogen, —C₁₋₂alkyl, —C₁₋₄alkoxy, —(C₁₋₂alkyl)-OH, —(C₁₋₂alkyl)-CO₂H, —(C₁₋₂alkyl)-O—(C₁₋₂alkyl), —(C₁₋₂alkyl)-O—(C₁₋₂alkyl)-OH, —(C₁₋₂alkyl)-O—(C₁₋₂alkyl)-CO₂H, —(C₁₋₂alkyl)-O—(C₁₋₂alkyl)-phenyl, —(C₁₋₂alkyl)-NR^{P}R^{Q}, —(C₁₋₂alkyl)-O—(C₁₋₂alkyl)-C(O)—NR^{P}R^{Q}, —CO₂H, —C(O)O—(C₁₋₂alkyl), —C(O)—NR^{P}R^{Q}, —C(O)-phenyl, C₃₋₆cycloalkyl, 1,2,3,5-tetrazol-4-yl and −(C₁₋₂alkyl)-1,2,3,5-tetrazol-4-yl;
wherein R^{P} and R^{Q} are each independently selected from the group consisting of hydrogen and methyl; 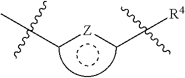
is selected from the group consisting of 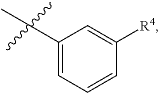
wherein Z is CH; 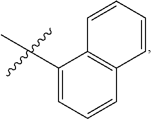
wherein Z is CH, R⁴ is H; 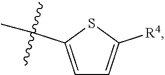
wherein Z is S; 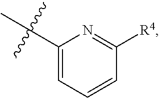
wherein Z is N; and 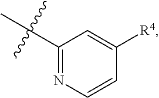
wherein Z is CH;
R⁴ is selected from the group consisting of hydrogen, halogen, hydroxy, —(C₁₋₂alkyl)-NR^{S}R^{T}, C₁₋₄alkoxy, —O—(C₁₋₂alkyl)-CN, —O—(C₁₋₂alkyl)-CO₂H, —O—(C₁₋₂alkyl)-C(O)—O—(C₁₋₂alkyl), —O-phenyl, —O—(C₁₋₂alkyl)-phenyl, —O—(C₁₋₂alkyl)-(1,2,3,5-tetrazol-4-yl), —O-(oxetan-3-yl), —O-(tetrahydro-furan-3-yl), —O—C(O)—(C₁₋₂alkyl), —O—C(O)—C₃₋₆cycloalkyl, —O—C(O)—NR^{S}R^{T}, —O—C(O)—(C₁₋₂alkyl)-O—C(O)—(C₁₋₂alkyl), —CO₂H, —C(O)—O—(C₁₋₄alkyl), —C(O)—NR^{S}R^{T}, —C(O)—NH-(phenyl), —C(O)—NH-(benzyl), —C(O)—NH—(C₁₋₂alkyl)-(pyridinyl), —C(O)—NH-((1R,2S,4S)-bicyclo[2.2.1]heptan-2-yl), —C(O)—NH-((1R,2R,4R)-bicyclo[2.2.1]heptan-2-yl), —NH—SO₂—(C₁₋₂alkyl) and pyrazol-1-yl;
wherein the phenyl, benzyl or pyridinyl, whether alone or as part of a substituent group is optionally substituted with one to two substituents independently selected from the group consisting of halogen and C₁₋₂alkyl;
and wherein R^{S} and R^{T} are each independently selected from the group consisting of hydrogen and C₁₋₄alkyl;
b is an integer from 0 to 1;
R⁵ is C₁₋₂alkoxy;
R⁶ and R⁷ are the same and are selected from the group consisting of hydrogen, C₁₋₂alkyl and hydroxy substituted C₁₋₂alkyl;
alternatively, R⁶ is hydrogen and R⁷ is selected from the group consisting of —(C₁₋₂alkyl)-CN, —(C₁₋₂alkyl)-OH, —(C₁₋₂alkyl)-CO₂H, —(C₁₋₂alkyl)-(1,2,3,5-tetrazol-4-yl) and −(C₁₋₂alkyl)-(isoindolin-2-yl-1,3-dione);
alternatively, R⁶ and R⁷ are taken together with the carbon atom to which they are bound to form a ring structure selected from the group consisting of cyclopent-1′1′-diyl, cyclohex-1′1′-diyl, cyclopent-3-en-1′1′-diyl, piperidin-4′,4′-diyl, tetrahydro-furan-3,3-diyl, tetrahydro-pyran-4′,4′-diyl, 2,3-dihydro-inden-1′1′-diyl and hexahydro-2H-cyclopenta[d]oxazol-4′,4′-diyl-2-one;
wherein the cyclopent-1′1′-diyl is optionally substituted with one to two substituents independently selected from the group consisting of hydroxy, C₁₋₂alkoxy, —CO₂H, —C(O)—NH₂, —NR^{X}R^{Y}, —NH—CO₂H and —NH—C(O)—O—(C₁₋₂alkyl); wherein R^{X} and R^{Y} are each independently selected from the group consisting of hydrogen and methyl;
or stereoisomer or pharmaceutically acceptable salt thereof.

4. The compound of claim 3, wherein
a is an integer from 0 to 1;
R¹ is selected from the group consisting of 5-fluoro, 7-fluoro, 6-(3-hydroxy-phenyl), 6-(4-hydroxy-phenyl), 6-(3-methoxy-phenyl), 6-(pyridin-2-yl), 6-(pyridin-3-yl), 6-(5-fluoro-pyridin-3-yl), 6-(5-t-butyl-pyridin-3-yl), 6-(5-methoxy-pyridin-3-yl), 6-(6-methoxy-pyridin-3-yl), 6-(6-(trifluoromethyl)-pyridin-3-yl), 6-(2-amino-pyridin-3-yl), 6-(6-(1-hydroxy-isopropyl)-pyridin-3-yl), 6-(pyridin-4-yl), 6-(2-t-butyl-pyridin-4-yl), 6-(4,6-dimethyl-pyridin-4-yl), 6-(pyrimidin-4-yl), 6-(pyrimidin-5-yl), 6-(pyrrolidin-3-yl), 6-(piperidin-3-yl), 6-(piperidin-4-yl), 6-(2,2,6,6-tetramethyl-piperidin-4-yl), 6-(piperazin-1-yl), 6-(4-methyl-piperazin-1-yl) and 6-(tetrahydro-pyran-4-yl);
R² is selected from the group consisting of pyrazol-4-yl, pyridin-3-yl, 6-(methyl-amino-carbonyl)-pyridin-3-yl, pyridin-4-yl, 6-(methyl-amino-carbonyl)-pyridin-4-yl, pyrimidin-2-yl, 2-amino-pyrimidin-4-yl, 2-(methyl-amino-carbonyl)-pyrimidin-4-yl, 1,2,3-triazol-4-yl, 2,4-dihydro-3H-1,2,4-triazol-4-yl-3-one, 1H-pyrrolo[2,3-b]pyridin-3-yl and 1H-pyrrolo[2,3-b]pyridin-4-yl;
R³ is selected from the group consisting of hydrogen, methyl, R-methyl, S-methyl, hydroxy-methyl-, R-(hydroxy-methyl-), S-(hydroxy-methyl-), S*-(hydroxy-methyl-), methoxy-methyl-, 2-hydroxy-ethoxy-methyl-, carboxy-methyl-, carboxy-methoxy-methyl-, amino-carbonyl-methoxy-methyl-, dimethylamino-methyl-, 2-carboxy-ethyl-, carboxy, R-carboxy, S-carboxy, methoxy-carbonyl-, S-(methoxy-carbonyl-), R-(methoxy-carbonyl-), R-cyclopropyl, benzyloxy-methyl-, 1,2,3,4-tetrazol-5-yl, 1,2,3,4-tetrazol-5-yl-methyl-, dimethylamino-carbonyl- and phenyl-carbonyl-; 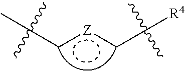
is selected from the group consisting of 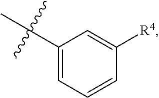
wherein Z is CH; 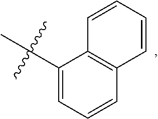
wherein Z is CH, R⁴ is H; 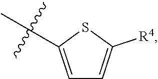
wherein Z is S; 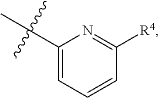
wherein Z is N; and 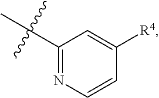
wherein Z is CH;
R⁴ is selected from the group consisting of hydrogen, fluoro, hydroxy, methyl, methoxy, ethoxy, isopropyloxy, phenyloxy, benzyloxy, oxetan-3-yl-oxy, tetrahydrofuran-3-yl-oxy-, carboxy, methoxy-carbonyl-, t-butoxy-carbonyl-, carboxy-methoxy-, methoxy-carbonyl-methoxy-, cyano-methoxy-, 1,2,3,5-tetrazol-4-yl-methoxy-, isopropyl-amino-carbonyl-, (3,4-difluoro-phenyl)-amino-carbonyl-, (2,6-dimethyl-benzyl)-amino-carbonyl-, methyl-carbonyl-oxy, methyl-carbonyl-oxy-methyl-carbonyl-oxy, amino-carbonyl-oxy-, cyclopropyl-carbonyl-oxy-, amino-methyl-, (4-methyl-pyridin-3-yl)-methyl-amino-carbonyl-, (1R,2S,4S)-bicyclo[2.2.1]hept-2-yl-amino-carbonyl-, (1R,2R,4R)-bicyclo[2.2.1]hept-2-yl-amino-carbonyl-, methyl-sulfonyl-amino- and pyrazol-1-yl;
b is an integer from 0 to 1;
R⁵ is 6-methoxy;
R⁶ and R⁷ are the same and are selected from the group consisting of hydrogen, methyl and 2-hydroxy-eth-1-yl;
alternatively, R⁶ is hydrogen and R⁷ is selected from the group consisting of 2-hydroxy-eth-1-yl, cyano-methyl-, carboxy-methyl-, S*-carboxy-methyl-, R*-carboxy-methyl-, (1,2,3,5-tetrazol-4-yl)-methyl- and (isoindolin-2-yl-1,3-dione)-ethyl-;
alternatively, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 2-carboxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 2-(carboxy-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4,4,-diyl, 2,3-dihydro-1H-inden-1′1′-diyl-4-ol and hexahydro-2H-cyclopenta[d]oxazol-4′,4′-diyl-2-one;
or pharmaceutically acceptable salt thereof.

5. The compound of claim 4, wherein
a is an integer from 0 to 1;
R¹ is selected from the group consisting of 5-fluoro, 7-fluoro, 6-(3-hydroxy-phenyl), 6-(4-hydroxy-phenyl), 6-(pyridin-3-yl), 6-(5-fluoro-pyridin-3-yl), 6-(5-methoxy-pyridin-3-yl), 6-(6-methoxy-pyridin-3-yl), 6-(2-amino-pyridin-3-yl), 6-(6-(1-hydroxy-isopropyl)-pyridin-3-yl), 6-(pyridin-4-yl), 6-(4,6-dimethyl-pyridin-4-yl), 6-(pyrimidin-5-yl), 6-(pyrrolidin-3-yl), 6-(piperazin-1-yl) and 6-(4-methyl-piperazin-1-yl);
R² is selected from the group consisting of pyrazol-4-yl, pyridin-3-yl, pyridin-4-yl, 2-amino-pyrimidin-4-yl and 1,2,3-triazol-4-yl;
R³ is selected from the group consisting of hydrogen, R-methyl, hydroxy-methyl-, S-(hydroxy-methyl-), S*-(hydroxy-methyl-), methoxy-methyl-, 2-hydroxy-ethoxy-methyl-, carboxy-methoxy-methyl-, amino-carbonyl-methoxy-methyl-, dimethylamino-methyl-, R-cyclopropyl, benzyloxy-methyl- and dimethylamino-carbonyl-; 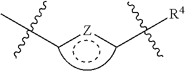
is selected from the group consisting of 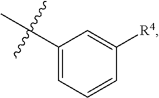
wherein Z is CH; 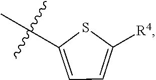
wherein Z is S; and 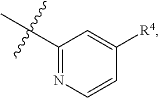
wherein Z is CH;
R⁴ is selected from the group consisting of hydrogen, fluoro, hydroxy, methoxy, ethoxy, isopropyloxy, phenyloxy, benzyloxy, t-butoxy-carbonyl-, carboxy-methoxy-, methoxy-carbonyl-methoxy-, cyano-methoxy-, 1,2,3,5-tetrazol-4-yl-methoxy-, isopropyl-amino-carbonyl-, (3,4-difluoro-phenyl)-amino-carbonyl-, (2,6-dimethyl-benzyl)-amino-carbonyl-, methyl-carbonyl-oxy, methyl-carbonyl-oxy-methyl-carbonyl-oxy, cyclopropyl-carbonyl-oxy-, (4-methyl-pyridin-3-yl)-methyl-amino-carbonyl-, (1R,2S,4S)-bicyclo[2.2.1]hept-2-yl-amino-carbonyl- and (1R,2R,4R)-bicyclo[2.2.1]hept-2-yl-amino-carbonyl-;
b is 0;
R⁶ and R⁷ are the same and are selected from the group consisting of hydrogen, methyl and 2-hydroxy-eth-1-yl;
alternatively, R⁶ is hydrogen and R⁷ is selected from the group consisting of 2-hydroxy-eth-1-yl, cyano-methyl-, carboxy-methyl-, S*-carboxy-methyl- and (1,2,3,5-tetrazol-4-yl)-methyl-;
alternatively, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4,4,-diyl and hexahydro-2H-cyclopenta[d]oxazol-4′,4′-diyl-2-one;
or pharmaceutically acceptable salt thereof.

6. The compound of claim 4, wherein
a is an integer from 0 to 1;
R¹ is selected from the group consisting of 5-fluoro, 7-fluoro, 6-(3-hydroxy-phenyl), 6-(4-hydroxy-phenyl), 6-(pyridin-3-yl), 6-(5-fluoro-pyridin-3-yl), 6-(5-methoxy-pyridin-3-yl), 6-(6-methoxy-pyridin-3-yl), 6-(2-amino-pyridin-3-yl), 6-(6-(1-hydroxy-isopropyl)-pyridin-3-yl), 6-(pyridin-4-yl), 6-(4,6-dimethyl-pyridin-4-yl), 6-(pyrimidin-5-yl), 6-(pyrrolidin-3-yl), 6-(piperazin-1-yl) and 6-(4-methyl-piperazin-1-yl);
R² is selected from the group consisting of pyrazol-4-yl, pyridin-3-yl, pyridin-4-yl, 2-amino-pyrimidin-4-yl and 1,2,3-triazol-4-yl;
R³ is selected from the group consisting of hydrogen, R-methyl, hydroxy-methyl-, S-(hydroxy-methyl-), S*-(hydroxy-methyl-), methoxy-methyl-, 2-hydroxy-ethoxy-methyl-, carboxy-methoxy-methyl-, amino-carbonyl-methoxy-methyl-, dimethylamino-methyl-, R-cyclopropyl and benzyloxy-methyl-; 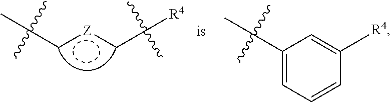
wherein Z is CH;
R⁴ is selected from the group consisting of fluoro, hydroxy, methoxy, ethoxy, isopropyloxy, phenyloxy, carboxy-methoxy-, methoxy-carbonyl-methoxy-, cyano-methoxy-, 1,2,3,5-tetrazol-4-yl-methoxy-, isopropyl-amino-carbonyl-, (3,4-difluoro-phenyl)-amino-carbonyl-, (2,6-dimethyl-benzyl)-amino-carbonyl-, methyl-carbonyl-oxy, methyl-carbonyl-oxy-methyl-carbonyl-oxy, cyclopropyl-carbonyl-oxy-, (4-methyl-pyridin-3-yl)-methyl-amino-carbonyl-, (1R,2S,4S)-bicyclo[2.2.1]hept-2-yl-amino-carbonyl- and (1R,2R,4R)-bicyclo[2.2.1]hept-2-yl-amino-carbonyl-;
b is 0;
R⁶ and R⁷ are the same and are selected from the group consisting of hydrogen, methyl and 2-hydroxy-eth-1-yl;
alternatively, R⁶ is hydrogen and R⁷ is selected from the group consisting of 2-hydroxy-eth-1-yl, cyano-methyl-, carboxy-methyl-, S*-carboxy-methyl- and (1,2,3,5-tetrazol-4-yl)-methyl-;
alternatively, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 2-amino-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, cyclohex-1′1′-diyl, 1′1′-diylpiperidin-4′,4′-diyl, tetrahydro-furan-3,3-diyl, tetrahydro-pyran-4′,4′-diyl and hexahydro-2H-cyclopenta[d]oxazol-4′,4′-diyl-2-one;
or pharmaceutically acceptable salt thereof.

7. The compound of claim 4, wherein
a is an integer from 0 to 1;
R¹ is selected from the group consisting of 5-fluoro, 7-fluoro, 6-(3-hydroxy-phenyl), 6-(pyridin-3-yl), 6-(5-fluoro-pyridin-3-yl), 6-(pyridin-4-yl), 6-(4,6-dimethyl-pyridin-4-yl), 6-(pyrimidin-5-yl), 6-(pyrrolidin-3-yl) and 6-(piperazin-1-yl);
R² is pyrazol-4-yl;
R³ is selected from the group consisting of hydrogen, R-methyl, hydroxy-methyl-, S-(hydroxy-methyl-), S*-(hydroxy-methyl-), methoxy-methyl-, 2-hydroxy-ethoxy-methyl- and amino-carbonyl-methoxy-methyl-; 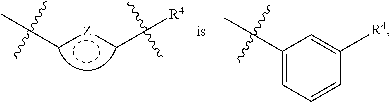
wherein Z is CH;
R⁴ is selected from the group consisting of hydroxy, methoxy, ethoxy, isopropyloxy, methoxy-carbonyl-methoxy-, cyano-methoxy-, isopropyl-amino-carbonyl-, (3,4-difluoro-phenyl)-amino-carbonyl-, (2,6-dimethyl-benzyl)-amino-carbonyl-, methyl-carbonyl-oxy-methyl-carbonyl-oxy, (4-methyl-pyridin-3-yl)-methyl-amino-carbonyl-, (1R,2S,4S)-bicyclo[2.2.1]hept-2-yl-amino-carbonyl- and (1R,2R,4R)-bicyclo[2.2.1]hept-2-yl-amino-carbonyl-;
b is 0;
R⁶ and R⁷ are the same and are selected from the group consisting of hydrogen and methyl;
alternatively, R⁶ is hydrogen and R⁷ is selected from the group consisting of 2-hydroxy-eth-1-yl and cyano-methyl-;
alternatively, R⁶ and R⁷ are taken together with the carbon atom to which they are bound to form a ring structure selected from the group consisting of cyclopent-1′1′-diyl, 3-hydroxy-cyclopent-1′1′-diyl, 2,3-dihydroxy-cyclopent-1′1′-diyl, 3,4-dihydroxy-cyclopent-1′1′-diyl, 3-carboxy-cyclopent-1′1′-diyl, 3-methoxy-cyclopent-1′1′-diyl, 3-(methyl-amino)-cyclopent-1′1′-diyl, 3-(methoxy-carbonyl-amino)-cyclopent-1′1′-diyl, 1-(amino-carbonyl)-cyclopent-1′1′-diyl, 3-(amino-carbonyl)-cyclopent-1′1′-diyl, cyclopent-3-en-1′1′-diyl, 1′1′-diylpiperidin-4′,4′-diyl, tetrahydro-furan-3,3-diyl and tetrahydro-pyran-4′,4′-diyl;
or pharmaceutically acceptable salt thereof.

8. The compound of claim 4, wherein
a is an integer from 0 to 1;
R¹ is selected from the group consisting of 5-fluoro, 7-fluoro, 6-(3-hydroxy-phenyl), 6-(pyridin-3-yl), 6-(4,6-dimethyl-pyridin-4-yl) and 6-(pyrimidin-5-yl);
R² is pyrazol-4-yl;
R³ is selected from the group consisting of hydrogen, R-methyl, hydroxy-methyl- and S*-(hydroxy-methyl-); 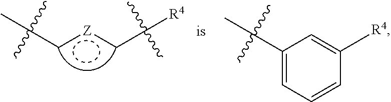
wherein Z is CH;
R⁴ is selected from the group consisting of hydroxy, methoxy, methoxy-carbonyl-methoxy-, cyano-methoxy-, isopropyl-amino-carbonyl-, (2,6-dimethyl-benzyl)-amino-carbonyl-, methyl-carbonyl-oxy-methyl-carbonyl-oxy, (4-methyl-pyridin-3-yl)-methyl-amino-carbonyl-, (1R,2S,4S)-bicyclo[2.2.1]hept-2-yl-amino-carbonyl- and (1R,2R,4R)-bicyclo[2.2.1]hept-2-yl-amino-carbonyl-;
b is 0;
R⁶ and R⁷ are the same and are selected from the group consisting of hydrogen and methyl;
alternatively, R⁶ is hydrogen and R⁷ is selected from the group consisting of 2-hydroxy-eth-1-yl and cyano-methyl-;
alternatively, R⁶ and R⁷ are taken together with the carbon atom to which they are bound to form a ring structure selected from the group consisting of cyclopent-1′1′-diyl, 3-carboxy-cyclopent-1′1′-diyl, 3-(methyl-amino)-cyclopent-1′1′-diyl, 3-(amino-carbonyl)-cyclopent-1′1′-diyl and 1′1′-diylpiperidin-4′,4′-diyl;
or pharmaceutically acceptable salt thereof.

9. A compound selected from the group consisting of
2-[(3-methoxyphenyl)methyl]-6-(1H-pyrazol-4-yl)isoindolin-1-one;
2-[(1R)-1-(3-methoxyphenyl)ethyl]-6-(1H-pyrazol-4-yl)isoindolin-1-one;
N-isopropyl-3-[[1-oxo-6-(1H-pyrazol-4-yl)isoindolin-2-yl]methyl]benzamide;
2-[(1R)-1-(3-ethoxyphenyl)ethyl]-6-(1H-pyrazol-4-yl)isoindolin-1-one;
7-fluoro-2-[(1R)-1-(3-methoxyphenyl)ethyl]-6-(1H-pyrazol-4-yl)isoindolin-1-one;
5-fluoro-2-[(3-methoxyphenyl)methyl]-6-(1H-pyrazol-4-yl)isoindolin-1-one;
2′-[(3-methoxyphenyl)methyl]-6′-(1H-pyrazol-4-yl)spiro[cyclopentane-1,3′-isoindoline]-1′-one;
2-[(1S*)-2-hydroxy-1-(3-methoxyphenyl)ethyl]-6-(1H-pyrazol-4-yl)isoindolin-1-one;
2-[(1R)-1-(3-methoxyphenyl)ethyl]-6-(1H-pyrazol-4-yl)-4-pyrimidin-5-yl-isoindolin-1-one;
2-[(1R)-1-(3-methoxyphenyl)ethyl]-4-piperazin-1-yl-6-(1H-pyrazol-4-yl)isoindolin-1-one;
and pharmaceutically acceptable salts thereof.

10. A pharmaceutical composition comprising a pharmaceutically acceptable carrier and a compound of claim 1.

11. A method of treating a disorder mediated by GRK2 activity, comprising administering to a subject in need thereof a therapeutically effective amount of the compound of claim 1.

12. The method of claim 11, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), end stage chronic kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m².

13. The method of claim 11, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, cardiac failure, cardiac hypertrophy, hypertension, angina, atherosclerosis, non-alcoholic steatohepatitis (NASH), non-alcoholic fatty liver disease (NAFLD), end stage chronic kidney disease, chronic kidney disease, acute renal failure, and a measured GFR equal or greater than 125 mL/min/1.73 m²

14. The method of claim 11, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, non-alcoholic steatohepatitis (NASH), non-alcoholic fatty liver disease (NAFLD), end stage chronic kidney disease, chronic kidney disease, acute renal failure, and a measured GFR equal or greater than 125 mL/min/1.73 m².
